(19) United States
(12) Patent Application Publication
(10) Pub. No.: US 2025/0152653 A1
(43) Pub. Date: May 15, 2025

(54) ASHWAGANDHA EXTRACTS AND FORMULATIONS THEREOF

(71) Applicant: ReBalance Health, Inc., Boulder, CO (US)

(72) Inventors: Justin HAI, Boulder, CO (US); Russell GLENN, San Diego, CA (US)

(21) Appl. No.: 18/730,195

(22) PCT Filed: Jan. 20, 2023
(86) PCT No.: PCT/US2023/061046

§ 371 Date: Jul. 18, 2024

Related U.S. Application Data
(60) Provisional application No. 63/301,965, filed on Jan. 21, 2022.

(51) Int. Cl. A61K 36/81; A61K 31/4045; A61K 31/685; A61K 33/06; A61K 36/068; A61K 47/18; A61K 47/46

(52) U.S. Cl. CPC A61K 36/81; A61K 31/4045; A61K 31/685; A61K 33/06; A61K 36/068; A61K 47/183; A61K 47/46

(57) ABSTRACT

Certain embodiments provide a composition comprising an organic extract and one or more excipients. For instance, certain compositions include an ashwagandha extract, a chelating agent, and an agent that enhances the delivery of one or more ingredients of the compositions. Non-limiting example compositions include ashwagandha extract, ethylenediaminetetraacetic acid (EDTA), and extracts of Panax notoginseng and Astragalus membranaceus. The compositions may further include one or more ingredients such as organic extract or powder (e.g., from herbs, roots, plants); vitamins; minerals; amino acids; adaptogens; and combinations of two or more thereof. Described herein are ashwagandha extract compositions formulated for oral delivery for use in a variety of applications, such as use against conditions such as low energy, anxiety, the inability to fall asleep, or a combination of one or more thereof.

### CROSS-REFERENCE TO RELATED APPLICATION

This application claims the benefit of U.S. Provisional Application 63/301,965 filed Jan. 21, 2022, the entirety of which is incorporated by reference herein.

### BACKGROUND

Ashwagandha, or Withania somnifera, is an herb comprising several biologically active chemical constituents including alkaloids, steroidal lactones, saponins, and anti-stress agents.

### SUMMARY

Embodiments of the present disclosure, in some aspects, provide nutraceutical compositions to support optimum performance and wellness for healthy living, mindfulness, and living a life of possibility. Example compositions comprise one or more extracts form an organic matter, such as ashwagandha.

Certain embodiments provide a composition comprising an organic extract and one or more excipients. For instance, certain compositions include an ashwagandha extract, a chelating agent, and an agent that enhances the delivery of one or more ingredients of the compositions. Non-limiting example compositions include ashwagandha extract, ethylenediaminetetraacetic acid (EDTA), and extracts of Panax notoginseng and Astragalus membranaceus. The compositions may further include one or more ingredients such as organic extract or powder (e.g., from herbs, roots, plants); vitamins; minerals; amino acids; adaptogens; and combinations of two or more thereof. Non-limiting ingredients of the compositions herein include ashwagandha (including extracts and powders thereof), EDTA, extracts of Panax notoginseng and Astragalus membranaceus (e.g., Astragin®), maca (Lepidium meyenii), Eurycoma longifolia, fenugreek, Tribulus terrestris, cordyceps mushroom, vitamin B6, nitric oxide, beet root powder, green tea extract, Panax ginseng, phosphatidylserine, sunflower powder, magnesium glycinate, zinc, Magnolia officinalis, ginger, horny goat weed, sclareolides, sage, diindolylmethane, bacopa monnieri, oat straw, D-aspartic acid, L-theanine, L-tryptophan, reishi mushroom, and melatonin. Further embodiments provide a method for taking a composition described herein.

The above features and methods are of representative embodiments only and are not intended to limit the scope and breadth of the claims.

In one aspect, provided is composition comprising an ashwagandha extract, a first excipient comprising an extract of Astragalus membranaceus and an extract of panax notoginseng, and a second excipient comprising a chelating agent. In some embodiments, the ashwagandha extract is present in the composition at about 20% to about 40% (w/w) or about 40% to about 60% (w/w); and/or wherein about 100 mg to about 400 mg or about 50 mg to about 250 mg of the ashwagandha extract is present in the composition. In some embodiments, the first excipient is present in the composition at about 1% to about 19% (w/w), about 5% to about 15% (w/w), or about 1% to about 10% (w/w); and/or wherein the composition comprises about 25 mg to about 75 mg, about 10 mg to about 50 mg, or about 10 mg to about 40 mg of the first excipient. In some embodiments, the second excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient. In some embodiments, the second excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient. In some embodiments, the chelating agent comprises ethylenediaminetetraacetic acid (EDTA), deferoxamine, deferasirox, deferiprone, penicillamine, trientine, dimercaprol, ethylenediamine, porphine, heme, sodium gluconate, tetrasodium glutamate diacetate, phytic acid, or a combination of two or more thereof. In some embodiments, the composition comprises (a) a melatonin, optionally present in the composition at about 0.01% (w/w) to about 2% (w/w), and optionally about 0.01 mg to about 5 mg of the melatonin is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; or (y) a combination of two or more selected from (a)-(x). In some embodiments, the composition comprises a flavoring agent, optionally present in the composition at about 1% to about 15% (w/w), and optionally about 4 mg to about 800 mg of the flavoring agent is present in the composition.

In one aspect, provided is composition comprising an ashwagandha extract and an excipient comprising an extract of Astragalus membranaceus and an extract of Panax notoginseng. In some embodiments, the ashwagandha extract is present in the composition at about 20% to about 40% (w/w) or about 40% to about 60% (w/w); and/or wherein about 100 mg to about 400 mg or about 50 mg to about 250 mg of the ashwagandha extract is present in the composition. In some embodiments, the excipient is present in the composition at about 1% to about 19% (w/w), about 5% to about 15% (w/w), or about 1% to about 10% (w/w); and/or wherein the composition comprises about 25 mg to about 75 mg, about 10 mg to about 50 mg, or about 10 mg to about 40 mg of the first excipient. In some embodiments, the composition comprises (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x). In some embodiments, the composition comprises a flavoring agent, optionally present in the composition at about 1% to about 15% (w/w), and optionally about 4 mg to about 800 mg of the flavoring agent is present in the composition.

In one aspect, provided is composition comprising an ashwagandha extract and an excipient comprising a chelating agent. In some embodiments, the ashwagandha extract is present in the composition at about 20% to about 40% (w/w) or about 40% to about 60% (w/w); and/or wherein about 100 mg to about 400 mg or about 50 mg to about 250 mg of the ashwagandha extract is present in the composition. In some embodiments, the excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient. In some embodiments, the chelating agent comprises ethylenediaminetetraacetic acid (EDTA), deferoxamine, deferasirox, deferiprone, penicillamine, trientine, dimercaprol, ethylenediamine, porphine, heme, sodium gluconate, tetrasodium glutamate diacetate, phytic acid, or a combination of two or more thereof. In some embodiments, the composition comprises (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x). In some embodiments, the composition comprises a flavoring agent, optionally present in the composition at about 1% to about 15% (w/w), and optionally about 4 mg to about 800 mg of the flavoring agent is present in the composition.

In one aspect, provided is composition comprising a first excipient comprising an extract of Astragalus membranaceus and an extract of Panax notoginseng, and a second excipient comprising a chelating agent. In some embodiments, the first excipient is present in the composition at about 1% to about 19% (w/w), about 5% to about 15% (w/w), or about 1% to about 10% (w/w); and/or wherein the composition comprises about 25 mg to about 75 mg, about 10 mg to about 50 mg, or about 10 mg to about 40 mg of the first excipient. In some embodiments, wherein the second excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient. In some embodiments, the chelating agent comprises ethylenediaminetetraacetic acid (EDTA), deferoxamine, deferasirox, deferiprone, penicillamine, trientine, dimercaprol, ethylenediamine, porphine, heme, sodium gluconate, tetrasodium glutamate diacetate, phytic acid, or a combination of two or more thereof. In some embodiments, the composition comprises (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x). In some embodiments, the composition comprises a flavoring agent, optionally present in the composition at about 1% to about 15% (w/w), and optionally about 4 mg to about 800 mg of the flavoring agent is present in the composition.

In some aspects or embodiments herein, the composition is (a) formulated in a tablet; (b) formulated in an oral dissolving tablet, optionally wherein the composition disintegrates in water in less than about 10 minutes, 9 minutes, 8 minutes, 7 minutes, 6 minutes, 5 minutes, 4 minutes, 3 minutes, 2 minutes, or 1 minute; (c) wherein the composition is prepared in a process comprising lyophilization; (d) wherein the composition is prepared in a unit having a weight of about 350 mg to about 1350 mg; or (e) a combination of two or more of (a) to (d).

In one aspect, provided is a method of modulating cortisol in a person, the method comprising taking a composition herein; optionally (a) wherein the delivery is oral; (b) wherein the composition increases energy, as compared to the person prior to taking the composition; (c) wherein the composition facilitates relaxation, as compared to the person prior to taking the composition; (d) wherein the composition improves sleep, as compared to the person prior to taking the composition; (e) wherein the composition is taken a regimen comprises two or more units, further optionally wherein each unit is taken daily or twice daily; further optionally wherein the regimen comprises taking the composition once daily for a week; (f) wherein the person is a female and the composition reduces a symptom of perimenopause or menopause in the female, as compared to the female prior to taking the composition; or (g) a combination of two or more of (a) to (f). In some embodiments, the composition is a composition of an aspect of embodiment herein.

In one aspect, provided is a method of improving the health of a person, the method comprising taking a composition herein; optionally (a) wherein the delivery is oral; (b) wherein the composition increases energy, as compared to the person prior to taking the composition; (c) wherein the composition facilitates relaxation, as compared to the person prior to taking the composition; (d) wherein the composition improves sleep, as compared to the person prior to taking the composition; (e) wherein the composition is taken a regimen comprises two or more units, further optionally wherein each unit is taken daily or twice daily; further optionally wherein the regimen comprises taking the composition once daily for a week; (f) wherein the person is a female and the composition reduces a symptom of perimenopause or menopause in the female, as compared to the female prior to taking the composition; or (g) a combination of two or more of (a) to (f). In some embodiments, the composition is a composition of an aspect of embodiment herein.

In one aspect, provided is a method of enhancing oral delivery of a composition comprising an ashwagandha extract, the method comprising combining with the ashwagandha extract (a) a first excipient comprising an extract of Astragalus membranaceus and an extract of Panax notoginseng, and/or (b) a second excipient comprising a chelating agent. In some embodiments, the composition is a composition of an aspect of embodiment herein.

In one aspect, provided is a method of enhancing oral delivery of an agent, the method comprising combining with the agent (a) a first excipient comprising an extract of Astragalus membranaceus and an extract of Panax notoginseng, and/or (b) a second excipient comprising a chelating agent; wherein the agent comprises: (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x). In some embodiments, the composition is a composition of an aspect of embodiment herein.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows the concentration of melatonin found in the blood of a person at 10-minute intervals for the first hour, 15-minute intervals for the second hour and 30-minute intervals for the third and fourth hour following the person taking a first composition comprising 5 mg of melatonin, and the person taking a second composition on another day comprising 5 mg of melatonin, as further described in Example 10.

FIG. 2 schematically illustrates a system for monitoring the health of a person, in accordance with an embodiment.

FIG. 3 illustrates a user interface, in accordance with an embodiment.

FIG. 4 illustrates a method of monitoring the health of a person, in accordance with an embodiment.

FIG. 5 shows a computer system that is programmed or otherwise configured to implement methods provided herein.

FIG. 6 is a flow chart of an example computer implemented method for assessing the health or wellbeing of a person based on information associated with a person.

FIG. 7 is a flow diagram of an example web-based computer system for monitoring and tracking the health and wellbeing of a person or persons and making recommendations based on the person's health.

FIGS. 8A-8O illustrate an example method of providing a health score and recommendation to a user.

FIG. 9 illustrates an example personalized email with results and recommendations generated in response to a user taking a health score quiz.

FIG. 10 illustrates an example visual score display.

### DETAILED DESCRIPTION

In some aspects, provided herein are compositions and oral formulations of a composition that may be used to improve the health of a person. In some aspects, formulations have increased oral bioavailability that provide increased efficiency for delivery of one or more ingredients of the composition. Example compositions comprise ashwagandha and one or more additional agents. Example compositions are formulated to modulate cortisol levels in a person. Non-limiting example benefits of embodiments of certain compositions provided herein include hormonal balancing, increased energy, facilitated relaxation, and improved sleep health.

### Cortisol

In one aspect, provided are compositions and formulations used to modulate cortisol levels in a person. Cortisol is a steroid hormone synthesized from cholesterol in the adrenal cortex. Specifically, the Zona fasciculata layer of the adrenal cortex. Secretion of cortisol is controlled by the hypothalamus, the pituitary gland, and the adrenal gland. These three are commonly known together as the HPA axis. Cortisol is known for being the stress hormone, but it is also responsible for much more. Without being bound by theory, cortisol has been shown to affect many functions in the human body. For example, cortisol has been shown to regulate the metabolism, control blood sugar levels, modulate inflammation in the body, play an important role in memory formation, play an important role in controlling blood pressure, and have controlling effects on salt and water balance. Moreover, in women cortisol levels may be important to the developing fetus during pregnancy. Cortisol levels thus may be instrumental in an individual's health and well-being. Furthermore, high cortisol levels have been associated with negative indications such as changes in a woman's libido and menstrual cycle, anxiety, and depression. High levels of cortisol may cause downstream negative effects in the body. High levels of cortisol may suppress the normal function of other hormones in the body. Other indications of high cortisol may include weight gain, high blood pressure, muscle weakness, fatigue, a weak immune system, difficulty healing wounds, thinning skin that is easily bruised, headaches, irritability, low libido, infertility, bone loss, insulin resistance leading to type 2 diabetes, negative effects on the frontal cortex affecting thinking and mood. Negative symptoms of low cortisol may include levels include fatigue, muscle or joint pain, low blood pressure, low blood sugar, nausea, vomiting, diarrhea, hyperpigmentation, irritability, depression, and hair loss. Use of compositions to modulate cortisol levels can mitigate these negative effects.

### Perimenopause and Menopause

In one aspect, provided are compositions and formulations that may be used to modulate changes in hormone levels, hot flashes (including shivering, sweating, and reddening of the skin), mood changes, and/or difficulty sleeping in perimenopausal and menopausal women. In some aspects, onset of menopause and/or perimenopause in women may be indicated by changes in hormone levels. In some aspects, onset of menopause and/or perimenopause in women may be indicated by age (e.g., between 47 and 54 years of age).

### Organic Extracts (e.g., Ashwagandha)

In one aspect, provided are compositions and formulations comprising an extract or powder from an organic material. As a non-limiting embodiment, provided are ashwagandha compositions. Ashwagandha, or Withania somnifera, is an herb that has been used in the traditional Indian medicine system, Aryuveda, for thousands of years. Ashwagandha is typically available as a fine powder and is commonly mixed into and taken with food. The herb has demonstrated anti-stress, anti-tumor, rejuvenation, anti-arthritic, and anti-neurodegenerative effects in scientific studies. Withania somnifera comprises several biologically active chemical constituents including alkaloids, steroidal lactones, saponins, and anti-stress agents. Alkaloids are cyclic organic compounds containing nitrogen in a negative oxidation state. Thousands of alkaloids are known, most having been isolated from plants. Numerous alkaloid compounds have application in the pharmaceutical field, both in their purified forms or in unpurified plant extract form. Many alkaloids derive bioactive properties in man, typically from their heterocyclic tertiary nitrogen. The alkaloid components of ashwagandha include isopelletierine, anaferine, cuseohygrine, and anahygrine. Steroidal lactones include withanolides. Withanolides are a group of naturally occurring 28 carbon steroids. One such withanolide, withanolide A, is isolated from the ashwagandha plant and has antioxidant and neuroprotective properties. Specifically, it has been shown to help promote neurite outgrowth in cultured neurons and in rodents. Another, withanolide B, contains enzymes that promote the production of nitric oxide, important for cellular signaling in humans. Withaferin A was the first withanolide-type constituent isolated from the withania somnifera (ashwagandha) plant. Withaferin A contains many of the bioactive components of the ashwagandha plant. Reports show that Witheraferin A has multiple potential health benefits such as anti-inflammatory, anti-diabetic, anti-cancer, and anti-angiogenesis effects. Withania somnifera has demonstrated anti-stress agents, such as sitoindosides and acylsterylglucosides. Anti-stress agents are compounds that mitigate stress-induced increases in physiological levels of certain compounds, such as urea nitrogen levels in blood. Saponins are a group of naturally occurring plant glycosides, characterized by their strong foam-forming properties in aqueous solution. Saponins include sitoindosides VII and VIII. In a non-limiting embodiment, ashwagandha is KSM-66®, which is a branded, full-spectrum extract, with the highest concentration of all major root only extracts. KSM-66® has at least 5% withanolides (% w/w). KSM-66® ashwagandha is produced using green chemistry extraction processes, without the use of chemical solvents. The KSM-66 extraction process uses the roots of the ashwagandha plant with no addition of leaves. Most scientific studies explore the therapeutic effects of the roots and not the leaves. The KSM-66 process produces a full-spectrum extract which maintains the balance of the efficacious compounds of the ashwagandha root in their natural proportions. The KSM-66® extraction process may entail pre-treating the ashwagandha roots with milk. Doing so promotes retention of both the hydrophilic and lipophilic components. Alternatively, the KSM-66® process may use a milk-free vegan process. The remainder of both processes may be done with green chemistry as described above.

Studies of KSM-66® ashwagandha extract demonstrate its natural properties. Specifically, root extract of ashwagandha has sleep-inducing potential. It is well-tolerated and improves sleep onset and sleep quality in persons with insomnia. Further, it has the potential to be used to improve sleep parameters for individuals with anxiety and insomnia. Furthermore, intake of an ashwagandha extract plays an important role in the modulation of cortisol levels in the human body. Ashwagandha intake has been shown to result in reduction of cortisol levels. Furthermore, ashwagandha is an adaptogen, meaning it enables the body to adapt to stress (e.g., by mitigating the cortisol or adrenal response). Thus, intake of ashwagandha may diminish the downstream effects cortisol has on the human body.

In some embodiments, about 25 mg to about 2500 mg of ashwagandha is present in a composition herein, e.g., about 25 mg to about 2000 mg, about 25 mg to about 1500 mg, about 25 mg to about 1000 mg, about 25 mg to about 500 mg, about 25 mg to about 450 mg, about 25 mg to about 400 mg, about 25 mg to about 350, about 25 mg to about 300 mg, about 25 mg to about 250 mg, about 25 mg to about 200 mg, about 25 mg to about 150 mg, about 25 mg to about 100 mg, about 25 mg to about 75 mg, about 25 mg to about 50 mg, about 50 mg to about 2500 mg, about 50 mg to about 2000 mg, about 50 mg to about 1500 mg, about 50 mg to about 1000 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 75 mg, about 100 mg to about 2500 mg, about 100 mg to about 2000 mg, about 100 mg to about 1500 mg, about 100 mg to about 1000 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 2500 mg, about 150 mg to about 2000 mg, about 150 mg to about 1500 mg, about 150 mg to about 1000 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 2500 mg, about 200 mg to about 2000 mg, about 200 mg to about 1500 mg, about 200 mg to about 1000 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 2500 mg, about 250 mg to about 2000 mg, about 250 mg to about 1500 mg, about 250 mg to about 1000 mg, about 250 mg to about 500 mg, about 250 mg to about 450 mg, about 250 mg to about 400 mg, about 250 mg to about 350 mg, about 250 mg to about 300 mg, about 300 mg to about 2500 mg, about 300 mg to about 2000 mg, about 300 mg to about 1500 mg, about 300 mg to about 1000 mg, about 300 mg to about 500 mg, about 300 mg to about 450 mg, about 300 mg to about 400 mg, about 300 mg to about 350 mg, about 350 mg to about 2500 mg, about 350 mg to about 2000 mg, about 350 mg to about 1500 mg, about 350 mg to about 1000 mg, about 350 mg to about 500 mg, about 350 mg to about 450 mg, about 350 mg to about 400 mg, about 400 mg to about 2500 mg, about 400 mg to about 2000 mg, about 400 mg to about 1500 mg, about 400 mg to about 1000 mg, about 400 mg to about 500 mg, about 400 mg to about 450 mg, about 450 mg to about 2500 mg, about 450 mg to about 2000 mg, about 450 mg to about 1500 mg, about 450 mg to about 1000 mg, about 450 mg to about 500 mg, about 500 mg to about 2500 mg, about 500 mg to about 2000 mg, about 500 mg to about 1500 mg, about 500 mg to about 1000 mg, about 1000 mg to about 2500 mg, about 1000 mg to about 2000 mg, about 1000 mg to about 1500 mg, about 1500 mg to about 2500 mg, about 1500 mg to about 2000 mg, about 2000 mg to about 2500 mg, about 25 mg to about 250 mg, about 150 to about 500 mg, about 150 mg to about 450 mg, about 50 mg to about 400 mg, about 50 mg to about 350, about 50 mg to about 300 mg, about 50 mg to about 250 mg, about 50 mg to about 200 mg, about 50 mg to about 150 mg, about 50 mg to about 100 mg, about 50 mg to about 75 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 1000 mg, about 1500 mg, about 2000 mg, or about 2500 mg.

In some embodiments, about means within 10% of the stated amount. For instance, an agent present in a composition at about 30% may comprise 27% to 33% of the agent.

In some embodiments, about 12.5 mg to about 1250 mg of ashwagandha is present in a composition herein, e.g., about 12.5 mg to about 1000 mg, about 12.5 mg to about 500 mg, about 12.5 mg to about 450 mg, about 12.5 mg to about 400 mg, about 12.5 mg to about 350 mg, about 12.5 to about 300 mg, about 12.5 to about 250 mg, about 12.5 to about 200 mg, about 12.5 to about 175 mg, about 12.5 mg to about 150 mg, about 12.5 mg to about 125 mg, about 12.5 mg to about 100 mg, about 12.5 mg to about 75 mg, about 12.5 to about 50 mg, about 12.5 to about 25 mg, about 25 mg to about 1250 mg, about 25 mg to about 1000 mg, about 25 mg to about 500 mg, about 25 mg to about 450 mg, about 25 mg to about 400 mg, about 25 mg to about 350 mg, about 25 to about 300 mg, about 25 to about 250 mg, about 25 to about 200 mg, about 25 to about 175 mg, about 25 mg to about 150 mg, about 25 mg to about 125 mg, about 25 mg to about 100 mg, about 25 mg to about 75 mg, about 25 to about 50 mg, about 50 mg to about 1250 mg, about 50 mg to about 1000 mg, about 50 mg to about 500 mg, about 50 mg to about 450 mg, about 50 mg to about 400 mg, about 50 mg to about 350 mg, about 50 to about 300 mg, about 50 to about 250 mg, about 50 to about 200 mg, about 50 to about 175 mg, about 50 mg to about 150 mg, about 50 mg to about 125 mg, about 50 mg to about 100 mg, about 50 mg to about 75 mg, about 75 mg to about 1250 mg, about 75 mg to about 1000 mg, about 75 mg to about 500 mg, about 75 mg to about 450 mg, about 75 mg to about 400 mg, about 75 mg to about 350 mg, about 75 to about 300 mg, about 75 to about 250 mg, about 75 to about 200 mg, about 75 to about 175 mg, about 75 mg to about 150 mg, about 75 mg to about 125 mg, about 75 mg to about 100 mg, about 100 mg to about 1250 mg, about 100 mg to about 1000 mg, about 100 mg to about 500 mg, about 100 mg to about 450 mg, about 100 mg to about 400 mg, about 100 mg to about 350 mg, about 100 to about 300 mg, about 100 to about 250 mg, about 100 to about 200 mg, about 100 to about 175 mg, about 100 mg to about 150 mg, about 100 mg to about 125 mg, about 125 mg to about 1250 mg, about 125 mg to about 1000 mg, about 125 mg to about 500 mg, about 125 mg to about 450 mg, about 125 mg to about 400 mg, about 125 mg to about 350 mg, about 125 to about 300 mg, about 125 to about 250 mg, about 125 to about 200 mg, about 125 to about 175 mg, about 125 mg to about 150 mg, about 150 mg to about 1250 mg, about 150 mg to about 1000 mg, about 150 mg to about 500 mg, about 150 mg to about 450 mg, about 150 mg to about 400 mg, about 150 mg to about 350 mg, about 150 to about 300 mg, about 150 to about 250 mg, about 150 to about 200 mg, about 150 to about 175 mg, about 175 mg to about 1250 mg, about 175 mg to about 1000 mg, about 175 mg to about 500 mg, about 175 mg to about 450 mg, about 175 mg to about 400 mg, about 175 mg to about 350 mg, about 175 to about 300 mg, about 175 to about 250 mg, about 175 to about 200 mg, about 200 mg to about 1250 mg, about 200 mg to about 1000 mg, about 200 mg to about 500 mg, about 200 mg to about 450 mg, about 200 mg to about 400 mg, about 200 mg to about 350 mg, about 200 to about 300 mg, about 200 to about 250 mg, about 250 mg to about 1250 mg, about 250 mg to about 1000 mg, about 250 mg to about 500 mg, about 250 mg to about 450 mg, about 250 mg to about 400 mg, about 250 mg to about 350 mg, about 250 to about 300 mg, about 300 mg to about 1250 mg, about 300 mg to about 1000 mg, about 300 mg to about 500 mg, about 300 mg to about 450 mg, about 300 mg to about 400 mg, about 300 mg to about 350 mg, about 350 mg to about 1250 mg, about 350 mg to about 1000 mg, about 350 mg to about 500 mg, about 350 mg to about 450 mg, about 350 mg to about 400 mg, about 400 mg to about 1250 mg, about 400 mg to about 1000 mg, about 400 mg to about 500 mg, about 400 mg to about 450 mg, about 450 mg to about 1250 mg, about 450 mg to about 1000 mg, about 450 mg to about 500 mg, about 500 mg to about 1250 mg, about 500 mg to about 1000 mg, about 1000 mg to about 1250 mg, about 12.5 mg, about 25 mg, about 50 mg, about 75 mg, about 100 mg, about 125 mg, about 150 mg, about 175 mg, about 200 mg, about 225 mg, about 250 mg, about 300 mg, about 350 mg, about 400 mg, about 450 mg, about 500 mg, about 1000 mg, or about 1250 mg.

In some embodiments, about 10% to about 40% (w/w) of a composition is ashwagandha, e.g., about 10% to about 35%, about 10% to about 30%, about 10% to about 25%, about 15% to about 35%, about 15% to about 30%, about 15% to about 25%, about 20% to about 35%, about 20% to about 30%, about 20% to about 25%, about 25% to about 40%, about 25% to about 35%, about 25% to about 30%, about 30% to about 40%, about 30% to about 35%, about 35% to about 40%, about 20%, about 25%, about 30%, about 35%, or about 40% of the composition is ashwagandha.

In some embodiments, about 20% to about 60% (w/w) of a composition is ashwagandha, e.g., about 20% to about 55%, about 20% to about 50%, about 20% to about 45%, about 20% to about 40%, about 20% to about 35%, about 20% to about 30%, about 20% to about 25%, about 25% to about 55%, about 25% to about 50%, about 25% to about 45%, about 25% to about 40%, about 25% to about 35%, about 25% to about 30%, about 30% to about 55%, about 30% to about 50%, about 30% to about 45%, about 30% to about 40%, about 30% to about 35%, about 35% to about 55%, about 35% to about 50%, about 35% to about 45%, about 35% to about 40%, about 40% to about 60%, about 40% to about 55%, about 40% to about 50%, about 40% to about 45%, about 45% to about 60%, about 45% to about 55%, about 45% to about 50%, about 50% to about 60%, about 50% to about 55%, about 55% to about 60%, about 40%, about 45%, about 50%, about 55%, or about 60% of the composition is ashwagandha.

Certain compositions herein are formulated to facilitate increased energy in a person. In some such cases, the amount of ashwagandha present in the composition is about 150 mg to about 350 mg, about 175 mg to about 325 mg, about 200 mg to about 300 mg, about 225 mg to about 275 mg, or about 250 mg. An example product is called RH1.

Certain compositions herein are formulated to facilitate increased relaxation in a person. In some such cases, the amount of ashwagandha present in the composition is about 150 mg to about 350 mg, about 175 mg to about 325 mg, about 200 mg to about 300 mg, about 225 mg to about 275 mg, or about 250 mg. An example product is called RH2.

Certain compositions herein are formulated to improve the sleep health of a person. In some such cases, the amount of ashwagandha present in the composition is about 75 mg to about 175 mg, about 100 mg to about 150 mg, or about 125 mg. An example product is called RH3.

As described above, intake of ashwagandha may lead to various effects relating to a person. For example, intake of ashwagandha may lead to improved ability to fall asleep, improved sleep, reduction of anxiety, modulation of cortisol levels, and regulated stress levels. Intake of ashwagandha may facilitate hormonal balance, which may lead to increased energy, increased relaxation, or both. While KSM-66® is described above as a source of ashwagandha, KSM-66® is exemplary and other sources of ashwagandha may be used.

### Maca

In one aspect, provided are compositions and formulations comprising maca. Maca (Lepidium meyenii) is a root, belonging to the cabbage family, native to the Andean region of Peru, where it has been cultivated for at least 2000 years. Maca is rich in fiber, essential amino acids, fatty acids, and other nutrients, including vitamins such as vitamin A, vitamin b6, manganese, copper, iron, and calcium. Further, the root contains bioactive compounds that have been shown to be responsible for therapeutic benefits to the human body, e.g., regulation of sexual dysfunction, memory enhancement, anti-depressant effects, antioxidant effects, anti-inflammatory effects, anti-cancer effects, neuroprotective effects, and skin protection. Experimental studies indicate the effects maca has on energy, mood, nutrition, fertility, hormone production, and memory. Furthermore, maca has been shown to improve sperm production, sperm motility and semen volume. Additionally, there are indications that maca may be used to reduce prostate size. The role maca plays in homeostatic balance is related to bone metabolism. Use of maca by menopausal women has been associated with improvements in sexual dysfunction and reduction in instances of anxiety and depression. Maca is used as a supplement to enhance fertility and improve libido as well as the effects listed above. Typically, maca is extracted and available in a powdered form.

In some embodiments, about 18 mg to about 2200 mg of maca is present in a composition herein, e.g., about 18 mg to about 2000 mg, about 18 mg to about 1500 mg, about 18 mg to about 1000 mg, about 18 mg to about 500 mg, about 18 mg to about 450 mg, about 18 mg to about 400 mg, about 18 mg to about 350 mg, about 18 mg to about 300 mg, about 18 mg to about 250 mg, about 18 mg to about 225 mg, about 18 mg to about 200 mg, about 18 mg to about 175 mg, about 18 mg to about 150 mg, about 18 mg to about 125 mg, about 18 mg to about 100 mg, about 18 mg to about 75 mg, about 18 mg to about 50 mg, about 18 mg to about 25 mg, about 25 mg to about 2200 mg, about 25 mg to about 2000 mg, about 25 mg to about 1500 mg, about 25 mg to about 1000 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 2200 mg, about 50 mg to about 2000 mg, about 50 mg to about 1500 mg, about 50 mg to about 1000 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 2200 mg, about 75 mg to about 2000 mg, about 75 mg to about 1500 mg, about 75 mg to about 1000 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 2200 mg, about 100 mg to about 2000 mg, about 100 mg to about 1500 mg, about 100 mg to about 1000 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 2200 mg, about 125 mg to about 2000 mg, about 125 mg to about 1500 mg, about 125 mg to about 1000 mg, about 125 mg to about 500 mg, about 125 mg to about 450 mg, about 125 mg to about 400 mg, about 125 mg to about 350 mg, about 125 mg to about 300 mg, about 125 mg to about 250 mg, about 125 mg to about 225 mg, about 125 mg to about 200 mg, about 125 mg to about 175 mg, about 125 mg to about 150 mg, about 150 mg to about 2200 mg, about 150 mg to about 2000 mg, about 150 mg to about 1500 mg, about 150 mg to about 1000 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 225 mg, about 150 mg to about 200 mg, about 150 mg to about 175 mg, about 175 mg to about 2200 mg, about 175 mg to about 2000 mg, about 175 mg to about 1500 mg, about 175 mg to about 1000 mg, about 175 mg to about 500 mg, about 175 mg to about 450 mg, about 175 mg to about 400 mg, about 175 mg to about 350 mg, about 175 mg to about 300 mg, about 175 mg to about 250 mg, about 175 mg to about 225 mg, about 175 mg to about 200 mg, about 200 mg to about 2200 mg, about 200 mg to about 2000 mg, about 200 mg to about 1500 mg, about 200 mg to about 1000 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 200 mg to about 225 mg, about 225 mg to about 2200 mg, about 225 mg to about 2000 mg, about 225 mg to about 1500 mg, about 225 mg to about 1000 mg, about 225 mg to about 500 mg, about 225 mg to about 450 mg, about 225 mg to about 400 mg, about 225 mg to about 350 mg, about 225 mg to about 300 mg, about 225 mg to about 250 mg, about 250 mg to about 2200 mg, about 250 mg to about 2000 mg, about 250 mg to about 1500 mg, about 250 mg to about 1000 mg, about 250 mg to about 500 mg, about 250 mg to about 450 mg, about 250 mg to about 400 mg, about 250 mg to about 350 mg, about 250 mg to about 300 mg, about 300 mg to about 2200 mg, about 300 mg to about 2000 mg, about 300 mg to about 1500 mg, about 300 mg to about 1000 mg, about 300 mg to about 500 mg, about 300 mg to about 450 mg, about 300 mg to about 400 mg, about 300 mg to about 350 mg, about 350 mg to about 2200 mg, about 350 mg to about 2000 mg, about 350 mg to about 1500 mg, about 350 mg to about 1000 mg, about 350 mg to about 500 mg, about 350 mg to about 450 mg, about 350 mg to about 400 mg, about 400 mg to about 2200 mg, about 400 mg to about 2000 mg, about 400 mg to about 1500 mg, about 400 mg to about 1000 mg, about 400 mg to about 500 mg, about 400 mg to about 450 mg, about 450 mg to about 2200 mg, about 450 mg to about 2000 mg, about 450 mg to about 1500 mg, about 450 mg to about 1000 mg, about 450 mg to about 500 mg, about 500 mg to about 2200 mg, about 500 mg to about 2000 mg, about 500 mg to about 1500 mg, about 500 mg to about 1000 mg, about 1000 mg to about 2200 mg, about 1000 mg to about 2000 mg, about 1000 mg to about 1500 mg, about 1500 mg to about 2200 mg, about 1500 mg to about 2000 mg, about 2000 mg to about 2200 mg, about 18 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 450 mg, about 500 mg, about 600 mg, about 700 mg, about 800 mg, about 900 mg, about 1000 mg, about 1250 mg, about 1500 mg, about 1750 mg, about 2000 mg, or about 2200 mg.

In some embodiments, about 10% to about 40% (w/w) of a composition is maca, e.g., about 10% to about 35%, about 10% to about 30%, about 10% to about 25%, about 10% to about 20%, about 15% to about 40%, about 15% to about 35%, about 15% to about 30%, about 15% to about 25%, about 15% to about 20%, about 20% to about 40%, about 20% to about 35%, about 20% to about 30%, about 20% to about 25%, about 25% to about 40%, about 25% to about 35%, about 25% to about 30%, about 30% to about 40%, about 30% to about 35%, about 15%, about 20%, about 25%, about 30%, or about 35% of the composition is maca.

Certain compositions herein are formulated to enhance fertility and improve libido in a person. In some such cases, the amount of maca present in the composition is about 120 mg to about 320 mg, about 145 mg to about 295 mg, about 170 mg to about 270 mg, about 195 mg to about 245 mg, or about 220 mg. In some cases, the amount of maca present in the composition is about 100 mg to about 300 mg, about 120 mg to about 280 mg, about 140 mg to about 260 mg, about 160 mg to about 240 mg, about 180 mg to about 220 mg, or about 190 mg. An example product is called RH1.

As described above, intake of maca may lead to various effects relating to a person. For example, maca has nutritional benefits such as containing fiber, essential amino acids, fatty acids, vitamins, manganese, copper, iron, and calcium. Intake of maca may further lead to other benefits such as improving or diminishing effects from sexual dysfunction, depression, inflammation, and cancer. Intake of maca may also lead to effects such as enhanced memory, reducing risk of disease by maca's antioxidant properties, and increased skin protection.

### Eurycoma Longifolia

In one aspect, provided are compositions comprising Eurycoma longifolia. Eurycoma longifolia (EL) is a male sexual health booster. Clinical studies have demonstrated its efficacy in managing male sexual disorders such as erectile dysfunction, infertility, low libido, and downregulated testosterone levels. The EL extract originates from the Eurycoma longifolia plant. The medicinal plant is from the Simaroubaceae plant family. EL is a shrub tree that grows to heights up to 10 meters with long green leaves. The medicinal component comes from the roots of the plant. The root is dried and ground up. It can be used in its raw form without further chemical processing, or further processed to obtain a medicinal extract. A wide range of chemical compounds have been identified in Eurycoma longifolia such as urycomanone, eurycomanol, eurycomalactone, canthine-6-one alkaloid, 9-hydroxycanthin-6-one, 14,15β-dihydroxyklaineanone, phenolic components, tannins, quanissoids, and triterpenes.

In some embodiments, about 10 mg to about 1125 mg of Eurycoma longifolia is present in a composition herein, e.g., about 10 mg to about 1000 mg, about 10 mg to about 750 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 125 mg, about 10 mg to about 100 mg, about 10 mg to about 75 mg, about 10 mg to about 50 mg, about 10 mg to about 25 mg, about 25 mg to about 1000 mg, about 25 mg to about 750 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 150 mg, about 25 mg to about 125 mg, about 25 mg to about 100 mg, about 25 mg to about 75 mg, about 25 mg to about 50 mg, about 50 mg to about 1000 mg, about 50 mg to about 750 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 125 mg, about 50 mg to about 100 mg, about 50 mg to about 75 mg, about 75 mg to about 1000 mg, about 75 mg to about 750 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 200 mg, about 75 mg to about 150 mg, about 75 mg to about 125 mg, about 75 mg to about 100 mg, about 100 mg to about 1000 mg, about 100 mg to about 750 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 100 mg to about 125 mg, about 125 mg to about 1000 mg, about 125 mg to about 750 mg, about 125 mg to about 500 mg, about 125 mg to about 450 mg, about 125 mg to about 400 mg, about 125 mg to about 350 mg, about 125 mg to about 300 mg, about 125 mg to about 250 mg, about 125 mg to about 200 mg, about 125 mg to about 150 mg, about 150 mg to about 1000 mg, about 150 mg to about 750 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 1000 mg, about 200 mg to about 750 mg, about 200 mg to about 500 mg, about 200 mg to about 450 mg, about 200 mg to about 400 mg, about 200 mg to about 350 mg, about 200 mg to about 300 mg, about 200 mg to about 250 mg, 250 mg to about 1000 mg, about 250 mg to about 750 mg, about 250 mg to about 500 mg, about 250 mg to about 450 mg, about 250 mg to about 400 mg, about 250 mg to about 350 mg, about 250 mg to about 300 mg, 300 mg to about 1000 mg, about 300 mg to about 750 mg, about 300 mg to about 500 mg, about 300 mg to about 450 mg, about 300 mg to about 400 mg, about 300 mg to about 350 mg, 350 mg to about 1000 mg, about 350 mg to about 750 mg, about 350 mg to about 500 mg, about 350 mg to about 450 mg, about 350 mg to about 400 mg, 400 mg to about 1000 mg, about 400 mg to about 750 mg, about 400 mg to about 500 mg, about 400 mg to about 450 mg, 450 mg to about 1000 mg, about 450 mg to about 750 mg, about 450 mg to about 500 mg, 500 mg to about 1000 mg, about 500 mg to about 750 mg, about 750 mg to about 1000 mg, about 10 mg, about 25 mg, about 50 mg, about 75 mg, about 100 mg, about 112.5 mg, about 125 mg, about 150 mg, about 175 mg, about 200 mg, about 225 mg, about 250 mg, or about 275 mg, about 300 mg, about 350 mg, about 400 mg, about 450 mg, about 500 mg, about 750 mg, or about 1000 mg.

In some embodiments, about 5% to about 20% (w/w) of a composition is Eurycoma longifolia, e.g., about 5% to about 15%, about 5% to about 10%, about 10% to about 20%, about 10% to about 15%, about 15% to about 20%, about 5%, about 10%, about 15%, about 20%, or about 25% of the composition is Eurycoma longifolia.

Certain compositions herein are formulated to improve sexual health in a male. In some such cases, the amount of Eurycoma longifolia present in the composition is about 50 mg to about 170 mg, about 70 mg to about 150 mg, about 90 mg to about 130 mg, or about 110 mg. An example product is called RH1.

As described above, intake of Eurycoma longifolia may lead to various effects for a person. For example, intake of Eurycoma longifolia may lead to diminishing symptoms related to erectile dysfunction and infertility. Intake of Eurycoma longifolia may also improve the ability to regulate libido and testosterone levels.

### Fenusreek

In one aspect, provided are compositions comprising fenugreek seed extract. Fenugreek seeds have been used historically as an herbal remedy mitigating nutritive and metabolic dysfunctions. The extract has been shown to inhibit fat accumulation. Fenugreek, or Trigonella foenum-graecum (TFG) seeds belong to the Fabaceae family. TFG extract has been used against diabetes and has been included as an active ingredient in weight loss medications. TFG extract has been shown to possess hypoglycemic, antioxidant, and hypolipidemic effects. One extract of TFG seeds, Testosurge®, has been shown to improve strength, body composition (e.g., fat burning), and hormonal profiles.

In some embodiments, about 10 mg to about 1750 mg of fenugreek seed extract (e.g., Testosurge®) is present in a composition herein, e.g., about 10 mg to about 1500 mg, about 10 mg to about 1250 mg, about 10 mg to about 1000 mg, about 10 mg to about 750 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 175 mg, about 10 mg to about 150 mg, about 10 mg to about 125 mg, about 10 mg to about 100 mg, about 10 mg to about 75 mg, about 10 mg to about 50 mg, about 10 mg to about 25 mg, about 25 mg to about 1750 mg, about 25 mg to about 1500 mg, about 25 mg to about 1250 mg, about 25 mg to about 1000 mg, about 25 mg to about 750 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 1750 mg, about 50 mg to about 1500 mg, about 50 mg to about 1250 mg, about 50 mg to about 1000 mg, about 50 mg to about 750 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 1750 mg, about 75 mg to about 1500 mg, about 75 mg to about 1250 mg, about 75 mg to about 1000 mg, about 75 mg to about 750 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 200 mg, about 75 mg to about 175 mg, about 75 mg to about 150 mg, about 75 mg to about 125 mg, about 75 mg to about 100 mg, about 100 mg to about 1750 mg, about 100 mg to about 1500 mg, about 100 mg to about 1250 mg, about 100 mg to about 1000 mg, about 100 mg to about 750 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 175 mg, about 100 mg to about 150 mg, about 100 mg to about 125 mg, about 125 mg to about 1750 mg, about 125 mg to about 1500 mg, about 125 mg to about 1250 mg, about 125 mg to about 1000 mg, about 125 mg to about 750 mg, about 125 mg to about 500 mg, about 125 mg to about 450 mg, about 125 mg to about 400 mg, about 125 mg to about 350 mg, about 125 mg to about 300 mg, about 125 mg to about 250 mg, about 125 mg to about 200 mg, about 125 mg to about 175 mg, about 125 mg to about 150 mg, about 150 mg to about 1750 mg, about 150 mg to about 1500 mg, about 150 mg to about 1250 mg, about 150 mg to about 1000 mg, about 150 mg to about 750 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 150 mg to about 175 mg, about 175 mg to about 1750 mg, about 175 mg to about 1500 mg, about 175 mg to about 1250 mg, about 175 mg to about 1000 mg, about 175 mg to about 750 mg, about 175 mg to about 500 mg, about 175 mg to about 450 mg, about 175 mg to about 400 mg, about 175 mg to about 350 mg, about 175 mg to about 300 mg, about 175 mg to about 250 mg, about 175 mg to about 200 mg, about 175 mg to about 1750 mg, about 175 mg to about 1500 mg, about 175 mg to about 1250 mg, about 175 mg to about 1000 mg, about 175 mg to about 750 mg, about 175 mg to about 500 mg, about 175 mg to about 450 mg, about 175 mg to about 400 mg, about 175 mg to about 350 mg, about 175 mg to about 300 mg, about 175 mg to about 250 mg, about 175 mg to about 200 mg, about 200 mg to about 1750 mg, about 200 mg to about 1500 mg, about 200 mg to about 1250 mg, about 200 mg to about 1000 mg, about 200 mg to about 750 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 1750 mg, about 250 mg to about 1500 mg, about 250 mg to about 1250 mg, about 250 mg to about 1000 mg, about 250 mg to about 750 mg, about 250 mg to about 500 mg, about 250 mg to about 450 mg, about 250 mg to about 400 mg, about 250 mg to about 350 mg, about 250 mg to about 300 mg, about 300 mg to about 1750 mg, about 300 mg to about 1500 mg, about 300 mg to about 1250 mg, about 300 mg to about 1000 mg, about 300 mg to about 750 mg, about 300 mg to about 500 mg, about 300 mg to about 450 mg, about 300 mg to about 400 mg, about 300 mg to about 350 mg, about 350 mg to about 1750 mg, about 350 mg to about 1500 mg, about 350 mg to about 1250 mg, about 350 mg to about 1000 mg, about 350 mg to about 750 mg, about 350 mg to about 500 mg, about 350 mg to about 450 mg, about 350 mg to about 400 mg, about 400 mg to about 1750 mg, about 400 mg to about 1500 mg, about 400 mg to about 1250 mg, about 400 mg to about 1000 mg, about 400 mg to about 750 mg, about 400 mg to about 500 mg, about 400 mg to about 450 mg, about 450 mg to about 1750 mg, about 450 mg to about 1500 mg, about 450 mg to about 1250 mg, about 450 mg to about 1000 mg, about 450 mg to about 750 mg, about 450 mg to about 500 mg, about 500 mg to about 1750 mg, about 500 mg to about 1500 mg, about 500 mg to about 1250 mg, about 500 mg to about 1000 mg, about 500 mg to about 750 mg, about 750 mg to about 1750 mg, about 750 mg to about 1500 mg, about 750 mg to about 1250 mg, about 750 mg to about 1000 mg, 1000 mg to about 1750 mg, about 1000 mg to about 1500 mg, about 1000 mg to about 1250 mg, 1250 mg to about 1750 mg, about 1250 mg to about 1500 mg, 1500 mg to about 1750 mg, about 10 mg, about 25 mg, about 50 mg, about 75 mg, about 100 mg, about 125 mg, about 150 mg, about 175 mg, about 200 mg, about 225 mg, about 250 mg, or about 275 mg, about 300 mg, about 350 mg, about 400 mg, about 450 mg, about 500 mg, about 750 mg, about 1000 mg, about 1250 mg, about 1500 mg, or about 1750 mg.

In some embodiments, about 5% to about 30% (w/w) of a composition is fenugreek extract (e.g., Testosurge®), e.g., about 5% to about 25%, about 5% to about 20%, about 5% to about 15%, about 5% to about 10%, about 10% to about 30%, about 10% to about 25%, about 10% to about 20%, about 10% to about 15%, about 15% to about 30%, about 15% to about 25%, about 15% to about 20%, about 20% to about 30%, about 20% to about 25%, about 25% to about 30%, about 5%, about 10%, about 15%, about 20%, about 25%, or about 30% of the composition is fenugreek seed extract (e.g., Testosurge®).

Certain compositions herein are formulated to improve metabolism and the hormonal profile in a person. In some such cases, the amount of fenugreek extract (e.g., Testosurge®) present in the composition is about 100 mg to about 220 mg, about 120 mg to about 200 mg, about 140 mg to about 180 mg, or about 170 mg. In some cases, the amount of fenugreek extract (e.g., Testosurge®) present in the composition is about 50 mg to about 170 mg, about 70 mg to about 150 mg, about 90 mg to about 130 mg, or about 110 mg. An example product is called RH1.

As described above, intake of fenugreek seed extract may lead to various effects for a person. For example, intake of fenugreek seed extract may inhibit fat accumulation and symptoms of diabetes. As another example, intake of fenugreek seed may promote weight loss, body composition, and increased strength of a person. While Testosurge® is described above an example of fenugreek extract, Testosurge® is exemplary and other sources of fenugreek extract may be used.

### Tribulus Terrestris

In one aspect, provided are compositions comprising Tribulus terrestris. Tribulus terrestris is an herbal plant of the zygophyllacae family. Extracts of the plant, from its aerial parts and fruits, have been used for pharmacological uses. Examples of extract properties include diuretic, tonic, and aphrodisiac properties.

In some embodiments, about 1 mg to about 125 mg of Tribulus terrestris is present in a composition herein, e.g., about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 125 mg, about 75 mg to about 100 mg, about 100 mg to about 125 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, or about 125 mg.

In some embodiments, about 0.1% to about 2.5% (w/w) of a composition is Tribulus terrestris, e.g., about 0.1% to about 2.5%, about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, about 2.1% to about 2.5%, about 0.1%, about 0.5%, about 0.9%, about 1.3%, about 1.7%, about 2.1%, or about 2.5% of the composition is Tribulus terrestris.

Certain compositions herein are formulated to improve the hormonal profile in a male. In some such cases, the amount of Tribulus terrestris present in the composition is about 5 mg to about 21 mg, about 9 mg to about 17 mg, about 10 mg to about 15 mg, or about 12.5 mg. An example product is called RH1.

As described above, intake of Tribulus terrestris may lead to various effects for a person. For example, intake of Tribulus terrestris may improve a person's salt levels (e.g., in relation to water levels) in the body. Intake of Tribulus terrestris may also lead to improved energy levels and libido of a person.

### Cordyceps Mushroom (e.g., CordycepsPrime™)

In one aspect, provided are compositions comprising cordyceps mushroom, or cordyceps sinensis (e.g., CordycepsPrime™). Cordyceps species have been known to exhibit a wide range of pharmacological effects including antioxidative, anti-inflammatory, anticancer, anti-obesity, and antidiabetic. The pharmacological potential of these plants has been demonstrated. Cordyceps mushrooms from CordycepsPrime™ are grown using unique fermentation technologies in nutrient-dense solution.

In some embodiments, about 0.5 mg to about 100 mg of cordyceps mushroom is present in a composition herein, e.g., about 0.5 mg to about 75 mg, about 0.5 mg to about 50 mg, about 0.5 mg to about 40 mg, about 0.5 mg to about 30 mg, about 0.5 mg to about 20 mg, about 0.5 mg to about 15 mg, about 0.5 mg to about 10 mg, about 0.5 mg to about 7.5 mg, about 0.5 mg to about 5 mg, about 0.5 mg to about 1 mg, about 1 mg to about 100 mg, about 1 mg to about 75 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 7.5 mg, about 1 mg to about 5 mg, about 5 mg to about 100 mg, about 5 mg to about 75 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 5 mg to about 7.5 mg, about 7.5 mg to about 100 mg, about 7.5 mg to about 75 mg, about 7.5 mg to about 50 mg, about 7.5 mg to about 40 mg, about 7.5 mg to about 30 mg, about 7.5 mg to about 20 mg, about 7.5 mg to about 15 mg, about 7.5 mg to about 10 mg, about 10 mg to about 100 mg, about 10 mg to about 75 mg, about 10 mg to about 50 mg, about 10 mg to about 40 mg, about 10 mg to about 30 mg, about 10 mg to about 20 mg, about 10 mg to about 15 mg, about 15 mg to about 100 mg, about 15 mg to about 75 mg, about 15 mg to about 50 mg, about 15 mg to about 40 mg, about 15 mg to about 30 mg, about 15 mg to about 20 mg, about 20 mg to about 100 mg, about 20 mg to about 75 mg, about 20 mg to about 50 mg, about 20 mg to about 40 mg, about 20 mg to about 30 mg, about 30 mg to about 100 mg, about 30 mg to about 75 mg, about 30 mg to about 50 mg, about 30 mg to about 40 mg, about 40 mg to about 100 mg, about 40 mg to about 75 mg, about 40 mg to about 50 mg, about 50 mg to about 100 mg, about 50 mg to about 75 mg, about 75 mg to about 100 mg, about 0.5 mg, about 1 mg, about 2 mg, about 3 mg, about 4 mg, about 5 mg, about 7.5 mg, about 10 mg, about 15 mg, about 20 mg, about 25 mg, about 30 mg, about 40 mg, about 50 mg, about 75 mg, or about 100 mg.

In some embodiments, about 0.1% to about 1.5% (w/w) of a composition is cordyceps mushroom, e.g., about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, about 0.5% to about 0.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.5%, about 1% to about 1.4%, about 1% to about 1.3%, about 1% to about 1.2%, about 1% to about 1.1%, about 1.1% to about 1.5%, about 1.1% to about 1.4%, about 1.1% to about 1.3%, about 1.1% to about 1.2%, about 1.2% to about 1.5%, about 1.2% to about 1.4%, about 1.2% to about 1.3%, about 1.3% to about 1.5%, about 1.3% to about 1.4%, about 1.4% to about 1.5%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% is cordyceps prime.

In some embodiments, about 15 mg to about 2000 mg of cordyceps mushroom is present in a composition herein, e.g., about 15 mg to about 1500 mg, about 15 mg to about 1000 mg, about 15 mg to about 500 mg, about 15 mg to about 400 mg, about 15 mg to about 300 mg, about 15 mg to about 200 mg, about 15 mg to about 175 mg, about 15 mg to about 150 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 2000 mg, about 20 mg to about 1500 mg, about 20 mg to about 1000 mg, about 20 mg to about 500 mg, about 20 mg to about 400 mg, about 20 mg to about 300 mg, about 20 mg to about 200 mg, about 20 mg to about 175 mg, about 20 mg to about 150 mg, about 20 mg to about 125 mg, about 20 mg to about 100 mg, about 20 mg to about 75 mg, about 20 mg to about 50 mg, about 20 mg to about 25 mg, about 25 mg to about 2000 mg, about 25 mg to about 1500 mg, about 25 mg to about 1000 mg, about 25 mg to about 500 mg, about 25 mg to about 400 mg, about 25 mg to about 300 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 2000 mg, about 50 mg to about 1500 mg, about 50 mg to about 1000 mg, about 50 mg to about 500 mg, about 50 mg to about 400 mg, about 50 mg to about 300 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 2000 mg, about 75 mg to about 1500 mg, about 75 mg to about 1000 mg, about 75 mg to about 500 mg, about 75 mg to about 400 mg, about 75 mg to about 300 mg, about 75 mg to about 200 mg, about 75 mg to about 175 mg, about 75 mg to about 150 mg, about 75 mg to about 125 mg, about 75 mg to about 100 mg, about 100 mg to about 2000 mg, about 100 mg to about 1500 mg, about 100 mg to about 1000 mg, about 100 mg to about 500 mg, about 100 mg to about 400 mg, about 100 mg to about 300 mg, about 100 mg to about 200 mg, about 100 mg to about 175 mg, about 100 mg to about 150 mg, about 100 mg to about 125 mg, about 125 mg to about 2000 mg, about 125 mg to about 1500 mg, about 125 mg to about 1000 mg, about 125 mg to about 500 mg, about 125 mg to about 400 mg, about 125 mg to about 300 mg, about 125 mg to about 200 mg, about 125 mg to about 175 mg, about 125 mg to about 150 mg, about 150 mg to about 2000 mg, about 150 mg to about 1500 mg, about 150 mg to about 1000 mg, about 150 mg to about 500 mg, about 150 mg to about 400 mg, about 150 mg to about 300 mg, about 150 mg to about 200 mg, about 150 mg to about 175 mg, about 175 mg to about 2000 mg, about 175 mg to about 1500 mg, about 175 mg to about 1000 mg, about 175 mg to about 500 mg, about 175 mg to about 400 mg, about 175 mg to about 300 mg, about 175 mg to about 200 mg, about 200 mg to about 2000 mg, about 200 mg to about 1500 mg, about 200 mg to about 1000 mg, about 200 mg to about 500 mg, about 200 mg to about 400 mg, about 200 mg to about 300 mg, about 300 mg to about 2000 mg, about 300 mg to about 1500 mg, about 300 mg to about 1000 mg, about 300 mg to about 500 mg, about 300 mg to about 400 mg, about 400 mg to about 2000 mg, about 400 mg to about 1500 mg, about 400 mg to about 1000 mg, about 400 mg to about 500 mg, about 500 mg to about 2000 mg, about 500 mg to about 1500 mg, about 500 mg to about 1000 mg, about 1000 mg to about 2000 mg, about 1000 mg to about 1500 mg, about 1500 mg to about 2000 mg, about 15 mg, about 20 mg, about 25 mg, about 50 mg, about 75 mg, about 100 mg, about 125 mg, about 150 mg, about 175 mg, about 185 mg, about 200 mg, about 300 mg, about 400 mg, about 500 mg, about 750 mg, about 1000 mg, about 1500 mg, or about 2000 mg.

In some embodiments, about 5% to about 35% (w/w) of a composition is cordyceps mushroom, e.g., about 5% to about 30%, about 5% to about 25%, about 5% to about 20%, about 5% to about 15%, about 5% to about 10%, about 10% to about 35%, about 10% to about 30%, about 10% to about 25%, about 10% to about 20%, about 10% to about 15%, about 15% to about 35%, about 15% to about 30%, about 15% to about 25%, about 15% to about 20%, about 20% to about 35%, about 20% to about 30%, about 20% to about 25%, about 25% to about 35%, about 25% to about 30%, about 30% to about 35%, about 5%, about 10%, about 15%, about 20%, about 25%, about 30%, or about 35%.

Certain compositions herein are formulated with components demonstrating anti-inflammatory, antioxidative, anticancer, anti-obesity, and antidiabetic effects. In some such cases, the amount of cordyceps mushroom present in the composition is about 2 mg to about 8 mg, about 3 mg to about 7 mg, about 3 mg to about 6 mg, or about 5 mg. In some cases, the amount of cordyceps mushroom present in the composition is about 7 mg to about 13 mg, about 8 mg to about 12 mg, about 9 mg to about 11 mg, or about 10 mg. An example product is called RH1. In other such cases, the amount of cordyceps mushroom present in the composition is about 120 mg to about 260 mg, about 140 mg to about 240 mg, about 160 mg to about 220 mg, about 180 mg to about 200 mg, or about 190 mg. An example product is called RH2.

As described above, intake of cordyceps mushroom may lead to various effects for a person. For example, intake of cordyceps mushroom may lead to reduction of disease and inflammation as well as reducing the risk of cancer, obesity, and diabetes. While CordycepsPrime™ is described above as a source of cordyceps mushroom, CordycepsPrime™ is exemplary and other cordyceps mushrooms may be used.

### Vitamins (e.g., Vitamin B6)

In one aspect, provided are compositions comprising a vitamin (e.g., vitamin B6). Non-limiting examples of vitamins include vitamins: A, C, D, E, K, B1, B2, B3, B5, B6, B7, B9, and B12. Additional vitamins include pyridoxine, cyanocobalamin, ascorbic acid, biotin niacin, folate, pantothenic acid, riboflavin, thiamine, choline, carnitine, and folic acid. Many vitamins are required for the body to work properly, and serve important functions and work as, as non-limiting examples, antioxidants, coagulants, and means to regulate basic cellular metabolism. In some embodiments, provided are compositions comprising vitamin B6. Vitamin B6 is an important compound for general cellular metabolism and is a potent antioxidant.

In some embodiments, about 0.1 mg to about 15 mg of a vitamin (e.g. vitamin B6) is present in a composition herein, e.g., about 0.1 mg to about 14 mg, about 0.1 mg to about 13 mg, about 0.1 mg to about 12 mg, about 0.1 mg to about 11 mg, about 0.1 mg to about 10 mg, about 0.1 mg to about 9 mg, about 0.1 mg to about 8 mg, about 0.1 mg to about 7 mg, about 0.1 mg to about 6 mg, about 0.1 mg to about 5 mg, about 0.1 mg to about 4 mg, about 0.1 mg to about 3 mg, about 0.1 mg to about 2.5 mg, about 0.1 mg to about 2 mg, about 0.1 mg to about 1.5 mg, about 0.1 mg to about 1 mg, about 0.1 mg to about 0.5 mg, about 0.5 mg to about 15 mg, about 0.5 mg to about 14 mg, about 0.5 mg to about 13 mg, about 0.5 mg to about 12 mg, about 0.5 mg to about 11 mg, about 0.5 mg to about 11 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1 mg to about 15 mg, about 1 mg to about 14 mg, about 1 mg to about 13 mg, about 1 mg to about 12 mg, about 1 mg to about 11 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2.5 mg, about 1 mg to about 2 mg, about 1 mg to about 1.5 mg, about 1.5 mg to about 15 mg, about 1.5 mg to about 14 mg, about 1.5 mg to about 13 mg, about 1.5 mg to about 12 mg, about 1.5 mg to about 11 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2 mg to about 15 mg, about 2 mg to about 14 mg, about 2 mg to about 13 mg, about 2 mg to about 12 mg, about 2 mg to about 11 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 2 mg to about 2.5 mg, about 2.5 mg to about 15 mg, about 2.5 mg to about 14 mg, about 2.5 mg to about 13 mg, about 2.5 mg to about 12 mg, about 2.5 mg to about 11 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 15 mg, about 3.5 mg to about 14 mg, about 3.5 mg to about 13 mg, about 3.5 mg to about 12 mg, about 3.5 mg to about 11 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, about 14.5 mg to about 15 mg, about 0.5 mg, about 1.5 mg, about 2.5 mg, about 3.5 mg, about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg.

In some embodiments, about 0.01% to about 1.5% (w/w) of a composition is a vitamin (e.g. vitamin B6), e.g., about 0.01% to about 1.4%, about 0.01% to about 1.3%, about 0.01% to about 1.2%, about 0.01% to about 1.1%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01 to about 0.1%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, about 0.5% to about 0.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.5%, about 1% to about 1.4%, about 1% to about 1.3%, about 1% to about 1.2%, about 1% to about 1.1%, about 1.1% to about 1.5%, about 1.1% to about 1.4%, about 1.1% to about 1.3%, about 1.1% to about 1.2%, about 1.2% to about 1.5%, about 1.2% to about 1.4%, about 1.2% to about 1.3%, about 1.3% to about 1.5%, about 1.3% to about 1.4%, about 1.4% to about 1.5%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% is a vitamin.

Certain compositions herein are formulated with components important for cell metabolism and having antioxidative properties. In some such cases, the amount of the vitamin (e.g., vitamin B6) present in the composition is about 0.5 mg to about 2.5 mg, about 0.8 mg to about 2.2 mg, about 1.0 mg to about 1.6 mg, about 1.1 mg to about 1.5 mg, about 1.2 mg to about 1.4 mg, about 2 mg, about 1.9 mg, or about 1.3 mg. An example product is called RH1.

As described above, intake of vitamins may lead to various effects for a person. For example, intake of vitamins may lead to reduction of the risk of certain diseases. Intake of vitamins may also improve the ability of a person to regulate his or her metabolism.

### Nitric Oxide (Beet Root Powder)

In one aspect, provided are compositions comprising a nitric oxide source (e.g., beet root powder). Beet root powder is used as a food supplement due to its health promoting and disease preventing functionalities. Ingestion of beet root powder can serve as a natural method of increasing in vivo nitric oxide availability. Doing so is a potential strategy for use against pathologies such as hypertension and endothelial dysfunction. Furthermore, nitric oxide in beet root powder has been shown to open up blood vessels, facilitating blood flow to the brain, resulting in improvements to cognition.

In some embodiments, about 0.5 mg to about 100 mg of nitric oxide, (e.g., beet root powder) is present in a composition herein, e.g., about 0.5 mg to about 50 mg, about 0.5 mg to about 40 mg, about 0.5 mg to about 30 mg, about 0.5 mg to about 20 mg, about 0.5 mg to about 10 mg, about 0.5 mg to about 7.5 mg, about 0.5 mg to about 5 mg, about 0.5 to about 1 mg, about 1 mg to about 100 mg, about 1 mg to about 50 mg, about 1 mg to about 40 mg, about 1 mg to about 30 mg, about 1 mg to about 20 mg, about 1 mg to about 10 mg, about 1 mg to about 7.5 mg, about 1 mg to about 5 mg, about 5 mg to about 100 mg, about 5 mg to about 50 mg, about 5 mg to about 40 mg, about 5 mg to about 30 mg, about 5 mg to about 20 mg, about 5 mg to about 10 mg, about 5 mg to about 7.5 mg, about 7.5 mg to about 100 mg, about 7.5 mg to about 50 mg, about 7.5 mg to about 40 mg, about 7.5 mg to about 30 mg, about 7.5 mg to about 20 mg, about 7.5 mg to about 10 mg, about 10 mg to about 100 mg, about 10 mg to about 50 mg, about 10 mg to about 40 mg, about 10 mg to about 30 mg, about 10 mg to about 20 mg, about 20 mg to about 100 mg, about 20 mg to about 50 mg, about 20 mg to about 40 mg, about 20 mg to about 30 mg, about 30 mg to about 100 mg, about 30 mg to about 50 mg, about 30 mg to about 40 mg, about 40 mg to about 100 mg, about 40 mg to about 50 mg, about 50 mg to about 100 mg, about 0.5 mg, about 1 mg, about 5 mg, about 7.5 mg, about 10 mg, about 20 mg, about 30 mg, about 40 mg, about 50 mg, about 75 mg, or about 100 mg.

In some embodiments, about 0.1% to about 1.5% (w/w) of a composition comprises nitric oxide (e.g., beet root powder), e.g., about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, about 0.5% to about 0.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.5%, about 1% to about 1.4%, about 1% to about 1.3%, about 1% to about 1.2%, about 1% to about 1.1%, about 1.1% to about 1.5%, about 1.1% to about 1.4%, about 1.1% to about 1.3%, about 1.1% to about 1.2%, about 1.2% to about 1.5%, about 1.2% to about 1.4%, about 1.2% to about 1.3%, about 1.3% to about 1.5%, about 1.3% to about 1.4%, about 1.4% to about 1.5%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% is beet root powder.

Certain compositions herein are formulated with components useful for managing pathologies such as hypertension and endothelial dysfunction. In some such cases, the amount of nitric oxide (e.g., beet root powder) present in the composition is about 2 mg to about 8 mg, about 3 mg to about 7 mg, about 3 mg to about 6 mg, or about 5 mg. In some cases, the amount of nitric oxide (e.g., beet root powder) present in the composition is about 7 mg to about 13 mg, about 8 mg to about 12 mg, about 9 mg to about 11 mg, or about 10 mg. An example product is called RH1.

As described above, intake of nitric oxide may lead to various effects for a person. For example, intake of nitric oxide may lead to reduction of the risk of certain diseases as well as the reduction of hypertension and endothelial dysfunction. As another example, intake of nitric oxide may lead to more blood flow to the brain and increased cognition.

### Green Tea Extract

In one aspect, provided are compositions comprising a tea extract. Non-limiting examples of tea extracts include black tea, oolong tea, and green tea. In some embodiments, the tea extract is a green tea extract. Green tea extract may be a decaffeinated green tea polyphenol mixture isolated from the plant from which it originates, Camellia sinensis. Green tea extract has antiviral, antioxidant, and potential chemopreventive activities. Green tea extract contains compounds such as antioxidant compounds, flavonoids, and polyphenols such as catechins.

In some embodiments, about 0.5 mg to about 100 mg of tea extract is present in a composition herein, e.g., about 0.5 mg to about 75 mg, about 0.5 mg to about 50 mg, about 0.5 mg to about 40 mg, about 0.5 mg to about 30 mg, about 0.5 mg to about 20 mg, about 0.5 mg to about 15 mg, about 0.5 mg to about 10 mg, about 0.5 mg to about 7.5 mg, about 0.5 mg to about 5 mg, about 0.5 mg to about 1 mg, about 1 mg to about 100 mg, about 1 mg to about 75 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 7.5 mg, about 1 mg to about 5 mg, about 5 mg to about 100 mg, about 5 mg to about 75 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 5 mg to about 7.5 mg, about 7.5 mg to about 100 mg, about 7.5 mg to about 75 mg, about 7.5 mg to about 50 mg, about 7.5 mg to about 40 mg, about 7.5 mg to about 30 mg, about 7.5 mg to about 20 mg, about 7.5 mg to about 15 mg, about 7.5 mg to about 10 mg, about 10 mg to about 100 mg, about 10 mg to about 75 mg, about 10 mg to about 50 mg, about 10 mg to about 40 mg, about 10 mg to about 30 mg, about 10 mg to about 20 mg, about 10 mg to about 15 mg, about 15 mg to about 100 mg, about 15 mg to about 75 mg, about 15 mg to about 50 mg, about 15 mg to about 40 mg, about 15 mg to about 30 mg, about 15 mg to about 20 mg, about 20 mg to about 100 mg, about 20 mg to about 75 mg, about 20 mg to about 50 mg, about 20 mg to about 40 mg, about 20 mg to about 30 mg, about 30 mg to about 100 mg, about 30 mg to about 75 mg, about 30 mg to about 50 mg, about 30 mg to about 40 mg, about 40 mg to about 100 mg, about 40 mg to about 75 mg, about 40 mg to about 50 mg, about 50 mg to about 100 mg, about 50 mg to about 75 mg, about 75 mg to about 100 mg, about 0.5 mg, about 1 mg, about 2 mg, about 3 mg, about 4 mg, about 5 mg, about 7.5 mg, about 10 mg, about 15 mg, about 20 mg, about 25 mg, about 30 mg, about 40 mg, about 50 mg, about 75 mg, or about 100 mg.

In some embodiments, about 0.1% to about 1.5% (w/w) of a composition is tea extract, e.g., about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, about 0.5% to about 0.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.5%, about 1% to about 1.4%, about 1% to about 1.3%, about 1% to about 1.2%, about 1% to about 1.1%, about 1.1% to about 1.5%, about 1.1% to about 1.4%, about 1.1% to about 1.3%, about 1.1% to about 1.2%, about 1.2% to about 1.5%, about 1.2% to about 1.4%, about 1.2% to about 1.3%, about 1.3% to about 1.5%, about 1.3% to about 1.4%, about 1.4% to about 1.5%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% is tea extract.

Certain compositions herein are formulated with components having antiviral, antioxidant, and potential chemopreventive activities. In some such cases, the amount of tea extract (e.g., green tea extract) present in the composition is about 2 mg to about 8 mg, about 3 mg to about 7 mg, about 3 mg to about 6 mg, or about 5 mg. In some cases, the amount of tea extract (e.g., green tea extract) present in the composition is about 7 mg to about 13 mg, about 8 mg to about 12 mg, about 9 mg to about 11 mg, or about 10 mg. An example product is called RH1.

As described above, intake of tea extract may lead to various effects for a person. For example, intake of tea extract may reduce the risk of contracting certain diseases.

### Panax Ginseng

In one aspect, provided are compositions comprising Panax ginseng. Panax ginseng is a plant native to China, Korea, and Russia. It is a commonly used herbal remedy that is derived from the roots of the plant. The active components of Panax ginseng are ginsenosides. Research and clinical studies demonstrate its beneficial effects, such as anti-inflammatory, antioxidant, and anticancer effects. Furthermore, it is primarily used to improve exercise performance, psychologic function, conditions associated with diabetes, and immune function.

In some embodiments, about 0.5 mg to about 50 mg of Panax ginseng is present in a composition herein, e.g., about 0.5 mg to about 40 mg, about 0.5 mg to about 30 mg, about 0.5 mg to about 20 mg, about 0.5 mg to about 15 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 50 mg, about 2 mg to about 40 mg, about 2 mg to about 30 mg, about 2 mg to about 20 mg, about 2 mg to about 15 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 50 mg, about 3 mg to about 40 mg, about 3 mg to about 30 mg, about 3 mg to about 20 mg, about 3 mg to about 15 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 50 mg, about 4 mg to about 40 mg, about 4 mg to about 30 mg, about 4 mg to about 20 mg, about 4 mg to about 15 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 5 mg to about 9 mg, about 5 mg to about 8 mg, about 5 mg to about 7 mg, about 5 mg to about 6 mg, about 6 mg to about 50 mg, about 6 mg to about 40 mg, about 6 mg to about 30 mg, about 6 mg to about 20 mg, about 6 mg to about 15 mg, about 6 mg to about 10 mg, about 6 mg to about 9 mg, about 6 mg to about 8 mg, about 6 mg to about 7 mg, about 7 mg to about 50 mg, about 7 mg to about 40 mg, about 7 mg to about 30 mg, about 7 mg to about 20 mg, about 7 mg to about 15 mg, about 7 mg to about 10 mg, about 7 mg to about 9 mg, about 7 mg to about 8 mg, about 8 mg to about 50 mg, about 8 mg to about 40 mg, about 8 mg to about 30 mg, about 8 mg to about 20 mg, about 8 mg to about 15 mg, about 8 mg to about 10 mg, about 8 mg to about 9 mg, about 9 mg to about 50 mg, about 9 mg to about 40 mg, about 9 mg to about 30 mg, about 9 mg to about 20 mg, about 9 mg to about 15 mg, about 9 mg to about 10 mg, about 10 mg to about 50 mg, about 10 mg to about 40 mg, about 10 mg to about 30 mg, about 10 mg to about 20 mg, about 10 mg to about 15 mg, about 15 mg to about 50 mg, about 15 mg to about 40 mg, about 15 mg to about 30 mg, about 15 mg to about 20 mg, about 20 mg to about 50 mg, about 20 mg to about 40 mg, about 20 mg to about 30 mg, about 30 mg to about 50 mg, about 30 mg to about 40 mg, about 40 mg to about 50 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 30 mg, about 40 mg, or about 50 mg.

In some embodiments, about 0.1% to about 1% (w/w) of a composition is Panax ginseng, e.g., about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, about 0.9% to about 1%, about 0.1%, about 0.2%, about 0.3%, about 0.4%, about 0.5%, about 0.6%, about 0.7%, about 0.8%, about 0.9%, or about 1% is Panax ginseng.

Certain compositions herein are formulated with components improving psychological function, exercise performance, and immune function. In some such cases, the amount of Panax ginseng present in the composition is about 2 mg to about 8 mg, about 3 mg to about 7 mg, about 3 mg to about 6 mg, or about 5 mg. An example product is called RH1.

As described above, intake of Panax ginseng may lead to various effects for a person. For example, intake of Panax ginseng may lead to a reduction of inflammation as well as reduce the risk of certain diseases and cancer. Intake of Panax ginseng may also improve symptoms of diabetes and may improve immune functions.

### Phosphatidylserine (Sunflower Powder)

In one aspect, provided are compositions comprising phosphatidylserine (e.g., sunflower powder). Phosphatidylserine can be derived from sunflower seeds, specifically sunflower lecithin. Research and clinical studies have demonstrated that phosphatidylserine improves memory and has improved symptoms of behavioral disorders such as ADHD in children.

In some embodiments, about 12.5 mg to about 1250 mg of phosphatidylserine (e.g., sunflower powder) is present in a composition herein, e.g., about 12.5 mg to about 1000 mg, about 12.5 mg to about 750 mg, about 12.5 mg to about 500 mg, about 12.5 mg to about 250 mg, about 12.5 mg to about 200 mg, about 12.5 mg to about 150 mg, about 12.5 mg to about 125 mg, about 12.5 mg to about 100 mg, about 12.5 mg to about 75 mg, about 12.5 mg to about 50 mg, about 12.5 mg to about 25 mg, about 25 mg to about 1250 mg, about 25 mg to about 1000 mg, about 25 mg to about 750 mg, about 25 mg to about 500 mg, about 25 mg to about 250 mg, about 25 mg to about 200 mg, about 25 mg to about 150 mg, about 25 mg to about 125 mg, about 25 mg to about 100 mg, about 25 mg to about 75 mg, about 25 mg to about 50 mg, about 50 mg to about 1250 mg, about 50 mg to about 1000 mg, about 50 mg to about 750 mg, about 50 mg to about 500 mg, about 50 mg to about 250 mg, about 50 mg to about 200 mg, about 50 mg to about 150 mg, about 50 mg to about 125 mg, about 50 mg to about 100 mg, about 50 mg to about 75 mg, about 75 mg to about 1250 mg, about 75 mg to about 1000 mg, about 75 mg to about 750 mg, about 75 mg to about 500 mg, about 75 mg to about 250 mg, about 75 mg to about 200 mg, about 75 mg to about 150 mg, about 75 mg to about 125 mg, about 75 mg to about 100 mg, about 100 mg to about 1250 mg, about 100 mg to about 1000 mg, about 100 mg to about 750 mg, about 100 mg to about 500 mg, about 100 mg to about 250 mg, about 100 mg to about 200 mg, about 100 mg to about 150 mg, about 100 mg to about 125 mg, about 125 mg to about 1250 mg, about 125 mg to about 1000 mg, about 125 mg to about 750 mg, about 125 mg to about 500 mg, about 125 mg to about 250 mg, about 125 mg to about 200 mg, about 125 mg to about 150 mg, about 150 mg to about 1250 mg, about 150 mg to about 1000 mg, about 150 mg to about 750 mg, about 150 mg to about 500 mg, about 150 mg to about 250 mg, about 150 mg to about 200 mg, 200 mg to about 1250 mg, about 200 mg to about 1000 mg, about 200 mg to about 750 mg, about 200 mg to about 500 mg, about 200 mg to about 250 mg, 250 mg to about 1250 mg, about 250 mg to about 1000 mg, about 250 mg to about 750 mg, about 250 mg to about 500 mg, 500 mg to about 1250 mg, about 500 mg to about 1000 mg, about 500 mg to about 750 mg, 750 mg to about 1250 mg, about 750 mg to about 1000 mg, about 1000 mg to about 1250 mg, about 12.5 mg, about 25 mg, about 50 mg, about 75 mg, about 100 mg, about 125 mg, about 150 mg, about 200 mg, about 250 mg, about 500 mg, about 750 mg, about 1000 mg, or about 1250 mg.

In some embodiments, about 5% (w/w) to about 40% (w/w) of a composition is phosphatidylserine (e.g., sunflower powder), e.g., about 5% (w/w) to about 35% (w/w), about 5% (w/w) to about 30% (w/w), about 5% (w/w) to about 25% (w/w), about 5% (w/w) to about 20% (w/w), about 5% (w/w) to about 15% (w/w), about 5% (w/w) to about 10% (w/w), about 10% (w/w) to about 40% (w/w), about 10% (w/w) to about 35% (w/w), about 10% (w/w) to about 30% (w/w), about 10% (w/w) to about 25% (w/w), about 10% (w/w) to about 20% (w/w), about 10% (w/w) to about 15% (w/w), about 15% (w/w) to about 40% (w/w), about 15% (w/w) to about 35% (w/w), about 15% (w/w) to about 30% (w/w), about 15% (w/w) to about 25% (w/w), about 15% (w/w) to about 20% (w/w), about 20% (w/w) to about 40% (w/w), about 20% (w/w) to about 35% (w/w), about 20% (w/w) to about 30% (w/w), about 20% (w/w) to about 25% (w/w), about 25% (w/w) to about 40% (w/w), about 25% (w/w) to about 35% (w/w), about 30% (w/w) to about 30% (w/w), about 30% (w/w) to about 40% (w/w), about 30% (w/w) to about 35% (w/w), about 35% (w/w) to about 40% (w/w), about 5% (w/w), about 10% (w/w), about 15% (w/w), about 20% (w/w), about 25% (w/w), about 30% (w/w), about 35% (w/w), or about 40% (w/w).

In some embodiments, about 1 mg to about 125 mg of phosphatidylserine (e.g., sunflower powder) is present in a composition herein, e.g., about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 100 mg, about 75 mg to about 100 mg, about 100 mg to about 125 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, or about 125 mg.

In some embodiments, about 0.5% to about 10% (w/w) of a composition is phosphatidylserine (e.g., sunflower powder), e.g., about 0.5% (w/w) to about 9% (w/w), about 0.5% (w/w) to about 8% (w/w), about 0.5% (w/w) to about 7% (w/w), about 0.5% (w/w) to about 6% (w/w), about 0.5% (w/w) to about 5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1.5% (w/w) to about 10% (w/w), about 1.5% (w/w) to about 9% (w/w), about 1.5% (w/w) to about 8% (w/w), about 1.5% (w/w) to about 7% (w/w), about 1.5% (w/w) to about 6% (w/w), about 1.5% (w/w) to about 5% (w/w), about 1.5% (w/w) to about 4% (w/w), about 1.5% (w/w) to about 3% (w/w), about 1.5% (w/w) to about 2% (w/w), about 2.5% (w/w) to about 10% (w/w), about 2.5% (w/w) to about 9% (w/w), about 2.5% (w/w) to about 8% (w/w), about 2.5% (w/w) to about 7% (w/w), about 2.5% (w/w) to about 6% (w/w), about 2.5% (w/w) to about 5% (w/w), about 2.5% (w/w) to about 4% (w/w), about 2.5% (w/w) to about 3% (w/w), about 3.5% (w/w) to about 10% (w/w), about 3.5% (w/w) to about 9% (w/w), about 3.5% (w/w) to about 8% (w/w), about 3.5% (w/w) to about 7% (w/w), about 3.5% (w/w) to about 6% (w/w), about 3.5% (w/w) to about 5% (w/w), about 3.5% (w/w) to about 4% (w/w), about 4.5% (w/w) to about 10% (w/w), about 4.5% (w/w) to about 9% (w/w), about 4.5% (w/w) to about 8% (w/w), about 4.5% (w/w) to about 7% (w/w), about 4.5% (w/w) to about 6% (w/w), about 4.5% (w/w) to about 5% (w/w), about 5.5% (w/w) to about 10% (w/w), about 5.5% (w/w) to about 9% (w/w), about 5.5% (w/w) to about 8% (w/w), about 5.5% (w/w) to about 7% (w/w), about 5.5% (w/w) to about 6% (w/w), about 6.5% (w/w) to about 10% (w/w), about 6.5% (w/w) to about 9% (w/w), about 6.5% (w/w) to about 8% (w/w), about 6.5% (w/w) to about 7% (w/w), about 7.5% (w/w) to about 10% (w/w), about 7.5% (w/w) to about 9% (w/w), about 7.5% (w/w) to about 8% (w/w), about 8.5% (w/w) to about 10% (w/w), about 8.5% (w/w) to about 9% (w/w), about 9.5% (w/w) to about 10% (w/w), about 0.5% (w/w), about 1.5% (w/w), about 2.5% (w/w), about 3.5% (w/w), about 4.5% (w/w), about 5.5% (w/w), about 6.5% (w/w), about 7.5% (w/w), about 8.5% (w/w), or about 9.5% (w/w) is phosphatidylserine (sunflower powder).

Certain compositions herein are formulated with components improving memory and cognitive function. In some such cases, the amount of phosphatidylserine (e.g., sunflower powder) present is about 50 mg to about 200 mg, about 75 mg to about 175 mg, about 100 mg to about 150 mg, or about 125 mg. In some such cases, the amount of phosphatidylserine (sunflower powder) present is about 5 mg to about 20 mg, about 7.5 mg to about 17.5 mg, about 10 mg to about 15 mg, or about 12.5 mg. Example products include RH2 and RH3.

As described above, intake of phosphatidylserine may lead to various effects for a person. For example, intake of phosphatidylserine may lead to improved memory and improved symptoms of behavioral disorders.

### Minerals (e.g., Magnesium Glycinate, Zinc)

In one aspect, provided are compositions comprising a mineral. Non-limiting examples of supplemental minerals include magnesium, calcium, choline, chromium, cesium, copper, fluoride, iodine, iron, manganese, molybdenum, potassium, selenium, and zinc. In some embodiments, provided are compositions comprising magnesium glycinate. Magnesium glycinate is a supplemental form of magnesium. Magnesium is essential for maintaining normal organ and cellular function. An imbalance of magnesium is associated with disorders such as skeletal deformities, metabolic syndrome, and cardiovascular diseases. Supplemental magnesium glycinate is formed from elemental magnesium and the amino acid, glycine. Magnesium glycinate has been shown to facilitate the production of GABA, and thereby may calm the nervous system. In some embodiments, provided are compositions comprising zinc. Zinc is a nutrient found throughout the human body and is important for immune system and metabolism function. Growth retardation, loss of appetite, an impaired immune system, hair loss, delayed sexual maturation, impotence, hypogonadism in males, taste abnormalities, diarrhea, mental lethargy, and delayed wound healing have all been associated with deficient zinc levels. Zinc deficiency in humans is prevalent, affecting more than 2 billion people. As a result, studies suggest use of zinc supplementation for the listed conditions.

In some embodiments, about 5 mg to about 500 mg of mineral (e.g., magnesium glycinate) is present in a composition herein, e.g., about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 500 mg, about 30 mg to about 450 mg, about 30 mg to about 400 mg, about 30 mg to about 350 mg, about 30 mg to about 300 mg, about 30 mg to about 250 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 150 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 150 mg, about 60 mg to about 100 mg, about 60 mg to about 90 mg, about 60 mg to about 80 mg, about 60 mg to about 70 mg, about 70 mg to about 500 mg, about 70 mg to about 450 mg, about 70 mg to about 400 mg, about 70 mg to about 350 mg, about 70 mg to about 300 mg, about 70 mg to about 250 mg, about 70 mg to about 200 mg, about 70 mg to about 150 mg, about 70 mg to about 100 mg, about 70 mg to about 90 mg, about 70 mg to about 80 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 150 mg, about 80 mg to about 100 mg, about 80 mg to about 90 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 90 mg to about 100 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 300 mg, about 300 mg to about 500 mg, about 300 mg to about 450 mg, about 300 mg to about 400 mg, about 300 mg to about 350 mg, about 350 mg to about 500 mg, about 300 mg to about 450 mg, about 300 mg to about 350 mg, about 350 mg to about 500 mg, about 350 mg to about 450 mg, about 350 mg to about 400 mg, about 400 mg to about 500 mg, about 400 mg to about 450 mg, about 450 mg to about 500 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg.

In some embodiments, about 0.5% to about 11% (w/w) of a composition is mineral (e.g., magnesium glycinate), e.g., about 0.5% (w/w) to about 10% (w/w), about 0.5% (w/w) to about 9% (w/w), about 0.5% (w/w) to about 8% (w/w), about 0.5% (w/w) to about 7% (w/w), about 0.5% (w/w) to about 6% (w/w), about 0.5% (w/w) to about 5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1.5% (w/w) to about 11% (w/w), about 1.5% (w/w) to about 10% (w/w), about 1.5% (w/w) to about 9% (w/w), about 1.5% (w/w) to about 8% (w/w), about 1.5% (w/w) to about 7% (w/w), about 1.5% (w/w) to about 6% (w/w), about 1.5% (w/w) to about 5% (w/w), about 1.5% (w/w) to about 4% (w/w), about 1.5% (w/w) to about 3% (w/w), about 1.5% (w/w) to about 2% (w/w), about 2.5% (w/w) to about 11% (w/w), about 2.5% (w/w) to about 10% (w/w), about 2.5% (w/w) to about 9% (w/w), about 2.5% (w/w) to about 8% (w/w), about 2.5% (w/w) to about 7% (w/w), about 2.5% (w/w) to about 6% (w/w), about 2.5% (w/w) to about 5% (w/w), about 2.5% (w/w) to about 4% (w/w), about 2.5% (w/w) to about 3% (w/w), about 3.5% (w/w) to about 11% (w/w), about 3.5% (w/w) to about 10% (w/w), about 3.5% (w/w) to about 9% (w/w), about 3.5% (w/w) to about 8% (w/w), about 3.5% (w/w) to about 7% (w/w), about 3.5% (w/w) to about 6% (w/w), about 3.5% (w/w) to about 5% (w/w), about 3.5% (w/w) to about 4% (w/w), about 4.5% (w/w) to about 11% (w/w), about 4.5% (w/w) to about 10% (w/w), about 4.5% (w/w) to about 9% (w/w), about 4.5% (w/w) to about 8% (w/w), about 4.5% (w/w) to about 7% (w/w), about 4.5% (w/w) to about 6% (w/w), about 4.5% (w/w) to about 5% (w/w), about 5.5% (w/w) to about 11% (w/w), about 5.5% (w/w) to about 10% (w/w), about 5.5% (w/w) to about 9% (w/w), about 5.5% (w/w) to about 8% (w/w), about 5.5% (w/w) to about 7% (w/w), about 5.5% (w/w) to about 6% (w/w), about 6.5% (w/w) to about 11% (w/w), about 6.5% (w/w) to about 10% (w/w), about 6.5% (w/w) to about 9% (w/w), about 6.5% (w/w) to about 8% (w/w), about 6.5% (w/w) to about 7% (w/w), about 7.5% (w/w) to about 11% (w/w), about 7.5% (w/w) to about 10% (w/w), about 7.5% (w/w) to about 9% (w/w), about 7.5% (w/w) to about 8% (w/w), about 8.5% (w/w) to about 11% (w/w), about 8.5% (w/w) to about 10% (w/w), about 8.5% (w/w) to about 9% (w/w), about 9.5% (w/w) to about 11% (w/w), about 9.5% (w/w) to about 10% (w/w), about 10.5% (w/w) to about 11% (w/w), about 0.5% (w/w), about 1.5% (w/w), about 2.5% (w/w), about 3.5% (w/w), about 4.5% (w/w), about 5.5% (w/w), about 6.5% (w/w), about 7.5% (w/w), about 8.5% (w/w), about 9.5% (w/w), or about 10.5% (w/w) is mineral (e.g., magnesium glycinate).

In some embodiments, about 0.5 mg to about 125 mg of mineral (e.g., zinc) is present in a composition herein, e.g., about 0.5 mg to about 100 mg, about 0.5 mg to about 75 mg, about 0.5 mg to about 50 mg, about 0.5 mg to about 25 mg, about 0.5 mg to about 20 mg, about 0.5 mg to about 15 mg, about 0.5 mg to about 12.5 mg, about 0.5 mg to about 10 mg, about 0.5 mg to about 7.5 mg, about 0.5 mg to about 5 mg, about 0.5 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 125 mg, about 75 mg to about 100 mg, about 100 mg to about 125 mg, about 0.5 mg, about 2.5 mg, about 5 mg, about 7.5 mg, about 8 mg, about 10 mg, about 11 mg, about 12.5 mg, about 15 mg, about 20 mg, about 25 mg, about 50 mg, about 75 mg, about 100 mg, or about 125 mg.

In some embodiments, about 0.1% to about 2% (w/w) of a composition is mineral (e.g., zinc), e.g., about 0.1% to about 1.9%, about 0.1% to about 1.8%, about 0.1% to about 1.7%, about 0.1% to about 1.6%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, 0.1% to about 0.6%, 0.1% to about 0.5%, 0.1% to about 0.4%, 0.1% to about 0.3%, 0.1% to about 0.2%, about 0.2% to about 2%, about 0.2% to about 1.9%, about 0.2% to about 1.8%, about 0.2% to about 1.7%, about 0.2% to about 1.6%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, 0.2% to about 0.6%, 0.2% to about 0.5%, 0.2% to about 0.4%, 0.2% to about 0.3%, about 0.3% to about 2%, about 0.3% to about 1.9%, about 0.3% to about 1.8%, about 0.3% to about 1.7%, about 0.3% to about 1.6%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, 0.3% to about 0.6%, 0.3% to about 0.5%, 0.3% to about 0.4%, about 0.4% to about 2%, about 0.4% to about 1.9%, about 0.4% to about 1.8%, about 0.4% to about 1.7%, about 0.4% to about 1.6%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, 0.4% to about 0.6%, 0.4% to about 0.5%, about 0.5% to about 2%, about 0.5% to about 1.9%, about 0.5% to about 1.8%, about 0.5% to about 1.7%, about 0.5% to about 1.6%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, 0.5% to about 0.6%, about 0.6% to about 2%, about 0.6% to about 1.9%, about 0.6% to about 1.8%, about 0.6% to about 1.7%, about 0.6% to about 1.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 2%, about 0.7% to about 1.9%, about 0.7% to about 1.8%, about 0.7% to about 1.7%, about 0.7% to about 1.6%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 2%, about 0.8% to about 1.9%, about 0.8% to about 1.8%, about 0.8% to about 1.7%, about 0.8% to about 1.6%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 2%, about 0.9% to about 1.9%, about 0.9% to about 1.8%, about 0.9% to about 1.7%, about 0.9% to about 1.6%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 2%, about 1% to about 1.9%, about 1% to about 1.8%, about 1% to about 1.7%, about 1% to about 1.6%, about 1% to about 1.5%, about 1% to about 1.4%, about 1% to about 1.3%, about 1% to about 1.2%, about 1% to about 1.1%, about 1.1% to about 2%, about 1.1% to about 1.9%, about 1.1% to about 1.8%, about 1.1% to about 1.7%, about 1.1% to about 1.6%, about 1.1% to about 1.5%, about 1.1% to about 1.4%, about 1.1% to about 1.3%, about 1.1% to about 1.2%, about 1.2% to about 2%, about 1.2% to about 1.9%, about 1.2% to about 1.8%, about 1.2% to about 1.7%, about 1.2% to about 1.6%, about 1.2% to about 1.5%, about 1.2% to about 1.4%, about 1.2% to about 1.3%, about 1.3% to about 2%, about 1.3% to about 1.9%, about 1.3% to about 1.8%, about 1.3% to about 1.7%, about 1.3% to about 1.6%, about 1.3% to about 1.5%, about 1.3% to about 1.4%, about 1.4% to about 2%, about 1.4% to about 1.9%, about 1.4% to about 1.8%, about 1.4% to about 1.7%, about 1.4% to about 1.6%, about 1.4% to about 1.5%, about 1.5% to about 2%, about 1.5% to about 1.9%, about 1.5% to about 1.8%, about 1.5% to about 1.7%, about 1.5% to about 1.6%, about 1.6% to about 2%, about 1.6% to about 1.9%, about 1.6% to about 1.8%, about 1.6% to about 1.7%, about 1.7% to about 2%, about 1.7% to about 1.9%, about 1.7% to about 1.8%, about 1.8% to about 2%, about 1.8% to about 1.9%, about 1.9% to about 2%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%. is mineral (e.g., zinc).

Certain compositions herein are formulated with components for maintaining normal organ and cellular function. In some such cases, the amount of mineral (e.g., magnesium glycinate) present in the composition is about 20 mg to about 80 mg, about 30 mg to about 70 mg, about 40 mg to about 60 mg, or about 50 mg. An example product is called RH2.

Certain compositions herein are formulated with components important to metabolism and immune system function. In some such cases, the amount of mineral (e.g., zinc) present in the composition is about 8 mg to about 14 mg, about 9 mg to about 13 mg, about 10 mg to about 12 mg, or about 11 mg. In other such cases, the amount of mineral (e.g., zinc) present in the composition is about 5 mg to about 11 mg, about 6 mg to about 10 mg, about 7 mg to about 9 mg, or about 8 mg. An example product is called RH2.

As described above, intake of certain minerals may lead to various effects for a person. For example, intake of certain minerals may improve the ability to maintain normal organ and cellular function, and may reduce the risk of skeletal deformities, metabolic syndrome, and cardiovascular diseases. Intake of certain minerals may also reduce the risk of growth retardation, hair loss, delayed sexual maturation, impotence, and hypogonadism, and may prevent or reduce the symptoms of loss of appetite, diarrhea, mental lethargy, and delayed wound healing. Intake of certain minerals may also improve immune system and metabolic functions of the person.

### Magnolia Officinalis

In one aspect, provided are compositions comprising Magnolia officinalis. Magnolia officinalis is an herb used in traditional medicine. It is derived from bark from the magnolia tree. The bark contains active compounds honokiol and magnolol. These compounds may help to reduce inflammation and stress. Studies have shown that Magnolia officinalis can relieve vasomotor symptoms, and help individuals manage weight and maintain oral health.

In some embodiments, about 5 mg to about 500 mg of Magnolia officinalis is present in a composition herein, e.g., about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 500 mg, about 30 mg to about 450 mg, about 30 mg to about 400 mg, about 30 mg to about 350 mg, about 30 mg to about 300 mg, about 30 mg to about 250 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 150 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 150 mg, about 60 mg to about 100 mg, about 60 mg to about 90 mg, about 60 mg to about 80 mg, about 60 mg to about 70 mg, about 70 mg to about 500 mg, about 70 mg to about 450 mg, about 70 mg to about 400 mg, about 70 mg to about 350 mg, about 70 mg to about 300 mg, about 70 mg to about 250 mg, about 70 mg to about 200 mg, about 70 mg to about 150 mg, about 70 mg to about 100 mg, about 70 mg to about 90 mg, about 70 mg to about 80 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 150 mg, about 80 mg to about 100 mg, about 80 mg to about 90 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 90 mg to about 100 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 300 mg, about 300 mg to about 500 mg, about 300 mg to about 450 mg, about 300 mg to about 400 mg, about 300 mg to about 350 mg, about 350 mg to about 500 mg, about 300 mg to about 450 mg, about 300 mg to about 350 mg, about 350 mg to about 500 mg, about 350 mg to about 450 mg, about 350 mg to about 400 mg, about 400 mg to about 500 mg, about 400 mg to about 450 mg, about 450 mg to about 500 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg.

In some embodiments, about 0.5% to about 11% (w/w) of a composition is Magnolia officinalis, e.g., about 0.5% (w/w) to about 10% (w/w), about 0.5% (w/w) to about 9% (w/w), about 0.5% (w/w) to about 8% (w/w), about 0.5% (w/w) to about 7% (w/w), about 0.5% (w/w) to about 6% (w/w), about 0.5% (w/w) to about 5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1.5% (w/w) to about 11% (w/w), about 1.5% (w/w) to about 10% (w/w), about 1.5% (w/w) to about 9% (w/w), about 1.5% (w/w) to about 8% (w/w), about 1.5% (w/w) to about 7% (w/w), about 1.5% (w/w) to about 6% (w/w), about 1.5% (w/w) to about 5% (w/w), about 1.5% (w/w) to about 4% (w/w), about 1.5% (w/w) to about 3% (w/w), about 1.5% (w/w) to about 2% (w/w), about 2.5% (w/w) to about 11% (w/w), about 2.5% (w/w) to about 10% (w/w), about 2.5% (w/w) to about 9% (w/w), about 2.5% (w/w) to about 8% (w/w), about 2.5% (w/w) to about 7% (w/w), about 2.5% (w/w) to about 6% (w/w), about 2.5% (w/w) to about 5% (w/w), about 2.5% (w/w) to about 4% (w/w), about 2.5% (w/w) to about 3% (w/w), about 3.5% (w/w) to about 11% (w/w), about 3.5% (w/w) to about 10% (w/w), about 3.5% (w/w) to about 9% (w/w), about 3.5% (w/w) to about 8% (w/w), about 3.5% (w/w) to about 7% (w/w), about 3.5% (w/w) to about 6% (w/w), about 3.5% (w/w) to about 5% (w/w), about 3.5% (w/w) to about 4% (w/w), about 4.5% (w/w) to about 11% (w/w), about 4.5% (w/w) to about 10% (w/w), about 4.5% (w/w) to about 9% (w/w), about 4.5% (w/w) to about 8% (w/w), about 4.5% (w/w) to about 7% (w/w), about 4.5% (w/w) to about 6% (w/w), about 4.5% (w/w) to about 5% (w/w), about 5.5% (w/w) to about 11% (w/w), about 5.5% (w/w) to about 10% (w/w), about 5.5% (w/w) to about 9% (w/w), about 5.5% (w/w) to about 8% (w/w), about 5.5% (w/w) to about 7% (w/w), about 5.5% (w/w) to about 6% (w/w), about 6.5% (w/w) to about 11% (w/w), about 6.5% (w/w) to about 10% (w/w), about 6.5% (w/w) to about 9% (w/w), about 6.5% (w/w) to about 8% (w/w), about 6.5% (w/w) to about 7% (w/w), about 7.5% (w/w) to about 11% (w/w), about 7.5% (w/w) to about 10% (w/w), about 7.5% (w/w) to about 9% (w/w), about 7.5% (w/w) to about 8% (w/w), about 8.5% (w/w) to about 11% (w/w), about 8.5% (w/w) to about 10% (w/w), about 8.5% (w/w) to about 9% (w/w), about 9.5% (w/w) to about 11% (w/w), about 9.5% (w/w) to about 10% (w/w), about 0.5% (w/w), about 1.5% (w/w), about 2.5% (w/w), about 3.5% (w/w), about 4.5% (w/w), about 5.5% (w/w), about 6.5% (w/w), about 7.5% (w/w), about 8.5% (w/w), about 9.5% (w/w), or about 10.5% (w/w) is Magnolia officinalis.

In some embodiments, about 1 mg to about 125 mg of Magnolia officinalis is present in a composition herein, e.g., about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 100 mg, about 75 mg to about 100 mg, about 100 mg to about 125 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, or about 125 mg.

In some embodiments, about 0.5% to about 10% (w/w) of a composition is Magnolia officinalis, e.g., about 0.5% (w/w) to about 9% (w/w), about 0.5% (w/w) to about 8% (w/w), about 0.5% (w/w) to about 7% (w/w), about 0.5% (w/w) to about 6% (w/w), about 0.5% (w/w) to about 5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1.5% (w/w) to about 10% (w/w), about 1.5% (w/w) to about 9% (w/w), about 1.5% (w/w) to about 8% (w/w), about 1.5% (w/w) to about 7% (w/w), about 1.5% (w/w) to about 6% (w/w), about 1.5% (w/w) to about 5% (w/w), about 1.5% (w/w) to about 4% (w/w), about 1.5% (w/w) to about 3% (w/w), about 1.5% (w/w) to about 2% (w/w), about 2.5% (w/w) to about 10% (w/w), about 2.5% (w/w) to about 9% (w/w), about 2.5% (w/w) to about 8% (w/w), about 2.5% (w/w) to about 7% (w/w), about 2.5% (w/w) to about 6% (w/w), about 2.5% (w/w) to about 5% (w/w), about 2.5% (w/w) to about 4% (w/w), about 2.5% (w/w) to about 3% (w/w), about 3.5% (w/w) to about 10% (w/w), about 3.5% (w/w) to about 9% (w/w), about 3.5% (w/w) to about 8% (w/w), about 3.5% (w/w) to about 7% (w/w), about 3.5% (w/w) to about 6% (w/w), about 3.5% (w/w) to about 5% (w/w), about 3.5% (w/w) to about 4% (w/w), about 4.5% (w/w) to about 10% (w/w), about 4.5% (w/w) to about 9% (w/w), about 4.5% (w/w) to about 8% (w/w), about 4.5% (w/w) to about 7% (w/w), about 4.5% (w/w) to about 6% (w/w), about 4.5% (w/w) to about 5% (w/w), about 5.5% (w/w) to about 10% (w/w), about 5.5% (w/w) to about 9% (w/w), about 5.5% (w/w) to about 8% (w/w), about 5.5% (w/w) to about 7% (w/w), about 5.5% (w/w) to about 6% (w/w), about 6.5% (w/w) to about 10% (w/w), about 6.5% (w/w) to about 9% (w/w), about 6.5% (w/w) to about 8% (w/w), about 6.5% (w/w) to about 7% (w/w), about 7.5% (w/w) to about 10% (w/w), about 7.5% (w/w) to about 9% (w/w), about 7.5% (w/w) to about 8% (w/w), about 8.5% (w/w) to about 10% (w/w), about 8.5% (w/w) to about 9% (w/w), about 9.5% (w/w) to about 10% (w/w), about 0.5% (w/w), about 1.5% (w/w), about 2.5% (w/w), about 3.5% (w/w), about 4.5% (w/w), about 5.5% (w/w), about 6.5% (w/w), about 7.5% (w/w), about 8.5% (w/w), or about 9.5% (w/w).

Certain compositions herein are formulated with components for reducing inflammation and stress. In some such cases, the amount of Magnolia officinalis present in the composition is about 20 mg to about 80 mg, about 30 mg to about 70 mg, about 40 mg to about 60 mg, or about 50 mg. An example product is called RH2.

Certain compositions herein are formulated to improve the sleep health of a person. In some such cases, the amount of Magnolia officinalis present in the composition is about 9.5 mg to about 15.5 mg, about 10.5 mg to about 14.5 mg, about 11.5 mg to about 13.5 mg, or about 12.5 mg. An example product is called RH3.

As described above, intake of Magnolia officinalis may lead to various effects for a person. For example, intake of Magnolia officinalis may reduce inflammation and stress as well as help regulate a person's weight and maintain a person's oral health.

### Ginger

In one aspect, provided are compositions comprising ginger. Ginger is a well-known plant used commonly as a flavoring agent and for traditional remedies. Classes of ginger compounds having bioactive properties include gingerols, shogaols, zingiberene, zingerone, terpenes and other vitamins and minerals. Gingerols are considered the primary components and have been shown to have associated bioactivities such as antioxidant, antimicrobial, and anti-neuroinflammation, among others.

In some embodiments, about 1 mg to about 125 mg of ginger is present in a composition herein, e.g., about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 100 mg, about 75 mg to about 100 mg, about 100 mg to about 125 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, or about 125 mg.

In some embodiments, about 0.1% to about 2.5% (w/w) of a composition is ginger, e.g., about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, about 2.1% to about 2.5%, about 0.1%, about 0.5%, about 0.9%, about 1.3%, about 1.7%, about 2.1%, or about 2.5% of the composition is ginger.

Certain compositions herein are formulated with components having antioxidant, antimicrobial, and anti-neuroinflammation properties. In some such cases, the amount of ginger present in the composition is about 5 mg to about 21 mg, about 9 mg to about 17 mg, about 10 mg to about 15 mg, or about 12.5 mg. An example product is called RH2.

As described above, intake of ginger may lead to various effects for a person. For example, intake of ginger may reduce the risk of certain diseases or infections as well as reducing neuroinflammation. Additionally, ginger may be used as a flavoring agent.

### Horny Goat Weed

In one aspect, provided are compositions comprising horny goat weed. Horny goat weed, or epimedium, is a Chinese herbal remedy. Most of its medicinal properties have been attributed to its flavonoid glycosides, especially Icariin. Horny goat weed has been used to combat diseases of the eye and kidney, impotence, arthritis, asthma, and hypertension.

In some embodiments, about 1 mg to about 125 mg of horny goat weed is present in a composition herein, e.g., about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 100 mg, about 75 mg to about 100 mg, about 100 mg to about 125 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, or about 125 mg.

In some embodiments, about 0.1% to about 2.5% (w/w) of a composition is horny goat weed, e.g., about 0.1% to about 2.5%, about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, about 2.1% to about 2.5%, about 0.1%, about 0.5%, about 0.9%, about 1.3%, about 1.7%, about 2.1%, or about 2.5% of the composition is horny goat weed.

Certain compositions herein are formulated with components used to combat diseases of the eye and kidney, impotence, arthritis, asthma, and hypertension. In some such cases, the amount of horny goat weed present in the composition is about 5 mg to about 21 mg, about 9 mg to about 17 mg, about 10 mg to about 15 mg, or about 12.5 mg. An example product is called RH2.

As described above, intake of horny goat week may lead to various effects for a person. For example, horny goat week may improve symptoms of certain eye and kidney diseases, arthritis, impotence, asthma, and hypertension.

### Sclareolides (Sage Leaf Powder)

In one aspect, provided are compositions comprising sclareolides (e.g., sage leaf powder). Sage leaf powder is an herb with various bioactivities and used in traditional remedies. Studies suggest sage, from the Salvia species, has been shown to be useful against minor common illnesses and may also provide novel natural uses against depression, dementia, diabetes, obesity, lupus, cancer, and cardiovascular disease.

In some embodiments, about 0.5 mg to about 50 mg of sclareolides (e.g., sage leaf powder) is present in a composition herein, e.g., about 0.5 mg to about 40 mg, about 0.5 mg to about 30 mg, about 0.5 mg to about 20 mg, about 0.5 mg to about 15 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 50 mg, about 2 mg to about 40 mg, about 2 mg to about 30 mg, about 2 mg to about 20 mg, about 2 mg to about 15 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 50 mg, about 3 mg to about 40 mg, about 3 mg to about 30 mg, about 3 mg to about 20 mg, about 3 mg to about 15 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 50 mg, about 4 mg to about 40 mg, about 4 mg to about 30 mg, about 4 mg to about 20 mg, about 4 mg to about 15 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 5 mg to about 9 mg, about 5 mg to about 8 mg, about 5 mg to about 7 mg, about 5 mg to about 6 mg, about 6 mg to about 50 mg, about 6 mg to about 40 mg, about 6 mg to about 30 mg, about 6 mg to about 20 mg, about 6 mg to about 15 mg, about 6 mg to about 10 mg, about 6 mg to about 9 mg, about 6 mg to about 8 mg, about 6 mg to about 7 mg, about 7 mg to about 50 mg, about 7 mg to about 40 mg, about 7 mg to about 30 mg, about 7 mg to about 20 mg, about 7 mg to about 15 mg, about 7 mg to about 10 mg, about 7 mg to about 9 mg, about 7 mg to about 8 mg, about 8 mg to about 50 mg, about 8 mg to about 40 mg, about 8 mg to about 30 mg, about 8 mg to about 20 mg, about 8 mg to about 15 mg, about 8 mg to about 10 mg, about 8 mg to about 9 mg, about 9 mg to about 50 mg, about 9 mg to about 40 mg, about 9 mg to about 30 mg, about 9 mg to about 20 mg, about 9 mg to about 15 mg, about 9 mg to about 10 mg, about 10 mg to about 50 mg, about 10 mg to about 40 mg, about 10 mg to about 30 mg, about 10 mg to about 20 mg, about 10 mg to about 15 mg, about 15 mg to about 50 mg, about 15 mg to about 40 mg, about 15 mg to about 30 mg, about 15 mg to about 20 mg, about 20 mg to about 50 mg, about 20 mg to about 40 mg, about 20 mg to about 30 mg, about 30 mg to about 50 mg, about 30 mg to about 40 mg, about 40 mg to about 50 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 30 mg, about 40 mg, or about 50 mg.

In some embodiments, about 0.01% to about 1.5% (w/w) of a composition is sclareolides (e.g., sage leaf powder), e.g., about 0.01% to about 1.4%, about 0.01% to about 1.3%, about 0.01% to about 1.2%, about 0.01% to about 1.1%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01 to about 0.1%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, about 0.5% to about 0.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.5%, about 1% to about 1.4%, about 1% to about 1.3%, about 1% to about 1.2%, about 1% to about 1.1%, about 1.1% to about 1.5%, about 1.1% to about 1.4%, about 1.1% to about 1.3%, about 1.1% to about 1.2%, about 1.2% to about 1.5%, about 1.2% to about 1.4%, about 1.2% to about 1.3%, about 1.3% to about 1.5%, about 1.3% to about 1.4%, about 1.4% to about 1.5%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% is sclareolides (e.g., sage leaf powder).

Certain compositions herein are formulated with components used against depression, dementia, diabetes, obesity, lupus, cancer, and cardiovascular disease. In some such cases, the amount of sclareolides (e.g., sage leaf powder) present in the composition is about 2 mg to about 8 mg, about 3 mg to about 7 mg, about 3 mg to about 6 mg, or about 5 mg. An example product is called RH2.

As described above, intake of sclareolides may lead to various effects for a person. For example, intake of sclareolides may improve the symptoms of common illnesses, depression, dementia, diabetes, obesity, lupus, cancer, and cardiovascular diseases.

### Diindolylmethane (DIM)

In one aspect, provided are compositions comprising DIM. DIM, or diindolylmethane, is a compound found in cruciferous vegetable such as broccoli, cauliflower, and cabbage. Studies suggest DIM may have anti-inflammatory and anti-cancer effects. Some studies show that DIM benefits some persons with cervical cancer and prostate cancer.

In some embodiments, about 0.5 mg to about 50 mg of DIM is present in a composition herein, e.g., about 0.5 mg to about 40 mg, about 0.5 mg to about 30 mg, about 0.5 mg to about 20 mg, about 0.5 mg to about 15 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 50 mg, about 2 mg to about 40 mg, about 2 mg to about 30 mg, about 2 mg to about 20 mg, about 2 mg to about 15 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 50 mg, about 3 mg to about 40 mg, about 3 mg to about 30 mg, about 3 mg to about 20 mg, about 3 mg to about 15 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 50 mg, about 4 mg to about 40 mg, about 4 mg to about 30 mg, about 4 mg to about 20 mg, about 4 mg to about 15 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 5 mg to about 9 mg, about 5 mg to about 8 mg, about 5 mg to about 7 mg, about 5 mg to about 6 mg, about 6 mg to about 50 mg, about 6 mg to about 40 mg, about 6 mg to about 30 mg, about 6 mg to about 20 mg, about 6 mg to about 15 mg, about 6 mg to about 10 mg, about 6 mg to about 9 mg, about 6 mg to about 8 mg, about 6 mg to about 7 mg, about 7 mg to about 50 mg, about 7 mg to about 40 mg, about 7 mg to about 30 mg, about 7 mg to about 20 mg, about 7 mg to about 15 mg, about 7 mg to about 10 mg, about 7 mg to about 9 mg, about 7 mg to about 8 mg, about 8 mg to about 50 mg, about 8 mg to about 40 mg, about 8 mg to about 30 mg, about 8 mg to about 20 mg, about 8 mg to about 15 mg, about 8 mg to about 10 mg, about 8 mg to about 9 mg, about 9 mg to about 50 mg, about 9 mg to about 40 mg, about 9 mg to about 30 mg, about 9 mg to about 20 mg, about 9 mg to about 15 mg, about 9 mg to about 10 mg, about 10 mg to about 50 mg, about 10 mg to about 40 mg, about 10 mg to about 30 mg, about 10 mg to about 20 mg, about 10 mg to about 15 mg, about 15 mg to about 50 mg, about 15 mg to about 40 mg, about 15 mg to about 30 mg, about 15 mg to about 20 mg, about 20 mg to about 50 mg, about 20 mg to about 40 mg, about 20 mg to about 30 mg, about 30 mg to about 50 mg, about 30 mg to about 40 mg, about 40 mg to about 50 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 30 mg, about 40 mg, or about 50 mg.

In some embodiments, about 0.01% to about 1.5% (w/w) of a composition is DIM, e.g., about 0.01% to about 1.4%, about 0.01% to about 1.3%, about 0.01% to about 1.2%, about 0.01% to about 1.1%, about 0.01% to about 1%, about 0.01% to about 0.9%, about 0.01% to about 0.8%, about 0.01% to about 0.7%, about 0.01% to about 0.6%, about 0.01% to about 0.5%, about 0.01% to about 0.4%, about 0.01% to about 0.3%, about 0.01% to about 0.2%, about 0.01 to about 0.1%, about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1.5%, about 0.5% to about 1.4%, about 0.5% to about 1.3%, about 0.5% to about 1.2%, about 0.5% to about 1.1%, about 0.5% to about 1%, about 0.5% to about 0.9%, about 0.5% to about 0.8%, about 0.5% to about 0.7%, about 0.5% to about 0.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.5%, about 1% to about 1.4%, about 1% to about 1.3%, about 1% to about 1.2%, about 1% to about 1.1%, about 1.1% to about 1.5%, about 1.1% to about 1.4%, about 1.1% to about 1.3%, about 1.1% to about 1.2%, about 1.2% to about 1.5%, about 1.2% to about 1.4%, about 1.2% to about 1.3%, about 1.3% to about 1.5%, about 1.3% to about 1.4%, about 1.4% to about 1.5%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% is DIM.

Certain compositions herein are formulated with components that have anti-inflammatory and anti-cancer effects. In some such cases, the amount of DIM present in the composition is about 2 mg to about 8 mg, about 3 mg to about 7 mg, about 3 mg to about 6 mg, or about 5 mg. An example product is called RH2.

As described above, intake of DIM may lead to various effects for a person. For example, intake of DIM may reduce inflammation as well as reduce the risk of or improve the symptoms of various cancers.

Bacopa (Bacopa monnieri)

In one aspect, provided are compositions comprising Bacopa monnieri (bacopa). Bacopa monnieri is a medicinal herb used in traditional Indian medicine system, Ayurveda. Bacopa is a creeping perennial with purple flowers and small oblong leaves, native to the warm wetlands of India and Australia. Bacopa is traditionally used for many ailments, but it is best known as a memory enhancer and neural tonic. Studies of bacopa demonstrate its antioxidant neuroprotection, acetylcholinesterase inhibition, choline acetyltransferase activation, beta-amyloid reduction, increased cerebral blood flow, and neurotransmitter modulation capabilities.

In some embodiments, about 1 mg to about 100 mg of bacopa is present in a composition herein, e.g., about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 100 mg, about 2.5 mg to about 90 mg, about 2.5 mg to about 80 mg, about 2.5 mg to about 70 mg, about 2.5 mg to about 60 mg, about 2.5 mg to about 50 mg, about 2.5 mg to about 40 mg, about 2.5 mg to about 30 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 15 mg, about 5 mg to about 12.5 mg, about 5 mg to about 10 mg, about 5 mg to about 7.5 mg, about 7.5 mg to about 100 mg, about 7.5 mg to about 90 mg, about 7.5 mg to about 80 mg, about 7.5 mg to about 70 mg, about 7.5 mg to about 60 mg, about 7.5 mg to about 50 mg, about 7.5 mg to about 40 mg, about 7.5 mg to about 30 mg, about 7.5 mg to about 20 mg, about 7.5 mg to about 15 mg, about 7.5 mg to about 12.5 mg, about 7.5 mg to about 10 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 10 mg to about 15 mg, about 10 mg to about 12.5 mg, about 12.5 mg to about 100 mg, about 12.5 mg to about 90 mg, about 12.5 mg to about 80 mg, about 12.5 mg to about 70 mg, about 12.5 mg to about 60 mg, about 12.5 mg to about 50 mg, about 12.5 mg to about 40 mg, about 12.5 mg to about 30 mg, about 12.5 mg to about 20 mg, about 12.5 mg to about 15 mg, about 15 mg to about 100 mg, about 15 mg to about 90 mg, about 15 mg to about 80 mg, about 15 mg to about 70 mg, about 15 mg to about 60 mg, about 15 mg to about 50 mg, about 15 mg to about 40 mg, about 15 mg to about 30 mg, about 15 mg to about 20 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 100 mg, about 50 mg to about 90 mg, about 50 mg to about 80 mg, about 50 mg to about 70 mg, about 50 mg to about 60 mg, about 60 mg to about 100 mg, about 60 mg to about 90 mg, about 60 mg to about 80 mg, about 60 mg to about 70 mg, about 70 mg to about 100 mg, about 70 mg to about 90 mg, about 70 mg to about 80 mg, about 80 mg to about 100 mg, about 80 mg to about 90 mg, about 90 mg to about 100 mg, about 1 mg, about 2.5 mg, about 5 mg, about 7.5 mg, about 10 mg, about 12.5 mg, about 15 mg, about 20 mg, about 25 mg, about 30 mg, about 40 mg, about 50 mg, about 60 mg, about 70 mg, about 80 mg, about 90 mg, or about 100 mg.

In some embodiments, about 0.2% to about 2% (w/w) of a composition comprises bacopa, e.g., about 0.2% to about 1.8%, about 0.2% to about 1.6%, about 0.2% to about 1.4%, about 0.2% to about 1.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, 0.2% to about 0.3%, about 0.3% to about 2%, about 0.3% to about 1.8%, about 0.3% to about 1.6%, about 0.3% to about 1.4%, about 0.3% to about 1.2%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 2%, about 0.4% to about 1.8%, about 0.4% to about 1.6%, about 0.4% to about 1.4%, about 0.4% to about 1.2%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 2%, about 0.5% to about 1.8%, about 0.5% to about 1.6%, about 0.5% to about 1.4%, about 0.5% to about 1.2%, about 0.5% to about 1%, about 0.5% to about 0.9%, about 0.5% to about 0.8%, about 0.5% to about 0.7%, about 0.5% to about 0.6%, about 0.6% to about 2%, about 0.6% to about 1.8%, about 0.6% to about 1.6%, about 0.6% to about 1.4%, about 0.6% to about 1.2%, about 0.6% to about 1%, about 0.6% to about 0.9%, about 0.6% to about 0.8%, about 0.6% to about 0.7%, about 0.7% to about 2%, about 0.7% to about 1.8%, about 0.7% to about 1.6%, about 0.7% to about 1.4%, about 0.7% to about 1.2%, about 0.7% to about 1%, about 0.7% to about 0.9%, about 0.7% to about 0.8%, about 0.8% to about 2%, about 0.8% to about 1.8%, about 0.8% to about 1.6%, about 0.8% to about 1.4%, about 0.8% to about 1.2%, about 0.8% to about 1%, about 0.8% to about 0.9%, about 0.9% to about 2%, about 0.9% to about 1.8%, about 0.9% to about 1.6%, about 0.9% to about 1.4%, about 0.9% to about 1.2%, about 0.9% to about 1%, about 1% to about 2%, about 1% to about 1.8%, about 1% to about 1.6%, about 1% to about 1.4%, about 1% to about 1.2%, about 1.2% to about 2%, about 1.2% to about 1.8%, about 1.2% to about 1.6%, about 1.2% to about 1.4%, about 1.4% to about 2%, about 1.4% to about 1.8%, about 1.4% to about 1.6%, about 1.6% to about 2%, about 1.6% to about 1.8%, about 1.8% to about 2%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, or about 2% is bacopa.

Certain compositions herein are formulated with components having memory enhancing properties. In some such cases, the amount of bacopa present in the composition is about 7 mg to about 13 mg, about 8 mg to about 12 mg, about 9 mg to about 11 mg, or about 10 mg. An example product is called RH1.

As described above, intake of bacopa may lead to various effects for a person. For example, intake of bacopa may lead to increased memory.

### Oat Straw

In one aspect, provided are compositions comprising oat straw. Oat straw, or green oat, is an extract of from the aerial parts of oat plants (Avena sativa). The tinctures and extracts are formulated from the plant while it is still green. Oat straw contains several bioactive compounds such as terpenes, phenolic acids, polyphenols like flavonoids and avenanthramides. Studies have shown oat straw's ability to improve cognitive function and modulate the physiological response to a stressor, i.e., improve memory and help assuage anxiety.

In some embodiments, about 1 mg to about 125 mg of oat straw is present in a composition herein, e.g., about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 100 mg, about 75 mg to about 100 mg, about 100 mg to about 125 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, or about 125 mg.

In some embodiments, about 0.1% to about 2.5% (w/w) of a composition is oat straw, e.g., about 0.1% to about 2.5%, about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, about 2.1% to about 2.5%, about 0.1%, about 0.5%, about 0.9%, about 1.3%, about 1.7%, about 2.1%, or about 2.5% of the composition is oat straw.

Certain compositions herein are formulated with components used to improve cognitive function and modulate the physiological response to stressors. In some such cases, the amount of oat straw present in the composition is about 5 mg to about 21 mg, about 9 mg to about 17 mg, about 10 mg to about 15 mg, or about 12.5 mg. An example product is called RH2.

As described above, intake of oat straw may lead to various effects for a person. For example, intake of oat straw may improve cognitive function and memory as well as reduce symptoms of anxiety.

### Amino Acids (e.g., D-Aspartic Acid, L-Theanine, L-Tryptophan)

In one aspect, provided are compositions comprising amino acids. Non-limiting examples of amino acids include D-aspartic acid, L-theanine, and L-tryptophan. Studies show regular essential amino acid intake is effective in reversing muscle catabolism, restoring immunological function, and promoting muscle anabolism.

D-aspartic acid is an amino acid that functions, according to research, in the development of the nervous system and in hormonal regulation. In some cases, D-aspartic acid has been shown to increase testosterone, making it useful for supplementation during resistance training in certain cases. Further, studies show that D-aspartic acid plays a crucial role in reproduction either due to its role as a neuromodulator or its involvement in biosynthesis and release of sexual hormones.

In some embodiments, about 0.5 mg to about 50 mg of D-aspartic acid is present in a composition herein, e.g., about 0.5 mg to about 40 mg, about 0.5 mg to about 30 mg, about 0.5 mg to about 20 mg, about 0.5 mg to about 15 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 50 mg, about 2 mg to about 40 mg, about 2 mg to about 30 mg, about 2 mg to about 20 mg, about 2 mg to about 15 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 50 mg, about 3 mg to about 40 mg, about 3 mg to about 30 mg, about 3 mg to about 20 mg, about 3 mg to about 15 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 50 mg, about 4 mg to about 40 mg, about 4 mg to about 30 mg, about 4 mg to about 20 mg, about 4 mg to about 15 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 5 mg to about 9 mg, about 5 mg to about 8 mg, about 5 mg to about 7 mg, about 5 mg to about 6 mg, about 6 mg to about 50 mg, about 6 mg to about 40 mg, about 6 mg to about 30 mg, about 6 mg to about 20 mg, about 6 mg to about 15 mg, about 6 mg to about 10 mg, about 6 mg to about 9 mg, about 6 mg to about 8 mg, about 6 mg to about 7 mg, about 7 mg to about 50 mg, about 7 mg to about 40 mg, about 7 mg to about 30 mg, about 7 mg to about 20 mg, about 7 mg to about 15 mg, about 7 mg to about 10 mg, about 7 mg to about 9 mg, about 7 mg to about 8 mg, about 8 mg to about 50 mg, about 8 mg to about 40 mg, about 8 mg to about 30 mg, about 8 mg to about 20 mg, about 8 mg to about 15 mg, about 8 mg to about 10 mg, about 8 mg to about 9 mg, about 9 mg to about 50 mg, about 9 mg to about 40 mg, about 9 mg to about 30 mg, about 9 mg to about 20 mg, about 9 mg to about 15 mg, about 9 mg to about 10 mg, about 10 mg to about 50 mg, about 10 mg to about 40 mg, about 10 mg to about 30 mg, about 10 mg to about 20 mg, about 10 mg to about 15 mg, about 15 mg to about 50 mg, about 15 mg to about 40 mg, about 15 mg to about 30 mg, about 15 mg to about 20 mg, about 20 mg to about 50 mg, about 20 mg to about 40 mg, about 20 mg to about 30 mg, about 30 mg to about 50 mg, about 30 mg to about 40 mg, about 40 mg to about 50 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 30 mg, about 40 mg, or about 50 mg.

In some embodiments, about 0.1% to about 1% (w/w) of a composition is Panax ginseng, e.g., about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, about 0.9% to about 1%, about 0.1%, about 0.2%, about 0.3%, about 0.4%, about 0.5%, about 0.6%, about 0.7%, about 0.8%, about 0.9%, or about 1% is D-aspartic acid.

Certain compositions herein are formulated with components that may supplement the production of testosterone and sexual hormones. In some such cases, the amount of D-aspartic acid present in the composition is about 2 mg to about 8 mg, about 3 mg to about 7 mg, about 3 mg to about 6 mg, or about 5 mg. An example product is called RH1.

As described above, intake of D-aspartic acid may lead to various effects for a person. For example, intake of D-aspartic acid may reverse muscle catabolism, restore immunological functions, and promote muscle anabolism. As another example, intake of D-aspartic acid may promote development of the nervous system and hormone regulation. Intake of D-aspartic acid may also increase testosterone in a person.

L-theanine is a unique amino acid found in green tea. L-theanine may modulate aspects of brain function in humans. Studies show that L-theanine has a direct effect on the brain and, at higher amounts, can relax the brain without inducing drowsiness. Further, it is used for potential antioxidant and relaxant effects. In vitro animal studies suggest the following properties: lipid-lowering, neuroprotective, anti-obesity, and anti-tumor.

In some embodiments, about 1 mg to about 100 mg of L-theanine is present in a composition herein, e.g., about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 100 mg, about 2.5 mg to about 90 mg, about 2.5 mg to about 80 mg, about 2.5 mg to about 70 mg, about 2.5 mg to about 60 mg, about 2.5 mg to about 50 mg, about 2.5 mg to about 40 mg, about 2.5 mg to about 30 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 15 mg, about 5 mg to about 12.5 mg, about 5 mg to about 10 mg, about 5 mg to about 7.5 mg, about 7.5 mg to about 100 mg, about 7.5 mg to about 90 mg, about 7.5 mg to about 80 mg, about 7.5 mg to about 70 mg, about 7.5 mg to about 60 mg, about 7.5 mg to about 50 mg, about 7.5 mg to about 40 mg, about 7.5 mg to about 30 mg, about 7.5 mg to about 20 mg, about 7.5 mg to about 15 mg, about 7.5 mg to about 12.5 mg, about 7.5 mg to about 10 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 10 mg to about 15 mg, about 10 mg to about 12.5 mg, about 12.5 mg to about 100 mg, about 12.5 mg to about 90 mg, about 12.5 mg to about 80 mg, about 12.5 mg to about 70 mg, about 12.5 mg to about 60 mg, about 12.5 mg to about 50 mg, about 12.5 mg to about 40 mg, about 12.5 mg to about 30 mg, about 12.5 mg to about 20 mg, about 12.5 mg to about 15 mg, about 15 mg to about 100 mg, about 15 mg to about 90 mg, about 15 mg to about 80 mg, about 15 mg to about 70 mg, about 15 mg to about 60 mg, about 15 mg to about 50 mg, about 15 mg to about 40 mg, about 15 mg to about 30 mg, about 15 mg to about 20 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 100 mg, about 50 mg to about 90 mg, about 50 mg to about 80 mg, about 50 mg to about 70 mg, about 50 mg to about 60 mg, about 60 mg to about 100 mg, about 60 mg to about 90 mg, about 60 mg to about 80 mg, about 60 mg to about 70 mg, about 70 mg to about 100 mg, about 70 mg to about 90 mg, about 70 mg to about 80 mg, about 80 mg to about 100 mg, about 80 mg to about 90 mg, about 90 mg to about 100 mg, about 1 mg, about 2.5 mg, about 5 mg, about 7.5 mg, about 10 mg, about 12.5 mg, about 15 mg, about 20 mg, about 25 mg, about 30 mg, about 40 mg, about 50 mg, about 60 mg, about 70 mg, about 80 mg, about 90 mg, or about 100 mg.

In some embodiments, about 0.1% to about 8% (w/w) of a composition is L-theanine, e.g., about 0.1% (w/w) to about 7% (w/w), about 0.1% (w/w) to about 6% (w/w), about 0.1% (w/w) to about 5% (w/w), about 0.1% (w/w) to about 4% (w/w), about 0.1% (w/w) to about 3% (w/w), about 0.1% (w/w) to about 2% (w/w), about 0.1% (w/w) to about 1% (w/w), about 1.1% (w/w) to about 8% (w/w), about 1.1% (w/w) to about 7% (w/w), about 1.1% (w/w) to about 6% (w/w), about 1.1% (w/w) to about 5% (w/w), about 1.1% (w/w) to about 4% (w/w), about 1.1% (w/w) to about 3% (w/w), about 1.1% (w/w) to about 2% (w/w), about 2.1% (w/w) to about 8% (w/w), about 2.1% (w/w) to about 7% (w/w), about 2.1% (w/w) to about 6% (w/w), about 2.1% (w/w) to about 5% (w/w), about 2.1% (w/w) to about 4% (w/w), about 2.1% (w/w) to about 3% (w/w), about 3.1% (w/w) to about 8% (w/w), about 3.1% (w/w) to about 7% (w/w), about 3.1% (w/w) to about 6% (w/w), about 3.1% (w/w) to about 5% (w/w), about 3.1% (w/w) to about 4% (w/w), about 4.1% (w/w) to about 8% (w/w), about 4.1% (w/w) to about 7% (w/w), about 4.1% (w/w) to about 6% (w/w), about 4.1% (w/w) to about 5% (w/w), about 5.1% (w/w) to about 8% (w/w), about 5.1% (w/w) to about 7% (w/w), about 5.1% (w/w) to about 6% (w/w), about 6.1% (w/w) to about 8% (w/w), about 6.1% (w/w) to about 7% (w/w), about 7.1% (w/w) to about 8% (w/w), about 0.1% (w/w), about 1.1% (w/w), about 2.1% (w/w), about 3.1% (w/w), about 4.1% (w/w), about 5.1% (w/w), about 6.1% (w/w), or about 7.1% (w/w) is L-theanine.

Certain compositions herein are formulated with components having relaxant and antioxidant properties. In some such cases, the amount of L-theanine present in the composition is about 7 mg to about 13 mg, about 8 mg to about 12 mg, about 9 mg to about 11 mg, or about 10 mg. An example product is called RH3.

As described above, intake of L-theanine may lead to various effects for a person. For example, intake of L-theanine may lead to increased brain relaxation without drowsiness. Intake of L-theanine may also reduce the risk of certain diseases.

L-tryptophan is an essential amino acid in humans. It is a precursor of serotonin, and therefore its intake is linked to the suppression of anxiety and depression. L-tryptophan cannot be synthesized by the body so it must be obtained from the diet. Healthy intake of L-tryptophan assures increased brain L-tryptophan levels and leads to enhanced serotonin synthesis. Studies show that L-tryptophan intake has been found to improve the mood of persons vulnerable to stress such as those that have experienced depression. It has also been reported that L-tryptophan improves the sleep of adults with sleep disorders.

In some embodiments, about 0.5 mg to about 75 mg of L-tryptophan is present in a composition herein, e.g., about 0.5 mg to about 50 mg, about 0.5 mg to about 25 mg, about 0.5 mg to about 20 mg, about 0.5 mg to about 15 mg, about 0.5 mg to about 10 mg, about 0.5 mg to about 7.5 mg, about 0.5 mg to about 5 mg, about 0.5 mg to about 2.5 mg, about 0.5 mg to about 1 mg, about 1 mg to about 75 mg, about 1 mg to about 50 mg, about 1 mg to about 25 mg, about 1 mg to about 20 mg, about 1 mg to about 15 mg, about 1 mg to about 10 mg, about 1 mg to about 7.5 mg, about 1 mg to about 5 mg, about 1 mg to about 2.5 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 10 mg, about 2.5 mg to about 7.5 mg, about 2.5 mg to about 5 mg, about 5 mg to about 75 mg, about 5 mg to about 50 mg, about 5 mg to about 25 mg, about 5 mg to about 20 mg, about 5 mg to about 15 mg, about 5 mg to about 10 mg, about 5 mg to about 7.5 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 10 mg, about 10 mg to about 75 mg, about 10 mg to about 50 mg, about 10 mg to about 25 mg, about 10 mg to about 20 mg, about 10 mg to about 15 mg, about 15 mg to about 75 mg, about 15 mg to about 50 mg, about 15 mg to about 25 mg, about 15 mg to about 20 mg, about 20 mg to about 75 mg, about 20 mg to about 50 mg, about 20 mg to about 25 mg, about 25 mg to about 75 mg, about 25 mg to about 50 mg, about 50 mg to about 75 mg, about 0.5 mg, about 1 mg, about 2.5 mg, about 5 mg, about 7.5 mg, about 10 mg, about 15 mg, about 20 mg, about 25 mg, about 50 mg, or about 75 mg.

In some embodiments, about 0.1% to about 7% (w/w) of a composition is L-tryptophan, e.g., about 0.1% (w/w) to about 6% (w/w), about 0.1% (w/w) to about 5% (w/w), about 0.1% (w/w) to about 4% (w/w), about 0.1% (w/w) to about 3% (w/w), about 0.1% (w/w) to about 2% (w/w), about 0.1% (w/w) to about 1% (w/w), about 1.1% (w/w) to about 7% (w/w), about 1.1% (w/w) to about 6% (w/w), about 1.1% (w/w) to about 5% (w/w), about 1.1% (w/w) to about 4% (w/w), about 1.1% (w/w) to about 3% (w/w), about 1.1% (w/w) to about 2% (w/w), about 2.1% (w/w) to about 7% (w/w), about 2.1% (w/w) to about 6% (w/w), about 2.1% (w/w) to about 5% (w/w), about 2.1% (w/w) to about 4% (w/w), about 2.1% (w/w) to about 3% (w/w), about 3.1% (w/w) to about 7% (w/w), about 3.1% (w/w) to about 6% (w/w), about 3.1% (w/w) to about 5% (w/w), about 3.1% (w/w) to about 4% (w/w), about 4.1% (w/w) to about 7% (w/w), about 4.1% (w/w) to about 6% (w/w), about 4.1% (w/w) to about 5% (w/w), about 5.1% (w/w) to about 7% (w/w), about 5.1% (w/w) to about 6% (w/w), about 6.1% (w/w) to about 7% (w/w), about 0.1% (w/w), about 1.1% (w/w), about 2.1% (w/w), about 3.1% (w/w), about 4.1% (w/w), about 5.1% (w/w), or about 6.1% (w/w) is L-tryptophan.

Certain compositions herein are formulated with components having anti-anxiety and anti-depression properties. In some such cases, the amount of L-tryptophan present in the composition is about 4 mg to about 11 mg, about 5 mg to about 10 mg, about 6 mg to about 9 mg, or about 7.5 mg. An example product is called RH3.

As described above, intake of L-tryptophan may lead to various effects for a person. For example, intake of L-tryptophan may reduce the symptoms of anxiety and depression as well as increased serotonin in a person. Intake of L-tryptophan may also lead to improved sleep for persons with sleep disorders.

### Reishi Mushroom

In one aspect, provided are compositions comprising reishi mushroom. Reishi mushroom, or Ganoderma lucidum, is a mushroom used in traditional Chinese medicine where it is believed to extend life and promote health. Studies show that reishi mushroom may have anticancer properties. Reishi mushrooms contain bioactive components such as terpenoids, steroids, phenols, nucleotides and their derivatives, glycoproteins, and polysaccharides. Mushroom proteins contain essential amino acids and are rich in lysine and leucine. Terpenoids, from the class of terpenes, are naturally occurring compounds that have been found to have anti-inflammatory, anti-tumorigenic and hypolipidemic activity.

In some embodiments, about 0.5 mg to about 50 mg of reishi mushroom is present in a composition herein, e.g., about 0.5 mg to about 40 mg, about 0.5 mg to about 30 mg, about 0.5 mg to about 20 mg, about 0.5 mg to about 15 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 50 mg, about 2 mg to about 40 mg, about 2 mg to about 30 mg, about 2 mg to about 20 mg, about 2 mg to about 15 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 50 mg, about 3 mg to about 40 mg, about 3 mg to about 30 mg, about 3 mg to about 20 mg, about 3 mg to about 15 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 50 mg, about 4 mg to about 40 mg, about 4 mg to about 30 mg, about 4 mg to about 20 mg, about 4 mg to about 15 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 5 mg to about 9 mg, about 5 mg to about 8 mg, about 5 mg to about 7 mg, about 5 mg to about 6 mg, about 6 mg to about 50 mg, about 6 mg to about 40 mg, about 6 mg to about 30 mg, about 6 mg to about 20 mg, about 6 mg to about 15 mg, about 6 mg to about 10 mg, about 6 mg to about 9 mg, about 6 mg to about 8 mg, about 6 mg to about 7 mg, about 7 mg to about 50 mg, about 7 mg to about 40 mg, about 7 mg to about 30 mg, about 7 mg to about 20 mg, about 7 mg to about 15 mg, about 7 mg to about 10 mg, about 7 mg to about 9 mg, about 7 mg to about 8 mg, about 8 mg to about 50 mg, about 8 mg to about 40 mg, about 8 mg to about 30 mg, about 8 mg to about 20 mg, about 8 mg to about 15 mg, about 8 mg to about 10 mg, about 8 mg to about 9 mg, about 9 mg to about 50 mg, about 9 mg to about 40 mg, about 9 mg to about 30 mg, about 9 mg to about 20 mg, about 9 mg to about 15 mg, about 9 mg to about 10 mg, about 10 mg to about 50 mg, about 10 mg to about 40 mg, about 10 mg to about 30 mg, about 10 mg to about 20 mg, about 10 mg to about 15 mg, about 15 mg to about 50 mg, about 15 mg to about 40 mg, about 15 mg to about 30 mg, about 15 mg to about 20 mg, about 20 mg to about 50 mg, about 20 mg to about 40 mg, about 20 mg to about 30 mg, about 30 mg to about 50 mg, about 30 mg to about 40 mg, about 40 mg to about 50 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 30 mg, about 40 mg, or about 50 mg.

In some embodiments, about 0.5% to about 5% (w/w) of a composition is reishi mushroom, e.g., about 0.5% to about 4.5%, about 0.5% to about 4%, about 0.5% to about 3.5%, about 0.5% to about 3%, about 0.5% to about 2.5%, about 0.5% to about 2%, about 0.5% to about 1.5%, about 0.5% to about 1%, about 1% to about 5%, about 1% to about 4.5%, about 1% to about 4%, about 1% to about 3.5%, about 1% to about 3%, about 1% to about 2.5%, about 1% to about 2%, about 1% to about 1.5%, about 1.5% to about 5%, about 1.5% to about 4.5%, about 1.5% to about 4%, about 1.5% to about 3.5%, about 1.5% to about 3%, about 1.5% to about 2.5%, about 1.5% to about 2%, about 2% to about 5%, about 2% to about 4.5%, about 2% to about 4%, about 2% to about 3.5%, about 2% to about 3%, about 2% to about 2.5%, about 2.5% to about 5%, about 2.5% to about 4.5%, about 2.5% to about 4%, about 2.5% to about 3.5%, about 2.5% to about 3%, about 3% to about 5%, about 3% to about 4.5%, about 3% to about 4%, about 3% to about 3.5%, about 3.5% to about 5%, about 3.5% to about 4.5%, about 3.5% to about 4%, about 4% to about 5%, about 4% to about 4.5%, about 4.5% to about 5%, about 0.5%, about 1%, about 1.5%, about 2%, about 2.5%, about 3%, about 3.5%, about 4%, about 4.5%, or about 5%.

Certain compositions herein are formulated with components having anti-inflammatory, anti-tumorigenic and/or hypolipidemic properties. In some such cases, the amount of reishi mushroom present in the composition is about 2 mg to about 8 mg, about 3 mg to about 7 mg, about 3 mg to about 6 mg, or about 5 mg. An example product is called RH3.

As described above, intake of reishi mushroom may lead to various effects for a person. For example, intake of reishi mushroom may reduce the risk of and/or reduce the symptoms of cancer. Intake of reishi mushroom may also reduce inflammation.

### Melatonin

In one aspect, provided are compositions comprising melatonin. Melatonin is a hormone secreted by the pineal gland. It regulates circadian rhythms such as neuroendocrine rhythms, sleep-wake rhythm, and body temperature cycles. This is done through its action on MT1 and MT2 receptors. Melatonin is commonly ingested to induce fatigue and decrease the time needed to fall asleep. It is commonly taken to realize these effects and improve sleep. Furthermore, studies show that use of melatonin by healthy adults shows promise against phase shifts from jet lag and improve insomnia.

In some embodiments, about 0.1 mg to about 25 mg of melatonin is present in a composition herein, e.g., about 0.1 mg to about 20 mg, about 0.1 mg to about 15 mg, about 0.1 mg to about 10 mg, about 0.1 mg to about 7.5 mg, about 0.1 mg to about 5 mg, about 0.1 mg to about 4 mg, about 0.1 mg to about 3 mg, about 0.1 mg to about 2.5 mg, about 0.1 mg to about 2 mg, about 0.1 mg to about 1.5 mg, about 0.1 mg to about 1 mg, about 0.1 mg to about 0.5 mg, about 0.1 mg to about 0.25 mg, about 0.25 mg to about 25 mg, about 0.25 mg to about 20 mg, about 0.25 mg to about 15 mg, about 0.25 mg to about 10 mg, about 0.25 mg to about 7.5 mg, about 0.25 mg to about 5 mg, about 0.25 mg to about 4 mg, about 0.25 mg to about 3 mg, about 0.25 mg to about 2.5 mg, about 0.25 mg to about 2 mg, about 0.25 mg to about 1.5 mg, about 0.25 mg to about 1 mg, about 0.25 mg to about 0.5 mg, about 0.5 mg to about 25 mg, about 0.5 mg to about 20 mg, about 0.5 mg to about 15 mg, about 0.5 mg to about 10 mg, about 0.5 mg to about 7.5 mg, about 0.5 mg to about 5 mg, about 0.5 mg to about 4 mg, about 0.5 mg to about 3 mg, about 0.5 mg to about 2.5 mg, about 0.5 mg to about 2 mg, about 0.5 mg to about 1.5 mg, about 0.5 mg to about 1 mg, about 1 mg to about 25 mg, about 1 mg to about 20 mg, about 1 mg to about 15 mg, about 1 mg to about 10 mg, about 1 mg to about 7.5 mg, about 1 mg to about 5 mg, about 1 mg to about 4 mg, about 1 mg to about 3 mg, about 1 mg to about 2.5 mg, about 1 mg to about 2 mg, about 1 mg to about 1.5 mg, about 1.5 mg to about 25 mg, about 1.5 mg to about 20 mg, about 1.5 mg to about 15 mg, about 1.5 mg to about 10 mg, about 1.5 mg to about 7.5 mg, about 1.5 mg to about 5 mg, about 1.5 mg to about 4 mg, about 1.5 mg to about 3 mg, about 1.5 mg to about 2.5 mg, about 1.5 mg to about 2 mg, about 2 mg to about 25 mg, about 2 mg to about 20 mg, about 2 mg to about 15 mg, about 2 mg to about 10 mg, about 2 mg to about 7.5 mg, about 2 mg to about 5 mg, about 2 mg to about 4 mg, about 2 mg to about 3 mg, about 2 mg to about 2.5 mg, about 2.5 mg to about 25 mg, about 2.5 mg to about 20 mg, about 2.5 mg to about 15 mg, about 2.5 mg to about 10 mg, about 2.5 mg to about 7.5 mg, about 2.5 mg to about 5 mg, about 2.5 mg to about 4 mg, about 2.5 mg to about 3 mg, about 3 mg to about 25 mg, about 3 mg to about 20 mg, about 3 mg to about 15 mg, about 3 mg to about 10 mg, about 3 mg to about 7.5 mg, about 3 mg to about 5 mg, about 3 mg to about 4 mg, about 4 mg to about 25 mg, about 4 mg to about 20 mg, about 4 mg to about 15 mg, about 4 mg to about 10 mg, about 4 mg to about 7.5 mg, about 4 mg to about 5 mg, about 5 mg to about 25 mg, about 5 mg to about 20 mg, about 5 mg to about 15 mg, about 5 mg to about 10 mg, about 5 mg to about 7.5 mg, about 7.5 mg to about 25 mg, about 7.5 mg to about 20 mg, about 7.5 mg to about 15 mg, about 7.5 mg to about 10 mg, about 10 mg to about 25 mg, about 10 mg to about 20 mg, about 10 mg to about 15 mg, about 15 mg to about 25 mg, about 15 mg to about 20 mg, about 20 mg to about 25 mg, about 0.1 mg, about 0.25 mg, about 0.5 mg, about 1 mg, about 1.5 mg, about 2 mg, about 2.5 mg, about 3 mg, about 4 mg, about 5 mg, about 7.5 mg, about 10 mg, about 12.5 mg, about 15 mg, about 20 mg, or about 25 mg.

In some embodiments, about 0.01% to about 2% (w/w) of a composition is melatonin, e.g., about 0.01% (w/w) to about 1.75% (w/w), about 0.01% (w/w) to about 1.5%, about 0.01% to about 1.25%, about 0.01% to about 1%, about 0.01% to about 0.75%, about 0.01% to about 0.5%, about 0.01% to about 0.25%, about 0.26% (w/w) to about 2% (w/w), about 0.26% (w/w) to about 1.75% (w/w), about 0.26% (w/w) to about 1.5% (w/w), about 0.26% (w/w) to about 1.25% (w/w), about 0.26% (w/w) to about 1% (w/w), about 0.26% (w/w) to about 0.75% (w/w), about 0.26% (w/w) to about 0.5% (w/w), about 0.51% (w/w) to about 2% (w/w), about 0.51% (w/w) to about 1.75% (w/w), about 0.51% (w/w) to about 1.5% (w/w), about 0.51% (w/w) to about 1.25% (w/w), about 0.51% (w/w) to about 1% (w/w), about 0.51% (w/w) to about 0.75% (w/w), about 0.76% (w/w) to about 2% (w/w), about 0.76% (w/w) to about 1.75% (w/w), about 0.76% (w/w) to about 1.5% (w/w), about 0.76% (w/w) to about 1.25% (w/w), about 0.76% (w/w) to about 1% (w/w), about 1.01% (w/w) to about 2% (w/w), about 1.01% (w/w) to about 1.75% (w/w), about 1.01% (w/w) to about 1.5% (w/w), about 1.01% (w/w) to about 1.25% (w/w), about 1.26% (w/w) to about 2% (w/w), about 1.26% (w/w) to about 1.75% (w/w), about 1.26% (w/w) to about 1.5% (w/w), about 1.51% (w/w) to about 2% (w/w), about 1.51% (w/w) to about 1.75% (w/w), about 1.76% (w/w) to about 2% (w/w), about 0.01% (w/w), about 0.26% (w/w), about 0.51% (w/w), about 0.76% (w/w), about 1.01% (w/w), about 1.26% (w/w), about 1.51% (w/w), or about 1.76% (w/w) is melatonin.

Certain compositions herein are formulated with components for regulation of circadian rhythms such as neuroendocrine rhythms, sleep-wake rhythm, or body temperature cycles. In some such cases, the amount of melatonin present in the composition is about 1 mg to about 4 mg, about 1.5 mg to about 3.5 mg, about 2 mg to about 3 mg, or about 2.5 mg. An example product is called RH3.

As described above, intake of melatonin may lead to various effects for a person. For example, intake of melatonin may induce desired fatigue and improve the ability of a person to fall asleep.

### Excipients

In one aspect, provided are compositions and formulations comprising one or more excipients. Excipients, include enhancing agents, and may be included in compositions herein and serve as vehicles for their delivery. An excipient may be stable and reproducible, have no unwished interactions with compositions, and non-toxic. An acceptable excipient may be an excipient that is useful in preparing a composition that is generally safe, non-toxic, and desirable, and includes excipients that are acceptable for veterinary use as well as for human use. The role of excipients may be to aid in the processing of composition delivery systems during composition manufacture. Excipients may protect, support, and/or enhance stability and bioavailability and composition delivery. The excipient may be an anti-caking agent. The excipient may be an adsorbent. The excipient may be a disintegrant. The excipient may be a glidant. The excipient may be a binder. The excipient may be a penetration enhancer. The excipient may include a chelating agent that assists in drug delivery by depleting Ca2+. Non-limiting examples of excipients used in formulations include magnesium stearate, microcrystalline cellulose, starch (corn), silicone/titanium dioxide, stearic acid, sodium starch glycolate, gelatin, talc, sucrose, calcium stearate, povidone, pregelantinized starch, HPMC, croscarmellose, hydroxypropyl cellulose, ethylcellulose, calcium phosphate, and crospovidone, glucose, lactose, sucrose, malt, rice, flour, chalk, silica gel, sodium stearate, glycerol monostearate, sodium chloride, dried skim milk, water, saline, dextrose, propylene glycol, glycerol, ethanol, mannitol, polysorbate, or the like, and combinations thereof. In addition, if desired, the composition can contain auxiliary substances such as wetting or emulsifying agents, pH buffering agents and the like which enhance or maintain the effectiveness of other ingredients. In some embodiments, acceptable excipients, including enhancing agents, can be used for effective delivery of the composition. Excipients may act as stabilizing agents in compositions and formulations of the present disclosure. Ingredients of compositions herein can be mixed with excipients which are acceptable and compatible with the ingredient and in amounts suitable for use in methods described herein. Such excipients may be solid, liquid, semisolid, or, in the case of an aerosol composition, gaseous. Compositions as described herein can include acceptable salts. Acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine, and the like. Liquid compositions can contain liquid phases in addition to and in the exclusion of water, for example, glycerin, vegetable oils such as cottonseed oil, and water-oil emulsions. Physiologically tolerable carriers are well known in the art.

In some embodiments, about 0.1 mg to about 5000 mg of one or more excipients is present in a composition herein, e.g., about 0.1 mg to about 4000 mg, about 0.1 mg to about 3000 mg, about 0.1 mg to about 2000 mg, about 0.1 mg to about 1000 mg, about 0.1 mg to about 750 mg, about 0.1 mg to about 500 mg, about 0.1 mg to about 450 mg, about 0.1 mg to about 400 mg, about 0.1 mg to about 350 mg, about 0.1 mg to about 300 mg, about 0.1 mg to about 250 mg, about 0.1 mg to about 225 mg, about 0.1 mg to about 200 mg, about 0.1 mg to about 175 mg, about 0.1 mg to about 150 mg, about 0.1 mg to about 125 mg, about 0.1 mg to about 100 mg, about 0.1 mg to about 75 mg, about 0.1 mg to about 50 mg, about 0.1 mg to about 25 mg, about 0.1 mg to about 20 mg, about 0.1 mg to about 15 mg, about 0.1 mg to about 10 mg, about 0.1 mg to about 5 mg, about 0.1 mg to about 4 mg, about 0.1 mg to about 3 mg, about 0.1 mg to about 2 mg, about 0.1 mg to about 1.5 mg, about 0.1 mg to about 1.25 mg, about 0.1 mg to about 1 mg, about 0.1 mg to about 0.75 mg, about 0.1 mg to about 0.5 mg, about 0.1 mg to about 0.25 mg, about 0.25 mg to about 5000 mg, about 0.25 mg to about 4000 mg, about 0.25 mg to about 3000 mg, about 0.25 mg to about 2000 mg, about 0.25 mg to about 1000 mg, about 0.25 mg to about 750 mg, about 0.25 mg to about 500 mg, about 0.25 mg to about 450 mg, about 0.25 mg to about 400 mg, about 0.25 mg to about 350 mg, about 0.25 mg to about 300 mg, about 0.25 mg to about 250 mg, about 0.25 mg to about 225 mg, about 0.25 mg to about 200 mg, about 0.25 mg to about 175 mg, about 0.25 mg to about 150 mg, about 0.25 mg to about 125 mg, about 0.25 mg to about 100 mg, about 0.25 mg to about 75 mg, about 0.25 mg to about 50 mg, about 0.25 mg to about 25 mg, about 0.25 mg to about 20 mg, about 0.25 mg to about 15 mg, about 0.25 mg to about 10 mg, about 0.25 mg to about 5 mg, about 0.25 mg to about 4 mg, about 0.25 mg to about 3 mg, about 0.25 mg to about 2 mg, about 0.25 mg to about 1.5 mg, about 0.25 mg to about 1.25 mg, about 0.25 mg to about 1 mg, about 0.25 mg to about 0.75 mg, about 0.25 mg to about 0.5 mg, about 0.5 mg to about 5000 mg, about 0.5 mg to about 4000 mg, about 0.5 mg to about 3000 mg, about 0.5 mg to about 2000 mg, about 0.5 mg to about 1000 mg, about 0.5 mg to about 750 mg, about 0.5 mg to about 500 mg, about 0.5 mg to about 450 mg, about 0.5 mg to about 400 mg, about 0.5 mg to about 350 mg, about 0.5 mg to about 300 mg, about 0.5 mg to about 250 mg, about 0.5 mg to about 225 mg, about 0.5 mg to about 200 mg, about 0.5 mg to about 175 mg, about 0.5 mg to about 150 mg, about 0.5 mg to about 125 mg, about 0.5 mg to about 100 mg, about 0.5 mg to about 75 mg, about 0.5 mg to about 50 mg, about 0.5 mg to about 25 mg, about 0.5 mg to about 20 mg, about 0.5 mg to about 15 mg, about 0.5 mg to about 10 mg, about 0.5 mg to about 5 mg, about 0.5 mg to about 4 mg, about 0.5 mg to about 3 mg, about 0.5 mg to about 2 mg, about 0.5 mg to about 1.5 mg, about 0.5 mg to about 1.25 mg, about 0.5 mg to about 1 mg, about 0.5 mg to about 0.75 mg, about 0.75 mg to about 5000 mg, about 0.75 mg to about 4000 mg, about 0.75 mg to about 3000 mg, about 0.75 mg to about 2000 mg, about 0.75 mg to about 1000 mg, about 0.75 mg to about 750 mg, about 0.75 mg to about 500 mg, about 0.75 mg to about 450 mg, about 0.75 mg to about 400 mg, about 0.75 mg to about 350 mg, about 0.75 mg to about 300 mg, about 0.75 mg to about 250 mg, about 0.75 mg to about 225 mg, about 0.75 mg to about 200 mg, about 0.75 mg to about 175 mg, about 0.75 mg to about 150 mg, about 0.75 mg to about 125 mg, about 0.75 mg to about 100 mg, about 0.75 mg to about 75 mg, about 0.75 mg to about 50 mg, about 0.75 mg to about 25 mg, about 0.75 mg to about 20 mg, about 0.75 mg to about 15 mg, about 0.75 mg to about 10 mg, about 0.75 mg to about 5 mg, about 0.75 mg to about 4 mg, about 0.75 mg to about 3 mg, about 0.75 mg to about 2 mg, about 0.75 mg to about 1.5 mg, about 0.75 mg to about 1.25 mg, about 0.75 mg to about 1 mg, about 1 mg to about 5000 mg, about 1 mg to about 4000 mg, about 1 mg to about 3000 mg, about 1 mg to about 2000 mg, about 1 mg to about 1000 mg, about 1 mg to about 750 mg, about 1 mg to about 500 mg, about 1 mg to about 450 mg, about 1 mg to about 400 mg, about 1 mg to about 350 mg, about 1 mg to about 300 mg, about 1 mg to about 250 mg, about 1 mg to about 225 mg, about 1 mg to about 200 mg, about 1 mg to about 175 mg, about 1 mg to about 150 mg, about 1 mg to about 125 mg, about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 10 mg, about 1 mg to about 5 mg, about 1 mg to about 4 mg, about 1 mg to about 3 mg, about 1 mg to about 2 mg, about 1 mg to about 1.5 mg, about 1 mg to about 1.25 mg, about 1.25 mg to about 5000 mg, about 1.25 mg to about 4000 mg, about 1.25 mg to about 3000 mg, about 1.25 mg to about 2000 mg, about 1.25 mg to about 1000 mg, about 1.25 mg to about 750 mg, about 1.25 mg to about 500 mg, about 1.25 mg to about 450 mg, about 1.25 mg to about 400 mg, about 1.25 mg to about 350 mg, about 1.25 mg to about 300 mg, about 1.25 mg to about 250 mg, about 1.25 mg to about 225 mg, about 1.25 mg to about 200 mg, about 1.25 mg to about 175 mg, about 1.25 mg to about 150 mg, about 1.25 mg to about 125 mg, about 1.25 mg to about 100 mg, about 1.25 mg to about 75 mg, about 1.25 mg to about 50 mg, about 1.25 mg to about 25 mg, about 1.25 mg to about 20 mg, about 1.25 mg to about 15 mg, about 1.25 mg to about 10 mg, about 1.25 mg to about 5 mg, about 1.25 mg to about 4 mg, about 1.25 mg to about 3 mg, about 1.25 mg to about 2 mg, about 1.25 mg to about 1.5 mg, about 1.5 mg to about 5000 mg, about 1.5 mg to about 4000 mg, about 1.5 mg to about 3000 mg, about 1.5 mg to about 2000 mg, about 1.5 mg to about 1000 mg, about 1.5 mg to about 750 mg, about 1.5 mg to about 500 mg, about 1.5 mg to about 450 mg, about 1.5 mg to about 400 mg, about 1.5 mg to about 350 mg, about 1.5 mg to about 300 mg, about 1.5 mg to about 250 mg, about 1.5 mg to about 225 mg, about 1.5 mg to about 200 mg, about 1.5 mg to about 175 mg, about 1.5 mg to about 150 mg, about 1.5 mg to about 125 mg, about 1.5 mg to about 100 mg, about 1.5 mg to about 75 mg, about 1.5 mg to about 50 mg, about 1.5 mg to about 25 mg, about 1.5 mg to about 20 mg, about 1.5 mg to about 15 mg, about 1.5 mg to about 10 mg, about 1.5 mg to about 5 mg, about 1.5 mg to about 4 mg, about 1.5 mg to about 3 mg, about 1.5 mg to about 2 mg, about 2 mg to about 5000 mg, about 2 mg to about 4000 mg, about 2 mg to about 3000 mg, about 2 mg to about 2000 mg, about 2 mg to about 1000 mg, about 2 mg to about 750 mg, about 2 mg to about 500 mg, about 2 mg to about 450 mg, about 2 mg to about 400 mg, about 2 mg to about 350 mg, about 2 mg to about 300 mg, about 2 mg to about 250 mg, about 2 mg to about 225 mg, about 2 mg to about 200 mg, about 2 mg to about 175 mg, about 2 mg to about 150 mg, about 2 mg to about 125 mg, about 2 mg to about 100 mg, about 2 mg to about 75 mg, about 2 mg to about 50 mg, about 2 mg to about 25 mg, about 2 mg to about 20 mg, about 2 mg to about 15 mg, about 2 mg to about 10 mg, about 2 mg to about 5 mg, about 2 mg to about 4 mg, about 2 mg to about 3 mg, about 3 mg to about 5000 mg, about 3 mg to about 4000 mg, about 3 mg to about 3000 mg, about 3 mg to about 2000 mg, about 3 mg to about 1000 mg, about 3 mg to about 750 mg, about 3 mg to about 500 mg, about 3 mg to about 450 mg, about 3 mg to about 400 mg, about 3 mg to about 350 mg, about 3 mg to about 300 mg, about 3 mg to about 250 mg, about 3 mg to about 225 mg, about 3 mg to about 200 mg, about 3 mg to about 175 mg, about 3 mg to about 150 mg, about 3 mg to about 125 mg, about 3 mg to about 100 mg, about 3 mg to about 75 mg, about 3 mg to about 50 mg, about 3 mg to about 25 mg, about 3 mg to about 20 mg, about 3 mg to about 15 mg, about 3 mg to about 10 mg, about 3 mg to about 5 mg, about 3 mg to about 4 mg, about 4 mg to about 5000 mg, about 4 mg to about 4000 mg, about 4 mg to about 3000 mg, about 4 mg to about 2000 mg, about 4 mg to about 1000 mg, about 4 mg to about 750 mg, about 4 mg to about 500 mg, about 4 mg to about 450 mg, about 4 mg to about 400 mg, about 4 mg to about 350 mg, about 4 mg to about 300 mg, about 4 mg to about 250 mg, about 4 mg to about 225 mg, about 4 mg to about 200 mg, about 4 mg to about 175 mg, about 4 mg to about 150 mg, about 4 mg to about 125 mg, about 4 mg to about 100 mg, about 4 mg to about 75 mg, about 4 mg to about 50 mg, about 4 mg to about 25 mg, about 4 mg to about 20 mg, about 4 mg to about 15 mg, about 4 mg to about 10 mg, about 4 mg to about 5 mg, about 5 mg to about 5000 mg, about 5 mg to about 4000 mg, about 5 mg to about 3000 mg, about 5 mg to about 2000 mg, about 5 mg to about 1000 mg, about 5 mg to about 750 mg, about 5 mg to about 500 mg, about 5 mg to about 450 mg, about 5 mg to about 400 mg, about 5 mg to about 350 mg, about 5 mg to about 300 mg, about 5 mg to about 250 mg, about 5 mg to about 225 mg, about 5 mg to about 200 mg, about 5 mg to about 175 mg, about 5 mg to about 150 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 10 mg, about 10 mg to about 5000 mg, about 10 mg to about 4000 mg, about 10 mg to about 3000 mg, about 10 mg to about 2000 mg, about 10 mg to about 1000 mg, about 10 mg to about 750 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 225 mg, about 10 mg to about 200 mg, about 10 mg to about 175 mg, about 10 mg to about 150 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 15 mg to about 5000 mg, about 15 mg to about 4000 mg, about 15 mg to about 3000 mg, about 15 mg to about 2000 mg, about 15 mg to about 1000 mg, about 15 mg to about 750 mg, about 15 mg to about 500 mg, about 15 mg to about 450 mg, about 15 mg to about 400 mg, about 15 mg to about 350 mg, about 15 mg to about 300 mg, about 15 mg to about 250 mg, about 15 mg to about 225 mg, about 15 mg to about 200 mg, about 15 mg to about 175 mg, about 15 mg to about 150 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 5000 mg, about 20 mg to about 4000 mg, about 20 mg to about 3000 mg, about 20 mg to about 2000 mg, about 20 mg to about 1000 mg, about 20 mg to about 750 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 225 mg, about 20 mg to about 200 mg, about 20 mg to about 175 mg, about 20 mg to about 150 mg, about 20 mg to about 125 mg, about 20 mg to about 100 mg, about 20 mg to about 75 mg, about 20 mg to about 50 mg, about 20 mg to about 25 mg, about 25 mg to about 5000 mg, about 25 mg to about 4000 mg, about 25 mg to about 3000 mg, about 25 mg to about 2000 mg, about 25 mg to about 1000 mg, about 25 mg to about 750 mg, about 25 mg to about 500 mg, about 25 mg to about 450 mg, about 25 mg to about 400 mg, about 25 mg to about 350 mg, about 25 mg to about 300 mg, about 25 mg to about 250 mg, about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 5000 mg, about 50 mg to about 4000 mg, about 50 mg to about 3000 mg, about 50 mg to about 2000 mg, about 50 mg to about 1000 mg, about 50 mg to about 750 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 5000 mg, about 75 mg to about 4000 mg, about 75 mg to about 3000 mg, about 75 mg to about 2000 mg, about 75 mg to about 1000 mg, about 75 mg to about 750 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 5000 mg, about 100 mg to about 4000 mg, about 100 mg to about 3000 mg, about 100 mg to about 2000 mg, about 100 mg to about 1000 mg, about 100 mg to about 750 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 225 mg, about 100 mg to about 200 mg, about 100 mg to about 175 mg, about 100 mg to about 150 mg, about 100 mg to about 125 mg, about 125 mg to about 5000 mg, about 125 mg to about 4000 mg, about 125 mg to about 3000 mg, about 125 mg to about 2000 mg, about 125 mg to about 1000 mg, about 125 mg to about 750 mg, about 125 mg to about 500 mg, about 125 mg to about 450 mg, about 125 mg to about 400 mg, about 125 mg to about 350 mg, about 125 mg to about 300 mg, about 125 mg to about 250 mg, about 125 mg to about 225 mg, about 125 mg to about 200 mg, about 125 mg to about 175 mg, about 125 mg to about 150 mg, about 150 mg to about 5000 mg, about 150 mg to about 4000 mg, about 150 mg to about 3000 mg, about 150 mg to about 2000 mg, about 150 mg to about 1000 mg, about 150 mg to about 750 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 225 mg, about 150 mg to about 200 mg, about 150 mg to about 175 mg, about 175 mg to about 5000 mg, about 175 mg to about 4000 mg, about 175 mg to about 3000 mg, about 175 mg to about 2000 mg, about 175 mg to about 1000 mg, about 175 mg to about 750 mg, about 175 mg to about 500 mg, about 175 mg to about 450 mg, about 175 mg to about 400 mg, about 175 mg to about 350 mg, about 175 mg to about 300 mg, about 175 mg to about 250 mg, about 175 mg to about 225 mg, about 175 mg to about 200 mg, about 200 mg to about 5000 mg, about 200 mg to about 4000 mg, about 200 mg to about 3000 mg, about 200 mg to about 2000 mg, about 200 mg to about 1000 mg, about 200 mg to about 750 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 200 mg to about 225 mg, about 225 mg to about 5000 mg, about 225 mg to about 4000 mg, about 225 mg to about 3000 mg, about 225 mg to about 2000 mg, about 225 mg to about 1000 mg, about 225 mg to about 750 mg, about 225 mg to about 500 mg, about 225 mg to about 450 mg, about 225 mg to about 400 mg, about 225 mg to about 350 mg, about 225 mg to about 300 mg, about 225 mg to about 250 mg, about 250 mg to about 5000 mg, about 250 mg to about 4000 mg, about 250 mg to about 3000 mg, about 250 mg to about 2000 mg, about 250 mg to about 1000 mg, about 250 mg to about 750 mg, about 250 mg to about 500 mg, about 250 mg to about 450 mg, about 250 mg to about 400 mg, about 250 mg to about 350 mg, about 250 mg to about 300 mg, about 5000 mg, about 300 mg to about 4000 mg, about 300 mg to about 3000 mg, about 300 mg to about 2000 mg, about 300 mg to about 1000 mg, about 300 mg to about 750 mg, about 300 mg to about 500 mg, about 300 mg to about 450 mg, about 300 mg to about 400 mg, about 300 mg to about 350 mg, about 5000 mg, about 350 mg to about 4000 mg, about 350 mg to about 3000 mg, about 350 mg to about 2000 mg, about 350 mg to about 1000 mg, about 350 mg to about 750 mg, about 350 mg to about 500 mg, about 350 mg to about 450 mg, about 350 mg to about 400 mg, about 400 mg to about 5000 mg, about 400 mg to about 4000 mg, about 400 mg to about 3000 mg, about 400 mg to about 2000 mg, about 400 mg to about 1000 mg, about 400 mg to about 750 mg, about 400 mg to about 500 mg, about 400 mg to about 450 mg, about 450 mg to about 5000 mg, about 450 mg to about 4000 mg, about 450 mg to about 3000 mg, about 450 mg to about 2000 mg, about 450 mg to about 1000 mg, about 450 mg to about 750 mg, about 450 mg to about 500 mg, about 500 mg to about 5000 mg, about 500 mg to about 4000 mg, about 500 mg to about 3000 mg, about 500 mg to about 2000 mg, about 500 mg to about 1000 mg, about 500 mg to about 750 mg, about 750 mg to about 5000 mg, about 750 mg to about 4000 mg, about 750 mg to about 3000 mg, about 750 mg to about 2000 mg, about 750 mg to about 1000 mg, about 1000 mg to about 5000 mg, about 1000 mg to about 4000 mg, about 1000 mg to about 3000 mg, about 1000 mg to about 2000 mg, about 2000 mg to about 5000 mg, about 2000 mg to about 4000 mg, about 2000 mg to about 3000 mg, about 3000 mg to about 5000 mg, about 3000 mg to about 4000 mg, about 4000 mg to about 5000 mg, about 0.1 mg, about 0.25 mg, about 0.5 mg, about 0.75 mg, about 1 mg, about 1.25 mg, about 1.5 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 300 mg, about 350 mg, about 400 mg, about 450 mg, about 500 mg, about 750 mg, about 1000 mg, about 2000 mg, about 3000 mg, about 4000 mg, or about 5000 mg.

In some embodiments, about 25 mg to about 2500 mg of an excipient (e.g., FUJI FMELT TYPE C) is present in a composition herein, e.g., about 25 mg to about 2000 mg, about 25 mg to about 1500 mg, about 25 mg to about 1000 mg, about 25 mg to about 500 mg, about 25 mg to about 450 mg, about 25 mg to about 400 mg, about 25 mg to about 350, about 25 mg to about 300 mg, about 25 mg to about 250 mg, about 25 mg to about 200 mg, about 25 mg to about 150 mg, about 25 mg to about 100 mg, about 25 mg to about 75 mg, about 25 mg to about 50 mg, about 50 mg to about 2500 mg, about 50 mg to about 2000 mg, about 50 mg to about 1500 mg, about 50 mg to about 1000 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 75 mg, about 100 mg to about 2500 mg, about 100 mg to about 2000 mg, about 100 mg to about 1500 mg, about 100 mg to about 1000 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 2500 mg, about 150 mg to about 2000 mg, about 150 mg to about 1500 mg, about 150 mg to about 1000 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 2500 mg, about 200 mg to about 2000 mg, about 200 mg to about 1500 mg, about 200 mg to about 1000 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 2500 mg, about 250 mg to about 2000 mg, about 250 mg to about 1500 mg, about 250 mg to about 1000 mg, about 250 mg to about 500 mg, about 250 mg to about 450 mg, about 250 mg to about 400 mg, about 250 mg to about 350 mg, about 250 mg to about 300 mg, about 300 mg to about 2500 mg, about 300 mg to about 2000 mg, about 300 mg to about 1500 mg, about 300 mg to about 1000 mg, about 300 mg to about 500 mg, about 300 mg to about 450 mg, about 300 mg to about 400 mg, about 300 mg to about 350 mg, about 350 mg to about 2500 mg, about 350 mg to about 2000 mg, about 350 mg to about 1500 mg, about 350 mg to about 1000 mg, about 350 mg to about 500 mg, about 350 mg to about 450 mg, about 350 mg to about 400 mg, about 400 mg to about 2500 mg, about 400 mg to about 2000 mg, about 400 mg to about 1500 mg, about 400 mg to about 1000 mg, about 400 mg to about 500 mg, about 400 mg to about 450 mg, about 450 mg to about 2500 mg, about 450 mg to about 2000 mg, about 450 mg to about 1500 mg, about 450 mg to about 1000 mg, about 450 mg to about 500 mg, about 500 mg to about 2500 mg, about 500 mg to about 2000 mg, about 500 mg to about 1500 mg, about 500 mg to about 1000 mg, about 1000 mg to about 2500 mg, about 1000 mg to about 2000 mg, about 1000 mg to about 1500 mg, about 1500 mg to about 2500 mg, about 1500 mg to about 2000 mg, about 2000 mg to about 2500 mg, about 25 mg to about 250 mg, about 150 to about 500 mg, about 150 mg to about 450 mg, about 50 mg to about 400 mg, about 50 mg to about 350, about 50 mg to about 300 mg, about 50 mg to about 250 mg, about 50 mg to about 200 mg, about 50 mg to about 150 mg, about 50 mg to about 100 mg, about 50 mg to about 75 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1250 mg, about 1500 mg, about 1750 mg, about 2000 mg, about 2250 mg, or about 2500 mg.

In some embodiments, about 10% to about 40% (w/w) of a composition is an excipient (e.g., FUJI FMELT TYPE C), e.g., about 10% to about 35%, about 10% to about 30%, about 10% to about 25%, about 10% to about 20%, about 15% to about 40%, about 15% to about 35%, about 15% to about 30%, about 15% to about 25%, about 15% to about 20%, about 20% to about 40%, about 20% to about 35%, about 20% to about 30%, about 20% to about 25%, about 25% to about 40%, about 25% to about 35%, about 25% to about 30%, about 30% to about 40%, about 30% to about 35%, about 15%, about 20%, about 25%, about 30%, or about 35% of the composition is an excipient (e.g., FUJI FMELT TYPE C).

Certain compositions herein are formulated to increase energy in a person. In some cases, the amount of an excipient (e.g., FUJI FMELT TYPE C) present in the composition is about 100 mg to about 400 mg, about 150 to about 350 mg, about 200 mg, or about 250 mg. An example product is called RH1.

Certain compositions herein are formulated to increase energy in a person. In some cases, the amount of an excipient (e.g., FUJI FMELT TYPE C) present in the composition is about 100 mg to about 400 mg, about 150 to about 350 mg, about 200 mg, or about 250 mg. An example product is called RH1.

Certain compositions herein are formulated to facilitate increased relaxation in a person. In some such cases, the amount of an excipient (e.g., FUJI FMELT TYPE C) present in the composition is about 250 mg to about 750 mg, about 400 mg to about 600 mg, about 450 mg to about 550 mg, or about 500 mg. An example product is called RH2.

In some embodiments, about 5 mg to about 500 mg of an excipient (e.g., silica) is present in a composition herein, e.g., about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 500 mg, about 30 mg to about 450 mg, about 30 mg to about 400 mg, about 30 mg to about 350 mg, about 30 mg to about 300 mg, about 30 mg to about 250 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 150 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 150 mg, about 60 mg to about 100 mg, about 60 mg to about 90 mg, about 60 mg to about 80 mg, about 60 mg to about 70 mg, about 70 mg to about 500 mg, about 70 mg to about 450 mg, about 70 mg to about 400 mg, about 70 mg to about 350 mg, about 70 mg to about 300 mg, about 70 mg to about 250 mg, about 70 mg to about 200 mg, about 70 mg to about 150 mg, about 70 mg to about 100 mg, about 70 mg to about 90 mg, about 70 mg to about 80 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 150 mg, about 80 mg to about 100 mg, about 80 mg to about 90 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 90 mg to about 100 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 300 mg, about 300 mg to about 500 mg, about 300 mg to about 450 mg, about 300 mg to about 400 mg, about 300 mg to about 350 mg, about 350 mg to about 500 mg, about 350 mg to about 450 mg, about 350 mg to about 400 mg, about 400 mg to about 500 mg, about 400 mg to about 450 mg, about 450 mg to about 500 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg.

In some embodiments, about 1% to about 10% (w/w) of a composition is an excipient (e.g., silica), e.g., about 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 3% to about 10%, about 3% to about 9%, about 3% to about 8%, about 3% to about 7%, about 3% to about 6%, about 3% to about 5%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, or about 2%, about 3%, about 4%, about 5%, about 6%, about 7%, about 8%, about 9%, or about 10% of the composition is an excipient (e.g., silica).

Certain compositions herein are formulated to facilitate increased relaxation in a person. In some such cases, the amount of an excipient (e.g., silica) present in the composition is about 20 mg to about 80 mg, about 30 mg to about 70 mg, about 40 mg to about 60 mg, or about 50 mg. An example product is called RH2.

### Delivery Enhancing Agents

Some embodiments of compositions and formulations herein comprise an agent that improves delivery of the composition in a person. One example is an isolated astragaloside compound (e.g., from Astragalus membranaceus var. mongholicus). For instance, AstraGin®. AstraGin® is composed of highly purified extracts of Panax notoginseng and Astragalus membranaceus. Delivery enhancing agents such as Astragin® may enhance the delivery of an ingredient herein, such as, without limitation, organic extracts (e.g., from herbs, roots, plants), vitamins, minerals, amino acids, adaptogens, ashwagandha (including extracts and powders thereof), EDTA, maca (Lepidium meyenii), Eurycoma longifolia, fenugreek, Tribulus terrestris, cordyceps mushroom, vitamin B6, nitric oxide, beet root powder, green tea extract, Panax ginseng, phosphatidylserine, sunflower powder, magnesium glycinate, zinc, Magnolia officinalis, ginger, horny goat weed, sclareolides, sage, diindolylmethane, Bacopa monnieri, oat straw, D-aspartic acid, L-theanine, L-tryptophan, reishi mushroom, and melatonin.

Further non-limiting examples of delivery-enhancing agents may include: alkyl saccharides, cyclopentadecalactone; sodium N-[8-(2-hydroxylbenzoyl)amino]caprylate (SNAC); medium chain fatty acids, salts, and derivatives; sodium caprate, modified release formulation; sodium caprylate suspension in hydrophobic medium with matrix forming polymer; protease inhibitor and omega 3 fatty acid; liquid mixed-micelle spray; combo of protease inhibitor, permeation enhancer, pH modifier, enteric coating; lipid polymer micelle; chitosan; dodecyl-2-N,N-dimethylamino propionate (DDAIP), and other agents known in the art. Other examples of delivery-enhancing agents may include aggregation inhibitory agents; charge-modifying agents; pH control agents; degradative enzyme inhibitory agents; mucolytic or mucus clearing agents; ciliostatic agents; membrane penetration-enhancing agents; modulatory agents of epithelial junction physiology; vasodilator agents; selective transport-enhancing agents; and stabilizing delivery vehicles, carriers, adhesives, support or complex-forming species. Additional delivery-enhancing agents may include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. In some cases, a delivery enhancing agent may comprise a membrane penetration enhancing agents, such as: a surfactant; a bile salt; a phospholipid additive, mixed micelle, liposome, or carrier; an alcohol; an enamine; an NO donor compound; a long-chain amphipathic molecule; a small hydrophobic penetration enhancer; sodium or a salicylic acid derivative; a glycerol ester of acetoacetic acid; a cyclodextrin or beta-cyclodextrin derivative; a medium-chain fatty acid; a chelating agent; an amino acid or salt thereof, an N-acetylamino acid or salt thereof, an enzyme degradative to a selected membrane component; an inhibitor of fatty acid synthesis; an inhibitor of cholesterol synthesis; and any combination of the membrane penetration enhancing agents recited above.

Delivery-enhancing agents may include agents which enhance the release or solubility (e.g., from a formulation delivery vehicle), diffusion rate, penetration capacity and timing, uptake, residence time, stability, effective half-life, peak or sustained concentration levels, clearance, and other desired delivery characteristics (e.g., as measured at the site of delivery, or at a selected target site of activity such as the bloodstream or central nervous system) of a compound. Enhancement of delivery may occur through one or more of a variety of mechanisms, including, for example, by increasing the diffusion, transport, persistence or stability of the compound, increasing membrane fluidity, modulating the availability or action of calcium and other ions that regulate intracellular or paracellular permeation, solubilizing membrane components (e.g., lipids), changing non-protein and protein sulfhydryl levels in tissues, increasing water flux across a surface, modulating epithelial junction physiology, reducing clearance rates, and other mechanisms.

In some embodiments, about 5 mg to about 500 mg of a delivery enhancing agent is present in a composition herein, e.g., about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 500 mg, about 30 mg to about 450 mg, about 30 mg to about 400 mg, about 30 mg to about 350 mg, about 30 mg to about 300 mg, about 30 mg to about 250 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 150 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 150 mg, about 60 mg to about 100 mg, about 60 mg to about 90 mg, about 60 mg to about 80 mg, about 60 mg to about 70 mg, about 70 mg to about 500 mg, about 70 mg to about 450 mg, about 70 mg to about 400 mg, about 70 mg to about 350 mg, about 70 mg to about 300 mg, about 70 mg to about 250 mg, about 70 mg to about 200 mg, about 70 mg to about 150 mg, about 70 mg to about 100 mg, about 70 mg to about 90 mg, about 70 mg to about 80 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 150 mg, about 80 mg to about 100 mg, about 80 mg to about 90 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 90 mg to about 100 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 300 mg, about 300 mg to about 500 mg, about 300 mg to about 450 mg, about 300 mg to about 400 mg, about 300 mg to about 350 mg, about 350 mg to about 500 mg, about 350 mg to about 450 mg, about 350 mg to about 400 mg, about 400 mg to about 500 mg, about 400 mg to about 450 mg, about 450 mg to about 500 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg.

In some embodiments, about 2.5 mg to about 250 mg of a delivery enhancing agent is present in a composition herein, e.g., 2.5 mg to about 200 mg, about 2.5 mg to about 150 mg, about 2.5 mg to about 100 mg, about 2.5 mg to about 75 mg, about 2.5 mg to about 50 mg, about 2.5 mg to about 25 mg, about 2.5 mg to about 15 mg, about 2.5 mg to 5 mg, about 5 mg to about 250 mg, 5 mg to about 200 mg, about 5 mg to about 150 mg, about 5 mg to about 100 mg, about 5 mg to about 75 mg, about 5 mg to about 50 mg, about 5 mg to about 25 mg, about 5 mg to about 15 mg, about 15 mg to about 250 mg, 15 mg to about 200 mg, about 15 mg to about 150 mg, about 15 mg to about 100 mg, about 15 mg to about 75 mg, about 15 mg to about 50 mg, about 15 mg to about 25 mg, about 25 mg to about 250 mg, 25 mg to about 200 mg, about 25 mg to about 150 mg, about 25 mg to about 100 mg, about 25 mg to about 75 mg, about 25 mg to about 50 mg, about 50 mg to about 250 mg, 50 mg to about 200 mg, about 50 mg to about 150 mg, about 50 mg to about 100 mg, about 50 mg to about 75 mg, about 75 mg to about 250 mg, 75 mg to about 200 mg, about 75 mg to about 150 mg, about 75 mg to about 100 mg, about 100 mg to about 250 mg, 100 mg to about 200 mg, about 100 mg to about 150 mg, about 150 mg to about 250 mg, about 150 mg to about 200 mg, about 200 mg to about 250 mg, about 2.5 mg, about 5 mg, about 10 mg, about 15 mg, about 20 mg, about 25 mg, about 30 mg, about 40 mg, about 50 mg, about 75 mg, about 100 mg, about 150 mg, about 200 mg, or about 250 mg.

In some embodiments, about 1% to about 10% (w/w) of a composition is a delivery enhancing agent, e.g., about 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 3% to about 10%, about 3% to about 9%, about 3% to about 8%, about 3% to about 7%, about 3% to about 6%, about 3% to about 5%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, or about 2%, about 3%, about 4%, about 5%, about 6%, about 7%, about 8%, about 9%, or about 10% of the composition is delivery enhancing agent.

In some embodiments, about 3.5% to about 15% (w/w) of a composition is a delivery enhancing agent, e.g., about 3.5% to about 14%, about 3.5% to about 13%, about 3.5% to about 12%, about 3.5% to about 11%, about 3.5% to about 10%, about 3.5% to about 9%, about 3.5% to about 8%, about 3.5% to about 7%, about 3.5% to about 6%, about 3.5% to about 5%, about 3.5% to about 4%, about 4% to about 15%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 4% to about 5%, about 5% to about 15%, about 5% to about 14%, about 5% to about 13%, about 5% to about 12%, about 5% to about 11%, about 5% to about 10%, about 6% to about 15%, about 6% to about 14%, about 6% to about 13%, about 6% to about 12%, about 6% to about 11%, about 6% to about 10%, about 7% to about 15%, about 7% to about 14%, about 7% to about 13%, about 7% to about 12%, about 7% to about 11%, about 7% to about 10%, about 8% to about 15%, about 8% to about 14%, about 8% to about 13%, about 8% to about 12%, about 8% to about 11%, about 8% to about 10%, about 9% to about 15%, about 9% to about 14%, about 9% to about 13%, about 9% to about 12%, about 9% to about 11%, about 9% to about 10%, about 5%, about 6%, about 7%, about 8%, about 9%, about 10%, about 11%, about 12%, about 13%, about 14%, or about 15% of the composition is delivery enhancing agent.

Certain compositions herein are formulated to facilitate increased energy in a person. In some such cases, the amount of a delivery enhancing agent present in the composition is about 25 mg to about 75 mg, about 40 mg to about 60 mg, about 45 mg to about 55 mg, or about 50 mg. An example product is called RH1.

Certain compositions herein are formulated to facilitate increased relaxation in a person. In some such cases, the amount of a delivery enhancing agent present in the composition is about 25 mg to about 75 mg, about 40 mg to about 60 mg, about 45 mg to about 55 mg, or about 50 mg. An example product is called RH2.

Certain compositions herein are formulated to improve the sleep health of a person. In some such cases, the amount of a delivery enhancing agent present in the composition is about 10 mg to about 40 mg, about 15 mg to about 35 mg, about 20 mg to about 30 mg, or about 25 mg. An example product is called RH3.

As described above, intake of AstraGin® may have various effects. For example, intake of AstraGin® may improve a person's ability to receive nutrients into their body. While AstraGin® is described above as an example of a delivery enhancing agent, AstraGin® is exemplary and other delivery enhancing agents may be used.

### Chelating Agent

Some embodiments of compositions and formulations herein comprise a chelating agent. Chelating agents are used to reduce levels of injurious heavy metals in blood and tissue. Chelating agents are classified depending upon the heavy metal they bind. In some instances, there are agents that have high specificity and bind to narrow classes of metals. In other cases, there are chelating agents that bind to multiple metals. Because of their affinity to bind to metals, chelating agents are used in medicine, the chemical industry, and in pharmaceutical technologies. A chelating agent is present in some embodiments for its detoxification properties. Specifically, the ability of chelating agents to bind heavy and toxic metals. Furthermore, chelating agents may be added to composition formulations because of their stability, compatibility, and low toxicity. Additionally, chelating agents may be used as disruptors to allow large molecules to enter the bloodstream. These properties may permit the chelating agents to enhance the action of preservative and antibacterial components and stabilize the action of antioxidants. Non-limiting examples include deferoxamine, deferasirox, deferiprone, penicillamine, trientine, dimercaprol, ethylenediamine, porphine, heme, sodium gluconate, tetrasodium glutamate diacetate, phytic acid, and ethylenediaminetetraacetic acid (EDTA). Some embodiments of compositions and formulations herein comprise ethylenediaminetetraacetic acid (EDTA).

In some embodiments, about 5 mg to about 500 mg of a chelating agent (e.g., EDTA) is present in a composition herein, e.g., about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 500 mg, about 30 mg to about 450 mg, about 30 mg to about 400 mg, about 30 mg to about 350 mg, about 30 mg to about 300 mg, about 30 mg to about 250 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 150 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 150 mg, about 60 mg to about 100 mg, about 60 mg to about 90 mg, about 60 mg to about 80 mg, about 60 mg to about 70 mg, about 70 mg to about 500 mg, about 70 mg to about 450 mg, about 70 mg to about 400 mg, about 70 mg to about 350 mg, about 70 mg to about 300 mg, about 70 mg to about 250 mg, about 70 mg to about 200 mg, about 70 mg to about 150 mg, about 70 mg to about 100 mg, about 70 mg to about 90 mg, about 70 mg to about 80 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 150 mg, about 80 mg to about 100 mg, about 80 mg to about 90 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 90 mg to about 100 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 300 mg, about 300 mg to about 500 mg, about 300 mg to about 450 mg, about 300 mg to about 400 mg, about 300 mg to about 350 mg, about 350 mg to about 500 mg, about 350 mg to about 450 mg, about 350 mg to about 400 mg, about 400 mg to about 500 mg, about 400 mg to about 450 mg, about 450 mg to about 500 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg.

In some embodiments, about 2.5 mg to about 25 mg of a chelating agent (e.g., EDTA) is present in a composition herein, e.g., 2.5 mg to about 200 mg, about 2.5 mg to about 150 mg, about 2.5 mg to about 100 mg, about 2.5 mg to about 75 mg, about 2.5 mg to about 50 mg, about 2.5 mg to about 25 mg, about 2.5 mg to about 15 mg, about 2.5 mg to 5 mg, about 5 mg to about 250 mg, 5 mg to about 200 mg, about 5 mg to about 150 mg, about 5 mg to about 100 mg, about 5 mg to about 75 mg, about 5 mg to about 50 mg, about 5 mg to about 25 mg, about 5 mg to about 15 mg, about 15 mg to about 250 mg, 15 mg to about 200 mg, about 15 mg to about 150 mg, about 15 mg to about 100 mg, about 15 mg to about 75 mg, about 15 mg to about 50 mg, about 15 mg to about 25 mg, about 25 mg to about 250 mg, 25 mg to about 200 mg, about 25 mg to about 150 mg, about 25 mg to about 100 mg, about 25 mg to about 75 mg, about 25 mg to about 50 mg, about 50 mg to about 250 mg, 50 mg to about 200 mg, about 50 mg to about 150 mg, about 50 mg to about 100 mg, about 50 mg to about 75 mg, about 75 mg to about 250 mg, 75 mg to about 200 mg, about 75 mg to about 150 mg, about 75 mg to about 100 mg, about 100 mg to about 250 mg, 100 mg to about 200 mg, about 100 mg to about 150 mg, about 150 mg to about 250 mg, about 150 mg to about 200 mg, about 200 mg to about 250 mg, about 2.5 mg, about 5 mg, about 10 mg, about 15 mg, about 20 mg, about 25 mg, about 30 mg, about 40 mg, about 50 mg, about 75 mg, about 100 mg, about 150 mg, about 200 mg, or about 250 mg.

In some embodiments, about 1% to about 10% (w/w) of a composition is a chelating agent (e.g., EDTA), e.g., about 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 3% to about 10%, about 3% to about 9%, about 3% to about 8%, about 3% to about 7%, about 3% to about 6%, about 3% to about 5%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, or about 2%, about 3%, about 4%, about 5%, about 6%, about 7%, about 8%, about 9%, or about 10%.

In some embodiments, about 3.5% to about 15% (w/w) of a composition is a chelating agent (e.g., EDTA), e.g., about 3.5% to about 14%, about 3.5% to about 13%, about 3.5% to about 12%, about 3.5% to about 11%, about 3.5% to about 10%, about 3.5% to about 9%, about 3.5% to about 8%, about 3.5% to about 7%, about 3.5% to about 6%, about 3.5% to about 5%, about 3.5% to about 4%, about 4% to about 15%, about 4% to about 14%, about 4% to about 13%, about 4% to about 12%, about 4% to about 11%, about 4% to about 10%, about 4% to about 9%, about 4% to about 8%, about 4% to about 7%, about 4% to about 6%, about 4% to about 5%, about 5% to about 15%, about 5% to about 14%, about 5% to about 13%, about 5% to about 12%, about 5% to about 11%, about 5% to about 10%, about 6% to about 15%, about 6% to about 14%, about 6% to about 13%, about 6% to about 12%, about 6% to about 11%, about 6% to about 10%, about 7% to about 15%, about 7% to about 14%, about 7% to about 13%, about 7% to about 12%, about 7% to about 11%, about 7% to about 10%, about 8% to about 15%, about 8% to about 14%, about 8% to about 13%, about 8% to about 12%, about 8% to about 11%, about 8% to about 10%, about 9% to about 15%, about 9% to about 14%, about 9% to about 13%, about 9% to about 12%, about 9% to about 11%, about 9% to about 10%, about 5%, about 6%, about 7%, about 8%, about 9%, about 10%, about 11%, about 12%, about 13%, about 14%, or about 15%.

In some embodiments, certain compositions herein are formulated to facilitate increased energy in a person. In some such cases, the amount of chelating agent present in the composition is about 25 mg to about 75 mg, about 40 mg to about 60 mg, about 45 mg to about 55 mg, or about 50 mg. An example product is called RH1.

In some embodiments, certain compositions herein are formulated to facilitate increased relaxation in a person. In some such cases, the amount of chelating agent present in the composition is about 25 mg to about 75 mg, about 40 mg to about 60 mg, about 45 mg to about 55 mg, or about 50 mg. An example product is called RH2.

In some embodiments, certain compositions herein are formulated to improve the sleep health of a person. In some such cases, the amount of chelating agent present in the composition is about 10 mg to about 40 mg, about 15 mg to about 35 mg, about 20 mg to about 30 mg, or about 25 mg. An example product is called RH3.

As described above, intake of a chelating agent (e.g., EDTA) may lead to various effects for a person. For example, intake of a chelating agent may reduce levels of heavy metals within a person as well as act as a disruptor to allow large molecules to enter the bloodstream.

### Sodium Starch Glycolate and Disintegrants

In one aspect, provided are compositions and formulations comprising a disintegrant. In some cases, the disintegrant comprises silicon dioxide (e.g., anhydrous silicon dioxide) and/or sodium starch glycolate. In one aspect, provided are compositions and formulations comprising sodium starch glycolate (e.g., EXPLOTAB®). In some embodiments, sodium starch glycolate is an excipient and/or enhancing agent, that is added to compositions to improve composition delivery. Sodium starch glycolate may be used for its super-disintegrant properties to promote rapid dissolution of solid compositions. For instance, it may be used as a dissolution excipient for compositions formulated into tablets and capsules. Its ability to rapidly absorb water results in swelling which leads to rapid disintegration of compositions containing sodium starch glycolate. Importantly, in some cases the hydrophilic properties of sodium starch glycolate are uninhibited in the presence of other hydrophobic excipients.

In some embodiments, about 5 mg to about 550 mg of disintegrant (e.g., anhydrous silicon dioxide) and/or sodium starch glycolate (e.g., EXPLOTAB®) is present in a composition herein, e.g., about 5 mg to about 500 mg, about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 550 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 550 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 550 mg, about 30 mg to about 500 mg, about 30 mg to about 450 mg, about 30 mg to about 400 mg, about 30 mg to about 350 mg, about 30 mg to about 300 mg, about 30 mg to about 250 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 550 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 150 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 550 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 60 mg to about 550 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 150 mg, about 60 mg to about 100 mg, about 60 mg to about 90 mg, about 60 mg to about 80 mg, about 60 mg to about 70 mg, about 70 mg to about 550 mg, about 70 mg to about 500 mg, about 70 mg to about 450 mg, about 70 mg to about 400 mg, about 70 mg to about 350 mg, about 70 mg to about 300 mg, about 70 mg to about 250 mg, about 70 mg to about 200 mg, about 70 mg to about 150 mg, about 70 mg to about 100 mg, about 70 mg to about 90 mg, about 70 mg to about 80 mg, about 80 mg to about 550 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 150 mg, about 80 mg to about 100 mg, about 80 mg to about 90 mg, about 90 mg to about 550 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 90 mg to about 100 mg, about 100 mg to about 550 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 550 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 550 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 550 mg, about 250 mg to about 500 mg, about 250 mg to about 450 mg, about 250 mg to about 400 mg, about 250 mg to about 350 mg, about 250 mg to about 300 mg, about 300 mg to about 550 mg, about 300 mg to about 500 mg, about 300 mg to about 450 mg, about 300 mg to about 400 mg, about 300 mg to about 350 mg, about 350 mg to about 550 mg, about 350 mg to about 500 mg, about 350 mg to about 450 mg, about 350 mg to about 400 mg, about 400 mg to about 550 mg, 400 mg to about 500 mg, about 400 mg to about 450 mg, about 450 mg to about 550 mg, about 450 mg to about 500 mg, about 500 mg to about 550 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, or about 500 mg.

In some embodiments, about 1.5 mg to about 175 mg of disintegrant (e.g., anhydrous silicon dioxide) and/or sodium starch glycolate (e.g., EXPLOTAB®) is present in a composition herein, e.g., about 1.5 mg to about 125 mg, about 1.5 mg to about 75 mg, about 1.5 mg to about 25 mg, about 1.5 mg to about 20 mg, about 1.5 mg to about 16 mg, about 1.5 mg to about 12 mg, about 1.5 mg to about 8 mg, about 1.5 mg to about 4 mg, about 1.5 mg to about 2 mg, about 2 mg to about 175 mg, about 2 mg to about 125 mg, about 2 mg to about 75 mg, about 2 mg to about 25 mg, about 2 mg to about 20 mg, about 2 mg to about 16 mg, about 2 mg to about 12 mg, about 2 mg to about 8 mg, about 2 mg to about 4 mg, about 4 mg to about 175 mg, about 4 mg to about 125 mg, about 4 mg to about 75 mg, about 4 mg to about 25 mg, about 4 mg to about 20 mg, about 4 mg to about 16 mg, about 4 mg to about 12 mg, about 4 mg to about 8 mg, about 8 mg to about 175 mg, about 8 mg to about 125 mg, about 8 mg to about 75 mg, about 8 mg to about 25 mg, about 8 mg to about 20 mg, about 8 mg to about 16 mg, about 8 mg to about 12 mg, about 12 mg to about 175 mg, about 12 mg to about 125 mg, about 12 mg to about 75 mg, about 12 mg to about 25 mg, about 12 mg to about 20 mg, about 12 mg to about 16 mg, about 16 mg to about 175 mg, about 16 mg to about 125 mg, about 16 mg to about 75 mg, about 16 mg to about 25 mg, about 16 mg to about 20 mg, about 20 mg to about 175 mg, about 20 mg to about 125 mg, about 20 mg to about 75 mg, about 20 mg to about 25 mg, about 25 mg to about 175 mg, about 25 mg to about 125 mg, about 25 mg to about 75 mg, about 75 mg to about 175 mg, about 75 mg to about 125 mg, about 125 mg to about 175 mg, about 1.5 mg, about 2 mg, about 4 mg, about 8 mg, about 12 mg, about 16 mg, about 20 mg, about 25 mg, about 75 mg, about 125 mg, or about 175 mg.

In some embodiments, about 1% to about 10% (w/w) of the a composition is disintegrant (e.g., anhydrous silicon dioxide) and/or sodium starch glycolate (e.g., EXPLOTAB®), e.g., about 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 3% to about 10%, about 3% to about 9%, about 3% to about 8%, about 3% to about 7%, about 3% to about 6%, about 3% to about 5%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, or about 2%, about 3%, about 4%, about 5%, about 6%, about 7%, about 8%, about 9%, or about 10% of the composition is enhancing agent.

Certain compositions herein are formulated to increase absorption and/or improve composition delivery with a disintegrant and/or sodium starch glycolate.

Certain compositions herein are formulated to increase energy in a person. In some cases, the amount of disintegrant and/or sodium starch glycolate present in the composition is about 1 mg to about 25, about 3 mg to about 15 mg, about 4 mg to about 10 mg, about 6 mg, or about 6.5 mg. In some such cases, the amount of disintegrant and/or sodium starch glycolate present in the composition is about 5 mg to about 105 mg, about 25 mg to about 85 mg, about 45 mg to about 65 mg, about 90 mg, or about 55 mg. An example product is called RH1.

Certain compositions herein are formulated to facilitate increased relaxation in a person. In some such cases, the amount of disintegrant and/or sodium starch glycolate present in the composition is about 25 mg to about 85 mg, about 45 mg to about 65 mg, or about 55 mg. An example product is called RH2.

Certain compositions herein are formulated to improve the sleep health of a person. In some such cases, the amount of disintegrant and/or sodium starch glycolate present in the composition is about 6 mg to about 26 mg, about 11 mg to about 21 mg, or about 16.25 mg. An example product is called RH3.

As described above, intake of disintegrants and/or sodium starch glycolate may lead to various effects for a person. For example, intake of disintegrants and/or sodium starch glycolate may lead to rapid disintegration of taken compositions, and therefore, may increase the speed of delivery of the taken compositions. While EXPLOTAB® is described above as a source of sodium starch glycolate, EXPLOTAB® is exemplary and other sources of sodium starch glycolate may be used.

### Silicon Dioxide, Adsorbents, and Glidants

In one aspect, provided are compositions and formulations comprising silicon dioxide, e.g., colloidal silicon dioxide. In some embodiments, the silicon dioxide is present as an anti-caking agent, adsorbent, disintegrant, and/or glidant to allow powder to flow freely when tablets are processed. In some embodiments, the silicon dioxide is a lubricant.

In some embodiments, about 0.3 mg to about 30 mg of adsorbent, glidant, and/or silicon dioxide is present in a composition herein, e.g., about 0.3 mg to about 20 mg, about 0.3 mg to about 10 mg, about 0.3 mg to about 7.5 mg, about 0.3 mg to about 5 mg, about 0.3 mg to about 4 mg, about 0.3 mg to about 3 mg, about 0.3 mg to about 2 mg, about 0.3 mg to about 1 mg, about 0.3 mg to about 0.5 mg, about 0.5 mg to about 30 mg, about 0.5 mg to about 20 mg, about 0.5 mg to about 10 mg, about 0.5 mg to about 7.5 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 30 mg, about 1 mg to about 20 mg, about 1 mg to about 10 mg, about 1 mg to about 7.5 mg, about 1 mg to about 5 mg, about 1 mg to about 4 mg, about 1 mg to about 3 mg, about 1 mg to about 2 mg, about 2 mg to about 30 mg, about 2 mg to about 20 mg, about 2 mg to about 10 mg, about 2 mg to about 7.5 mg, about 2 mg to about 5 mg, about 2 mg to about 4 mg, about 2 mg to about 3 mg, about 3 mg to about 30 mg, about 3 mg to about 20 mg, about 3 mg to about 10 mg, about 3 mg to about 7.5 mg, about 3 mg to about 5 mg, about 3 mg to about 4 mg, about 4 mg to about 30 mg, about 4 mg to about 20 mg, about 4 mg to about 10 mg, about 4 mg to about 7.5 mg, about 4 mg to about 5 mg, about 5 mg to about 30 mg, about 5 mg to about 20 mg, about 5 mg to about 10 mg, about 5 mg to about 7.5 mg, about 7.5 mg to about 30 mg, about 7.5 mg to about 20 mg, about 7.5 mg to about 10 mg, about 10 mg to about 30 mg, about 10 mg to about 20 mg, about 20 mg to about 30 mg, about 0.3 mg, about 0.5 mg, about 1 mg, about 2 mg, about 3 mg, about 4 mg, about 5 mg, about 7.5 mg, about 10 mg, about 20 mg, or about 30 mg.

In some embodiments, about 0.1 mg to about 10 mg of adsorbent, glidant, and/or silicon dioxide is present in a composition herein, e.g., about 0.1 mg to about 7.5 mg, about 0.1 mg to about 5 mg, about 0.1 mg to about 2.5 mg, about 0.1 mg to about 1 mg, about 0.1 mg to about 0.9 mg, about 0.1 mg to about 0.8 mg, about 0.1 mg to about 0.7 mg, about 0.1 mg to about 0.6 mg, 0.1 mg to about 0.5 mg, about 0.1 mg to about 0.4 mg, about 0.1 mg to about 0.3 mg, about 0.1 mg to about 0.2 mg, about 0.2 mg to about 10 mg, about 0.2 mg to about 7.5 mg, about 0.2 mg to about 5 mg, about 0.2 mg to about 2.5 mg, about 0.2 mg to about 1 mg, about 0.2 mg to about 0.9 mg, about 0.2 mg to about 0.8 mg, about 0.2 mg to about 0.7 mg, about 0.2 mg to about 0.6 mg, 0.2 mg to about 0.5 mg, about 0.2 mg to about 0.4 mg, about 0.2 mg to about 0.3 mg, about 0.3 mg to about 10 mg, about 0.3 mg to about 7.5 mg, about 0.3 mg to about 5 mg, about 0.3 mg to about 2.5 mg, about 0.3 mg to about 1 mg, about 0.3 mg to about 0.9 mg, about 0.3 mg to about 0.8 mg, about 0.3 mg to about 0.7 mg, about 0.3 mg to about 0.6 mg, 0.3 mg to about 0.5 mg, about 0.3 mg to about 0.4 mg, about 0.4 mg to about 10 mg, about 0.4 mg to about 7.5 mg, about 0.4 mg to about 5 mg, about 0.4 mg to about 2.5 mg, about 0.4 mg to about 1 mg, about 0.4 mg to about 0.9 mg, about 0.4 mg to about 0.8 mg, about 0.4 mg to about 0.7 mg, about 0.4 mg to about 0.6 mg, 0.4 mg to about 0.5 mg, about 0.5 mg to about 10 mg, about 0.5 mg to about 7.5 mg, about 0.5 mg to about 5 mg, about 0.5 mg to about 2.5 mg, about 0.5 mg to about 1 mg, about 0.5 mg to about 0.9 mg, about 0.5 mg to about 0.8 mg, about 0.5 mg to about 0.7 mg, about 0.5 mg to about 0.6 mg, about 0.6 mg to about 10 mg, about 0.6 mg to about 7.5 mg, about 0.6 mg to about 5 mg, about 0.6 mg to about 2.5 mg, about 0.6 mg to about 1 mg, about 0.6 mg to about 0.9 mg, about 0.6 mg to about 0.8 mg, about 0.6 mg to about 0.7 mg, about 0.7 mg to about 10 mg, about 0.7 mg to about 7.5 mg, about 0.7 mg to about 5 mg, about 0.7 mg to about 2.5 mg, about 0.7 mg to about 1 mg, about 0.7 mg to about 0.9 mg, about 0.7 mg to about 0.8 mg, about 0.8 mg to about 10 mg, about 0.8 mg to about 7.5 mg, about 0.8 mg to about 5 mg, about 0.8 mg to about 2.5 mg, about 0.8 mg to about 1 mg, about 0.8 mg to about 0.9 mg, about 0.9 mg to about 10 mg, about 0.9 mg to about 7.5 mg, about 0.9 mg to about 5 mg, about 0.9 mg to about 2.5 mg, about 0.9 mg to about 1 mg, about 1 mg to about 10 mg, about 1 mg to about 7.5 mg, about 1 mg to about 5 mg, about 1 mg to about 2.5 mg, about 2.5 mg to about 10 mg, about 2.5 mg to about 7.5 mg, about 2.5 mg to about 5 mg, about 5 mg to about 10 mg, about 5 mg to about 7.5 mg, about 7.5 mg to about 10 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2.5 mg, about 5 mg, about 7.5 mg, or about 10 mg.

In some embodiments, about 0.1% to about 2.5% (w/w) of a composition is adsorbent, glidant, and/or silicon dioxide, e.g., about 0.1% to about 2%, about 0.1% to about 1.5%, about 0.1% to about 1%, about 0.1% to about 0.75%, about 0.1% to about 0.5%, about 0.1% to about 0.25%, about 0.25% to about 2.5%, about 0.25% to about 2%, about 0.25% to about 1.5%, about 0.25% to about 1%, about 0.25% to about 0.75%, about 0.25% to about 0.5%, about 0.5% to about 2.5%, about 0.5% to about 2%, about 0.5% to about 1.5%, about 0.5% to about 1%, about 0.5% to about 0.75%, about 0.75% to about 2.5%, about 0.75% to about 2%, about 0.75% to about 1.5%, about 0.75% to about 1%, about 1% to about 2.5%, about 1% to about 2%, about 1% to about 1.5%, about 1.5% to about 2.5%, about 1.5% to about 2%, about 2% to about 2.5%, about 0.1%, about 0.25%, about 0.5%, about 0.75%, about 1%, about 1.5%, about 2%, or about 2.5%.

Certain compositions herein are formulated to facilitate increased energy in a person. In some such cases, the amount of adsorbent, glidant, and/or silicon dioxide in the composition is about 0.5 mg to about 5.5 mg, about 2 mg to about 4 mg, or about 3 mg. An example product is called RH1.

Certain compositions herein are formulated to facilitate increased relaxation in a person. In some such cases, the amount of adsorbent, glidant, and/or silicon dioxide in the composition is about 0.5 mg to about 5.5 mg, about 2 mg to about 4 mg, or about 3 mg. An example product is called RH2.

Certain compositions herein are formulated to improve the sleep health of a person. In some such cases, the amount of adsorbent, glidant, and/or silicon dioxide present in the composition is about 0.25 mg to about 1.75 mg, about 0.75 mg to about 1.25 mg, about 1 mg. An example product is called RH3.

As described above, silicon dioxide may be present as an anti-caking agent to aid in the processing of tablets. In some cases, silicon dioxide may act as a lubricant to improve the manufacturing of tablets.

### Cellulose and Binders/Diluents

In one aspect, provided are compositions and formulations comprising a binder and/or diluent. In one aspect, provided are compositions and formulations comprising cellulose, such as microcrystalline cellulose. In some cases, cellulose is a binder/diluent. The binder, diluent, and/or cellulose may be used to enhance stability of compositions and extend composition release.

In some embodiments, about 10 mg to about 1500 mg of a binder, diluent, and/or cellulose (e.g., microcrystalline cellulose) is present in a composition herein, e.g., about 10 mg to about 1250 mg, about 10 mg to about 1000 mg, about 10 mg to about 750 mg, about 10 mg to about 500 mg, about 10 mg to about 250 mg, about 10 mg to about 150 mg, about 10 mg to about 125 mg, about 10 mg to about 100 mg, about 10 mg to about 75 mg, about 10 mg to about 50 mg, about 10 mg to about 25 mg, about 25 mg to about 1500 mg, about 25 mg to about 1250 mg, about 25 mg to about 1000 mg, about 25 mg to about 750 mg, about 25 mg to about 500 mg, about 25 mg to about 250 mg, about 25 mg to about 150 mg, about 25 mg to about 125 mg, about 25 mg to about 100 mg, about 25 mg to about 75 mg, about 25 mg to about 50 mg, about 50 mg to about 1500 mg, about 50 mg to about 1250 mg, about 50 mg to about 1000 mg, about 50 mg to about 750 mg, about 50 mg to about 500 mg, about 50 mg to about 250 mg, about 50 mg to about 150 mg, about 50 mg to about 125 mg, about 50 mg to about 100 mg, about 50 mg to about 75 mg, about 75 mg to about 1500 mg, about 75 mg to about 1250 mg, about 75 mg to about 1000 mg, about 75 mg to about 750 mg, about 75 mg to about 500 mg, about 75 mg to about 250 mg, about 75 mg to about 150 mg, about 75 mg to about 125 mg, about 75 mg to about 100 mg, about 100 mg to about 1500 mg, about 100 mg to about 1250 mg, about 100 mg to about 1000 mg, about 100 mg to about 750 mg, about 100 mg to about 500 mg, about 100 mg to about 250 mg, about 100 mg to about 150 mg, about 100 mg to about 125 mg, about 125 mg to about 1500 mg, about 125 mg to about 1250 mg, about 125 mg to about 1000 mg, about 125 mg to about 750 mg, about 125 mg to about 500 mg, about 125 mg to about 250 mg, about 125 mg to about 150 mg, about 150 mg to about 1500 mg, about 150 mg to about 1250 mg, about 150 mg to about 1000 mg, about 150 mg to about 750 mg, about 150 mg to about 500 mg, about 150 mg to about 250 mg, about 250 mg to about 1500 mg, about 250 mg to about 1250 mg, about 250 mg to about 1000 mg, about 250 mg to about 750 mg, about 250 mg to about 500 mg, about 500 mg to about 1500 mg, about 500 mg to about 1250 mg, about 500 mg to about 1000 mg, about 500 mg to about 750 mg, about 750 mg to about 1500 mg, about 750 mg to about 1250 mg, about 750 mg to about 1000 mg, about 1000 mg to about 1500 mg, about 1000 mg to about 1250 mg, about 1250 mg to about 1500 mg, about 10 mg, about 25 mg, about 50 mg, about 75 mg, about 100 mg, about 125 mg, about 150 mg, about 250 mg, about 500 mg, about 750 mg, about 1000 mg, about 1250 mg, or about 1500 mg.

In some embodiments, about 4 mg to about 400 mg of a binder, diluent, and/or cellulose (e.g., microcrystalline cellulose) is present in a composition herein, e.g., about 4 mg to about 200 mg, about 4 mg to about 160 mg, about 4 mg to about 120 mg, about 4 mg to about 80 mg, about 4 mg to about 60 mg, about 4 mg to about 52 mg, about 4 mg to about 44 mg, about 4 mg to about 40 mg, about 4 mg to about 36 mg, about 4 mg to about 32 mg, about 4 mg to about 28 mg, about 4 mg to about 24 mg, about 4 mg to about 20 mg, about 4 mg to about 16 mg, about 4 mg to about 12 mg, about 4 mg to about 8 mg, about 8 mg to about 400 mg, about 8 mg to about 200 mg, about 8 mg to about 160 mg, about 8 mg to about 120 mg, about 8 mg to about 80 mg, about 8 mg to about 60 mg, about 8 mg to about 52 mg, about 8 mg to about 44 mg, about 8 mg to about 40 mg, about 8 mg to about 36 mg, about 8 mg to about 32 mg, about 8 mg to about 28 mg, about 8 mg to about 24 mg, about 8 mg to about 20 mg, about 8 mg to about 16 mg, about 8 mg to about 12 mg, about 12 mg to about 400 mg, about 12 mg to about 200 mg, about 12 mg to about 160 mg, about 12 mg to about 120 mg, about 12 mg to about 80 mg, about 12 mg to about 60 mg, about 12 mg to about 52 mg, about 12 mg to about 44 mg, about 12 mg to about 40 mg, about 12 mg to about 36 mg, about 12 mg to about 32 mg, about 12 mg to about 28 mg, about 12 mg to about 24 mg, about 12 mg to about 20 mg, about 12 mg to about 16 mg, about 16 mg to about 400 mg, about 16 mg to about 200 mg, about 16 mg to about 160 mg, about 16 mg to about 120 mg, about 16 mg to about 80 mg, about 16 mg to about 60 mg, about 16 mg to about 52 mg, about 16 mg to about 44 mg, about 16 mg to about 40 mg, about 16 mg to about 36 mg, about 16 mg to about 32 mg, about 16 mg to about 28 mg, about 16 mg to about 24 mg, about 16 mg to about 20 mg, about 20 mg to about 400 mg, about 20 mg to about 200 mg, about 20 mg to about 160 mg, about 20 mg to about 120 mg, about 20 mg to about 80 mg, about 20 mg to about 60 mg, about 20 mg to about 52 mg, about 20 mg to about 44 mg, about 20 mg to about 40 mg, about 20 mg to about 36 mg, about 20 mg to about 32 mg, about 20 mg to about 28 mg, about 20 mg to about 24 mg, about 24 mg to about 400 mg, about 24 mg to about 200 mg, about 24 mg to about 160 mg, about 24 mg to about 120 mg, about 24 mg to about 80 mg, about 24 mg to about 60 mg, about 24 mg to about 52 mg, about 24 mg to about 44 mg, about 24 mg to about 40 mg, about 24 mg to about 36 mg, about 24 mg to about 32 mg, about 24 mg to about 28 mg, about 28 mg to about 400 mg, about 28 mg to about 200 mg, about 28 mg to about 160 mg, about 28 mg to about 120 mg, about 28 mg to about 80 mg, about 28 mg to about 60 mg, about 28 mg to about 52 mg, about 28 mg to about 44 mg, about 28 mg to about 40 mg, about 28 mg to about 36 mg, about 28 mg to about 32 mg, about 32 mg to about 400 mg, about 32 mg to about 200 mg, about 32 mg to about 160 mg, about 32 mg to about 120 mg, about 32 mg to about 80 mg, about 32 mg to about 60 mg, about 32 mg to about 52 mg, about 32 mg to about 44 mg, about 32 mg to about 40 mg, about 32 mg to about 36 mg, about 36 mg to about 400 mg, about 36 mg to about 200 mg, about 36 mg to about 160 mg, about 36 mg to about 120 mg, about 36 mg to about 80 mg, about 36 mg to about 60 mg, about 36 mg to about 52 mg, about 36 mg to about 44 mg, about 36 mg to about 40 mg, about 40 mg to about 400 mg, about 40 mg to about 200 mg, about 40 mg to about 160 mg, about 40 mg to about 120 mg, about 40 mg to about 80 mg, about 40 mg to about 60 mg, about 40 mg to about 52 mg, about 40 mg to about 44 mg, about 44 mg to about 400 mg, about 44 mg to about 200 mg, about 44 mg to about 160 mg, about 44 mg to about 120 mg, about 44 mg to about 80 mg, about 44 mg to about 60 mg, about 44 mg to about 52 mg, about 52 mg to about 400 mg, about 52 mg to about 200 mg, about 52 mg to about 160 mg, about 52 mg to about 120 mg, about 52 mg to about 80 mg, about 52 mg to about 60 mg, about 60 mg to about 400 mg, about 60 mg to about 200 mg, about 60 mg to about 160 mg, about 60 mg to about 120 mg, about 60 mg to about 80 mg, about 80 mg to about 400 mg, about 80 mg to about 200 mg, about 80 mg to about 160 mg, about 80 mg to about 120 mg, about 120 mg to about 400 mg, about 120 mg to about 200 mg, about 120 mg to about 160 mg, about 160 mg to about 400 mg, about 160 mg to about 200 mg, about 200 mg to about 400 mg, about 4 mg, about 8 mg, about 12 mg, about 16 mg, about 20 mg, about 24 mg, about 28 mg, about 32 mg, about 36 mg, about 40 mg, about 44 mg, about 52 mg, about 60 mg, about 80 mg, about 120 mg, about 160 mg, about 200 mg, or about 400 mg.

In some embodiments, about 1% to about 21% (w/w) of a composition is a binder, diluent, and/or cellulose (e.g., microcrystalline cellulose), e.g., about 1% to about 16%, about 1% to about 11%, about 1% to about 6%, about 6% to about 21%, about 6% to about 16%, about 6% to about 11%, about 11% to about 21%, about 11% to about 16%, about 16% to about 21%, about 1%, about 6%, about 11%, about 16%, or about 21%.

In some embodiments, about 6% to about 24% (w/w) of a composition is a binder, diluent, and/or cellulose (e.g., microcrystalline cellulose), e.g., about 6% to about 18%, about 6% to about 15%, about 6% to about 12%, about 6% to about 9%, about 9% to about 24%, about 9% to about 18%, about 9% to about 15%, about 9% to about 12%, about 12% to about 24%, about 12% to about 18%, about 12% to about 15%, about 15% to about 24%, about 15%, about 18%, about 18% to about 24%, about 6%, about 9%, about 12%, about 15%, about 18%, or about 24%.

Certain compositions herein are formulated to facilitate increased energy in a person. In some such cases, the amount of binder, diluent, and/or cellulose in the composition is about 116 mg to about 148 mg, about 124 mg to about 140 mg, or about 132 mg. In other such cases, the amount of binder, diluent, and/or cellulose in the composition is about 120 mg to about 150 mg, about 130 mg to about 140 mg, or about 135 mg. An example product is called RH1.

Certain compositions herein are formulated to facilitate increased relaxation in a person. In some such cases, the amount of binder, diluent, and/or cellulose in the composition is about 115 mg to about 135 mg, about 120 mg to about 130 mg, or about 125 mg. In other such cases, the amount of binder, diluent, and/or cellulose in the composition is about 112 mg to about 132 mg, about 117 mg to about 127 mg, or about 122 mg. An example product is called RH2.

Certain compositions herein are formulated to improve the sleep health of a person. In some such cases, the amount of binder, diluent, and/or cellulose in the composition is about 36 mg to about 44 mg, about 38 mg to about 42 mg, or about 40 mg. An example product is called RH3.

As described above, intake of cellulose or other binders/diluents may lead to various effects, such as enhancing the stability of a composition or extending the release of a composition that a person has taken.

### Sweetening Agent

In one aspect, provided are compositions and formulations comprising a sweetening agent. Non-limiting examples of sweetening agents include acesulfame, aspartame, dextrates, dextrose, erythritol, fructose, galactose, maltitol, maltose, mannitol, saccharin, sorbitol, sucralose, sugar, syrup, and tagatose. In some embodiments, the sweetening agent comprises sucralose.

In some embodiments, about 2 mg to about 250 mg of sweetening agent is present in a composition herein, e.g., about 2 mg to about 200 mg, about 2 mg to about 150 mg, about 2 mg to about 100 mg, about 2 mg to about 75 mg, about 2 mg to about 50 mg, about 2 mg to about 40 mg, about 2 mg to about 30 mg, about 2 mg to about 25 mg, about 2 mg to about 20 mg, about 2 mg to about 15 mg, about 2 mg to about 10 mg, about 2 mg to about 8 mg, about 2 mg to about 6 mg, about 2 mg to about 4 mg, about 4 mg to about 250 mg, about 4 mg to about 200 mg, about 4 mg to about 150 mg, about 4 mg to about 100 mg, about 4 mg to about 75 mg, about 4 mg to about 50 mg, about 4 mg to about 40 mg, about 4 mg to about 30 mg, about 4 mg to about 25 mg, about 4 mg to about 20 mg, about 4 mg to about 15 mg, about 4 mg to about 10 mg, about 4 mg to about 8 mg, about 4 mg to about 6 mg, about 6 mg to about 250 mg, about 6 mg to about 200 mg, about 6 mg to about 150 mg, about 6 mg to about 100 mg, about 6 mg to about 75 mg, about 6 mg to about 50 mg, about 6 mg to about 40 mg, about 6 mg to about 30 mg, about 6 mg to about 25 mg, about 6 mg to about 20 mg, about 6 mg to about 15 mg, about 6 mg to about 10 mg, about 6 mg to about 8 mg, about 8 mg to about 250 mg, about 8 mg to about 200 mg, about 8 mg to about 150 mg, about 8 mg to about 100 mg, about 8 mg to about 75 mg, about 8 mg to about 50 mg, about 8 mg to about 40 mg, about 8 mg to about 30 mg, about 8 mg to about 25 mg, about 8 mg to about 20 mg, about 8 mg to about 15 mg, about 8 mg to about 10 mg, about 10 mg to about 250 mg, about 10 mg to about 200 mg, about 10 mg to about 150 mg, about 10 mg to about 100 mg, about 10 mg to about 75 mg, about 10 mg to about 50 mg, about 10 mg to about 40 mg, about 10 mg to about 30 mg, about 10 mg to about 25 mg, about 10 mg to about 20 mg, about 10 mg to about 15 mg, about 15 mg to about 250 mg, about 15 mg to about 200 mg, about 15 mg to about 150 mg, about 15 mg to about 100 mg, about 15 mg to about 75 mg, about 15 mg to about 50 mg, about 15 mg to about 40 mg, about 15 mg to about 30 mg, about 15 mg to about 25 mg, about 15 mg to about 20 mg, about 20 mg to about 250 mg, about 20 mg to about 200 mg, about 20 mg to about 150 mg, about 20 mg to about 100 mg, about 20 mg to about 75 mg, about 20 mg to about 50 mg, about 20 mg to about 40 mg, about 20 mg to about 30 mg, about 20 mg to about 25 mg, about 25 mg to about 250 mg, about 25 mg to about 200 mg, about 25 mg to about 150 mg, about 25 mg to about 100 mg, about 25 mg to about 75 mg, about 25 mg to about 50 mg, about 25 mg to about 40 mg, about 25 mg to about 30 mg, about 30 mg to about 250 mg, about 30 mg to about 200 mg, about 30 mg to about 150 mg, about 30 mg to about 100 mg, about 30 mg to about 75 mg, about 30 mg to about 50 mg, about 30 mg to about 40 mg, about 40 mg to about 250 mg, about 40 mg to about 200 mg, about 40 mg to about 150 mg, about 40 mg to about 100 mg, about 40 mg to about 75 mg, about 40 mg to about 50 mg, about 50 mg to about 250 mg, about 50 mg to about 200 mg, about 50 mg to about 150 mg, about 50 mg to about 100 mg, about 50 mg to about 75 mg, about 75 mg to about 250 mg, about 75 mg to about 200 mg, about 75 mg to about 150 mg, about 75 mg to about 100 mg, about 100 mg to about 250 mg, about 100 mg to about 200 mg, about 100 mg to about 150 mg, about 150 mg to about 250 mg, about 150 mg to about 200 mg, about 200 mg to about 250 mg, about 2 mg, about 4 mg, about 6 mg, about 8 mg, ab out 10 mg, about 15 mg, about 20 mg, about 25 mg, about 50 mg, about 75 mg, about 100 mg, about 150 mg, about 200 mg, or about 250 mg.

In some embodiments, about 0.5 mg to about 70 mg of sweetening agent is present in a composition herein, e.g., about 0.5 mg to about 50 mg, about 0.5 mg to about 30 mg, about 0.5 mg to about 25 mg, about 0.5 mg to about 20 mg, about 0.5 mg to about 15 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 70 mg, about 1 mg to about 50 mg, about 1 mg to about 30 mg, about 1 mg to about 25 mg, about 1 mg to about 20 mg, about 1 mg to about 15 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 70 mg, about 2 mg to about 50 mg, about 2 mg to about 30 mg, about 2 mg to about 25 mg, about 2 mg to about 20 mg, about 2 mg to about 15 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 70 mg, about 3 mg to about 50 mg, about 3 mg to about 30 mg, about 3 mg to about 25 mg, about 3 mg to about 20 mg, about 3 mg to about 15 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 70 mg, about 4 mg to about 50 mg, about 4 mg to about 30 mg, about 4 mg to about 25 mg, about 4 mg to about 20 mg, about 4 mg to about 15 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 70 mg, about 5 mg to about 50 mg, about 5 mg to about 30 mg, about 5 mg to about 25 mg, about 5 mg to about 20 mg, about 5 mg to about 15 mg, about 5 mg to about 10 mg, about 5 mg to about 9 mg, about 5 mg to about 8 mg, about 5 mg to about 7 mg, about 5 mg to about 6 mg, about 6 mg to about 70 mg, about 6 mg to about 50 mg, about 6 mg to about 30 mg, about 6 mg to about 25 mg, about 6 mg to about 20 mg, about 6 mg to about 15 mg, about 6 mg to about 10 mg, about 6 mg to about 9 mg, about 6 mg to about 8 mg, about 6 mg to about 7 mg, about 7 mg to about 70 mg, about 7 mg to about 50 mg, about 7 mg to about 30 mg, about 7 mg to about 25 mg, about 7 mg to about 20 mg, about 7 mg to about 15 mg, about 7 mg to about 10 mg, about 7 mg to about 9 mg, about 7 mg to about 8 mg, about 8 mg to about 70 mg, about 8 mg to about 50 mg, about 8 mg to about 30 mg, about 8 mg to about 25 mg, about 8 mg to about 20 mg, about 8 mg to about 15 mg, about 8 mg to about 10 mg, about 8 mg to about 9 mg, about 9 mg to about 70 mg, about 9 mg to about 50 mg, about 9 mg to about 30 mg, about 9 mg to about 25 mg, about 9 mg to about 20 mg, about 9 mg to about 15 mg, about 9 mg to about 10 mg, about 10 mg to about 70 mg, about 10 mg to about 50 mg, about 10 mg to about 30 mg, about 10 mg to about 25 mg, about 10 mg to about 20 mg, about 10 mg to about 15 mg, about 15 mg to about 70 mg, about 15 mg to about 50 mg, about 15 mg to about 30 mg, about 15 mg to about 25 mg, about 15 mg to about 20 mg, about 20 mg to about 70 mg, about 20 mg to about 50 mg, about 20 mg to about 30 mg, about 20 mg to about 25 mg, about 25 mg to about 70 mg, about 25 mg to about 50 mg, about 25 mg to about 30 mg, about 30 mg to about 70 mg, about 30 mg to about 50 mg, about 50 mg to about 70 mg, about 0.5 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 50 mg, about or about 70 mg. In some embodiments, about 0.5% to about 5% (w/w) of the composition is sweetening agent, e.g., about 0.5% to about 4%, about 0.5% to about 3%, about 0.5% to about 2.5%, about 0.5% to about 2%, about 0.5% to about 1.5%, about 0.5% to about 1%, about 1% to about 5%, about 1% to about 4%, about 1% to about 3%, about 1% to about 2.5%, about 1% to about 2%, about 1% to about 1.5%, about 1.5% to about 5%, about 1.5% to about 4%, about 1.5% to about 3%, about 1.5% to about 2.5%, about 1.5% to about 2%, about 2% to about 5%, about 2% to about 4%, about 2% to about 3%, about 2% to about 2.5%, about 2.5% to about 5%, about 2.5% to about 4%, about 2.5% to about 3%, about 3% to about 5%, about 3% to about 4%, about 4% to about 5%, about 0.5%, about 1%, about 1.5%, about 2%, about 2.5%, about 3%, about 3.5%, about 4%, about 4.5%, or about 5%.

Certain compositions herein are formulated to facilitate increased energy in a person. In some such cases, the amount of sweetening agent in the composition is about 18 mg to about 26 mg, about 20 mg to about 24 mg, or about 22 mg. In other such cases, the amount of sweetening agent in the composition is about 20 mg to about 28 mg, about 22 mg to about 26 mg, or about 24 mg. An example product is called RH1.

Certain compositions herein are formulated to facilitate increased relaxation in a person. In some such cases, the amount of sweetening agent in the composition is about 19 mg to about 27 mg, about 21 mg to about 25 mg, or about 23 mg. In other such cases, the amount of sweetening agent in the composition is about 18 mg to about 26 mg, about 20 mg to about 24 mg, or about 22 mg. An example product is called RH2.

Certain compositions herein are formulated to improve the sleep health of a person. In some such cases, the amount of sweetening agent present in the composition is about 5 mg to about 9 mg, about 6 mg to about 8 mg, or about 7 mg. An example product is called RH3.

As described above, sweetening agents may be added to compositions in order to make the composition sweeter, and, thus, may improve the taste of the composition to various persons.

### Flavoring Agents

In one aspect, provided are compositions are formulations comprising a flavoring agent (e.g., peppermint flavor dried P-183, another mint flavoring agent, methanol crystals, natural flavoring (e.g., flavor of a fruit such as grapefruit and/or apple), a peppermint oil, a spearmint oil blend, a peppermint/spearmint oil blend, a fruit extract (e.g., monkfruit), a sugar substitute (e.g., STEVIA), or a flavoring agent with another flavor). In some embodiments, the flavoring agent may provide a specific flavor for a tablet.

In some embodiments, about 4 mg to about 850 mg of a flavoring agent (e.g., peppermint flavor dried P-183 and/or menthol crystal, a natural flavor, a peppermint oil blend, a spearmint oil blend, a peppermint/spearmint oil blend, a fruit extract (e.g., monkfruit), a sugar substitute (e.g., STEVIA)) is present in a composition herein, e.g., about 4 mg to about 800 mg, about 4 mg to about 750 mg, about 4 mg to about 700 mg, about 4 mg to about 650 mg, about 4 mg to about 600 mg, about 4 mg to about 550 mg, about 4 mg to about 500 mg, about 4 mg to about 450 mg, about 4 mg to about 400 mg, about 4 mg to about 350 mg, about 4 mg to about 300 mg, about 4 mg to about 250 mg, about 4 mg to about 200 mg, about 4 mg to about 160 mg, about 4 mg to about 120 mg, about 4 mg to about 80 mg, about 4 mg to about 60 mg, about 4 mg to about 52 mg, about 4 mg to about 44 mg, about 4 mg to about 40 mg, about 4 mg to about 36 mg, about 4 mg to about 32 mg, about 4 mg to about 28 mg, about 4 mg to about 24 mg, about 4 mg to about 20 mg, about 4 mg to about 16 mg, about 4 mg to about 12 mg, about 4 mg to about 8 mg, about 8 mg to about 850 mg, about 8 mg to about 800 mg, about 8 mg to about 750 mg, about 8 mg to about 700 mg, about 8 mg to about 650 mg, about 8 mg to about 600 mg, about 8 mg to about 550 mg, about 8 mg to about 500 mg, about 8 mg to about 450 mg, about 8 mg to about 400 mg, about 8 mg to about 350 mg, about 8 mg to about 300 mg, about 8 mg to about 250 mg, about 8 mg to about 200 mg, about 8 mg to about 160 mg, about 8 mg to about 120 mg, about 8 mg to about 80 mg, about 8 mg to about 60 mg, about 8 mg to about 52 mg, about 8 mg to about 44 mg, about 8 mg to about 40 mg, about 8 mg to about 36 mg, about 8 mg to about 32 mg, about 8 mg to about 28 mg, about 8 mg to about 24 mg, about 8 mg to about 20 mg, about 8 mg to about 16 mg, about 8 mg to about 12 mg, about 12 mg to about 850 mg, about 12 mg to about 800 mg, about 12 mg to about 750 mg, about 12 mg to about 700 mg, about 12 mg to about 650 mg, about 12 mg to about 600 mg, about 12 mg to about 550 mg, about 12 mg to about 500 mg, about 12 mg to about 450 mg, about 12 mg to about 400 mg, about 12 mg to about 350 mg, about 12 mg to about 300 mg, about 12 mg to about 250 mg, about 12 mg to about 200 mg, about 12 mg to about 160 mg, about 12 mg to about 120 mg, about 12 mg to about 80 mg, about 12 mg to about 60 mg, about 12 mg to about 52 mg, about 12 mg to about 44 mg, about 12 mg to about 40 mg, about 12 mg to about 36 mg, about 12 mg to about 32 mg, about 12 mg to about 28 mg, about 12 mg to about 24 mg, about 12 mg to about 20 mg, about 12 mg to about 16 mg, about 16 mg to about 850 mg, about 16 mg to about 800 mg, about 16 mg to about 750 mg, about 16 mg to about 700 mg, about 16 mg to about 650 mg, about 16 mg to about 600 mg, about 16 mg to about 550 mg, about 16 mg to about 500 mg, about 16 mg to about 450 mg, about 16 mg to about 400 mg, about 16 mg to about 350 mg, about 16 mg to about 300 mg, about 16 mg to about 250 mg, about 16 mg to about 200 mg, about 16 mg to about 160 mg, about 16 mg to about 120 mg, about 16 mg to about 80 mg, about 16 mg to about 60 mg, about 16 mg to about 52 mg, about 16 mg to about 44 mg, about 16 mg to about 40 mg, about 16 mg to about 36 mg, about 16 mg to about 32 mg, about 16 mg to about 28 mg, about 16 mg to about 24 mg, about 16 mg to about 20 mg, about 20 mg to about 850 mg, about 20 mg to about 800 mg, about 20 mg to about 750 mg, about 20 mg to about 700 mg, about 20 mg to about 650 mg, about 20 mg to about 600 mg, about 20 mg to about 550 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 160 mg, about 20 mg to about 120 mg, about 20 mg to about 80 mg, about 20 mg to about 60 mg, about 20 mg to about 52 mg, about 20 mg to about 44 mg, about 20 mg to about 40 mg, about 20 mg to about 36 mg, about 20 mg to about 32 mg, about 20 mg to about 28 mg, about 20 mg to about 24 mg, about 24 mg to about 850 mg, about 24 mg to about 800 mg, about 24 mg to about 750 mg, about 24 mg to about 700 mg, about 24 mg to about 650 mg, about 24 mg to about 600 mg, about 24 mg to about 550 mg, about 24 mg to about 500 mg, about 24 mg to about 450 mg, about 24 mg to about 400 mg, about 24 mg to about 350 mg, about 24 mg to about 300 mg, about 24 mg to about 250 mg, about 24 mg to about 200 mg, about 24 mg to about 160 mg, about 24 mg to about 120 mg, about 24 mg to about 80 mg, about 24 mg to about 60 mg, about 24 mg to about 52 mg, about 24 mg to about 44 mg, about 24 mg to about 40 mg, about 24 mg to about 36 mg, about 24 mg to about 32 mg, about 24 mg to about 28 mg, about 28 mg to about 850 mg, about 28 mg to about 800 mg, about 28 mg to about 750 mg, about 28 mg to about 700 mg, about 28 mg to about 650 mg, about 28 mg to about 600 mg, about 28 mg to about 550 mg, about 28 mg to about 500 mg, about 28 mg to about 450 mg, about 28 mg to about 400 mg, about 28 mg to about 350 mg, about 28 mg to about 300 mg, about 28 mg to about 250 mg, about 28 mg to about 200 mg, about 28 mg to about 160 mg, about 28 mg to about 120 mg, about 28 mg to about 80 mg, about 28 mg to about 60 mg, about 28 mg to about 52 mg, about 28 mg to about 44 mg, about 28 mg to about 40 mg, about 28 mg to about 36 mg, about 28 mg to about 32 mg, about 32 mg to about 850 mg, about 32 mg to about 800 mg, about 32 mg to about 750 mg, about 32 mg to about 700 mg, about 32 mg to about 650 mg, about 32 mg to about 600 mg, about 32 mg to about 550 mg, about 32 mg to about 500 mg, about 32 mg to about 450 mg, about 32 mg to about 400 mg, about 32 mg to about 350 mg, about 32 mg to about 300 mg, about 32 mg to about 250 mg, about 32 mg to about 200 mg, about 32 mg to about 160 mg, about 32 mg to about 120 mg, about 32 mg to about 80 mg, about 32 mg to about 60 mg, about 32 mg to about 52 mg, about 32 mg to about 44 mg, about 32 mg to about 40 mg, about 32 mg to about 36 mg, about 36 mg to about 850 mg, about 36 mg to about 800 mg, about 36 mg to about 750 mg, about 36 mg to about 700 mg, about 36 mg to about 650 mg, about 36 mg to about 600 mg, about 36 mg to about 550 mg, about 36 mg to about 500 mg, about 36 mg to about 450 mg, about 36 mg to about 400 mg, about 36 mg to about 350 mg, about 36 mg to about 300 mg, about 36 mg to about 250 mg, about 36 mg to about 200 mg, about 36 mg to about 160 mg, about 36 mg to about 120 mg, about 36 mg to about 80 mg, about 36 mg to about 60 mg, about 36 mg to about 52 mg, about 36 mg to about 44 mg, about 36 mg to about 40 mg, about 40 mg to about 850 mg, about 40 mg to about 800 mg, about 40 mg to about 750 mg, about 40 mg to about 700 mg, about 40 mg to about 650 mg, about 40 mg to about 600 mg, about 40 mg to about 550 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 160 mg, about 40 mg to about 120 mg, about 40 mg to about 80 mg, about 40 mg to about 60 mg, about 40 mg to about 52 mg, about 40 mg to about 44 mg, about 52 mg to about 850 mg, about 52 mg to about 800 mg, about 52 mg to about 750 mg, about 52 mg to about 700 mg, about 52 mg to about 650 mg, about 52 mg to about 600 mg, about 52 mg to about 550 mg, about 52 mg to about 500 mg, about 52 mg to about 450 mg, about 52 mg to about 400 mg, about 52 mg to about 350 mg, about 52 mg to about 300 mg, about 52 mg to about 250 mg, about 52 mg to about 200 mg, about 52 mg to about 160 mg, about 52 mg to about 120 mg, about 52 mg to about 80 mg, about 52 mg to about 60 mg, about 60 mg to about 850 mg, about 60 mg to about 800 mg, about 60 mg to about 750 mg, about 60 mg to about 700 mg, about 60 mg to about 650 mg, about 60 mg to about 600 mg, about 60 mg to about 550 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 160 mg, about 60 mg to about 120 mg, about 60 mg to about 80 mg, about 80 mg to about 850 mg, about 80 mg to about 800 mg, about 80 mg to about 750 mg, about 80 mg to about 700 mg, about 80 mg to about 650 mg, about 80 mg to about 600 mg, about 80 mg to about 550 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 160 mg, about 80 mg to about 120 mg, about 120 mg to about 850 mg, about 120 mg to about 800 mg, about 120 mg to about 750 mg, about 120 mg to about 700 mg, about 120 mg to about 650 mg, about 120 mg to about 600 mg, about 120 mg to about 550 mg, about 120 mg to about 500 mg, about 120 mg to about 450 mg, about 120 mg to about 400 mg, about 120 mg to about 350 mg, about 120 mg to about 300 mg, about 120 mg to about 250 mg, about 120 mg to about 200 mg, about 120 mg to about 160 mg, about 160 mg to about 850 mg, about 160 mg to about 800 mg, about 160 mg to about 750 mg, about 160 mg to about 700 mg, about 160 mg to about 650 mg, about 160 mg to about 600 mg, about 160 mg to about 550 mg, about 160 mg to about 500 mg, about 160 mg to about 450 mg, about 160 mg to about 400 mg, about 160 mg to about 350 mg, about 160 mg to about 300 mg, about 160 mg to about 250 mg, about 160 mg to about 200 mg, about 200 mg to about 850 mg, about 200 mg to about 800 mg, about 200 mg to about 750 mg, about 200 mg to about 700 mg, about 200 mg to about 650 mg, about 200 mg to about 600 mg, about 200 mg to about 550 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 850 mg, about 250 mg to about 800 mg, about 250 mg to about 750 mg, about 250 mg to about 700 mg, about 250 mg to about 650 mg, about 250 mg to about 600 mg, about 250 mg to about 550 mg, about 250 mg to about 500 mg, about 250 mg to about 450 mg, about 250 mg to about 400 mg, about 250 mg to about 350 mg, about 250 mg to about 300 mg, about 300 mg to about 850 mg, about 300 mg to about 800 mg, about 300 mg to about 750 mg, about 300 mg to about 700 mg, about 300 mg to about 650 mg, about 300 mg to about 600 mg, about 300 mg to about 550 mg, about 300 mg to about 500 mg, about 300 mg to about 450 mg, about 300 mg to about 400 mg, about 300 mg to about 350 mg, about 350 mg to about 850 mg, about 350 mg to about 800 mg, about 350 mg to about 750 mg, about 350 mg to about 700 mg, about 350 mg to about 650 mg, about 350 mg to about 600 mg, about 350 mg to about 550 mg, about 350 mg to about 500 mg, about 350 mg to about 450 mg, about 350 mg to about 400 mg, about 400 mg to about 850 mg, about 400 mg to about 800 mg, about 400 mg to about 750 mg, about 400 mg to about 700 mg, about 400 mg to about 650 mg, about 400 mg to about 600 mg, about 400 mg to about 550 mg, about 400 mg to about 500 mg, about 400 mg to about 450 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 450 mg to about 550 mg, about 450 mg to about 500 mg, about 500 mg to about 850 mg, about 500 mg to about 800 mg, about 500 mg to about 750 mg, about 500 mg to about 700 mg, about 500 mg to about 650 mg, about 500 mg to about 600 mg, about 500 mg to about 550 mg, about 550 mg to about 850 mg, about 550 mg to about 800 mg, about 550 mg to about 750 mg, about 550 mg to about 700 mg, about 550 mg to about 650 mg, about 550 mg to about 600 mg, about 600 mg to about 850 mg, about 600 mg to about 800 mg, about 600 mg to about 750 mg, about 600 mg to about 700 mg, about 600 mg to about 650 mg, about 650 mg to about 850 mg, about 650 mg to about 800 mg, about 650 mg to about 750 mg, about 650 mg to about 700 mg, about 700 mg to about 850 mg, about 700 mg to about 800 mg, about 700 mg to about 750 mg, about 750 mg to about 850 mg, about 750 mg to about 800 mg, about 800 mg to about 850 mg, about 4 mg, about 8 mg, about 12 mg, about 16 mg, about 20 mg, about 24 mg, about 28 mg, about 32 mg, about 36 mg, about 40 mg, about 44 mg, about 52 mg, about 60 mg, about 80 mg, about 120 mg, about 160 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, or about 850 mg.

In some embodiments, about 1 mg to about 250 mg of a flavoring agent (e.g., peppermint flavor dried P-183 and/or menthol crystal, natural flavoring (e.g., flavor of a fruit such as grapefruit and/or apple), a peppermint oil blend, a spearmint oil blend, a peppermint oil/spearmint oil blend, a fruit extract (e.g., monkfruit), a sugar substitute (e.g., STEVIA)) is present in a composition herein, e.g., about 1 mg to about 200 mg, about 1 mg to about 150 mg, about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 250 mg, about 2.5 mg to about 200 mg, about 2.5 mg to about 150 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 250 mg, about 5 mg to about 200 mg, about 5 mg to about 150 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 250 mg, about 7.5 mg to about 200 mg, about 7.5 mg to about 150 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 250 mg, about 10 mg to about 200 mg, about 10 mg to about 150 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 250 mg, about 12.5 mg to about 200 mg, about 12.5 mg to about 150 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 250 mg, about 15 mg to about 200 mg, about 15 mg to about 150 mg, about 15 mg to about 100 mg, about 15 mg to about 75 mg, about 15 mg to about 50 mg, about 15 mg to about 25 mg, about 15 mg to about 20 mg, about 20 mg to about 250 mg, about 20 mg to about 200 mg, about 20 mg to about 150 mg, about 20 mg to about 100 mg, about 20 mg to about 75 mg, about 20 mg to about 50 mg, about 20 mg to about 25 mg, about 25 mg to about 250 mg, about 25 mg to about 200 mg, about 25 mg to about 150 mg, about 25 mg to about 100 mg, about 25 mg to about 75 mg, about 25 mg to about 50 mg, about 50 mg to about 250 mg, about 50 mg to about 200 mg, about 50 mg to about 150 mg, about 50 mg to about 100 mg, about 50 mg to about 75 mg, about 75 mg to about 250 mg, about 75 mg to about 200 mg, about 75 mg to about 150 mg, about 75 mg to about 100 mg, about 100 mg to about 250 mg, about 100 mg to about 200 mg, about 100 mg to about 150 mg, about 150 mg to about 250 mg, about 150 mg to about 200 mg, about 200 mg to about 250 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, about 150 mg, about 200 mg, or about 250 mg.

In some embodiments, about 1% to about 15% (w/w) of a composition is a flavoring agent (e.g., peppermint flavor dried P-183 and/or menthol crystal, natural flavoring (e.g., flavor of a fruit such as grapefruit and/or apple), a peppermint oil blend, a spearmint oil blend, a peppermint oil/spearmint oil blend, a fruit extract (e.g., monkfruit), a sugar substitute (e.g., STEVIA)), e.g., about 1% to about 12.5%, about 1% to about 10%, about 1% to about 9%, about 1% to about 8%, about 1% to about 7%, about 1% to about 6%, about 1% to about 5%, about 1% to about 4.5%, about 1% to about 4%, about 1% to about 3.5%, about 1% to about 3%, about 1% to about 2.5%, about 1% to about 2%, about 1% to about 1.5%, about 1.5% to about 15%, about 1.5% to about 12.5%, about 1.5% to about 10%, about 1.5% to about 9%, about 1.5% to about 8%, about 1.5% to about 7%, about 1.5% to about 6%, about 1.5% to about 5%, about 1.5% to about 4.5%, about 1.5% to about 4%, about 1.5% to about 3.5%, about 1.5% to about 3%, about 1.5% to about 2.5%, about 1.5% to about 2%, about 2% to about 15%, about 2% to about 12.5%, about 2% to about 10%, about 2% to about 9%, about 2% to about 8%, about 2% to about 7%, about 2% to about 6%, about 2% to about 5%, about 2% to about 4.5%, about 2% to about 4%, about 2% to about 3.5%, about 2% to about 3%, about 2% to about 2.5%, about 2.5% to about 15%, about 2.5% to about 12.5%, about 2.5% to about 10%, about 2.5% to about 9%, about 2.5% to about 8%, about 2.5% to about 7%, about 2.5% to about 6%, about 2.5% to about 5%, about 2.5% to about 4.5%, about 2.5% to about 4%, about 2.5% to about 3.5%, about 2.5% to about 3%, about 3% to about 15%, about 3% to about 12.5%, about 3% to about 10%, about 3% to about 9%, about 3% to about 8%, about 3% to about 7%, about 3% to about 6%, about 3% to about 5%, about 3% to about 4.5%, about 3% to about 4%, about 3% to about 3.5%, about 3.5% to about 15%, about 3.5% to about 12.5%, about 3.5% to about 10%, about 3.5% to about 9%, about 3.5% to about 8%, about 3.5% to about 7%, about 3.5% to about 6%, about 3.5% to about 5%, about 3.5% to about 4.5%, about 3.5% to about 4%, about 4% to about 15%, about 4% to about 12.5%, about 4% to about 10%, about 4% to about 9%, about 4% to about 8%, about 4% to about 7%, about 4% to about 6%, about 4% to about 5%, about 4% to about 4.5%, about 4.5% to about 15%, about 4.5% to about 12.5%, about 4.5% to about 10%, about 4.5% to about 9%, about 4.5% to about 8%, about 4.5% to about 7%, about 4.5% to about 6%, about 4.5% to about 5%, about 5% to about 15%, about 5% to about 12.5%, about 5% to about 10%, about 5% to about 9%, about 5% to about 8%, about 5% to about 7%, about 5% to about 6%, about 6% to about 15%, about 6% to about 12.5%, about 6% to about 10%, about 6% to about 9%, about 6% to about 8%, about 6% to about 7%, about 7% to about 15%, about 7% to about 12.5%, about 7% to about 10%, about 7% to about 9%, about 7% to about 8%, about 8% to about 15%, about 8% to about 12.5%, about 8% to about 10%, about 8% to about 9%, about 9% to about 15%, about 9% to about 12.5%, about 9% to about 10%, about 10% to about 15%, about 10% to about 12.5%, about 12.5% to about 15%, about 1%, about 1.5%, about 2%, about 2.5%, about 3%, about 3.5%, about 4%, about 4.5%, about 5%, about 6%, about 7%, about 8%, about 9%, about 10%, about 12.5%, or about 15%.

Certain compositions herein are formulated to facilitate increased energy in a person. In some such cases, the amount of flavoring agent in the composition is about 45 mg to about 125 mg, about 55 mg to about 115 mg, about 65 mg to about 105 mg, about 75 mg to about 95 mg, or about 85 mg. In some such cases, the amount of flavoring agent (e.g., a fruit extract) in the composition is about 20 mg to about 80 mg, about 30 mg to about 70 mg, about 40 mg to about 60 mg, or about 50 mg. In some such cases, the amount of flavoring agent (e.g., a sugar substitute) in the composition is about 10 mg to about 40 mg, about 15 mg to about 35 mg, about 10 mg to about 30 mg, or about 25 mg. In some such cases, the amount of natural flavoring in the composition is about 0.5 mg to about 3 mg, about 0.75 mg to about 2.5 mg, about 1 mg, or about 2 mg. An example product is called RH1.

Certain compositions herein are formulated to facilitate increased relaxation in a person. In some such cases, the amount of flavoring agent in the composition is about 45 mg to about 125 mg, about 55 mg to about 115 mg, about 65 mg to about 105 mg, about 75 mg to about 95 mg, or about 85 mg. In some such cases, the amount of a flavoring agent (e.g., a fruit extract) in the composition is about 30 mg to about 90 mg, about 40 mg to about 80 mg, about 50 mg to about 70 mg, or about 60 mg. In some such cases, the amount of a flavoring agent (e.g., menthol crystal) in the composition is about 40 mg to about 100 mg, about 50 mg to about 90 mg, about 60 mg to about 80 mg, or about 70 mg. In some such cases, the amount of natural flavoring in the composition is about 0.5 mg to about 5 mg, about 1 mg to about 4.5 mg, about 1.5 mg to about 4 mg, about 2 mg to about 3.5 mg, or about 3 mg. An example product is called RH2.

Certain compositions herein are formulated to improve the sleep health of a person. In some such cases, the amount of flavoring agent present in the composition is about 20 mg to about 28 mg, about 22 mg to about 26 mg, or about 24 mg. An example product is called RH3.

As described above, flavoring agents such as peppermint flavor dried P-183 may be added to compositions in order to increase the presence of a certain flavor in the composition, and, thus, may improve the taste of the composition to various persons. Menthol crystals may also be used as a flavoring agent. Some compositions comprise peppermint flavor dried P-183 and menthol crystals.

### Starch Excipient

In one aspect, provided are compositions and formulations comprising a starch excipient (e.g., starcap 1500.) In some embodiments, the starch excipient is present as a filling agent to make sure tablets are full. In some embodiments, the starch excipient may be a pregelatinized starch that has been cooked and dried. A starch excipient may be used for a variety of roles. For example, a starch excipient may act as a diluent, disintegrant, glidant, and/or binder.

In some embodiments, about 5 mg to about 500 mg of a starch excipient (e.g., starcap 1500) is present in a composition herein, e.g., about 5 mg to about 450 mg, about 5 mg to about 400 mg, about 5 mg to about 350 mg, about 5 mg to about 300 mg, about 5 mg to about 250 mg, about 5 mg to about 200 mg, about 5 mg to about 150 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 500 mg, about 10 mg to about 450 mg, about 10 mg to about 400 mg, about 10 mg to about 350 mg, about 10 mg to about 300 mg, about 10 mg to about 250 mg, about 10 mg to about 200 mg, about 10 mg to about 150 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 500 mg, about 20 mg to about 450 mg, about 20 mg to about 400 mg, about 20 mg to about 350 mg, about 20 mg to about 300 mg, about 20 mg to about 250 mg, about 20 mg to about 200 mg, about 20 mg to about 150 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 500 mg, about 30 mg to about 450 mg, about 30 mg to about 400 mg, about 30 mg to about 350 mg, about 30 mg to about 300 mg, about 30 mg to about 250 mg, about 30 mg to about 200 mg, about 30 mg to about 150 mg, about 30 mg to about 100 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 500 mg, about 40 mg to about 450 mg, about 40 mg to about 400 mg, about 40 mg to about 350 mg, about 40 mg to about 300 mg, about 40 mg to about 250 mg, about 40 mg to about 200 mg, about 40 mg to about 150 mg, about 40 mg to about 100 mg, about 40 mg to about 90 mg, about 40 mg to about 80 mg, about 40 mg to about 70 mg, about 40 mg to about 60 mg, about 40 mg to about 50 mg, about 50 mg to about 500 mg, about 50 mg to about 450 mg, about 50 mg to about 400 mg, about 50 mg to about 350 mg, about 50 mg to about 300 mg, about 50 mg to about 250 mg, about 50 mg to about 200 mg, about 50 mg to about 150 mg, about 50 mg to about 100 mg, about 50 mg to about 90 mg, about 50 mg to about 80 mg, about 50 mg to about 70 mg, about 50 mg to about 60 mg, about 60 mg to about 500 mg, about 60 mg to about 450 mg, about 60 mg to about 400 mg, about 60 mg to about 350 mg, about 60 mg to about 300 mg, about 60 mg to about 250 mg, about 60 mg to about 200 mg, about 60 mg to about 150 mg, about 60 mg to about 100 mg, about 60 mg to about 90 mg, about 60 mg to about 80 mg, about 60 mg to about 70 mg, about 70 mg to about 500 mg, about 70 mg to about 450 mg, about 70 mg to about 400 mg, about 70 mg to about 350 mg, about 70 mg to about 300 mg, about 70 mg to about 250 mg, about 70 mg to about 200 mg, about 70 mg to about 150 mg, about 70 mg to about 100 mg, about 70 mg to about 90 mg, about 70 mg to about 80 mg, about 80 mg to about 500 mg, about 80 mg to about 450 mg, about 80 mg to about 400 mg, about 80 mg to about 350 mg, about 80 mg to about 300 mg, about 80 mg to about 250 mg, about 80 mg to about 200 mg, about 80 mg to about 150 mg, about 80 mg to about 100 mg, about 80 mg to about 90 mg, about 90 mg to about 500 mg, about 90 mg to about 450 mg, about 90 mg to about 400 mg, about 90 mg to about 350 mg, about 90 mg to about 300 mg, about 90 mg to about 250 mg, about 90 mg to about 200 mg, about 90 mg to about 150 mg, about 90 mg to about 100 mg, about 100 mg to about 500 mg, about 100 mg to about 450 mg, about 100 mg to about 400 mg, about 100 mg to about 350 mg, about 100 mg to about 300 mg, about 100 mg to about 250 mg, about 100 mg to about 200 mg, about 100 mg to about 150 mg, about 150 mg to about 500 mg, about 150 mg to about 450 mg, about 150 mg to about 400 mg, about 150 mg to about 350 mg, about 150 mg to about 300 mg, about 150 mg to about 250 mg, about 150 mg to about 200 mg, about 200 mg to about 500 mg, about 200 mg to about 450 mg, about 200 mg to about 400 mg, about 200 mg to about 350 mg, about 200 mg to about 300 mg, about 200 mg to about 250 mg, about 250 mg to about 500 mg, about 250 mg to about 450 mg, about 250 mg to about 400 mg, about 250 mg to about 350 mg, about 250 mg to about 300 mg, about 300 mg to about 500 mg, about 300 mg to about 450 mg, about 300 mg to about 400 mg, about 300 mg to about 350 mg, about 350 mg to about 500 mg, about 350 mg to about 450 mg, about 350 mg to about 400 mg, about 400 mg to about 500 mg, about 400 mg to about 450 mg, about 450 mg to about 500 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, or about 500 mg.

In some embodiments, about 1 mg to about 125 mg of a starch excipient (e.g., starcap 1500) is present in a composition herein, e.g., about 1 mg to about 100 mg, about 1 mg to about 75 mg, about 1 mg to about 50 mg, about 1 mg to about 25 mg, about 1 mg to about 20 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2.5 mg to about 125 mg, about 2.5 mg to about 100 mg, about 2.5 mg to about 75 mg, about 2.5 mg to about 50 mg, about 2.5 mg to about 25 mg, about 2.5 mg to about 20 mg, about 2.5 mg to about 15 mg, about 2.5 mg to about 12.5 mg, about 2.5 mg to about 10 mg, about 2.5 mg to about 7.5 mg, about 2.5 mg to about 5 mg, about 5 mg to about 125 mg, about 5 mg to about 100 mg, about 5 mg to about 75 mg, about 5 mg to about 50 mg, about 5 mg to about 25 mg, about 5 mg to about 20 mg, about 5 mg to about 15 mg, about 5 mg to about 12.5 mg, about 5 mg to about 10 mg, about 5 mg to about 7.5 mg, about 7.5 mg to about 125 mg, about 7.5 mg to about 100 mg, about 7.5 mg to about 75 mg, about 7.5 mg to about 50 mg, about 7.5 mg to about 25 mg, about 7.5 mg to about 20 mg, about 7.5 mg to about 15 mg, about 7.5 mg to about 12.5 mg, about 7.5 mg to about 10 mg, about 10 mg to about 125 mg, about 10 mg to about 100 mg, about 10 mg to about 75 mg, about 10 mg to about 50 mg, about 10 mg to about 25 mg, about 10 mg to about 20 mg, about 10 mg to about 15 mg, about 10 mg to about 12.5 mg, about 12.5 mg to about 125 mg, about 12.5 mg to about 100 mg, about 12.5 mg to about 75 mg, about 12.5 mg to about 50 mg, about 12.5 mg to about 25 mg, about 12.5 mg to about 20 mg, about 12.5 mg to about 15 mg, about 15 mg to about 125 mg, about 15 mg to about 100 mg, about 15 mg to about 75 mg, about 15 mg to about 50 mg, about 15 mg to about 25 mg, about 15 mg to about 20 mg, about 20 mg to about 125 mg, about 20 mg to about 100 mg, about 20 mg to about 75 mg, about 20 mg to about 50 mg, about 20 mg to about 25 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 125 mg, about 75 mg to about 100 mg, about 100 mg to about 125 mg, about 1 mg, about 2.5 mg, about 5 mg, about 7.5 mg, about 10 mg, about 12.5 mg, about 15 mg, about 20 mg, about 25 mg, about 50 mg, about 75 mg, about 100 mg, or about 125 mg. In some embodiments, about 1% to about 7% (w/w) of the composition is starch excipient (e.g., starcap 1500), e.g., about 1% to about 6%, about 1% to about 5%, about 1% to about 4.5%, about 1% to about 4%, about 1% to about 3.5%, about 1% to about 3%, 1% to about 2.5%, about 1% to about 2%, about 1% to about 1.5%, 1.5% to about 7%, about 1.5% to about 6%, about 1.5% to about 5%, about 1.5% to about 4.5%, about 1.5% to about 4%, about 1.5% to about 3.5%, about 1.5% to about 3%, 1.5% to about 2.5%, about 1.5% to about 2%, 2% to about 7%, about 2% to about 6%, about 2% to about 5%, about 2% to about 4.5%, about 2% to about 4%, about 2% to about 3.5%, about 2% to about 3%, 2% to about 2.5%, 2.5% to about 7%, about 2.5% to about 6%, about 2.5% to about 5%, about 2.5% to about 4.5%, about 2.5% to about 4%, about 2.5% to about 3.5%, about 2.5% to about 3%, 3% to about 7%, about 3% to about 6%, about 3% to about 5%, about 3% to about 4.5%, about 3% to about 4%, about 3% to about 3.5%, 3.5% to about 7%, about 3.5% to about 6%, about 3.5% to about 5%, about 3.5% to about 4.5%, about 3.5% to about 4%, 4% to about 7%, about 4% to about 6%, about 4% to about 5%, about 4% to about 4.5%, 4.5% to about 7%, about 4.5% to about 6%, about 4.5% to about 5%, about 5% to about 7%, about 5% to about 6%, about 6% to about 7%, about 1%, about 1.5%, about 2%, about 2.5%, about 3%, about 3.5%, about 4%, about 4.5%, about 5%, about 6%, or about 7%.

Certain compositions herein are formulated to facilitate increased energy in a person. In some such cases, the amount of starch excipient in the composition is about 2 mg to about 15 mg, about 5 mg to about 10 mg, or about 8 mg. In some such cases, the amount of starch excipient in the composition is about 40 mg to about 60 mg, about 45 mg to about 55 mg, or about 50 mg. An example product is called RH1.

Certain compositions herein are formulated to facilitate increased relaxation in a person. In some such cases, the amount of starch excipient in the composition is about 40 mg to about 60 mg, about 45 mg to about 55 mg, or about 50 mg. An example product is called RH2.

Certain compositions herein are formulated to improve the sleep health of a person. In some such cases, the amount of starch excipient present in the composition is about 9 mg to about 14 mg, about 10 mg to about 13 mg, about 11 mg to about 12 mg, or about 11.5 mg. An example product is called RH3.

As described above, starch excipients may act as filling agents, and thus, may be added to compositions in order fill a tablet or add weight to the tablet. While starcap 1500 is described above as a starch excipient, starcap 1500 is exemplary and other starch excipients, such as blends of corn starch and pregelatinized starch, may be used.

### Magnesium Stearate

In one aspect, provided are compositions and formulations comprising magnesium stearate. In some embodiments, the magnesium stearate is present as a flow agent that keeps the ingredients of a tablet separated.

In some embodiments, about 0.5 mg to about 60 mg of magnesium stearate is present in a composition herein, e.g., about 0.5 mg to about 50 mg, about 0.5 mg to about 40 mg, about 0.5 mg to about 30 mg, about 0.5 mg to about 20 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 0.5 mg to about 1 mg, about 1 mg to about 60 mg, about 1 mg to about 50 mg, about 1 mg to about 40 mg, about 1 mg to about 30 mg, about 1 mg to about 20 mg, about 1 mg to about 10 mg, about 1 mg to about 9 mg, about 1 mg to about 8 mg, about 1 mg to about 7 mg, about 1 mg to about 6 mg, about 1 mg to about 5 mg, about 1 mg to about 4 mg, about 1 mg to about 3 mg, about 1 mg to about 2 mg, about 2 mg to about 60 mg, about 2 mg to about 50 mg, about 2 mg to about 40 mg, about 2 mg to about 30 mg, about 2 mg to about 20 mg, about 2 mg to about 10 mg, about 2 mg to about 9 mg, about 2 mg to about 8 mg, about 2 mg to about 7 mg, about 2 mg to about 6 mg, about 2 mg to about 5 mg, about 2 mg to about 4 mg, about 2 mg to about 3 mg, about 3 mg to about 60 mg, about 3 mg to about 50 mg, about 3 mg to about 40 mg, about 3 mg to about 30 mg, about 3 mg to about 20 mg, about 3 mg to about 10 mg, about 3 mg to about 9 mg, about 3 mg to about 8 mg, about 3 mg to about 7 mg, about 3 mg to about 6 mg, about 3 mg to about 5 mg, about 3 mg to about 4 mg, about 4 mg to about 60 mg, about 4 mg to about 50 mg, about 4 mg to about 40 mg, about 4 mg to about 30 mg, about 4 mg to about 20 mg, about 4 mg to about 10 mg, about 4 mg to about 9 mg, about 4 mg to about 8 mg, about 4 mg to about 7 mg, about 4 mg to about 6 mg, about 4 mg to about 5 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 5 mg to about 9 mg, about 5 mg to about 8 mg, about 5 mg to about 7 mg, about 5 mg to about 6 mg, about 6 mg to about 60 mg, about 6 mg to about 50 mg, about 6 mg to about 40 mg, about 6 mg to about 30 mg, about 6 mg to about 20 mg, about 6 mg to about 10 mg, about 6 mg to about 9 mg, about 6 mg to about 8 mg, about 6 mg to about 7 mg, about 7 mg to about 60 mg, about 7 mg to about 50 mg, about 7 mg to about 40 mg, about 7 mg to about 30 mg, about 7 mg to about 20 mg, about 7 mg to about 10 mg, about 7 mg to about 9 mg, about 7 mg to about 8 mg, about 8 mg to about 60 mg, about 8 mg to about 50 mg, about 8 mg to about 40 mg, about 8 mg to about 30 mg, about 8 mg to about 20 mg, about 8 mg to about 10 mg, about 8 mg to about 9 mg, about 9 mg to about 60 mg, about 9 mg to about 50 mg, about 9 mg to about 40 mg, about 9 mg to about 30 mg, about 9 mg to about 20 mg, about 9 mg to about 10 mg, about 10 mg to about 60 mg, about 10 mg to about 50 mg, about 10 mg to about 40 mg, about 10 mg to about 30 mg, about 10 mg to about 20 mg, about 20 mg to about 60 mg, about 20 mg to about 50 mg, about 20 mg to about 40 mg, about 20 mg to about 30 mg, about 30 mg to about 60 mg, about 30 mg to about 50 mg, about 30 mg to about 40 mg, about 40 mg to about 60 mg, about 40 mg to about 50 mg, about 50 mg to about 60 mg, about 0.5 mg, about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, or about 60 mg.

In some embodiments, about 0.1 mg to about 15 mg of magnesium stearate is present in a composition herein, e.g., about 0.1 mg to about 10 mg, about 0.1 mg to about 7.5 mg, about 0.1 mg to about 5 mg, about 0.1 mg to about 2.5 mg, about 0.1 mg to about 2 mg, about 0.1 mg to about 1.5 mg, about 0.1 mg to about 1.25 mg, about 0.1 mg to about 1 mg, about 0.1 mg to about 0.75 mg, about 0.1 mg to about 0.5 mg, about 0.1 mg to about 0.25 mg, about 0.25 mg to about 15 mg, about 0.25 mg to about 10 mg, about 0.25 mg to about 7.5 mg, about 0.25 mg to about 5 mg, about 0.25 mg to about 2.5 mg, about 0.25 mg to about 2 mg, about 0.25 mg to about 1.5 mg, about 0.25 mg to about 1.25 mg, about 0.25 mg to about 1 mg, about 0.25 mg to about 0.75 mg, about 0.25 mg to about 0.5 mg, about 0.5 mg to about 15 mg, about 0.5 mg to about 10 mg, about 0.5 mg to about 7.5 mg, about 0.5 mg to about 5 mg, about 0.5 mg to about 2.5 mg, about 0.5 mg to about 2 mg, about 0.5 mg to about 1.5 mg, about 0.5 mg to about 1.25 mg, about 0.5 mg to about 1 mg, about 0.5 mg to about 0.75 mg, about 0.75 mg to about 15 mg, about 0.75 mg to about 10 mg, about 0.75 mg to about 7.5 mg, about 0.75 mg to about 5 mg, about 0.75 mg to about 2.5 mg, about 0.75 mg to about 2 mg, about 0.75 mg to about 1.5 mg, about 0.75 mg to about 1.25 mg, about 0.75 mg to about 1 mg, about 1 mg to about 15 mg, about 1 mg to about 10 mg, about 1 mg to about 7.5 mg, about 1 mg to about 5 mg, about 1 mg to about 2.5 mg, about 1 mg to about 2 mg, about 1 mg to about 1.5 mg, about 1 mg to about 1.25 mg, about 1.25 mg to about 15 mg, about 1.25 mg to about 10 mg, about 1.25 mg to about 7.5 mg, about 1.25 mg to about 5 mg, about 1.25 mg to about 2.5 mg, about 1.25 mg to about 2 mg, about 1.25 mg to about 1.5 mg, about 1.5 mg to about 15 mg, about 1.5 mg to about 10 mg, about 1.5 mg to about 7.5 mg, about 1.5 mg to about 5 mg, about 1.5 mg to about 2.5 mg, about 1.5 mg to about 2 mg, about 2 mg to about 15 mg, about 2 mg to about 10 mg, about 2 mg to about 7.5 mg, about 2 mg to about 5 mg, about 2 mg to about 2.5 mg, about 2.5 mg to about 15 mg, about 2.5 mg to about 10 mg, about 2.5 mg to about 7.5 mg, about 2.5 mg to about 5 mg, about 5 mg to about 15 mg, about 5 mg to about 10 mg, about 5 mg to about 7.5 mg, about 7.5 mg to about 15 mg, about 7.5 mg to about 10 mg, about 10 mg to about 15 mg, about 0.1 mg, about 0.25 mg, about 0.5 mg, about 0.75 mg, about 1 mg, about 1.25 mg, about 1.5 mg, about 2 mg, about 2.5 mg, about 5 mg, about 7.5 mg, about 10 mg, or about 15 mg.

In some embodiments, about 0.1% to about 2% (w/w) of a composition is magnesium stearate, e.g., about 0.1% to about 1.5%, about 0.1% to about 1%, about 0.1% to about 0.75%, about 0.1% to about 0.5%, about 0.1% to about 0.4%, about 0.1% to about 0.3%, about 0.1% to about 0.2%, about 0.2% to about 2%, about 0.2% to about 1.5%, about 0.2% to about 1%, about 0.2% to about 0.75%, about 0.2% to about 0.5%, about 0.2% to about 0.4%, about 0.2% to about 0.3%, about 0.3% to about 2%, about 0.3% to about 1.5%, about 0.3% to about 1%, about 0.3% to about 0.75%, about 0.3% to about 0.5%, about 0.3% to about 0.4%, about 0.4% to about 2%, about 0.4% to about 1.5%, about 0.4% to about 1%, about 0.4% to about 0.75%, about 0.4% to about 0.5%, about 0.5% to about 2%, about 0.5% to about 1.5%, about 0.5% to about 1%, about 0.5% to about 0.75%, about 0.75% to about 2%, about 0.75% to about 1.5%, about 0.75% to about 1%, about 1% to about 2%, about 1% to about 1.5%, about 1.5% to about 2%, about 0.1%, about 0.2%, about 0.3%, about 0.4%, about 0.5%, about 0.75%, about 1%, about 1.5%, or about 2%.

Certain compositions herein are formulated to facilitate increased energy in a person. In some such cases, the amount of magnesium stearate in the composition is about 2 mg to about 10 mg, about 4 mg to about 8 mg, about 8 mg, about 7 mg, or about 6 mg. An example product is called RH1.

Certain compositions herein are formulated to facilitate increased relaxation in a person. In some such cases, the amount of magnesium stearate in the composition is about 2 mg to about 10 mg, about 4 mg to about 8 mg, about 8 mg, about 7 mg, or about 6 mg. An example product is called RH2.

Certain compositions herein are formulated to improve the sleep health of a person. In some such cases, the amount of magnesium stearate in the composition is about 0.5 mg to about 2.5 mg, about 1 mg to about 2 mg, or about 1.5 mg. An example product is called RH3.

As described above, magnesium stearate may be added to compositions as a flow agent, and thus, may keep the ingredients of the composition separated.

### Compositions

In one aspect, provided herein are compositions comprising one or more ingredients herein, e.g., an organic extract or powder (e.g., from herbs, roots, plants), vitamins, minerals, amino acids, adaptogens, ashwagandha (including extracts and powders thereof), EDTA, extracts of Panax notoginseng and Astragalus membranaceus (e.g., Astragin®), maca (Lepidium meyenii), Eurycoma longifolia, fenugreek, Tribulus terrestris, cordyceps mushroom, vitamin B6, nitric oxide, beet root powder, green tea extract, Panax ginseng, phosphatidylserine, sunflower powder, magnesium glycinate, zinc, Magnolia officinalis, ginger, horny goat weed, sclareolides, sage, diindolylmethane, Bacopa monnieri, oat straw, D-aspartic acid, L-theanine, L-tryptophan, reishi mushroom, and melatonin. In some embodiments, the composition comprises ashwagandha extract (e.g., as described herein) that is useful in a variety of applications including, but not limited to, for use to modulate cortisol levels in a human person in order to address energy levels, relaxation levels, and sleep health.

In various embodiments, the compositions are formulated for delivery via any route known in the art.

In various aspects, the composition is formulated for oral delivery. Oral delivery may provide a means for introduction to the mouth and/or ingestion of compositions in the presence of one or more delivery-enhancing agents. Delivery-enhancing agents may include delivery-enhancing agents as described herein (e.g., AstraGin®, EDTA). A composition with delivery-enhancing properties may include a combination of ashwagandha, EDTA, and extracts of Panax notoginseng and Astragalus membranaceus (e.g., Astragin®).

In some embodiments, the means for introduction to the mouth and/or ingestion by a human person includes a lozenge containing one or more ingredients herein, e.g., ashwagandha extract. The lozenge may dissolve in a human person's mouth. In other embodiments, the means for introduction to the mouth and/or ingestion includes a pill or tablet containing the ashwagandha extract. The pill or tablet may be swallowed by the person. In some embodiments, the lozenge, pill or tablet comprises a combination of ashwagandha, EDTA, and extracts of Panax notoginseng and Astragalus membranaceus (e.g., Astragin®).

In various embodiments, compositions herein may include one or more acceptable excipients (e.g., sodium starch glycolate, colloidal silicon dioxide, microcrystalline cellulose, sucralose, peppermint flavor dried P-183, starcap 1500, and/or magnesium stearate) along with another ingredient herein (e.g., ashwagandha extract, chelating agent, delivery enhancing agent, cordyceps species, phosphatidylserine, supplemental mineral, compound derived from the magnolia tree, ginger, horny goat weed, sclareolides, DIM, zinc, extraction from aerial parts of oat plants, Eurycoma longifolia, fenugreek seed extract, Tribulus terrestris, tea extract, vitamin, nitric oxide source, amino acid, Panax ginseng, maca, Bacopa monieri, dihydrohonokiol-B, L-theanine, L-tryptophan, reishi mushroom, white willow bark, melatonin).

In various embodiments, compositions herein may include one or more acceptable carriers along with another ingredient herein (e.g., ashwagandha extract, excipient, chelating agent, delivery enhancing agent, cordyceps species, phosphatidylserine, supplemental mineral, compound derived from the magnolia tree, ginger, horny goat weed, sclareolides, DIM, zinc, extraction from aerial parts of oat plants, Eurycoma longifolia, fenugreek seed extract, Tribulus terrestris, tea extract, vitamin, nitric oxide source, amino acid, Panax ginseng, maca, bacopa monieri, dihydrohonokiol-B, L-theanine, L-tryptophan, reishi mushroom, white willow bark, melatonin). In some cases, an acceptable carrier may be an acceptable material, composition, or vehicle that is involved in carrying or transporting the ingredient from one tissue, organ, or portion of the body to another tissue, organ, or portion of the body. For example, the carrier may be a liquid or solid filler, diluent, excipient, solvent, or encapsulating material, or a combination thereof. Each component of the carrier must be compatible with the other ingredients of the formulation. It must also be suitable for use in contact with any tissues or organs with which it may come in contact, meaning that it does not carry a risk of toxicity, irritation, allergic response, immunogenicity, or any other complication that excessively outweighs its therapeutic benefits.

The amount of ingredient (e.g., ashwagandha extract, excipient, chelating agent, delivery enhancing agent, cordyceps species, phosphatidylserine, supplemental mineral, compound derived from the magnolia tree, ginger, horny goat weed, sclareolides, DIM, zinc, extraction from aerial parts of oat plants, Eurycoma longifolia, fenugreek seed extract, Tribulus terrestris, tea extract, vitamin, nitric oxide source, amino acid, Panax ginseng, maca, bacopa monieri, dihydrohonokiol-B, L-theanine, L-tryptophan, reishi mushroom, white willow bark, melatonin) used in methods herein may depend on the person and desired effect, and can be determined by one of skill in the art.

### Non-Limiting Example Compositions

In certain embodiments, an organic extract, such as ashwagandha extract, is formulated in a composition with one or more additional agents. The one or more agents may be present in the composition for a beneficial effect in the person, e.g., increased energy, reduced time needed to fall asleep, reduced anxiety. Non-limiting examples of agents include excipients, chelating agents, delivery enhancing agents, cordyceps species, phosphatidylserine, supplemental minerals, compounds derived from the magnolia tree, ginger, horny goat weed, sclareolides, DIM, zinc, extractions from aerial parts of a oat plant, Eurycoma longifolia, fenugreek seed extract, Tribulus terrestris, tea extract, one or more vitamins, nitric oxide source, amino acids, Panax ginseng, maca, Bacopa monieri, dihydrohonokiol-B, L-theanine, L-tryptophan, reishi mushroom, white willow bark, or melatonin.

In certain embodiments, provided herein are compositions comprising an organic extract and a delivery agent. In some embodiments, the delivery agent comprises extracts of panax notoginseng and Astragalus membranaceus (e.g., Astragin®). In some embodiments, the delivery agent comprises a chelating agent. In some embodiments, a chelating agent is provided as an excipient. In some embodiments, provided herein are compositions comprising about 100 mg to about 400 mg of an organic extract. In some cases the organic extract is ashwagandha extract (e.g., KSM-66®). In some embodiments, provided herein are compositions comprising about 25 mg to about 75 mg of a chelating agent. In some cases the chelating agent is EDTA. In some embodiments, provided herein are compositions comprising about 25 mg to about 75 mg of a delivery agent. In some embodiments, the delivery agent comprises extracts of Panax notoginseng and Astragalus membranaceus (e.g., Astragin®). In some embodiments, the composition comprises about 100 mg to about 400 mg of an organic extract, about 25 mg to about 75 mg of a chelating agent, and about 25 to about 75 of a delivery agent. In some embodiments, the composition comprises about 100 mg to about 400 mg of ashwagandha extract (e.g., KSM-66®), about 25 mg to about 75 mg of EDTA, and about 25 to about 75 of extracts of Panax notoginseng and Astragalus membranaceus (e.g., Astragin®). Example compositions may be used to delivery one or more additional ingredients, e.g., maca (Lepidium meyenii), Eurycoma longifolia, fenugreek, Tribulus terrestris, cordyceps mushroom, vitamin B6, nitric oxide, beet root powder, green tea extract, Panax ginseng, phosphatidylserine, sunflower powder, magnesium glycinate, zinc, Magnolia officinalis, ginger, horny goat weed, sclareolides, sage, diindolylmethane, bacopa monnieri, oat straw, D-aspartic acid, L-theanine, L-tryptophan, reishi mushroom, and melatonin.

In one embodiment, ashwagandha extract is formulated in a composition for oral delivery to modulate cortisol levels. It can have ashwagandha extract present in the composition from about 10% to about 40% (w/w). About 100 mg to about 400 mg of ashwagandha extract (e.g., KSM-66®) may be present in the composition. The composition may have one or more excipients present in the composition from about 0.1% (w/w) to about 50% (w/w). About 1 mg to about 900 mg of the one or more excipients may be present in the composition. The composition may have a chelating agent (e.g., EDTA) present in the composition from about 1% (w/w) to about 10% (w/w). About 25 mg to about 75 mg of the chelating agent may be present in the composition. The composition may have a delivery enhancing agent (e.g., AstraGin®) from about 1% (w/w) to about 10% (w/w). About 25 mg to about 75 mg of the delivery enhancing agent may be present in the composition. The composition may have maca present in the composition from about 10% (w/w) to about 35% (w/w). About 100 mg to about 400 mg of maca may be present in the composition. The composition may have Eurycoma longifolia (in some cases also referred to as tongkat ali) present in the composition from about 5% (w/w) to about 25% (w/w). About 50 mg to about 225 mg of Eurycoma longifolia may be present in the composition. The composition may have fenugreek seed extract (e.g., Testosurge®) present in the composition from about 5% (w/w) to about 25% (w/w). About 50 mg to about 225 mg of the fenugreek seed extract may be present in the composition. The composition may have Tribulus terrestris present in the composition from about 0.1% (w/w) to about 2.5% (w/w). About 1 mg to about 25 mg of Tribulus terrestris may be present in the composition. The composition may have a cordyceps species (e.g., CordycepsPrime®) in the composition from about 0.1% (w/w) to about 1.5% (w/w). About 2.5 mg to about 15 mg of the cordyceps species can be present in the composition. The composition may have a nitric oxide source (e.g., beet root powder) present in the composition from about 0.1% (w/w) to about 2% (w/w). About 2.5 mg to about 15 mg of the nitric oxide source may be present in the composition. The composition may have one or more vitamins (e.g., vitamin B6) present in the composition from about 0.05% (w/w) to about 1% (w/w). About 0.5 mg to about 3 mg of the vitamin may be present in the composition. The composition may have a tea extract (e.g., green tea extract) present in the composition from about 0.1% (w/w) to about 1.5% (w/w). About 2.5 mg to about 15 mg of the tea extract may be present in the composition. The composition may have one or more amino acids (e.g., D-aspartic acid) present in the composition from about 0.1% (w/w) to about 1.5% (w/w). About 2.5 mg to about 15 mg of the amino acid may be present in the composition. The composition may have Panax ginseng present in the composition from about 0.1% (w/w) to about 1.5% (w/w). About 2.5 mg to about 15 mg of the Panax ginseng may be present in the composition. The composition may be RH1. In some embodiments, the composition is a taken by a male. In some embodiments, the composition has one or more excipients, e.g., sodium starch glycolate, colloidal silicon dioxide, microcrystalline cellulose, sucralose, peppermint flavor (e.g., spray dried P-183, natural S.F.), menthol crystals, starcap 1500, or magnesium stearate, or two or more thereof.

In one embodiment, ashwagandha extract is formulated in a composition for oral delivery to modulate cortisol levels. It can have ashwagandha extract present in the composition from about 10% to about 40% (w/w). About 100 mg to about 400 mg of ashwagandha extract (e.g., KSM-66®) may be present in the composition. The composition may have one or more excipients present in the composition from about 0.1% (w/w) to about 50% (w/w). About 1 mg to about 900 mg of the one or more excipients may be present in the composition. The composition may have a chelating agent (e.g., EDTA) present in the composition from about 1% (w/w) to about 10% (w/w). About 25 mg to about 75 mg of the chelating agent may be present in the composition. The composition may have a delivery enhancing agent (e.g., AstraGin®) from about 1% (w/w) to about 10% (w/w). About 25 mg to about 75 mg of the delivery enhancing agent may be present in the composition. The composition may have maca present in the composition from about 10% (w/w) to about 35% (w/w). About 100 mg to about 400 mg of maca may be present in the composition. The composition may have Eurycoma longifolia (in some cases also referred to as tongkat ali) present in the composition from about 5% (w/w) to about 25% (w/w). About 50 mg to about 225 mg of Eurycoma longifolia may be present in the composition. The composition may have fenugreek seed extract (e.g., Testosurge®) present in the composition from about 5% (w/w) to about 25% (w/w). About 50 mg to about 225 mg of the fenugreek seed extract may be present in the composition. The composition may have Tribulus terrestris present in the composition from about 0.1% (w/w) to about 2.5% (w/w). About 1 mg to about 25 mg of Tribulus terrestris may be present in the composition. The composition may have a cordyceps species (e.g., CordycepsPrime®) in the composition from about 0.1% (w/w) to about 1.5% (w/w). About 2.5 mg to about 15 mg of the cordyceps species can be present in the composition. The composition may have a nitric oxide source (e.g., beet root powder) present in the composition from about 0.1% (w/w) to about 2% (w/w). About 2.5 mg to about 15 mg of the nitric oxide source may be present in the composition. The composition may have one or more vitamins (e.g., vitamin B6) present in the composition from about 0.05% (w/w) to about 1% (w/w). About 0.5 mg to about 3 mg of the vitamin may be present in the composition. The composition may have a tea extract (e.g., green tea extract) present in the composition from about 0.1% (w/w) to about 1.5% (w/w). About 2.5 mg to about 15 mg of the tea extract may be present in the composition. The composition may have one or more amino acids (e.g., D-aspartic acid) present in the composition from about 0.1% (w/w) to about 1.5% (w/w). About 2.5 mg to about 15 mg of the amino acid may be present in the composition. The composition may have Panax ginseng present in the composition from about 0.1% (w/w) to about 1.5% (w/w). About 2.5 mg to about 15 mg of the Panax ginseng may be present in the composition. The composition may have silicon dioxide (e.g., anhydrous silicon dioxide (Compact Cell)) present in the composition from about 1% (w/w) to about 10% (w/w). About 15 mg to about 100 mg of the silicon dioxide (e.g., anhydrous silicon dioxide (Compact Cell)) may be present in the composition. The composition may have an excipient (e.g., FUJI FMELT TYPE C) present in the composition from about 10% (w/w) to about 35% (w/w). About 100 mg to about 400 mg of the disintegrant may be present in the composition. The composition may be RH1. In some embodiments, the composition is a taken by a male. In some embodiments, the composition has one or more excipients, e.g., sodium starch glycolate, colloidal silicon dioxide, microcrystalline cellulose, sucralose, flavor (e.g., spray dried P-183, natural S.F., natural flavoring (e.g., grapefruit or apple), a peppermint oil, a spearmint oil blend, a peppermint/spearmint oil blend, a fruit extract (e.g., monkfruit), a sugar substitute (e.g., STEVIA), or a flavoring agent with another flavor), menthol crystals, starcap 1500, or magnesium stearate, or two or more thereof.

In one embodiment, ashwagandha extract is formulated in a composition for oral delivery. The composition may have ashwagandha extract (e.g., KSM-66®) present in the composition from about 10% to about 40% (w/w). About 100 mg to about 400 mg of ashwagandha extract may be present in the composition. The composition may have one or more excipients present in the composition from about 0.1% (w/w) to about 50% (w/w). About 1 mg to about 900 mg of the one or more excipients may be present in the composition. The composition may have a chelating agent (e.g., EDTA) present in the composition from about 1% (w/w) to about 10% (w/w). About 25 mg to about 75 mg of the chelating agent may be present in the composition. The composition may have a delivery enhancing agent (e.g., AstraGin®) from about 1% (w/w) to about 10% (w/w). About 25 mg to about 75 mg of the delivery enhancing agent may be present in the composition. The composition may have maca present in the composition from about 10% (w/w) to about 35% (w/w). About 100 mg to about 400 mg of maca may be present in the composition. The composition may have fenugreek seed extract (e.g., Testosurge®) present in the composition from about 5% (w/w) to about 25% (w/w). About 75 mg to about 350 mg of the fenugreek seed extract may be present in the composition. The composition may have one or more vitamins (e.g., vitamin B6) present in the composition from about 0.01% (w/w) to about 1.5% (w/w). About 0.5 mg to about 6 mg of the one or more vitamins may be present in the composition. The composition may have a cordyceps species (e.g., CordycepsPrime®) in the composition from about 0.5% (w/w) to about 1.6% (w/w). About 5 mg to about 20 mg of the cordyceps species may be present in the composition. The composition may have a tea extract (e.g., green tea extract) present in the composition from about 0.5% (w/w) to about 1.6% (w/w). About 5 mg to about 20 mg of the tea extract may be present in the composition. The composition may have a nitric oxide source (e.g., beet root powder) present in the composition from about 0.5% (w/w) to about 1.6% (w/w). About 5 mg to about 20 mg of the nitric oxide source may be present in the composition. The composition may have bacopa present in the composition from about 0.5% (w/w) to about 1.6% (w/w). About 5 mg to about 20 mg of the bacopa may be present in the composition. The composition may have an amino acid (e.g., D-aspartic acid) present in the composition from about 0.1% (w/w) to about 1% (w/w). About 2.5 mg to about 10 mg of the amino acid may be present in the composition. The composition may have Panax ginseng present in the composition from about 0.1% (w/w) to about 1% (w/w). About 2.5 mg to about 10 mg of the Panax ginseng may be present in the composition. The composition may be RH1. In some embodiments, the composition is a taken by a female. In some embodiments, the composition has one or more excipients, e.g., sodium starch glycolate, colloidal silicon dioxide, microcrystalline cellulose, sucralose, peppermint flavor (e.g., spray dried P-183, natural S.F.), menthol crystals, starcap 1500, or magnesium stearate, or two or more thereof.

In one embodiment, ashwagandha extract is formulated in a composition for oral delivery. The composition may have ashwagandha extract (e.g., KSM-66®) present in the composition from about 10% to about 40% (w/w). About 100 mg to about 400 mg of ashwagandha extract can be present in the composition. The composition may have one or more excipients present in the composition from about 0.1% (w/w) to about 50% (w/w). About 1 mg to about 900 mg of the one or more excipients may be present in the composition. The composition may have a chelating agent (e.g., EDTA) present in the composition from about 1% (w/w) to about 10% (w/w). About 25 mg to about 75 mg of the chelating agent may be present in the composition. The composition may have a delivery enhancing agent (e.g., AstraGin®) from about 1% (w/w) to about 10% (w/w). About 25 mg to about 75 mg of the delivery enhancing agent may be present in the composition. The composition may have a cordyceps species (e.g., CordycepsPrime™) present in the composition from about 5% (w/w) to about 25% (w/w). About 100 mg to about 300 mg of the cordyceps species may be present in the composition. The composition may have phosphatidylserine present (e.g., Sunflower Powder) in the composition from about 5% (w/w) to about 20% (w/w). About 75 mg to about 175 mg of the phosphatidylserine may be present in the composition. The composition may have a supplemental mineral (e.g., Magnesium Glycinate) present in the composition from about 1% (w/w) to about 10% (w/w). About 25 mg to about 75 mg of the supplemental mineral may be present in the composition. The composition may have a compound derived from a magnolia tree (e.g., Magnolia officinalis) in the composition from about 1% (w/w) to about 10% (w/w). About 10 mg to about 90 mg of said compound derived from a magnolia tree may be present in the composition. The composition may have ginger present in the composition from about 0.1% (w/w) to about 2.5% (w/w). About 5 mg to about 25 mg of the ginger may be present in the composition. The composition may have horny goat weed present in the composition from about 0.1% (w/w) to about 2.5% (w/w). About 5 mg to about 25 mg of the horny goat weed may be present in the composition. The composition may have sclareolides (e.g., Sage Leaf Powder) present in the composition from about 0.1% (w/w) to about 1% (w/w). About 2.5 mg to about 10 mg of the sclareolides may be present in the composition. The composition may have diindolylmethane (DIM) present in the composition from about 0.1% (w/w) to about 1% (w/w). About 2.5 mg to about 10 mg of the DIM may be present in the composition. The composition may have zinc present in the composition from about 0.1% (w/w) to about 2% (w/w). About 5 mg to about 20 mg of the zinc can be present in the composition. The composition may be RH2. In some embodiments, the composition is a taken by a male. In some embodiments, the composition has one or more excipients, e.g., sodium starch glycolate, colloidal silicon dioxide, microcrystalline cellulose, sucralose, peppermint flavor (e.g., spray dried P-183, natural S.F.), menthol crystals, starcap 1500, or magnesium stearate, or two or more thereof.

In one embodiment, ashwagandha extract is formulated in a composition for oral delivery. The composition may have ashwagandha extract (e.g., KSM-66®) present in the composition from about 10% to about 40% (w/w). About 100 mg to about 400 mg of ashwagandha extract can be present in the composition. The composition may have one or more excipients present in the composition from about 0.1% (w/w) to about 50% (w/w). About 1 mg to about 900 mg of the one or more excipients may be present in the composition. The composition may have a chelating agent (e.g., EDTA) present in the composition from about 1% (w/w) to about 10% (w/w). About 25 mg to about 75 mg of the chelating agent may be present in the composition. The composition may have a delivery enhancing agent (e.g., AstraGin®) from about 1% (w/w) to about 10% (w/w). About 25 mg to about 75 mg of the delivery enhancing agent may be present in the composition. The composition may have a cordyceps species (e.g., CordycepsPrime™) present in the composition from about 5% (w/w) to about 25% (w/w). About 100 mg to about 300 mg of the cordyceps species may be present in the composition. The composition may have phosphatidylserine present (e.g., Sunflower Powder) in the composition from about 5% (w/w) to about 20% (w/w). About 75 mg to about 175 mg of the phosphatidylserine may be present in the composition. The composition may have a supplemental mineral (e.g., Magnesium Glycinate) present in the composition from about 1% (w/w) to about 10% (w/w). About 25 mg to about 75 mg of the supplemental mineral may be present in the composition. The composition may have a compound derived from a magnolia tree (e.g., Magnolia officinalis) in the composition from about 1% (w/w) to about 10% (w/w). About 10 mg to about 90 mg of said compound derived from a magnolia tree may be present in the composition. The composition may have ginger present in the composition from about 0.1% (w/w) to about 2.5% (w/w). About 5 mg to about 25 mg of the ginger may be present in the composition. The composition may have horny goat weed present in the composition from about 0.1% (w/w) to about 2.5% (w/w). About 5 mg to about 25 mg of the horny goat weed may be present in the composition. The composition may have sclareolides (e.g., Sage Leaf Powder) present in the composition from about 0.1% (w/w) to about 1% (w/w). About 2.5 mg to about 10 mg of the sclareolides may be present in the composition. The composition may have diindolylmethane (DIM) present in the composition from about 0.1% (w/w) to about 1% (w/w). About 2.5 mg to about 10 mg of the DIM may be present in the composition. The composition may have zinc present in the composition from about 0.1% (w/w) to about 2% (w/w). About 5 mg to about 20 mg of the zinc can be present in the composition. The composition may be RH2. In some embodiments, the composition is a taken by a male. In some embodiments, the composition has one or more excipients, e.g., sodium starch glycolate, colloidal silicon dioxide, microcrystalline cellulose, sucralose, peppermint flavor (e.g., spray dried P-183, natural S.F.), menthol crystals, starcap 1500, natural flavoring (e.g., apple or grapefruit), silica, FUJI FMELT TYPE C, a fruit extract (e.g., monkfruit), or magnesium stearate, or two or more thereof.

In one embodiment, an ashwagandha extract is formulated in a composition for oral delivery. The composition may have ashwagandha extract (e.g., KSM-66®) present in the composition from about 10% to about 40% (w/w). About 100 mg to about 400 mg of ashwagandha extract may be present in the composition. The composition may have one or more excipients present in the composition from about 0.1% (w/w) to about 50% (w/w). About 1 mg to about 900 mg of the one or more excipients may be present in the composition. The composition may have a chelating agent (e.g., EDTA) present in the composition from about 1% (w/w) to about 10% (w/w). About 25 mg to about 75 mg of the chelating agent may be present in the composition. The composition may have a delivery enhancing agent (e.g., AstraGin®) from about 1% (w/w) to about 10% (w/w). About 25 mg to about 75 mg of the delivery enhancing agent may be present in the composition. The composition may have a cordyceps species (e.g., CordycepsPrime™) present in the composition from about 5% (w/w) to about 25% (w/w). About 100 mg to about 300 mg of the cordyceps species may be present in the composition. The composition may have phosphatidylserine (e.g., Sunflower Powder) present in the composition from about 5% (w/w) to about 20% (w/w). About 75 mg to about 175 mg of the phosphatidylserine may be present in the composition. The composition may have a supplemental mineral (e.g., Magnesium Glycinate) present in the composition from about 1% (w/w) to about 10% (w/w). About 25 mg to about 75 mg of the supplemental mineral may be present in the composition. The composition may have a compound derived from a magnolia tree (e.g., Magnolia officinalis) in the composition from about 1% (w/w) to about 10% (w/w). About 25 mg to about 75 mg of the compound derived from a magnolia tree can be present in the composition. The composition may have ginger present in the composition from about 0.1% (w/w) to about 2.5% (w/w). About 5 mg to about 25 mg of the ginger may be present in the composition. The composition may have an extraction from aerial parts of oat plants from about 0.1% (w/w) to about 2.5% (w/w). About 5 mg to about 25 mg of the extraction from aerial parts of oat plants (e.g., oat straw) may be present in the composition. The composition may have sclareolides (e.g., sage leaf powder) present in the composition from about 0.1% (w/w) to about 1% (w/w). About 2.5 mg to about 10 mg of the sclareolides may be present in the composition. The composition may have diindolylmethane (DIM) present in the composition from about 0.1% (w/w) to about 1% (w/w). About 2.5 mg to about 10 mg of the DIM may be present in the composition. The composition may have zinc present in the composition from about 0.1% (w/w) to about 1.5% (w/w). About 2.5 mg to about 15 mg of the zinc may be present in the composition. The composition may be RH2. In some embodiments, the composition is a taken by a female. In some embodiments, the composition has one or more excipients, e.g., sodium starch glycolate, colloidal silicon dioxide, microcrystalline cellulose, sucralose, peppermint flavor (e.g., spray dried P-183, natural S.F.), menthol crystals, starcap 1500, or magnesium stearate, or two or more thereof.

In one embodiment, an ashwagandha extract is formulated in a composition for oral delivery. The composition may have ashwagandha extract (e.g., KSM-66®) present in the composition from about 10% to about 40% (w/w). About 100 mg to about 400 mg of ashwagandha extract may be present in the composition. The composition may have one or more excipients present in the composition from about 0.1% (w/w) to about 50% (w/w). About 1 mg to about 900 mg of the one or more excipients may be present in the composition. The composition may have a chelating agent (e.g., EDTA) present in the composition from about 1% (w/w) to about 10% (w/w). About 25 mg to about 75 mg of the chelating agent may be present in the composition. The composition may have a delivery enhancing agent (e.g., AstraGin®) from about 1% (w/w) to about 10% (w/w). About 25 mg to about 75 mg of the delivery enhancing agent may be present in the composition. The composition may have a cordyceps species (e.g., CordycepsPrime™) present in the composition from about 5% (w/w) to about 25% (w/w). About 100 mg to about 300 mg of the cordyceps species may be present in the composition. The composition may have phosphatidylserine (e.g., Sunflower Powder) present in the composition from about 5% (w/w) to about 20% (w/w). About 75 mg to about 175 mg of the phosphatidylserine may be present in the composition. The composition may have a supplemental mineral (e.g., Magnesium Glycinate) present in the composition from about 1% (w/w) to about 10% (w/w). About 25 mg to about 75 mg of the supplemental mineral may be present in the composition. The composition may have a compound derived from a magnolia tree (e.g., Magnolia officinalis) in the composition from about 1% (w/w) to about 10% (w/w). About 25 mg to about 75 mg of the compound derived from a magnolia tree can be present in the composition. The composition may have ginger present in the composition from about 0.1% (w/w) to about 2.5% (w/w). About 5 mg to about 25 mg of the ginger may be present in the composition. The composition may have an extraction from aerial parts of oat plants from about 0.1% (w/w) to about 2.5% (w/w). About 5 mg to about 25 mg of the extraction from aerial parts of oat plants (e.g., oat straw) may be present in the composition. The composition may have sclareolides (e.g., sage leaf powder) present in the composition from about 0.1% (w/w) to about 1% (w/w). About 2.5 mg to about 10 mg of the sclareolides may be present in the composition. The composition may have diindolylmethane (DIM) present in the composition from about 0.1% (w/w) to about 1% (w/w). About 2.5 mg to about 10 mg of the DIM may be present in the composition. The composition may have zinc present in the composition from about 0.1% (w/w) to about 1.5% (w/w). About 2.5 mg to about 15 mg of the zinc may be present in the composition. The composition may be RH2. In some embodiments, the composition is a taken by a female. In some embodiments, the composition has one or more excipients, e.g., sodium starch glycolate, colloidal silicon dioxide, microcrystalline cellulose, sucralose, peppermint flavor (e.g., spray dried P-183, natural S.F.), menthol crystals, starcap 1500, natural flavoring (e.g., apple or grapefruit), silica, FUJI FMELT TYPE C, a fruit extract (e.g., monkfruit), or magnesium stearate, or two or more thereof.

In one embodiment, an ashwagandha extract is formulated in a composition for oral delivery. It can have ashwagandha extract (e.g., KSM-66®) present in the composition from about 25% to about 50% (w/w). About 75 mg to about 250 mg of ashwagandha extract may be present in the composition. The composition may have one or more excipients present in the composition from about 0.1% (w/w) to about 55% (w/w). About 0.1 mg to about 225 mg of the one or more excipients may be present in the composition. The composition may have a chelating agent (e.g., EDTA) present in the composition from about 3% (w/w) to about 15% (w/w). About 10 mg to about 40 mg of the chelating agent may be present in the composition. The composition may have a delivery enhancing agent (e.g., AstraGin®) from about 3% (w/w) to about 15% (w/w). About 10 mg to about 40 mg of the delivery enhancing agent may be present in the composition. The composition may have phosphatidylserine (e.g., Sunflower Powder) from about 1% (w/w) to about 10% (w/w). About 5 mg to about 25 mg of the phosphatidylserine may be present in the composition. The composition may have a compound derived from a magnolia tree (e.g., magnolia officinalis) in the composition from about 1% (w/w) to about 10% (w/w). About 5 mg to about 25 mg of the compound derived from a magnolia tree can be present in the composition. The composition may have L-theanine from about 1% (w/w) to about 8% (w/w). About 5 mg to about 15 mg of the L-theanine may be present in the composition. The composition may have L-tryptophan from about 1% (w/w) to about 7% (w/w). About 2.5 mg to about 15 mg of the L-tryptophan may be present in the composition. The composition may have reishi mushroom from about 0.5% (w/w) to about 3% (w/w). About 2.5 mg to about 10 mg of the reishi mushroom may be present in the composition. The composition may have melatonin from about 0.1% (w/w) to about 2% (w/w). About 1 mg to about 5 mg of the melatonin may be present in the composition. The composition may be RH3. In some embodiments, the composition is a taken by a male. In some embodiments, the composition is taken by a female. In some embodiments, the composition has one or more excipients, e.g., sodium starch glycolate, colloidal silicon dioxide, microcrystalline cellulose, sucralose, peppermint flavor (e.g., spray dried P-183, natural S.F.), menthol crystals, starcap 1500, or magnesium stearate, or two or more thereof.

### Tablet Formulation

In some aspects, compositions herein may be formulated in a solid formation, such as a tablet, that may be received orally. In some embodiments, the tablet may be a lozenge or pill. In other embodiments, compositions herein may be formulated in a liquid formation that may be received orally. In some embodiments the formulation may comprise one or more ingredients (e.g., ashwagandha extract, cordyceps species, phosphatidylserine, supplemental mineral, compound derived from the magnolia tree, ginger, horny goat weed, sclareolides, DIM, zinc, extraction from aerial parts of oat plants, Eurycoma longifolia, fenugreek seed extract, Tribulus terrestris, tea extract, vitamin, nitric oxide source, amino acid, Panax ginseng, maca, bacopa monieri, L-theanine, L-tryptophan, reishi mushroom, white willow bark, melatonin, or a combination of one or more thereof), one or more excipients (e.g., sodium starch glycolate, one or more flavoring agents, one or more sweetening agents, one or more adsorbents, glidants, and/or silicon dioxide, one or more of binders, diluents, and/or cellulose, one or more starch excipients, magnesium stearate, and/or menthol crystals), a chelating agent (e.g., EDTA), and a delivery enhancing agent (e.g., AstraGin®).

Oral formulations described herein can be formulated into tablets with methods well known in the art.

### Systems

In certain aspects, a system is provided herein that in some embodiments is used with a composition herein. In some cases, the system is used prior to taking the composition. In some cases, the system is used after taking the composition. In some cases, the system is used while taking the composition. In some cases, the system is used multiple times, for instance, more than once a week, more than once a day, e.g., 2, 3, 4, 5, or 6 times a day. In some embodiments, the system measures or receives one or more physiological features of a person, e.g., cortisol, biotin (vitamin B7), ketones, vitamin C, vitamin B9, creatinine, magnesium, water, vitamin B12, calcium, sodium, and combinations of two or more thereof. In some embodiments, the system receives one or more factors regarding the person's lifestyle, e.g., sleep, libido, exercise, and the like. In some cases, the system provides feedback, e.g., makes one or more lifestyle recommendations. In some cases, the system provides feedback, e.g., provides a score or other indication of the person's cortisol levels. A non-limiting example recommendation is whether to take the composition. Other non-limiting recommendations include increasing water intake, sleep, dietary recommendations, and the like. Feedback/recommendations for a person may be determined based on one or more factors regarding the person. In some embodiments, the one or more factors regarding the person may be determined based on received information regarding the person. The received information may include data regarding the health, lifestyle, activities, or personal information of the person. The data may be input or provided by the person. The one or more factors regarding the person may also be determined based on a comparison of the received information and data. In some cases, the received information may contain one or more of the one or more factors. Recommendations for a person may also be determined based on information received regarding one or more physiological features of the person, e.g., cortisol levels, pH, metabolite concentration in urine, and the like. Received information is described further herein.

In one embodiment, a human may analyze the received information and provide feedback such as determine a recommendation. The recommendation may include a lifestyle adjustment, dietary adjustment, and/or whether or not take a certain composition, e.g., a composition herein. The feedback and/or recommendation may be based on the received information and data.

In some embodiments, the system may determine feedback (e.g., a recommendation) based on the received information using a server (e.g., a physical processing device or a virtual server) or a computing device (e.g., a laptop, smartphone, or another processing device) of the system. In those embodiments, the received information may be compared to the data and the feedback (e.g., recommendation) may be determined based on the comparison. The data may be received by the server or computing device or may be stored in a database on the server or computing device. In some cases, the server or computing device may receive the information from a processing device associated with the information.

In some embodiments, the received information may be received from the person or from a processing device (e.g., a computing device) associated with the person. The information may be input by the person to the processing device, which then may provide the information to a human or processing device. In embodiments with the processing device associated with the person, the processing device associated with the person may further receive feedback, such as a recommendation, e.g., lifestyle, diet, supplement, from the processing device. The recommendation may indicate that the person should take a first product (e.g., RH1), a second product (e.g., RH2), and a third product (RH3), or any combination thereof.

In some embodiments, the feedback (e.g., recommendation) is made using the received information and the data. As described above, in some embodiments, recommendations are determined based on one or more factors. The one or more factors may include cortisol levels of the person. The cortisol levels may be an average of two or more measurements of the person's cortisol, e.g., measured the same day. In an example, the cortisol levels or one or more other factors are measured 2, 3, 4, 5, or 6 times a day. The cortisol levels or other factors may be averaged from the 2, 3, 4, 5, or 6 measurements. The cortisol levels of the person may include current cortisol levels of the person as well as desired cortisol levels of the person. The cortisol levels of the person may be determined based on the received information.

In some embodiments, a second feedback (e.g., recommendation) may be provided based on additional information, edited information, or re-input information. In those embodiments, the person may have the option to input additional information, edit information, or re-input information in order to receive the second recommendation.

As described above, the feedback (e.g., recommendation) may be determined based on one or more factors (e.g., one or more cortisol levels of a person). The one or more factors may be determined based on the received information, which may be input by a person. Additionally, the one or more factors may include one or more relevant pieces of the received information. For example, as described below, if the received information includes the person's date of birth, one of the one or more factors may be the date of birth itself, and one of the one or more factors may be the age range determined based on the date of birth.

As described above, in some embodiments, information about the person is received as input. The information may be received from the person or from a processing device associated with the person. Non-limiting examples of information received by the system include the age and/or date of birth of the person, sleep data of the person, diet of the person, libido level of the person, mental clarity of the person, fatigue levels of the person, mood of the person, daily exercise of the person, level of energy of the person during exercise, daily stress levels of the person, measured cortisol levels of the person in the morning, measured cortisol levels of the person at midday, measured cortisol levels of the person in the evening, measured cortisol levels of the person at night, and averaged cortisol levels of the person from two or more measurements in a day.

Determining the feedback (e.g., recommendation) may be based on one or more factors associated with the age of the person, such as an age range that the person belongs to. For example, information specifying the date of birth or the age of the person may be received. Relevant age ranges may be determined, and the recommendation may be based on those age ranges, such as age ranges of between 18 and 29 years, between 30 and 39 years, between 40 and 49 years, between 50 and 59 years, or between 60 and 70 years. While certain age ranges are described, these ranges are exemplary, and different ranges may be used.

The feedback (e.g., recommendation) may be further determined based on one or more factors associated with the daily sleep of the person. For example, information specifying that the person sleeps for a specified average amount of time each night, and that specified average amount of time each night may fall within a specified range may be received. Specified ranges may include less than about 4 hours, from about 4 hours to about 5 hours, from about 5 hours to about 6 hours, from about 6 hours to about 7 hours, from about 7 hours to about 8 hours, from about 8 hours to about 9 hours, from about 9 hours to about 10 hours, or more than about 10 hours. While certain ranges of sleep are described, these ranges are exemplary, and different ranges may be used.

The feedback (e.g., recommendation) may be further determined based on one or more factors associated with the daily exercise of the person. For example, information specifying that the person exercises for a specified average amount of time each day or night, and that specified average amount of time exercised may fall within a specified range may be received. Specified ranges may include no daily exercise, about 30 minutes of daily exercise, about 1 hour of daily exercise, about 1.5 hours of daily exercise, or about 2 hours or more of daily exercise. While certain ranges of daily, these ranges are exemplary, and different ranges may be used. Additionally, the person may input information specifying that the person did a specific exercise, such as running, lifting weights, swimming, boxing, soccer, or other various exercises, on each day. While certain exercises are described, these exercises are exemplary, and different exercises may be performed and input.

The feedback (e.g., recommendation) may be further determined based on one or more factors associated with the diet of the person. For example, the person may input information specifying the number of meals per day the person eats, such as two or three meals a day. As another example, the person may input an amount of calories that the person intakes per day, such as 2000 calories per day or 3000 calories per day. As yet another example, the person may input the type of food eaten each day, such as eggs or spinach. While certain amounts of meals, amounts of calories, and types of food are described, these are exemplary, and other amounts or types may be used. In some embodiments, the received information may indicate that the person considers his or her diet to be considerably unhealthy, unhealthy, average, healthy, or considerably healthy. While only certain health levels attributed to a person's diet are described, these levels are exemplary, and other levels or methods of describing a person's diet may be used.

The feedback (e.g., recommendation) may also be determined based on one or more factors associated with subjective information (e.g., information based on the person's feeling or belief) received, such as libido level of the person, mental clarity of the person, fatigue levels of the person, mood of the person, level of energy of the person during exercise, and daily stress levels of the person.

For example, the feedback (e.g., recommendation) may be determined based on one or more factors indicating the overall libido of the person. For example, the received information may indicate that the person feels or believes his or her libido is no libido, libido less than the average man or woman, libido of the average man or woman, more libido than the average man or woman, or substantially more libido than the average man or woman. While certain levels of libido are described, these are exemplary, and other information about libido may be used.

As another example, the feedback (e.g., recommendation) may be determined based on one or more factors indicating the daily mental clarity of the person. For example, the received information may indicate that the person feels or believes his or her mental clarity is no mental clarity, mental clarity less than the average man or woman, mental clarity of the average man or woman, more mental clarity than the average man or woman, or substantially more mental clarity than the average man or woman. While certain levels of mental clarity are described, these are exemplary, and other information about mental clarity may be used.

As yet another example, the feedback (e.g., recommendations) may be determined based on one or more factors indicating the daily fatigue level of the person. For example, the received information may indicate that the person feels or believes his or her fatigue level is no fatigue, about a fatigue level less than the average man or woman, about a fatigue level equal to the average man or woman, about a fatigue level greater than the average man or woman, or about a fatigue level substantially greater than the average man or woman. While certain fatigue levels are described, these are exemplary, and other information about a person's fatigue may be used.

As even another example, feedback (e.g., recommendations) may be determined based on one or more factors indicating the daily mood of the person. For example, the received information may indicate that the person feels or believes his or her mood is a mood substantially worse than the average man or woman, a mood worse than the average man or woman, a mood the same as the average man or woman, a mood better than the average man or woman, or a mood substantially better than the average man or woman. While certain moods are described, these are exemplary, and other information about a person's fatigue may be used.

As yet another example, feedback (e.g., recommendations) may be determined based on one or more factors indicating the daily stress level of the person. For example, the received information may indicate that the person feels or believes his or her daily stress level is no daily stress, about a daily stress level less than the average man or woman, about a daily stress level equal to the average man or woman, about a daily stress level greater than the average man or woman, or about a daily stress level substantially greater than the average man or woman. While certain stress levels are described, these are exemplary, and other information about a person's stress level may be used.

As one more example, feedback (e.g., recommendations) may be determined based on one or more factors indicating the energy levels of the person during workouts. For example, the received information may indicate that the person feels or believes his or her energy level is substantially less than the average man or woman, about an energy level less than the average man or woman, about an energy level about equal to the average man or woman, about an energy level greater than the average man or woman, or about an energy level substantially greater than the average man or woman. While certain energy levels are described, these are exemplary, and other information about a person's energy level may be used.

In some examples, feedback (e.g., recommendations) may include a score. The score may be calculated by the system based on the one or more factors and information about the person regarding those one or more factors. The score may further be determined using weights associated with each of the factors. In some embodiments, higher scores may indicate that the person should not make a lifestyle, dietary, and/or nutrient adjustment, and/or should maintain a lifestyle, diet, and/or nutrient intake, while lower scores may indicate that the person should make the adjustment(s), and vice versa.

In some embodiments the score is an indication of a person's cortisol levels. The score may be calculated by the system based on the one or more factors and information about the person regarding those one or more factors. The score may further be determined using weights associated with each of the factors. In some embodiments, higher scores may indicate that the person should not make a lifestyle, dietary, and/or nutrient adjustment, and/or should maintain a lifestyle, diet, and/or nutrient intake, while lower scores may indicate that the person should make the adjustment(s), and vice versa.

In some embodiments, the algorithm for calculating the user's cortisol score may be fixed for each user. In some embodiments, the algorithm may take into consideration one or more factors to determine the health score calculations. The one or more user input information or data may be used to determine and/or adjust the weighted values used to calculate the score. In some embodiments, the algorithm may adjust the impact of one or more factors in response to a user input. A non-limiting example is if the users age is 60+, the weight of the user's libido may be less than the weight of a user libido for a user in the age range of 20-29. In another example, for a 60+ year old user, sleep maybe be weighted higher than for a younger user (e.g., user 20-29 years, old, a user 30-39 years old, a user 40-49 years old, or a user 50-59 years old).

In some embodiment the algorithm can receive input data from a user in response to approximately 12 sections of health factor questions, each with various levels of weighted scores based on a determined influence the one or more health factors on a user's overall health and wellbeing. Cortisol can be a strong indicator of a user's health and wellbeing and therefore the algorithm can be configured to weight the answers and data from the user such that a health score of the user is a direct indication of a user's cortisol level and outputting a score and feedback to the user corresponding to their cortisol The score and feedback being easier for a user to understand compared to the inconsistent cortisol monitoring methods currently used.

Below describes a non-limiting example of how the algorithm can weight different sections of user's answers and input to determine an accurate cortisol level related health score.

One of the one or more section can be sleep. Sleep is a critical factor to maintaining appropriate hormone levels. The proper circadian rhythm and sleep regimen incorporates light, deep and REM sleep repeated numerous times (three stage sleep state) in a 7-9 hour period for all adults. The majority of our hormones are generated during the deep and REM cycles. Therefore, we weighted sleep heavily in the algorithm. Note that sleep aids knock the user out but do not induce the correct three stage sleep state and hence, most adults do not obtain the ideal 7-9 hours of three stage sleep, with or without sleep aids, causing hormone generation to be sub-optimum. Therefore, the algorithm may be configured to weight a user's answer regarding their sleep data and information more highly than answers regarding other health factors. A non-limiting example being, if a user identifies that they are getting more than 9 hours of sleep, they will get a lower score, as studies and data suggest that this is indicative of an unhealthy sleep pattern. If a user identifies that they are getting less than 7 hours of sleep, they will get a decreasing score for each hour less than 7-9 hours of sleep. Studies and data suggest that obtaining less than 5 hours of sleep this is indicative of an unhealthy sleep pattern and underling medical issues are of concern, therefore if a user selects less than 5 hours of sleep, they will obtain a negative score that in turn will be weighted in accordance with our proprietary algorithm.

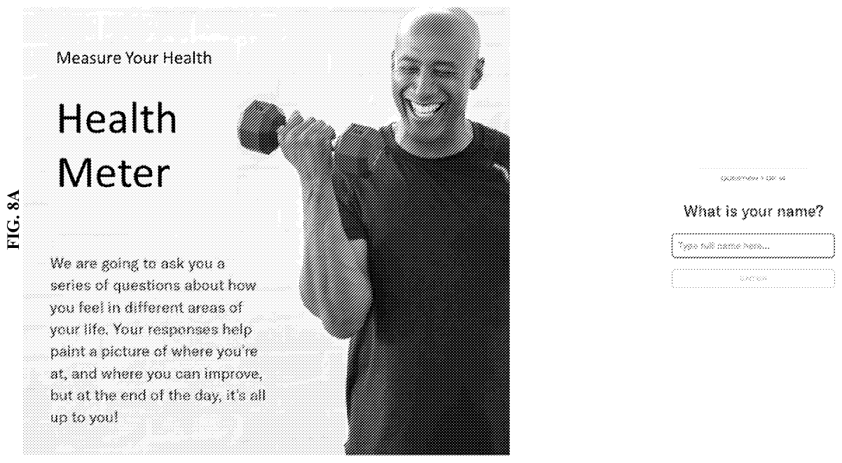

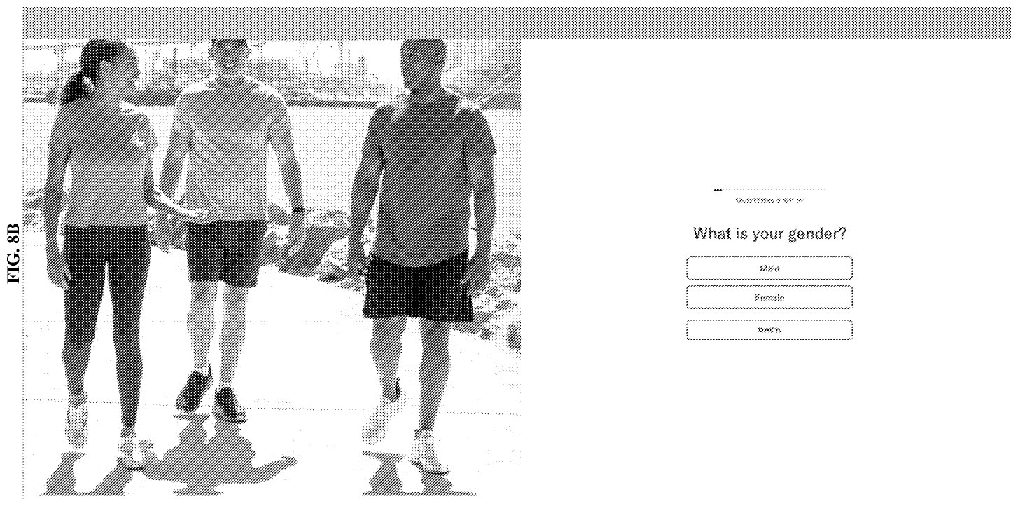

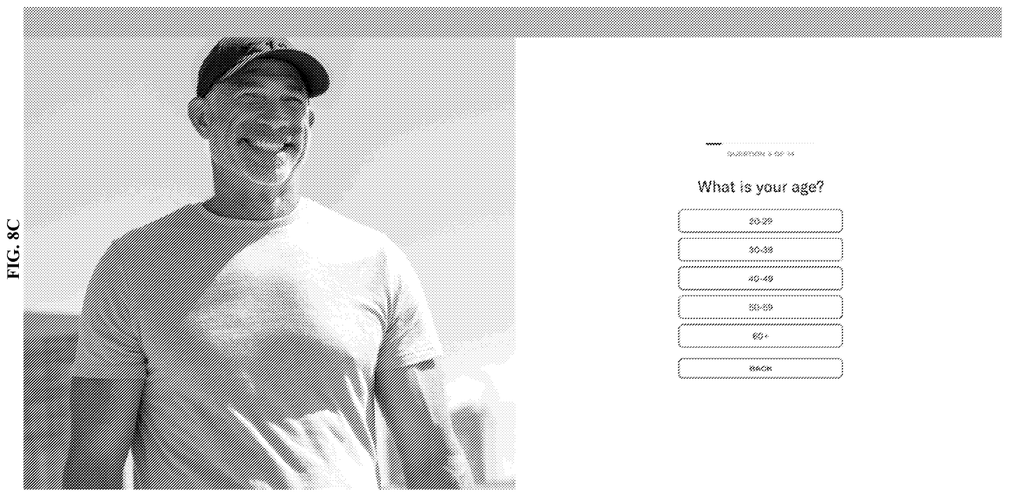

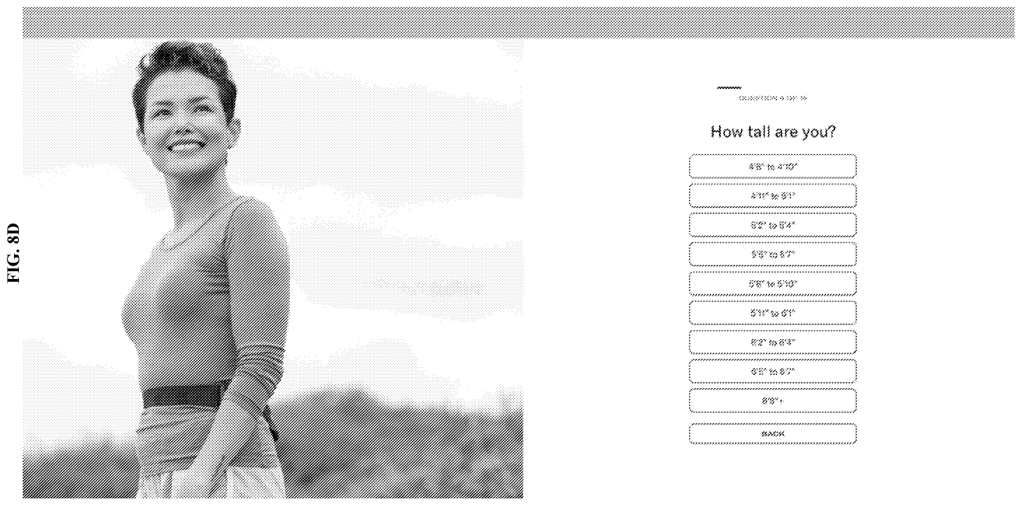

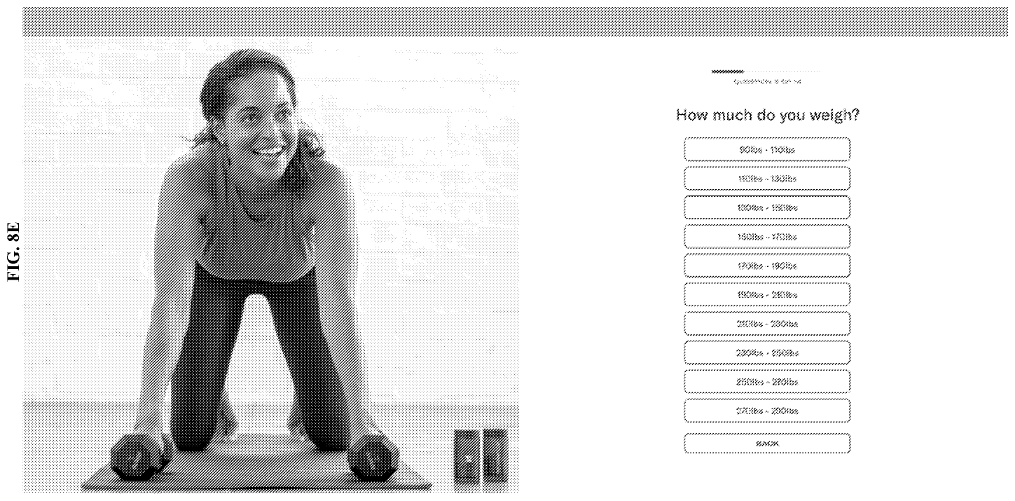

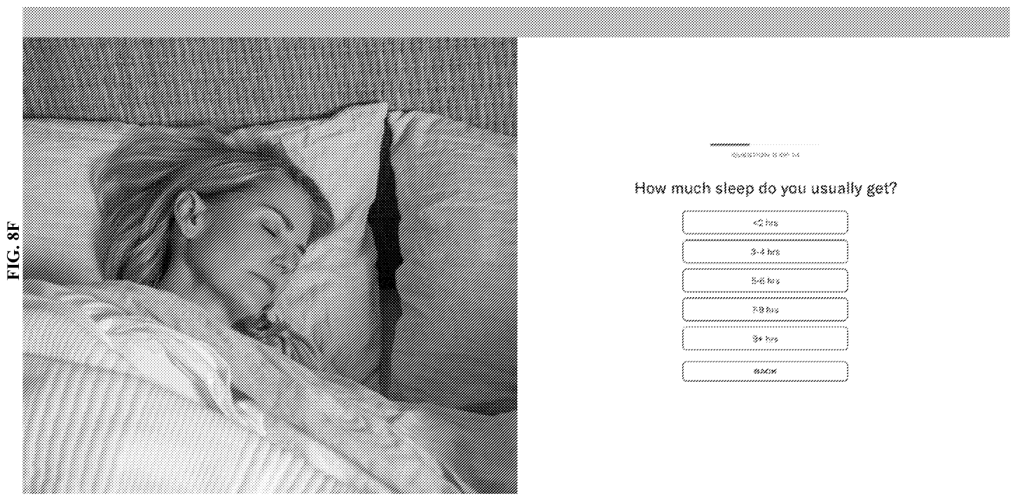

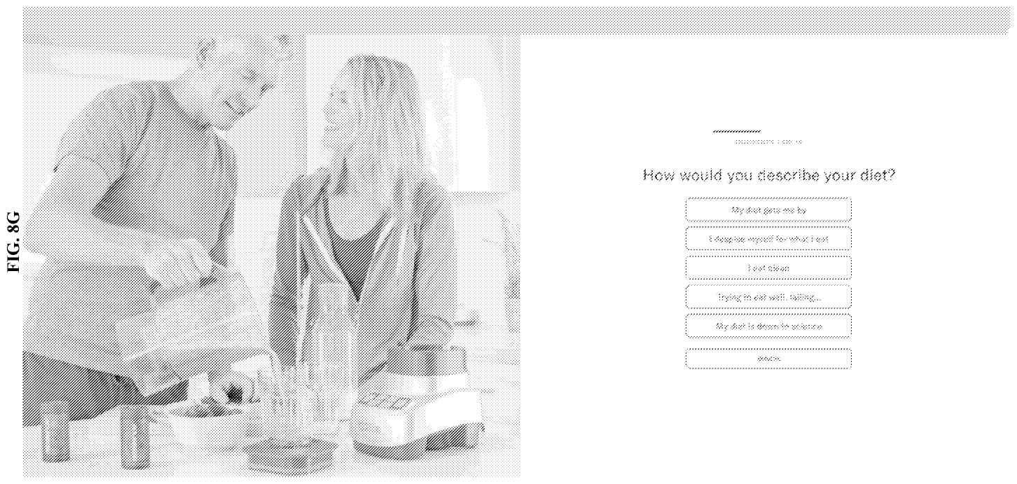

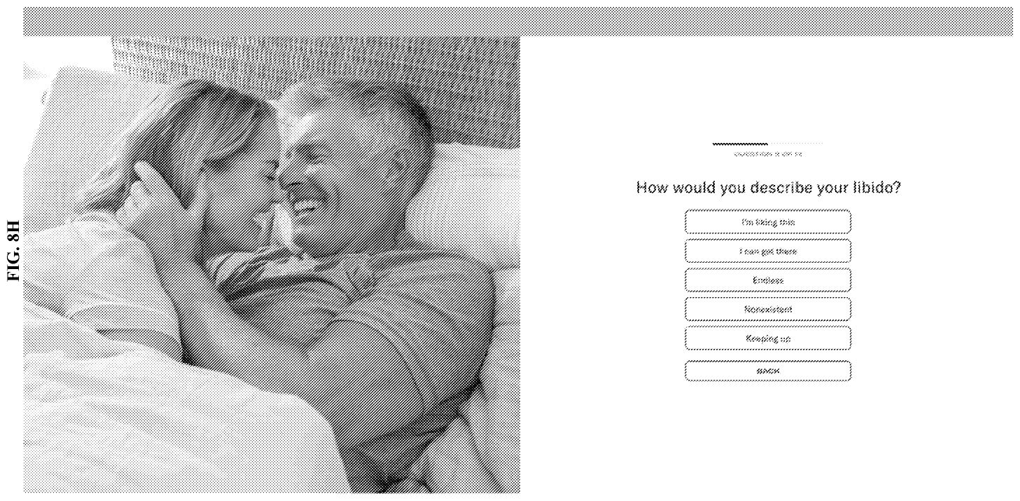

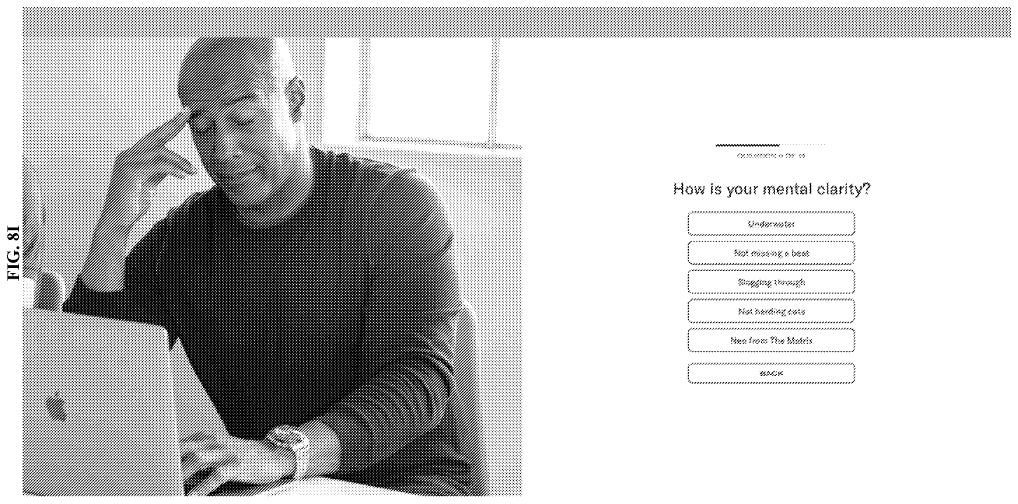

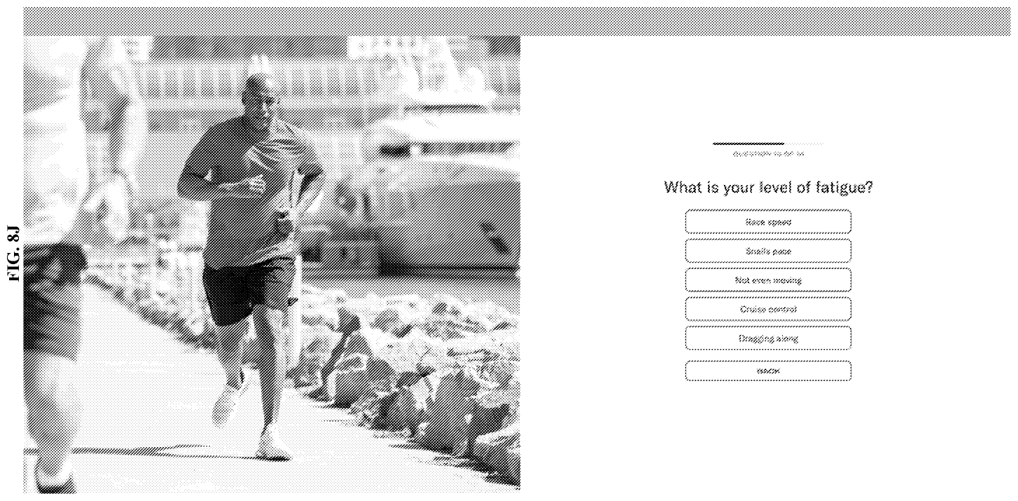

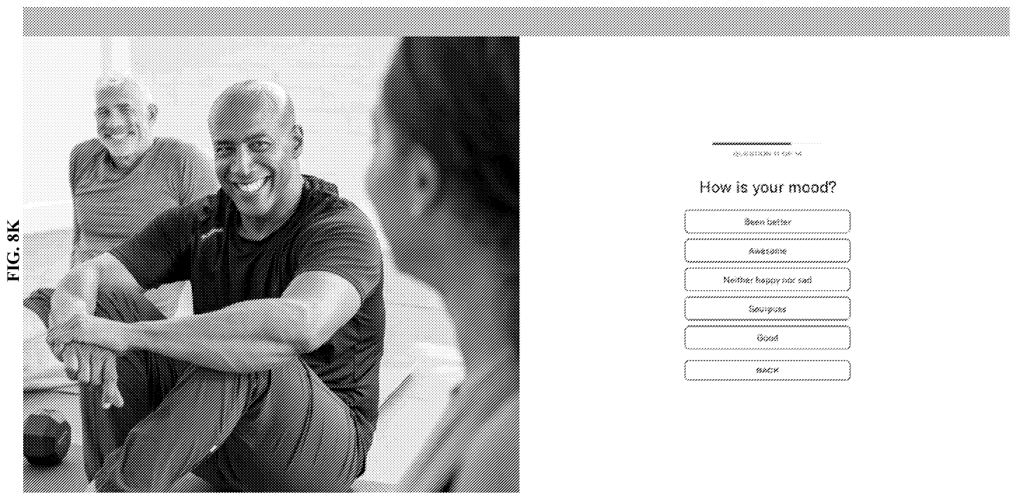

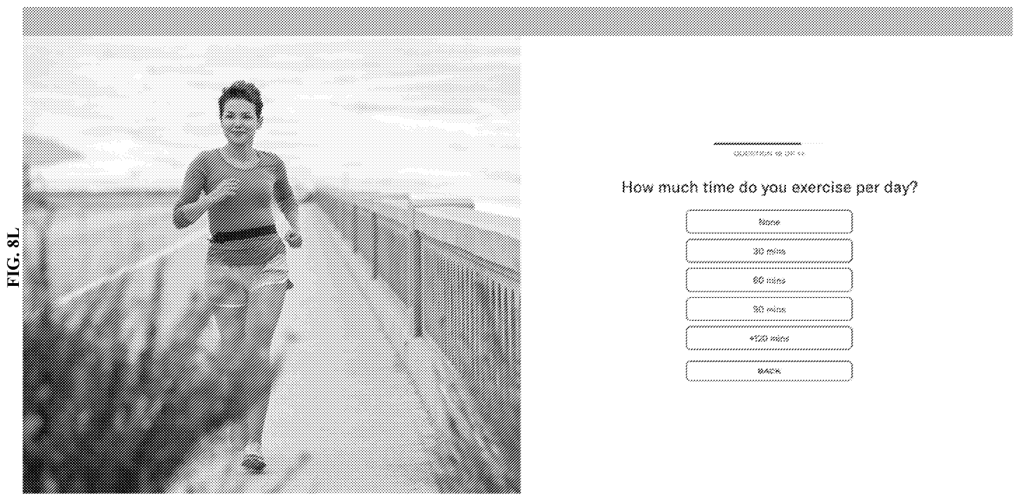

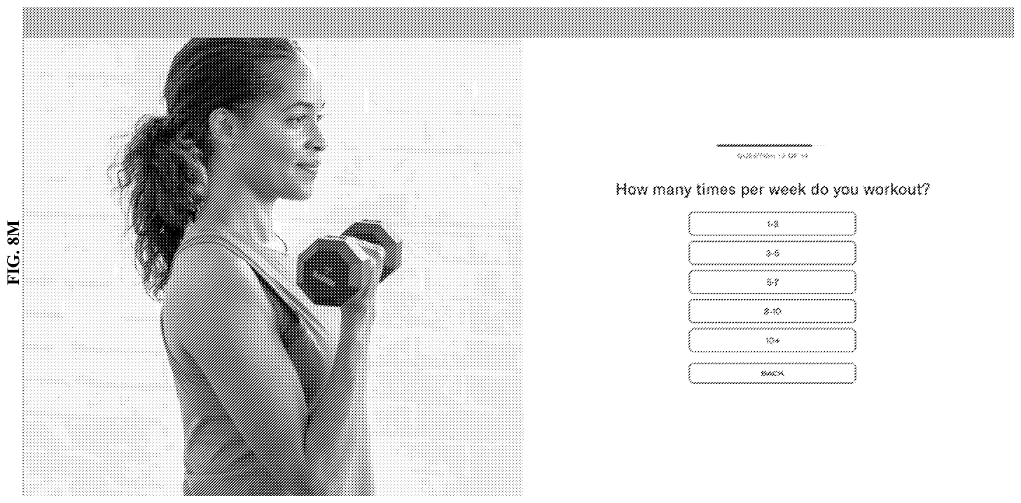

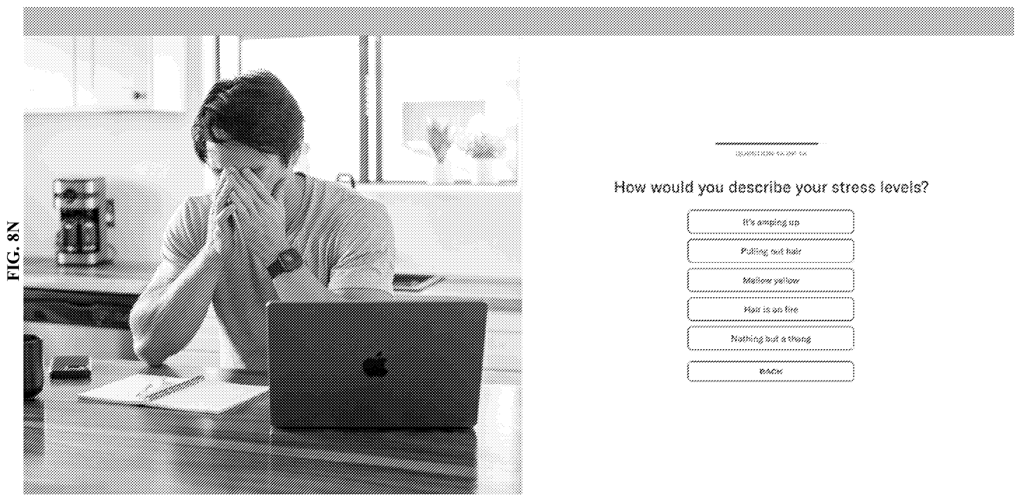

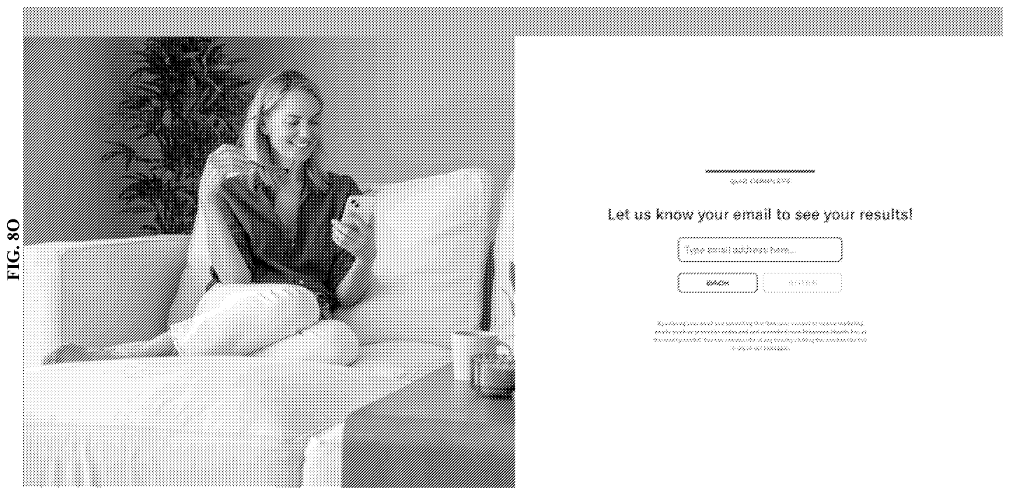

Another section of the one or more section can be weight. This section can ask the user for weight related information and data to determine a modified BMI index. The algorithm can receive the user data in response to health-related questions presented to the user, such as the examples illustrated in FIGS. 8B-8N, to determine a user's BMI index. The algorithm can be configured to determine a modified BMI index based off the user's BMI index, to be a more effective indicator of a user's health compared to a traditional BMI index. The BMI index may be modified in response to a user's bone and muscle mass weight variation or any other factor that can affect a BMI index. A score may then be determined based off a modified BMI index and can be applied to a weighted calculation and incorporated into the overall scoring with “normal” receiving a higher score and “obese” receiving a lower score. It can be determined that weight has a smaller influence on a user's overall health and wellbeing and/or cortisol level and therefore be weighted less than a score corresponding to the sleep related answers and inputs. In some embodiments, the algorithm can offer a multiple-choice format with an unlimited collection of answers offered up in a randomly generated display order to encourage the user to answer as authentically as possible and without the option to game the system. The algorithm can show multiple-choice answers in a random order for one or more section having a score corresponding to each of the answers of the one or more section. The one or more section and can each be given a corresponding weight to determine of the overall score. The determined weight of each one or more section corresponding directly to the determined influence on the user' health and wellbeing. An example being a sleep section score may be determined to be a factor that has a large influence on a user's overall health and wellbeing, because of its relationship to hormone production, as explained above, and receive a weight between 25-70% of the overall score; wherein a weight section score is determined to have less influence, and receive a weight between about 1-15% of the overall score; wherein a stress section score can be determined to have more influence than a weight section score but less influence than a sleep section score and receive a weight between about 5-25% of the overall score, wherein one or more additional section are presented answered, used and weighted to determine the overall score. In a non-limiting example, the score can directly correspond to a user's cortisol level, wherein the weight of each section is determined based on the sections determined influence on a user's cortisol level.

In some embodiments, one or more factors may be determined based on information about the person received by the system (e.g., received by the processing device). In some embodiments, the user may input the information to a user interface. In some embodiments, a person provides a level of physiological feature, such as, without limitation, cortisol, biotin (vitamin B7), ketones, vitamin C, vitamin B9, creatinine, magnesium, water, vitamin B12, calcium, or sodium, or combinations of two or more thereof, to the system. In some embodiments, a computing device inputs a level of a physiological feature, such as, without limitation, cortisol, biotin (vitamin B7), ketones, vitamin C, vitamin B9, creatinine, magnesium, water, vitamin B12, calcium, or sodium, or combinations of two or more thereof, to the system. Non-limiting examples of levels includes levels of a physiological feature of the person measured in the morning, levels of a physiological feature of the person measured at midday, levels of a physiological feature of the person measured in the evening, and levels of a physiological feature of the person measured at night. Non-limiting example a physiological features include cortisol. While levels measured in the morning, at midday, in the evening, and at night are described, these are exemplary times and levels of a physiological feature measured at other times may be used. In some embodiments, levels of a physiological feature are measured 2, 3, 4, 5, or 6 times a day, and two or more of these levels are averaged and provided to the interface.

In a non-limiting example embodiment, the system is used to determine whether a person should take an example product called RH1. In a non-limiting example embodiment, the system is used to determine an effect of taking an example product called RH1. In a non-limiting example embodiment, the system is used to determine whether a person should take an example product called RH2. In a non-limiting example embodiment, the system is used to determine an effect of taking an example product called RH2. In a non-limiting example embodiment, the system used to determine whether a person should take an example product called RH3. In a non-limiting example embodiment, the system is used to determine an effect of taking an example product called RH3.

In one aspect, a system for providing recommendations to a person based on information determined about a person's health data is provided. In some embodiments, the system for providing recommendations to a person based on information determined about a person's health may be a part of the system previously disclosed herein.

In some embodiments, the system for providing recommendations may receive information (e.g., indicators of various levels of one or more substances in a person's blood or urine) about a person and/or one or more samples (e.g., a urine sample or a blood sample) associated with a person. If one or more samples are received, the system for providing recommendations may analyze the one or more samples in order to determine information about the person (e.g., the various levels of one or more substances in the person's blood or urine or the pH of the person's blood or urine).

The system may then analyze the information about the person in order to determine a recommendation. The recommendation may indicate one or more changes that the person should make (e.g., a change in the person's daily habits, a change in the person's diet, a change in the person's sleep habits, and/or a change in the amount of a product the person is taking). The recommendation may also indicate that one or more various levels of one or more substances in a person's body is not a recommended level (e.g., at a higher level or a lower level than recommended). The one or more substances may include biotin (vitamin B7), ketones, vitamin C, vitamin B9, creatinine, cortisol, magnesium, water, vitamin B12, calcium, and/or sodium.

The system may then determine the recommendation based on one or more levels of the one or more substances. In some embodiments, the recommendation may indicate that one or more levels of the one or more substances is higher or lower than a recommended level, that the person should adjust one or more aspect of the person's lifestyle for various purposes (e.g., to change the one or more levels of the one or more substances into a recommended range), and/or that the person should take or stop taking one or more products (e.g., RH1, RH2, and/or RH3).

The recommendation may indicate that the person should take one or more products. For example, the recommendation may indicate that the person should take a first product (e.g., RH1), a second product (e.g., RH2), and a third product (e.g., RH3). In some aspects, the compositions of RH1, RH2, and/or RH3 may cause the levels of the one or more substances to rise or fall towards a recommended range.

Thus, after receiving information about a person and/or samples from a person, the system may determine a recommendation based on the information and/or samples. The system may then provide the recommendation to the person (e.g., to a computing device associated with the person). A non-limiting example of a sample is urine, e.g., information from the urine may related to an amount of a metabolite in the urine sample from the person.

In some embodiments, the person may provide multiple sets of health data. Further, in those embodiments, the person may provide each set of the multiple sets of health data at different times throughout a day. The different times throughout the day may be immediately after waking up, four to six hours after waking up, four to six hours before going to sleep, or just before going to sleep. While the times of after waking up, four to six hours after waking up, four to six hours before going to sleep, or just before going to sleep are described, these times are exemplary and other times may be used. In those embodiments, the multiple sets of health data may be manipulated (e.g. averaged, grouped, and or transformed) in order to determine an optimized data set that is then used to determine the recommendation.

### Additional System Embodiments

In general, assessing an individual's health and providing a recommendation regarding their health is not a one-size-fits-all process. Many aspects surrounding the individual's health may constantly change, making it difficult to determine if those aspects are associated with a healthy lifestyle. Additionally, there can be an innumerable number of aspects contribute to the individual's health quality, and thus, can make it nearly impossible to determine what aspects of the individual's lifestyle lead to the quality of their health, whether good or bad.

In particular, a person's cortisol level is difficult to determine and effectively track. Current methods for determining a person's cortisol levels include taking a blood sample and testing the person's blood; however, this is invasive and unreliable as it only provides a snapshot of the patient's cortisol levels at that specific moment in time and is unable to take into account the innumerable factors and variables that effect person's cortisol levels at any particular moment, e.g., stressful experiences that day or foods eaten prior to the test. Therefore, identifying outlier sample data and interpreting the results can be challenging. Due to the invasive and time-consuming nature of these tests, it is difficult and uncomfortable to preform multiple test over a single day or week, making it hard to track a person's cortisol over a period of time. Another method involves testing urine samples to determine cortisol levels of a person. While this may allow for more effective tracking of cortisol levels over a time period (e.g., a day or a week), the user is only able to test at times when the person needs to urinate. Further, this method can be uncomfortable and awkward if a person is required to carry around sample bottles throughout the day and collect and store samples while at work or in public. This method can also lead to inaccurate representation of a person's normal cortisol levels as the person may be pressured to drink more water to collect more samples over a time period, affecting the hydration levels of the person and potentially skewing the cortisol data. Thus, a need exists in the art for assessing an individual's health, including methods and systems for an accurate and/or broadly representative measurement of an individual's cortisol level and using aspects associated with the individual's health to determine what aspects of the individual's health are beneficial or detrimental, along with providing recommendations on how to change (or not change) one's lifestyle in relation to those aspects.

Systems and methods described herein provide for assessing the health or wellbeing of a person based on information associated with or derived from the person. As described above, innumerable aspects may surround an individual's health; however, the systems and methods described herein provide for determining a score to provide an accurate assessment of the individual's health as well as recommendations to improve it based on one or more factors surrounding those aspects. The systems and methods provide for combining both subjective and objective information associated with the person in order to account for both physical and nonphysical aspects of the individual's health. Additionally, the subjective and objective information may indicate one or more deficiencies of the individual's health, such as high cortisol levels, and thus, the system and methods include feedback, such as recommending that the individual make a lifestyle, diet, and/or nutrient adjustment to combat those deficiencies. As a non-limiting example, the recommendation is to take a nutrient composition comprising a modulator of cortisol. The recommendation may be in response to determining a higher than desired cortisol level. In a non-limiting embodiment, the composition comprises ashwagandha extract.

In some embodiments, the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

In some embodiments, the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Information

Systems and methods as described herein may take as input information about a person. The information may comprise data associated with one or more people (e.g., a user). The information may be provided by the user. In some cases, information may be stored in one or more databases. The information stored in the one or more databases may comprise information associated with one or more additional persons other than the user. Alternatively, or additionally, the information stored in the one or more databases may comprise information associated with the user. The stored information may include past scores and/or recommendations.

The information (e.g., data) may be indicative of one or more factors associated with the person. The one or more factors may be determined based on the information provided by the person. Alternatively, or additionally, the one or more factors may include one or more relevant pieces of the provided information. In an example, if the received information includes the person's date of birth, one of the one or more factors may be the date of birth itself, and one of the one or more factors may be the age range determined based on the date of birth.

As described above, in some cases, information about the person may be input. The information may be received from the person or from a computing device associated with the person. Non-limiting examples of provided information includes the age and/or date of birth of the person, sleep data of the person, diet of the person, libido level of the person, mental clarity of the person, fatigue levels of the person, mood of the person, daily exercise of the person, level of energy of the person during exercise, daily stress levels of the person, measured cortisol levels of the person in the morning, measured cortisol levels of the person at midday, measured cortisol levels of the person in the evening, and measured cortisol levels of the person at night.

In some cases, information about the person or factors thereof may be associated with objective information, such as the age and/or date of birth of the person, sleep data of the person, diet of the person, and measured levels of a substance in a sample associated with the person (e.g., measured cortisol levels of the person in the morning, measured cortisol levels of the person at midday, measured cortisol levels of the person in the evening, and measured cortisol levels of the person at night). In some cases, measured levels may be averaged from one or more measurements taken throughout the day, e.g., taken 2, 3, 4, 5, or 6 times a day, such as 4 times a day.

In some cases, information about the person or factors thereof may be associated with the age of the person, such as an age range that the person belongs to. For example, information specifying the date of birth or the age of the person may be received. Relevant age ranges may be determined, and the recommendation may be based on those age ranges, such as age ranges of between 18 and 29 years, between 30 and 39 years, between 40 and 49 years, between 50 and 59 years, or between 60 and 70 years. While certain age ranges are described, these ranges are exemplary, and different ranges may be used.

In some cases, information about the person or factors thereof may be associated with the daily sleep of the person. For example, information specifying that the person sleeps for a specified average amount of time each night, and that specified average amount of time each night may fall within a specified range may be received. Specified ranges may include less than about 4 hours, from about 4 hours to about 5 hours, from about 5 hours to about 6 hours, from about 6 hours to about 7 hours, from about 7 hours to about 8 hours, from about 8 hours to about 9 hours, from about 9 hours to about 10 hours, or more than about 10 hours. While certain ranges of sleep are described, these ranges are exemplary, and different ranges may be used.

In some cases, information about the person or factors thereof may be associated with the daily exercise of the person. For example, information specifying that the person exercises for a specified average amount of time each day or night, and that specified average amount of time exercised may fall within a specified range may be received. Specified ranges may include no daily exercise, about 30 minutes of daily exercise, about 1 hour of daily exercise, about 1.5 hours of daily exercise, or about 2 hours or more of daily exercise. While certain ranges of daily exercise are described, these ranges are exemplary, and different ranges may be used. Additionally, the person may input information specifying that the person did a specific exercise, such as running, lifting weights, swimming, boxing, soccer, or other various exercises, on each day. While certain exercises are described, these exercises are exemplary, and different exercises may be performed and input.

In some cases, information about the person or factors thereof may be associated with the diet of the person. For example, the person may input information specifying the number of meals per day the person eats, such as two or three meals a day. As another example, the person may input an number of calories that the person intakes per day, such as 2000 calories per day or 3000 calories per day. As yet another example, the person may input the type of food eaten each day, such as eggs or spinach. While certain amounts of meals, amounts of calories, and types of food are described, these are exemplary, and other amounts or types may be used. In some embodiments, the received information may indicate that the person considers his or her diet to be considerably unhealthy, unhealthy, average, healthy, or considerably healthy. While only certain health levels attributed to a person's diet are described, these levels are exemplary, and other levels or methods of describing a person's diet may be used.

In some cases, information about the person or factors thereof may be associated with data indicative of a level of one or more substances in a sample associated with the person. In some cases, the sample is a bodily fluid associated with the person. In some cases, the bodily fluid may be blood, saliva, or urine. The one or more substances may include biotin (vitamin B7), ketones, vitamin C, vitamin B9, creatinine, cortisol, magnesium, water, vitamin B12, calcium, and/or sodium. The data indicating levels of one or more substances in the sample may be from one or more timepoints. In some cases, the data are taken at one time point. Alternatively, the data may be taken at 2, 3, 4, 5, 6, 7, 8, 9, 10, 11, 12, 13, 14, 15, 16, 17, 18, 19, 20, or more timepoints. The timepoints may be spread over any appropriate length of time. In some cases, the timepoints occur within one day. In some cases, the timepoints occur within one week. In some cases, the timepoints occur within one month. In some cases, the timepoints occur within one year or longer.

In some cases, the input may comprise the sample itself or a fraction thereof. The sample may be processed (e.g., assayed, analyzed, separated, isolated, stored, or the like) before or after being input into a system as described herein. In such cases, the methods and systems described herein may be configured to determine the level or one or more substances in the (processed) sample or portion thereof.

In an example, cortisol level data associated with the person is provided. Non-limiting examples of cortisol levels include cortisol levels of the person measured in the morning, cortisol levels of the person measured at midday, cortisol levels of the person measured in the evening, and cortisol levels of the person measured at night. While cortisol levels measured in the morning, at midday, in the evening, and at night are described, these are exemplary times and cortisol levels measured at other times may be used. In some cases, cortisol levels may be averaged from one or more measurements taken throughout the day, e.g., taken 2, 3, 4, 5, or 6 times a day, such as 4 times a day.

In some cases, information about the person or factors thereof may be associated with subjective information (e.g., information based on the person's feeling or belief) provided, such as libido level of the person, mental clarity of the person, fatigue levels of the person, mood of the person, level of energy of the person during exercise, and daily stress levels of the person.

For example, information about the person or factors thereof may indicate the overall libido of the person. For example, the provided information may indicate that the person feels or believes his or her libido is no libido, libido less than the average man or woman, libido of the average man or woman, more libido than the average man or woman, or substantially more libido than the average man or woman. While certain levels of libido are described, these are exemplary, and other information about libido may be used.

As another example, the information about the person or factors thereof may indicate the daily mental clarity of the person. For example, the provided information may indicate that the person feels or believes his or her mental clarity is no mental clarity, mental clarity less than the average man or woman, mental clarity of the average man or woman, more mental clarity than the average man or woman, or substantially more mental clarity than the average man or woman. While certain levels of mental clarity are described, these are exemplary, and other information about mental clarity may be used.

As yet another example information about the person or factors thereof may indicate the daily fatigue level of the person. For example, the provided information may indicate that the person feels or believes his or her fatigue level is no fatigue, about a fatigue level less than the average man or woman, about a fatigue level equal to the average man or woman, about a fatigue level greater than the average man or woman, or about a fatigue level substantially greater than the average man or woman. While certain fatigue levels are described, these are exemplary, and other information about a person's fatigue may be used.

As even another example, information about the person or factors thereof may indicate the daily mood of the person. For example, the provided information may indicate that the person feels or believes his or her mood is a mood substantially worse than the average man or woman, a mood worse than the average man or woman, a mood the same as the average man or woman, a mood better than the average man or woman, or a mood substantially better than the average man or woman. While certain moods are described, these are exemplary, and other information about a person's fatigue may be used.

As yet another example, information about the person or factors thereof may indicate the daily stress level of the person. For example, the received information may indicate that the person feels or believes his or her daily stress level is no daily stress, about a daily stress level less than the average man or woman, about a daily stress level equal to the average man or woman, about a daily stress level greater than the average man or woman, or about a daily stress level substantially greater than the average man or woman. While certain stress levels are described, these are exemplary, and other information about a person's stress level may be used.

As one more example, information about the person or factors thereof may indicate the energy levels of the person during workouts. For example, the received information may indicate that the person feels or believes his or her energy level is substantially less than the average man or woman, about an energy level less than the average man or woman, about an energy level about equal to the average man or woman, about an energy level greater than the average man or woman, or about an energy level substantially greater than the average man or woman. While certain energy levels are described, these are exemplary, and other information about a person's energy level may be used.

### Systems for Monitoring and Providing Feedback Based on Person's Health

An aspect of the present disclosure provides a system for monitoring the health of a user by receiving and processing information associated with the person. The system may accept input regarding the user, process or analyze the input, and produce output indicative of a health state of the user. Alternatively, or additionally, the system may provide feedback, such as an indication of cortisol level and/or one or more recommendations to the user based on the processed input. In a non-limiting embodiment, the system or method comprises using an algorithm to collect, process, and analyze the user information and data and generate and produce one or more outputs. The outputs may include but are not limited to one or more scores and/or one or more recommendations. In an embodiment, the algorithm may be a machine learning algorithm or the like.

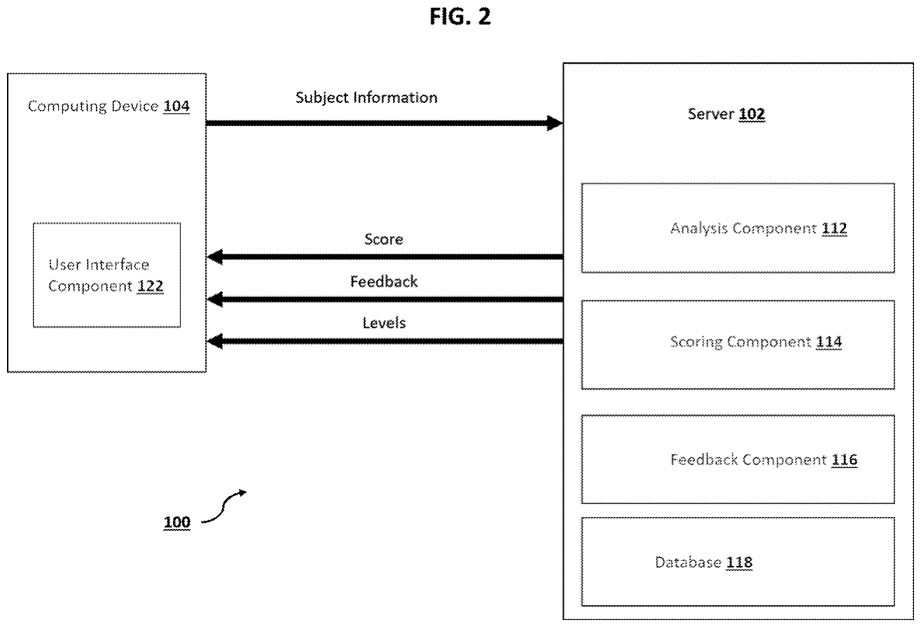

The system may comprise one or more computing devices (e.g., computing device 104 of FIG. 2) associated with the user. The one or more computing devices may comprise a user interface component. The user interface component may accept input from the user and based on the input generate an output indicative of the health or wellbeing of the user. Input may be received from and output displayed to the user on the user interface component.

The system may comprise a server (e.g., server 102 of FIG. 2). The server may receive the input, process the input, provide the output, or any combination thereof. The server may comprise one or more components or modules. The one or more components or modules may comprise an analysis component (e.g., analysis component 112 of FIG. 2). The analysis component may perform a processing, analysis, or comparison of the input. The one or more components or modules may comprise a scoring component (e.g., scoring component of FIG. 2). The scoring component may generate a score indicative of the health or wellbeing of the person based on the input or processed input. The score may be indicative of the person's holistic health or wellbeing, or the score may be indicated of one aspect or factor of the person's health or wellbeing. In a non-limiting example, the score may be indicative of specifically a person's cortisol level. The score may be a numerical representation of the person's health or wellbeing. The score may be a numerical representation of the person's holistic health or wellbeing, or the score may be indicative of one aspect or factor of the person's health or wellbeing. In a non-liming example, the score may be indicative of specifically a person's cortisol level. The one or more components or modules may comprise a feedback component (e.g., feedback component 116 of FIG. 2). The feedback component may generate feedback based on the input or processed input. The feedback may comprise a recommendation to the user (e.g., to take a composition as described elsewhere herein, to make a lifestyle change). The feedback may comprise a visual indication to the user. In some cases, the visual feedback may comprise a color change, a particular text output, a numerical output, or any combination thereof. The one or more components or modules may comprise a database (e.g., database 118 of FIG. 2). The database may store data about the user or one or more additional persons. In some cases, the analysis unit may process, analyze, or compare the input or processed input based on data contained in the database. In some cases, the scoring component or feedback component may generate the score or feedback, respectively, based on data contained in the database. The score or feedback may be based on a comparison.

The system may provide an output comprising a score. The output or score may be feedback. The score may summarize the state of the person's health or wellbeing. Alternatively, or additionally, the score may characterize a specific factor of aspect of the person's health or wellbeing. In a non-limiting example, the score may characterize specifically a person's cortisol level. The score may be numerical and may correspond to a discrete scale (e.g., whole numbers from 0 to 10 or from 0 to 100) or a continuous scale (e.g., any number from 0 to 10 or 0 to 100). Alternatively, or additionally, the output may provide feedback. The feedback may be a visual indication to the user. In some cases, the visual feedback may comprise a color change, a particular text output, a numerical output, or any combination thereof. The feedback may comprise one or more text-based indicators corresponding to a category (e.g., low, medium, high; or bad, poor, fair, good, excellent) characterizing the health or wellbeing of the person.

In some cases, for a 0-100 scale, a score between 0-10, 0-20, 0-30, 0-40, 0-50, 0-60, 0-70, or 0-80 may indicate a high cortisol level; a score between 0-90, 10-90, 20-90, 30-90, 40-90, 50-90, 60-90, 70-90 or 80-90, may indicate a medium cortisol level; a score between 50-100, 60-100, 70-100, 80-100, 90-100 may indicate a low cortisol level.

In some cases, for a 0-100 scale, a score between 0-10, 0-20, 0-30, 0-40, 0-50, 0-60, 0-70, or 0-80 may indicate a bad or poor cortisol level; a score between 0-90, 10-90, 20-90, 30-90,40-90, 50-90,60-90, 70-90 or 80-90, may indicate a fair or good cortisol level; a score between 50-100, 60-100, 70-100, 80-100, 90-100 may indicate an excellent cortisol level.

The feedback may comprise one or more recommendations to the person. The one or more recommendations may comprise a lifestyle, diet, and/or nutrient adjustment. The one or more recommendations may comprise a lifestyle recommendation. The lifestyle recommendations may comprise one or more changes to the person's exercise or sleep behavior. The one or more recommendations may comprise a diet adjustment, such as recommending a person intake more vegetables, protein, or the like. The one or more recommendations may comprise a nutrient adjustment. As a non-limiting example, the recommendation is to take a vitamin, mineral, or nutraceutical. The nutraceutical may modulate cortisol levels. The nutraceutical may comprise ashwagandha extract.

In some cases, (e.g., those in which a sample is input into the system) the feedback may comprise one or more levels of a substance in the sample associated with the person. For example, cortisol, biotin (vitamin B7), ketones, vitamin C, vitamin B9, creatinine, magnesium, water, vitamin B12, calcium, and/or sodium.

FIG. 2 illustrates an example system 100 for monitoring the health of a user in accordance with the disclosure. The system 100 may comprise a server 102 in communication with a computing device 104. The server 102 may comprise one or more modules or components illustrated in FIG. 2 (e.g., analysis component 112, scoring component 114, feedback component 116, and database 118).

The computing device 104 may comprise a user interface component 122. The user interface component 122 may comprise a graphical user interface (GUI), a web-based user interface, a command line interface, a menu-driven interface, a touchscreen GUI, or any combination thereof. Through the user interface component 122, a user may input information about a person and receive output based on the input.

In this depicted embodiment, the server 102 comprises an analysis component 112. The analysis component 112 may process or analyze one or more pieces of information. The analysis component may identify and remove outliers in the input information, compare the input or processed input to goal information, analyze the input information compared to goal information, analyze the input information (e.g., a sample) for the level of one or more substances in the sample, or any combination thereof. In cases where a database 118 is present, the analysis component 114 analyze the information based on data in the database 108.

In some embodiments, the analysis component 112 may exclude one or more outlier aspects. A user may provide one or more datasets from multiple points throughout the day. For example, a user may provide a urine or blood sample, or information regarding a urine or blood sample, at four times throughout the day. The analysis component 112 may determine information directly from the urine or blood samples or may receive the information regarding the urine or blood samples. The analysis component 112 may then further recognize one or more outlier aspects regarding each dataset by determining what aspects are primarily affected by the time that the sample was taken, and thus, are unreliable. For example, the analysis component 112 may receive four datasets, concerning samples taken right when the user wakes up, four hours after the user wakes up, four hours before the user goes to sleep, and right before the user goes to sleep. The information may show that the hydration levels of the user are extremely low after the user wakes up, but are at normal levels for the rest of the datasets. The analysis component 112 may recognize that the hydration level in the dataset collected right after the user wakes up are extremely low because the user has not been able to drink for the past eight hours (e.g., because the user was asleep), and may label the data point as an outlier. Thus, when the analysis component sends data the scoring component 114 and feedback component 116, it may not send the outlier data points.

In this depicted embodiment, the server 102 comprises a scoring component 114. The scoring component 114 may process information to generate a score indicative of the health state or wellbeing of the person. In some cases, the scoring component 114 may operate on information that has been processed or analyzed by the analysis component 112. Alternatively, or additionally, the scoring component 114 may operate on the raw input information. In cases where a database 118 is present, the scoring component 114 may produce a score based on the data in the database 118.

In this depicted embodiment, the server 102 comprises a feedback component 116. The feedback component may generate one or more outputs sent to the user interface component 122 to provide feedback to the user based on the information. The feedback may comprise a recommendation to the user. The feedback may comprise one or more recommendations, such as a lifestyle, diet, and/or nutrient adjustment. The lifestyle recommendations may comprise one or more changes to the person's exercise or sleep behavior. The one or more recommendations may comprise a diet adjustment, such as recommending a person intake more vegetables, protein, or the like. The one or more recommendations may comprise a nutrient adjustment. As a non-limiting example, the recommendation is to take a vitamin, mineral, or nutraceutical. The nutraceutical may modulate cortisol levels. The nutraceutical may comprise ashwagandha extract.

In this depicted embodiment, the server 102 comprises a database 118. The database 118 may contain information from one or more users. The database 118 may contain information not associated with a particular person, such as goal values for aspects or factors associated with a user. For example, a goal value for aspects or factors may be a goal time for exercise each day or a goal cortisol level. In some cases, the database 118 may contain data used by the analysis component 112, the scoring component 114, or the feedback component 116. In an example, the analysis component 112 may use information stored in the database to identify one or more outlier values in the input information and remove the outlier(s) from the input. In another example, the scoring component 114 may compare the input information or processed information from the analysis component 112 to other information in the database to determine a score for the user. In some cases, the database 118 may contain goal information, such as the goal values described above. The goal information may be used by the score component to generate a score indicative of the health state of the user. The score may be generated by comparing the goal information to the information. The goal information may be used by the feedback component to generate feedback based on the health state of the user. The feedback may be generated by comparing the goal information to the information.

The score may indicate one or more recommendations. The recommendations may include lifestyle changes, dietary changes, and/or nutrient adjustments, such as exercising more, drinking more water, or eating less unhealthy foods. The recommendations may also include a recommendation to take a product. In some embodiments, the product contains ashwagandha product. In some embodiments, the product also contains a chelating agent (e.g., EDTA) and a delivery enhancing agent (AstraGin®). The product may also contain various ingredients, such as MACA, Eurycoma longifolia, a fenugreek extract source (e.g., Testosurge®), Tribulus terrestris, cordyceps mushroom (e.g., Cordyceps Prime™), one or more vitamins (e.g., vitamin B6), a nitric oxide source (e.g., beet root powder), a green tea extract, one or more amino acids (e.g., D-Aspartic Acid, L-theanine, and/or L-tryptophan), a Panax ginseng source, sodium starch glycolate, colloidal silicon dioxide, microcrystalline dioxide, a sweetening agent (e.g., sucralose), one or more flavoring agents (e.g., peppermint flavor spray dried P-183, natural peppermint flavor, menthol crystals, or another flavoring agent), a starch excipient (e.g., starcap 1500), magnesium stearate, bacopa, phosphatidylserine, magnesium glycinate, Magnolia officinalis, ginger, horny goat weed, sclareolides (e.g., sage powder), DIM, one or more minerals (e.g., zinc), an oat straw source, reishi mushroom, and/or melatonin.

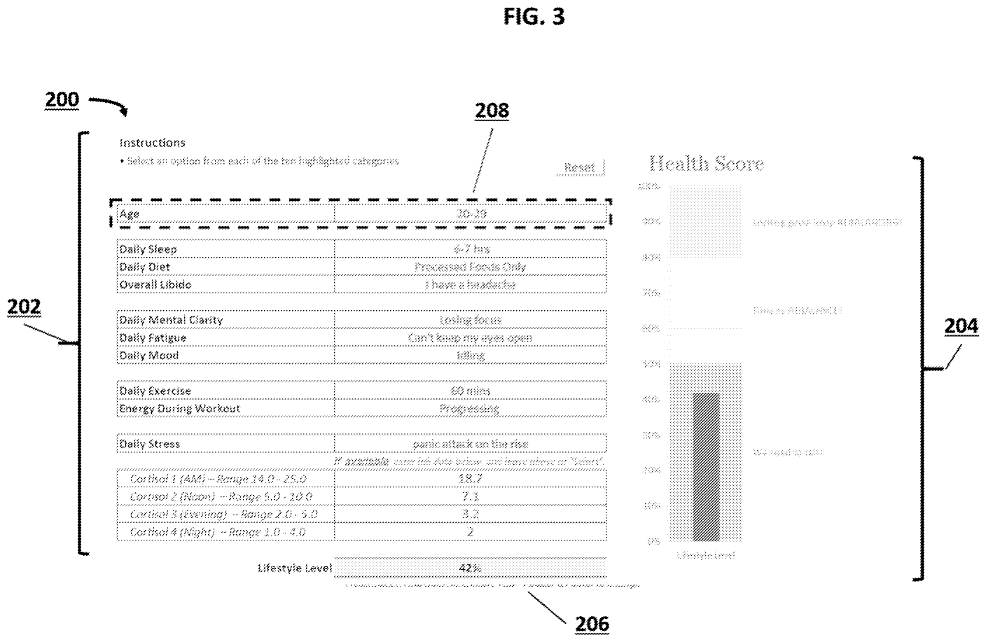

FIG. 3 illustrates an example user interface 200. The user interface 200 comprises an input pane 202 with an input row 208, feedback output pane 204, and score output pane 206. The input pane 200 may prompt the user to provide information related to the age of the person, sleep data of the person, diet of the person, libido level of the person, mental clarity of the person, fatigue level of the person, mood of the person, exercise level of the person, level of energy during exercise of the person, daily stress levels of the person, or levels of one or more substances (e.g., cortisol) measured (e.g., at one or more timepoints) in a sample associated with the person.

The input pane 200 may comprise one or more input rows 208. Input row 208 may receive input regarding the user. In some embodiments, the input regarding the user may be associated with the user's health. The input row 208 may be directed to particular pieces of information about the person as discussed above. Additionally, in some embodiments, any number of input rows may be used. The input pane 200 may prompt the user to select form a predetermined list for each piece of data requested. Alternatively, or additionally, the input pane 200 may prompt the user to supply the data as a free entry, e.g., a numerical value.

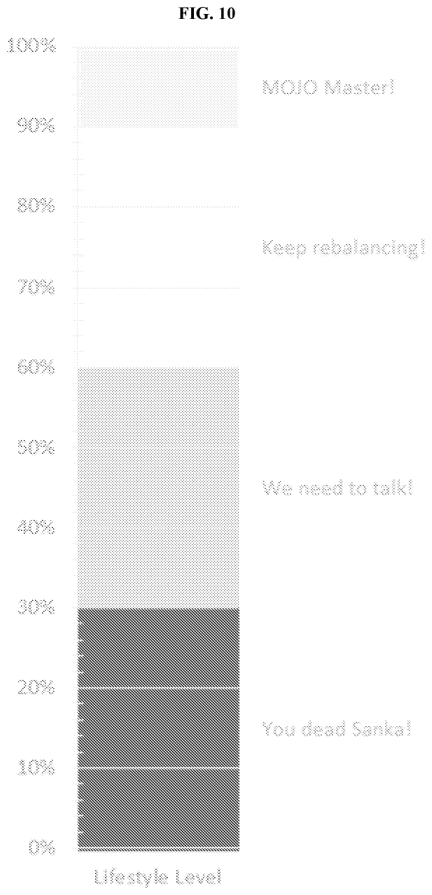

The user interface 200 may comprise a feedback output pane 204. The feedback output pane may display to the user feedback based on the one or more input values. The feedback may comprise visual feedback. The visual feedback may comprise a color or color change. Alternatively, or additionally, the visual feedback may comprise textual feedback which summarizes an assessment of the person's health or wellbeing. FIG. 10 provide one possible example of the described visual feedback for summarizing an assessments of a person's health or well being. The feedback may comprise one or more recommendations, such as a lifestyle, diet, and/or nutrient adjustment. The lifestyle recommendations may comprise one or more changes to the person's exercise or sleep behavior. The one or more recommendations may comprise a diet adjustment, such as recommending a person intake more vegetables, protein, or the like. The one or more recommendations may comprise a nutrient adjustment. As a non-limiting example, the recommendation is to take a vitamin, mineral, or nutraceutical. The nutraceutical may modulate cortisol levels. The nutraceutical may comprise ashwagandha extract. In cases where a sample associated with the user or a portion thereof is provided as input, the feedback output pane may additionally provide output indicative of the level of one or more substances (e.g., metabolite or hormone, such as biotin (vitamin B7), ketones, vitamin C, vitamin B9, creatinine, cortisol, magnesium, water, vitamin B12, calcium, or sodium).

The user interface 200 may comprise a score output pane 206. The score output pane may display to the user a score based on the one or more input values. The score may comprise a numerical result. The score may summarize the state of the person's health or wellbeing. The score may be numerical and may correspond to a discrete scale (e.g., whole numbers from 0 to 10 or from 0 to 100) or a continuous scale (e.g., any number from 0 to 10 or 0 to 100).

### Methods Monitoring and Providing Feedback Based on a Person's Health

An aspect of the present disclosure provides for methods of monitoring the health of a user by receiving and processing information associated with the user. The method may comprise receiving information associated with the user. The method may comprise analyzing or processing the information, such as to remove outlier information. The raw or processed information may then be analyzed. The methods may be implemented by systems as described elsewhere herein.

The method may comprise an operation of accepting input from a user. The user may provide the input through a user interface, such as a user interface associated with a computing device. The information may comprise information related to the age of the person, sleep data of the person, diet of the person, libido level of the person, mental clarity of the person, fatigue level of the person, mood of the person, exercise level of the person, level of energy during exercise of the person, daily stress levels of the person, or levels of one or more substances (e.g., cortisol) measured (e.g., at one or more timepoints) in one or more samples (e.g., a bodily fluid, such as blood, saliva, or urine) associated with the person.

The method may comprise an operation of preprocessing the input. The preprocessing operation may apply one or more transformations to the input data. The preprocessing operation may discard spurious or outlier information. Outlier information may be determined by comparing the input information to one or more references. The references may correspond to information associated with the person or one or more other persons. The references may be stored in a database. The references may correspond to information associated with the person from one or more other timepoints. The preprocessing operation may further standardize the encoding of information depending on the user input or source of the data.

The method may comprise an operation of comparing the input (processed) information to goal information. The goal information may be stored in a database. The goal information may be selected based on the user input such that the goal information is specific to an aspect or factor of the person. For example, the goal information may be selected based on the age, sex, activity level, stress level, diet, level or one or more substances (e.g., cortisol) associated with the person, or any combination thereof.

The method may comprise an operation of generating a score based on the comparison to the goal information. The score may express how close the input information is to the goal information. In some cases, a relatively higher score may mean that the input information is relatively closer to the goal information. Alternatively, in some cases, a lower score may mean that the input information is relatively closer to the goal information. Closeness may be assessed in any suitable way depending on the type of information. For example, in a case wherein the input and corresponding goal information may be expressed numerically, closeness may be determined by determining a difference between the goal and the input information. In another example, when the input and corresponding goal information are categorical data, the categorical data may be converted to a numerical representation and the determination of closeness made based on the corresponding numerical representation.

The operation of generating the score may comprise combining one or more different measures of closeness corresponding to different types of information (e.g., diet, exercise level, cortisol level, etc.) of the same or different modalities (e.g., categorical, numerical). The results of the different comparisons may be combined into one score to be output to the user. The different comparisons may be combined by any suitable averaging operation, such as an arithmetic mean, weighted arithmetic mean, geometric mean, weighted geometric mean, harmonic mean, quadratic mean, generalized mean, or any combination thereof. The result of the averaging operation may be scaled or transformed to make the score lie on a certain interval (e.g., 0 to 1, 0 to 100) or otherwise have a certain property (e.g., rounded or truncated to a whole number).

The method may comprise an operation of generating feedback based on the comparison to the goal information. The feedback may comprise visual feedback summarizing the overall health of the person. Alternatively, the feedback may summarize only one factor (e.g., diet, exercise, cortisol) or subset of factors corresponding to the input information. The feedback may indicate that one or more of the user's behaviors should be maintained. Alternatively, the feedback may indicate the person should adjust one or more changes to their behavior or lifestyle, diet, and/or nutrient intake (e.g., changes in daily habits, change in the person's diet, change in the person's sleep habit).

The feedback may comprise a recommendation to the user. The recommendation may indicate one or more changes that the person should make (e.g., changes in daily habits, change in the person's diet, change in the person's sleep habit, or change in the amount of a composition the person should take). The recommendation may be based on one or more levels of the one or more substances in the sample(s) associated with the user. In such cases, the recommendation may indicate that one or more levels of the one or more substances is higher or lower than a recommended level, that the person should adjust one or more aspect of the person's lifestyle, diet, and/or nutrient intake for various purposes (e.g., to change the one or more levels of the one or more substances into a recommended range), and/or that the person should take or stop taking one or more products (e.g., a composition comprising ashwagandha extract).

The method may comprise an operation of outputting the score or feedback to the user. The score or feedback may be output to the same user interface device through which the user input the information. Alternatively, the score or feedback may be output to another computing device not associated with (e.g., separate from) the user. For example, the score or feedback may be output to another individual associated with the person, such as a physician.

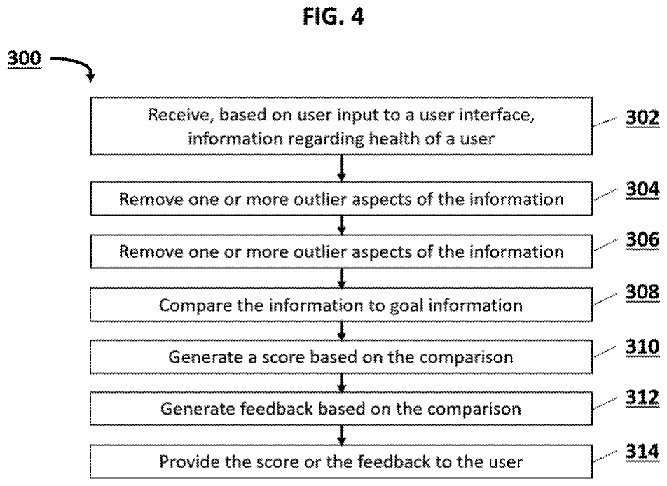

FIG. 4 illustrates an example method 300 for monitoring the health of a person (e.g., such as a user) and providing a recommendation to the user in accordance with the disclosure. The method 300 may comprise operations of (a) receiving, based on user input to a user interface, information regarding the health of the user 302; (b) removing one or more outlier aspects of a first subset of the information based on a characteristic of the one or more outlier aspects 304; (c) removing a one or more outlier aspects of a second subset of the information based on a characteristic of the one or more outlier aspects 306; (d) comparing the information to goal information 308; (e) generating a score based on the comparison 310; (f) generating feedback based on the comparison 312; and (g) providing the score or feedback to the user 314.

In some embodiments, a machine learning algorithm may perform one or more of the steps of (a) receiving, information regarding the health of the user 302; (b) removing one or more outlier aspects of a first subset of the information based on a characteristic of the one or more outlier aspects 304; (c) removing a one or more outlier aspects of a second subset of the information based on a characteristic of the one or more outlier aspects 306; (d) comparing the information to goal information 308; (e) generating a score based on the comparison 310; (f) generating feedback based on the comparison 312; and (g) providing the score or feedback to the user 314. The machine learning algorithm may be stored on a memory. The machine learning algorithm may be a supervised machine learning algorithm.

In some embodiments, the system may determine that a person should take the composition based on the received information using a server (e.g., a physical processing device or a virtual server) or a computing device (e.g., a laptop, smartphone, or another processing device) of the system. In those embodiments, the received information to the clinical data may be compared and whether the composition should be taken may be determined based on the comparison. The clinical data may be received by the server or computing device or may be stored in a database on the server or computing device. In some cases, the server or computing device may receive the clinical information from a processing device associated with the clinical information.

In some embodiments, the recommendation is made using the received information and the clinical data. As described above, in some embodiments, recommendations are determined based one or more factors. The one or more factors may include cortisol levels of the person. The cortisol levels of the person may include current cortisol levels of the person as well as desired cortisol levels of the person. The cortisol levels of the person may be determined based on the received information. The information received may be a cortisol level from 1, 2, 3, 4, 5, or 6 levels tested in a day. The cortisol level may be an average.

In some embodiments, a second recommendation may be provided based on additional information, edit information, or re-input information. In those embodiments, the person may have the option to input additional information, edit information, or re-input information to receive the second recommendation.

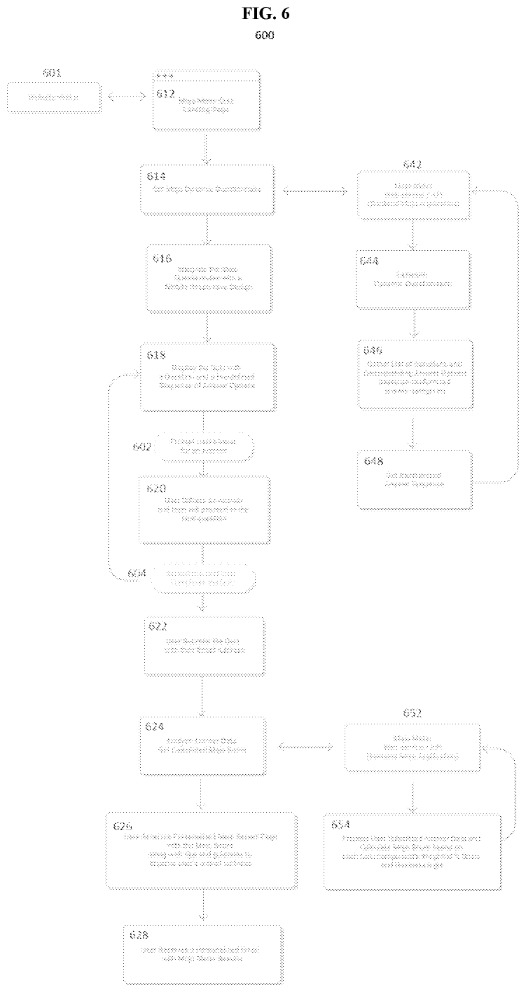

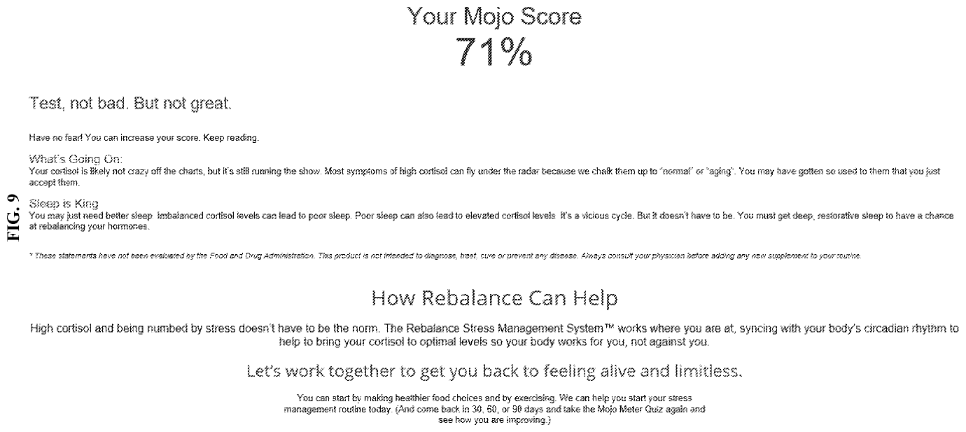

FIG. 6 illustrates an example of a computer implemented method for monitoring the health of a person (e.g., such as a user) and providing feedback such as an indication of cortisol and/or a recommendation to the user 600 in accordance with the disclosure. The method may comprise at least one or more steps of: a) prompting a user to access a website landing page 612, wherein the website is integrated into a mobile responsive design 618, such as, a mobile webpage; b) providing a dynamic questionnaire 614 to the user; c) displaying a health assessment quiz on the webpage 618, wherein the quiz provides a user with health related questions and optionally a pre-defined sequence of answer options per question; d) providing a prompt from the webpage, for the user to answer a question 602; e) receiving a user responses to the one or more questions 620, the response comprising, health information and data, wherein the input can either be enter by the user or selected from a pre-determined list of answer options; f) submitting the one or more user responses 622 to be analyzed; g) analyzing the one or more responses 624 h) outputting one or more personalized scores and/or recommendations to the user in response to the provided health information and data 626; and optionally i) sending an email of the one or more personalized scores and/or recommendations to the user's email, one example is illustrated in FIG. 9. Optionally, steps 618, 602, and 620 are repeated until each question is answered by the user 604.

In some embodiments, providing the dynamic questionnaire 614 may comprise accessing through web service 642 a questionnaire. As illustrated in FIG. 6, providing the dynamic questionnaire 642 may comprise one or more operations of generating the dynamic questionnaire 644, gathering a list of questions and corresponding answer options 646, and getting a randomized answer sequence 648. The web service 642 may then forward the dynamic questionnaire to the user in operation 614.

In some embodiments, analyzing the one or more responses 624 may comprise accessing through web service 652 a module which implements an operation of processing the user submitted answer data 654 in order to return one or more personalizes scores and/or recommendations to the user.

In some embodiments, the computer implemented system and/or computer implemented method comprises a) generating a dynamic questionnaire, in response to a user accessing the website; b) gathering a list of questions and corresponding answer options based on randomized answer categories; c) providing a list of questions and corresponding answer options to the user on the website; d) prompting the user to input an answer for each provided question; and/or e) processing the user submitted answer data and calculate a score based on the users answers and/or data.

As described above, in some cases, the provided questions may ask for a person's health information and data regarding one or more factors. Non-limiting examples of questions include asking for the person's age, daily sleep, daily diet, libido level, mental clarity, daily fatigue levels, daily mood, daily exercise, level of energy during exercise, daily stress levels, measured cortisol levels in the morning, measured cortisol levels at midday, measured cortisol levels in the evening, and measured cortisol levels at night. In some embodiments, recommended regimens may change based on new incoming information received by the system.

FIGS. 8A-8O illustrate an example system for monitoring a health score and making recommendations based of a user's health information in one example of the present disclosure. FIG. 8A illustrates a prompt to a user to the user to be begin a questionnaire by inputting their name. By way of nonlimiting example, the prompt may comprise a clickable web object comprising text or an image, a button or more than one button, or the like. In some cases, the prompt comprises a clickable web object comprising a button. In some cases, the prompt may comprise a field for user input. The field may be a text field, checkbox, radio button, drop-down list or other graphical control element to direct user input. As illustrated in FIG. 8A, the prompt may comprise a text box for the user to input biographical data (e.g., a name of the user). When the user selects the prompt, the system may direct the user to another prompt to indicate the subject in need of mental crisis assistance. The prompt may be on another page on the GUI or a web page. The prompt may comprise an additional button and/or additional button or text input field.

FIGS. 8B-8N show examples of health-related question prompts and accompanied pre-selected answer choices for a user to answer. In some cases, the prompt comprises presenting a landing web page to the user. The user may indicate their answer to the displayed question by selecting the appropriate answer choice from the prompt. Once the user has indicated their answer to the displayed question, the user is then directed to another web page which prompts the user to answer another health-related question. These questions and answers prompts can comprise one or more of the illustrated questions in FIGS. 8A-8N and or one or more questions described within the present application. The list of possible questions and answer prompts are not limited to those described in the present application and it should be understood that any health-related question and possible answer may be asked as is known in the art.

As illustrated in FIGS. 8B-8N, any graphical control element may be employed with systems of the disclosure to prompt the user for input. In some cases, the graphical control element is a radio button. In some cases, the graphical control element is a text field. In some cases, the graphical control element is a drop-down menu. In some cases, the graphical control element comprises more than one of these graphical control elements. Further, the possible graphical control elements are not limited to those described in the present application.

FIG. 8O show examples of a prompt for a user to enter an email after answering the health related questions to receive the result which may include a health score that is indicative of the users cortisol level or health and feedback such as one or more recommendations.

The feedback may comprise visual feedback summarizing the overall health of the person. Alternatively, the feedback may summarize only one factor (e.g., diet, exercise, cortisol) or subset of factors corresponding to the input information. The feedback may indicate that one or more of the user's behaviors should be maintained. Alternatively, the feedback may indicate the person should adjust one or more changes to their behavior or lifestyle, diet, and/or nutrient intake (e.g., changes in daily habits, change in the person's diet, change in the person's sleep habit).

The feedback may comprise a recommendation to the user. The recommendation may indicate one or more changes that the person should make (e.g., changes in daily habits, change in the person's diet, change in the person's sleep habit, or change in the amount of a composition the person should take). The recommendation may be based on one or more levels of the one or more substances in the sample(s) associated with the user. In such cases, the recommendation may indicate that one or more levels of the one or more substances is higher or lower than a recommended level, that the person should adjust one or more aspect of the person's lifestyle, diet, and/or nutrient intake for various purposes (e.g., to change the one or more levels of the one or more substances into a recommended range), and/or that the person should take or stop taking one or more products (e.g., a composition comprising ashwagandha extract).

The method may comprise an operation of outputting the score or feedback to the user through an email message. The score or feedback may be output to the same user interface device through which the user input the information. Alternatively, the score or feedback may be output to another computing device not associated with (e.g., separate from) the user. For example, the score or feedback may be output to another individual associated with the person, such as a physician.

In any one of the embodiments described above the calculated score may be a representation of a person's cortisol level.

### Computer Systems

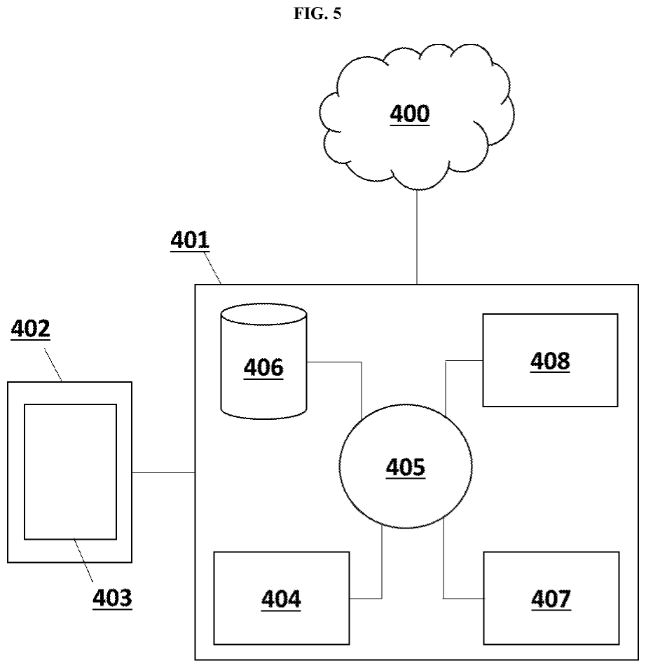

FIG. 5 shows a computer system 401 suitable for implementing the machine learning model methods described herein. The computer system 401 may process various aspects of information of the present disclosure, such as, for example, information about a person and score and recommendation determination. The computer system 401 may be an electronic device. The electronic device may be a mobile electronic device.

The computer system 401 may comprise a central processing unit (CPU, also “processor” and “computer processor” herein) 405, which may be a single core or multi core processor, or a plurality of processor for parallel processing. The computer system 401 may further comprise memory or memory locations 404 (e.g., random-access memory, read-only memory, flash memory), electronic storage unit 406 (e.g., hard disk), communications interface 408 (e.g., network adapter) for communicating with one or more other devices, and peripheral devices 407, such as cache, other memory, data storage and/or electronic display adapters. The memory 404, storage unit 406, interface 408, and peripheral devices 407 are in communication with the CPU 405 through a communication bus (solid lines), such as a motherboard. The storage unit 406 may be a data storage unit (or a data repository) for storing data. The computer system 401 may be operatively coupled to a computer network (“network”) 400 with the aid of the communication interface 408. The network 400 may be the Internet, an internet and/or extranet, or an intranet and/or extranet that is in communication with the Internet. The network 400 may, in some case, be a telecommunication and/or data network. The network 400 may include one or more computer servers, which may enable distributed computing, such as cloud computing. The network 400, in some cases with the aid of the computer system 401, may implement a peer-to-peer network, which may enable devices coupled to the computer system 401 to behave as a client or a server.

The CPU 405 may execute a sequence of machine-readable instructions, which may be embodied in a program or software. The instructions may be directed to the CPU 405, which may subsequently program or otherwise configured the CPU 405 to implement methods of the present disclosure. Examples of operations performed by the CPU 405 may include fetch, decode, execute, and writeback.

The CPU 405 may be part of a circuit, such as an integrated circuit. One or more other components of the system 401 may be included in the circuit. In some cases, the circuit is an application specific integrated circuit (ASIC).

The storage unit 406 may store files, such as drivers, libraries and saved programs. The storage unit 406 may store information from one or more persons and programs that act on the information. The computer system 401, in some cases may include one or more additional data storage units that are external to the computer system 401, such as located on a remote server that is in communication with the computer system 401 through an intranet or the internet.

Methods as described herein may be implemented by way of machine (e.g., computer processor) executable code stored on an electronic storage location of the computer device 401, such as, for example, on the memory 404 or electronic storage unit 406. The machine executable or machine-readable code may be provided in the form of software. During use, the code may be executed by the processor 405. In some instances, the code may be retrieved from the storage unit 406 and stored on the memory 404 for ready access by the processor 405. In some instances, the electronic storage unit 406 may be precluded, and machine-executable instructions are stored on memory 404.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 401, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage’ media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media include dynamic memory, such as main memory of such a computer platform. Tangible transmission media includes coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 402 that comprises a user interface (UI) 403 for inputting donor parameters and viewing the association of a donor's parameters with a generated model. Examples of UI's include, without limitation, a graphical user interface (GUI) and web-based user interface.

Methods and systems of the present disclosure can be implemented by way of one or more algorithms and with instructions provided with one or more processors as disclosed herein. An algorithm can be implemented by way of software upon execution by the central processing unit 405. The algorithm can be, for example, random forest, graphical models, support vector machine or other.

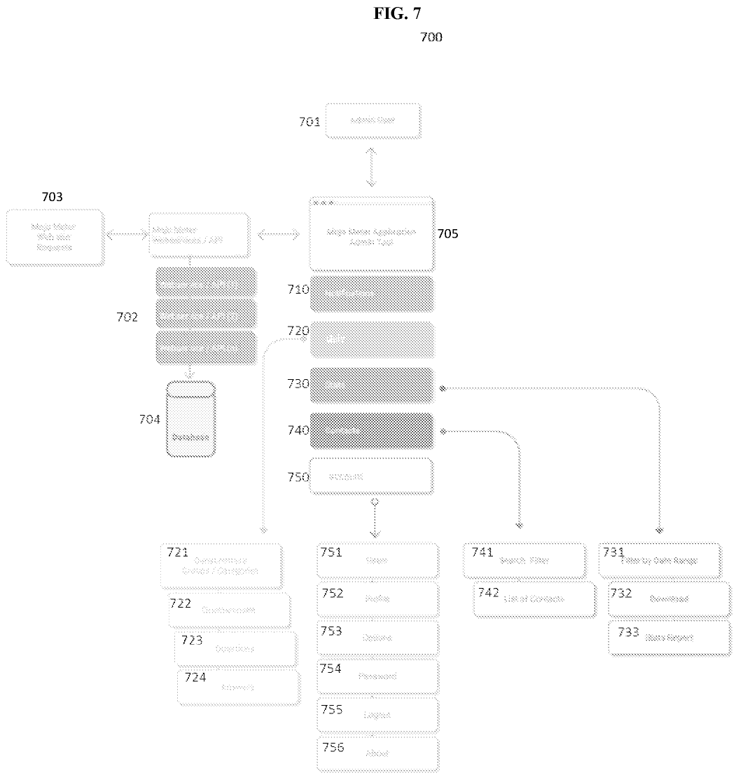

FIG. 7 shows a web-based computer system 700 configured to implement the methods of the present disclosure. The methods may comprise, by way of non-limiting example, monitoring, tracking and/or making recommendations based on person's health in accordance with the disclosure. The system 700 may comprise a web service 702 in communication with a database 704. The web service 702 may be configured to permit communication between a user (e.g., providing a request on a website) 703 and server 705 (as well as data in database 704). The server 705 may comprise one or more modules 710, 720, 730, 740, and 750 or components illustrated in FIG. 7 (e.g., notification module 710, quiz module 720, stats module 730, contacts module 740, and an account module 750). The notification module 710 may be configured for providing information and recommendation to users. The notifications may be output as an email to one or more users as described above. In some embodiments, the information and recommendations to the user are in response to results generated by the stats module 730.

In some embodiments, the notification module 710 may be configured to receive the question prompts for the health quiz, generated by the quiz module 720 and display the question prompts to a user and/or prompt the user for an input. In a non-limiting embodiment, the quiz module 720 may be configured to perform operations, retrieve and/or generate elements such as questionnaire categories 721, questionnaires 722, one or more questions 723, and/or one or more answers 724 corresponding to the one or more questions 723. The quiz module 720 may be configured to provide to the user (e.g., health-related) questions prompts and/or pre-selected answer choices, such as those described elsewhere herein. The questions prompts and/or preselected answer choices can be generated and displayed to a user on a webpage as illustrated in FIGS. 8A-8O.

The quiz module 720 may be configured to generate a dynamic questionnaire and health assessment quiz that is presented to a user; wherein the quiz provides a user with health-related question prompts and optionally a pre-defined sequence of answer options per question; and/or receive a user input, i.e. health information and data and/or answers, in response to the one or more question prompts of the quiz. The quiz module 720 may be configured to provide the input data to the stats module 730.

In a non-limiting embodiment, the stats module 730 may be configured to perform operations such as filtering information 731, wherein the data is filtered by at least one of a date range, a score range, one or more health factors and/or health information or data. The information and data may be filter by any other well known or obvious factor or category. The stat module 730 may also perform operations to receive and/or generate one or more stats report 733. The stat module 730 may also perform operations and/or generate one or more downloads 732. The stat module may be configured to implement one or more operations associated with scoring a user input to (e.g., a health-related) questionnaire as described elsewhere herein.

The stats module 730 may be configured to receive input information and data from the quiz module 720 and output information such as a stats report 736 to the notification module 710 to present information to the user in response to their answers and/or store the stats report in a database 704.

In a non-limiting embodiment, the contacts module 740 may be configured to perform operations to receive and/or generate lists of contacts 742. The contacts module 740 may be configured to perform operations such as filtering searches 741, wherein the search is filtered by at least one of a contact name, age range, a score range, one or more health factors and/or health information or data. The contact name, age range, score range, or one or more health factors and/or health information or data may be provided by the user 703, as illustrated in FIGS. 8A-8O, or accessed from database 704. The search may be filtered by any other well-known or obvious factor or category.

In a non-limiting embodiment, the account module 750 may be configured to perform operations and functions for anything related to one or more user information and access to the system. This information and access includes but is not limited to, users 751, profile 752, options 753, password 754, logout 755 and about 756. The information can be associated with the user accessing server 705 through the web service 702. Alternatively, the information can be associated with another user or users. The account module or any module is not limited in its function and each module may include and perform one or more of the elements and functions from any other module or not listed above.

### Example System Embodiments

1. A method for recommending whether to make a lifestyle, diet, and/or nutrient adjustment, comprising: receiving information regarding health of a user, wherein a first subset of the information is subjective and a second subset of the information is objective; removing one or more outlier aspects of the second subset of the information based on a characteristic of the one or more outlier aspects to create a third subset of the information; comparing the third subset of information to a goal subset of information; analyzing the first subset of information based on the goal subset of information; generating a score based on the comparing the third subset of the information to the goal subset of information and analyzing the first subset of information based on the goal subset of information; generating feedback associated with the adjustment based on the comparing the third subset of information to the goal subset of information and analyzing the first subset of information based on the goal subset of information; and providing the score and the feedback to the user.
2. The method of embodiment 1, wherein the second subset of the information comprises age of the user, sleep data of the user, diet of the user, or measured levels of a substance in one or more sample associated with the user.
3. The method of embodiment 2, wherein the second subset comprises one or more cortisol levels in the one or more samples associated with user.
4. The method of embodiment 3, wherein the one or more cortisol levels are measured over a day.
5. The method of embodiment 4, wherein the one or more cortisol levels are measured at least four times in the day, optionally wherein the at least four levels are averaged.
6. The method any one of embodiments 1-5, wherein the first subset of the information comprises libido level of the user, mental clarity of the user, fatigue levels of the user, mood of the user, level of energy of the user during exercise, or daily stress levels of the user.
7. The method of any one of embodiments 1-6, wherein the feedback comprises a recommended lifestyle change.
8. The method of embodiment 7, wherein the lifestyle change comprises a change to the user's diet, exercise, or sleep behavior.
9. A system for monitoring the health of a person, the system comprising: a processor; and a non-transitory computer-readable medium comprising software configured to cause the processor to: receive an input comprising subjective information and objective information regarding a user; compare the subjective information and objective information regarding the user to goal information; generate an output based on the subjective information, objective information, and goal information, wherein the output comprises a recommendation to the person to make a lifestyle, diet, and/or nutrient adjustment; and provide the output to a computing device associated with the user.
10. The system of embodiment 9, wherein the recommendation further comprises a recommended lifestyle change.
11. The system of embodiment 10, wherein the recommended lifestyle change comprises a change to the person's exercise or sleep behavior.
12. The system of any one of embodiments 9-11, wherein the objective information comprises a level of a substance in a sample associated with the user.
13. The method of embodiment 12, wherein the input comprises one or more cortisol levels in one or more samples associated with the person.
14. The method of embodiment 13, wherein the objective information comprises one or more cortisol levels of the user.
15. The method of embodiment 14, wherein the one or more cortisol levels of the objective information are measured at least four times a day, optionally wherein the at least four levels measured are averaged.
16. The method of embodiment 16, wherein the one or more cortisol levels of the objective information comprise four cortisol levels of the user, wherein the four cortisol levels of the user are each taken at a different time of day.
17. The system of any one of embodiments 9-15, wherein the output further comprises a score associated with the health of the user and the recommended lifestyle change.
18. A computer implemented method of tracking cortisol and/or providing feedback to a user, comprising:
(a) providing a questionnaire, wherein the questionnaire comprises a first health related question and a second health related question; (b) displaying the first and second questions to the user (e.g., simultaneously or sequentially); (c) receiving from a user answer data in response to the first question; (d) receiving from a user answer data in response to the second question; (e) applying an algorithm configured to perform steps of: (i) analyzing the received first and second use answer data; (ii) generating a health score based on the analyzed answer data and/or generating feedback associated with the generated health score or based on the analyzed answer data; and (iii) outputting the health score and/or the feedback to the user or a third party; optionally wherein the first health related question is subjective and the second health related question is objective, wherein the first health related question is subjective and the second health related question is subjective, or the first health related question is objective and the second health related question is objective; optionally wherein the first health related question is part of a first subset of health related questions and the second health related question is part of a second subset of health related questions; and optionally wherein if the health score and/or the feedback is output to a third party, the third party provides a recommendation to the user, further optionally wherein the third party is a nutritionist, clinician, or other healthcare provider.
19. The method of embodiment 18, wherein the second health related question is part of the second subset of health related questions related to information comprising age of the user, sleep data of the user, diet of the user, or measured levels of a substance in one or more sample associated with the user, or a combination of two or more thereof.
20. The method of embodiment 19, wherein the second subset of health related questions related to information comprising one or more cortisol levels in the one or more samples associated with user.
21. The method any one of embodiments 18-20, wherein the first health related question is part of the first subset of health related questions related to information comprising libido level of the user, mental clarity of the user, fatigue levels of the user, mood of the user, level of energy of the user during exercise, or daily stress levels of the user, or a combination of two or more thereof.
22. The method of any one of embodiments 18-21, wherein feedback is generated and the feedback comprises a recommended lifestyle change.
23. The method of embodiment 22, wherein the lifestyle change comprises a change to the user's diet, exercise, or sleep behavior.
24. The method of any one of embodiments 18-23, wherein the health score is generated and wherein the health score is a direct representation of a user's cortisol level.
25. The method of any one of embodiments 18-24, wherein the computer implemented method comprises an algorithm that is a machine learning algorithm.
26. The method of any one of embodiments 18-25, wherein the health score is generated and the feedback comprises a visual representation of the generated health score.
27. The method of embodiment 26, wherein the visual representation is displayed on a user interface.
28. The method of any one of embodiments 26-27, wherein the visual representation comprises one or more of a chart, a meter, a graph, an image, or text.
29. The method of any one of embodiments 18-28, wherein the computer implemented method comprises an algorithm and the algorithm applies a weighted mathematical formula to the first and second user answer data to determine a score.
30. The method of embodiment 29, wherein the weighted formula is determined based the answer data received in response to one or more of the first and/or second questions.
31. The method of any one of embodiments 18-30, providing the feedback (e.g., recommendation whether to make a lifestyle, diet, and/or nutrient adjustment).

### Combination Regimen

In one aspect, provided is a combination regimen of compositions used by a person. Non-limiting examples of product compositions used in the combination regimen include RH1, RH2, and/or RH3. In some embodiments, the person uses the combination regimen in a time period. Non-limiting examples of time periods of the combination regimen include about once a day, about once every two days, about once every three days, about once every four days, about once every 5 days, about once every 6 days, about once every week, about once every two weeks, about once every three weeks, or about once every 1 month. In a nonlimiting example embodiment, the combination regimen is used by the person once every day. In some embodiments, the person uses a composition of the combination regimen at a time point in the day of the person. Non-limiting examples of a time point in the day of the person include within about the first twelve hours of the day of the person (e.g., within about the first twelve hours after the person awakes), within about the second 12 hours of the day of the person, before the person goes to bed for the night, within about the first hour of the day of the person, within about the second hour of the day of the person, within about the third hour of the day of the person, within about the third hour of the day of the person, within about the fourth hour of the day of the person, within about the fifth hour of the day of the person, within about the sixth hour of the day of the person, within about the seventh hour of the day of the person, within about the eighth hour of the day of the person, within about the ninth hour of the day of the person, within about the tenth hour of the day of the person, within about the eleventh hour of the day of the person, within about the twelfth hour of the day of the person, within about the thirteenth hour of the day of the person, within about the fourteenth hour of the day of the person, within about the fifteenth hour of the day of the person, within about the sixteenth hour of the day of the person, within about the seventeenth hour of the day of the person, within about the eighteenth hour of the day of the person, within about the nineteenth hour of the day of the person, within about the twentieth hour of the day of the person, within about the twenty-first hour of the day of the person, within about the twenty-second hour of the day of the person, within about the twenty-third hour of the day of the person, within about the twenty-fourth hour of the day of the person, within about the first twelve hours of the day of the person, within about the second twelve hours of the day of the person, about two hours before the final meal of the day of the person, about 1 hour before the final meal of the day of the person, with the final meal of the day of the person, about 1 hour after the final meal of the day of the person, about 2 hours after the final meal of the day of the person, about 2 hours before the person goes to sleep for the night, about 1 hour and 45 minutes before the person goes to sleep for the night, about 1 hour and 30 minutes before the person goes to sleep for the night, about 1 hour and 15 minutes before the person goes to sleep for the night, about 1 hour before the person goes to sleep for the night, about 45 minutes before the person goes to sleep for the night, about 30 minutes before the person goes to sleep for the night, and about 15 minutes before the person goes to sleep for the night. In some embodiments, the person may use a composition of the combination regimen at a first time point, a composition of the combination regimen at a second time point, a composition of the combination regimen at a third time point, a composition of the combination regimen at a fourth time point, a composition of the combination regimen at a fifth time point, a composition of the combination regimen at a sixth time point, a composition of the combination regimen at a seventh time point, a composition of the combination regimen at an eighth time point, a composition of the combination regimen at a ninth time point, a composition of the combination regimen at a tenth time point, or a combination of one or more thereof. In a non-limiting example embodiment, the person uses a composition of the combination regimen at a first time point, a composition of the combination regimen at a second time point, and a composition of the combination regimen at a third time point. In some embodiments, a different composition of the combination regimen is used by the person at a different time point. In some embodiments, an first example product (e.g., RH1) is used by the person at a first time point, a second example product (e.g., RH2) is used by the person at a second time point, and a third example product (e.g., RH3) is used by the person at a third time point. In a nonlimiting example embodiment, the person uses the RH1 product within about the first 12 hours of the day of the person, the person uses the RH2 product within about the second twelve hours of the day of the person, and the person uses the RH3 product before the person goes to sleep for the night. In a further nonlimiting example embodiment, the person uses the RH1 product within about the first hour of the day of the person, the person uses the RH2 product about one hour after the final meal of the day of the person, and the person uses the RH3 product about 30 minutes before the person goes to sleep for the night.

In one aspect, the person uses the RH1 example product in the first 12 hours of the day of the person. Non-limiting examples of a RH1 example product are shown in Example 1 and Example 2.

In one aspect, the person uses the RH2 example product in the second 12 hours of the day of the person. Non-limiting examples of a RH2 example product are shown in Example 3 and Example 4.

In one aspect, the person uses the RH3 example product before the person goes to sleep for the night. Non-limiting example RH3 example products are shown in Example 5.

### Effect of Improved Health

Embodiments comprising compositions herein are adapted to have one or more effects when used by the person. In some aspects, compositions herein are formulated to modulate cortisol levels in a person and an effect may be associated with the modulated cortisol levels. Effects may be improvements in the health of the person, such as balancing hormones (e.g., cortisol or testosterone), improving the immune system of the person, and/or improving the person's hGH production. Effects may further include increasing energy in the person, facilitating relaxation of the person, increasing, or decreasing the libido of the person, improving the mental clarity of the person, decreasing the stress of the person, decreasing fatigue of the person, and/or improving the mood of the person.

The person may have been previously identified as suffering from a condition. In other embodiments, the person may not have previously been identified as suffering from a condition. In yet another embodiment, the person may not have previously been identified as having a condition but may be at risk of developing the condition. In still another embodiment, the person may have a condition but may not have been identified as having the condition. In further still another embodiment, the person may suffer from a symptom related to a condition described herein.

In some embodiments, an effective amount refers to an amount of an ingredient/agent and/or composition effective for use against a condition in a person. In some cases, an effective amount reduces the severity of symptoms of the condition.

Some embodiments include a method for modulating cortisol levels by taking compositions containing ashwagandha extract, as described below. Those embodiments may include taking the compositions containing ashwagandha contract for a specific duration and/or with specific amounts, as described below. Those embodiments may include taking one composition or more than one composition, as described below.

Non-limiting conditions for which an ashwagandha extract composition can be used for may include hormonal imbalance of one or more hormones in a person, low energy levels of a person, and poor sleep health of a person. In some cases, low or poor refers to a level that is below an average level of healthy persons. In some cases, low or poor refers to a level that is below the level of a healthy person. A healthy person may share one or more characteristics (e.g., age, age range, exercise habits, weight, height, sleep habits, dietary habits, or other characteristics) with the person with the condition.

The compositions may be delivered in certain amount. This amount will vary depending upon a variety of factors, including but not limited to the characteristics of the composition, (including activity, bioavailability), the physiological condition of the person (including age, sex, disease type and stage, general physical condition, responsiveness to a given composition), the nature of the carrier or carriers in the formulation, and the route of delivery. The amount may be determined by monitoring a person's response to delivery of a composition and adjusting the amount taken accordingly. Examples of amounts of the composition taken at a given time (e.g., once a day) may be from about 100 mg to about 22500 mg, e.g., about 100 mg to about 20000 mg, about 100 mg to about 17500 mg, about 100 mg to about 15000 mg, about 100 mg to about 12500 mg, about 100 mg to about 10000 mg, about 100 mg to about 7500 mg, about 100 mg to about 5000 mg, about 100 mg to about 2500 mg, about 100 mg to about 2250 mg, about 100 mg to about 2000 mg, about 100 mg to about 1750 mg, about 100 mg to about 1500 mg, about 100 mg to about 1250 mg, about 100 mg to about 1000 mg, about 100 mg to about 750 mg, about 100 mg to about 500 mg, about 100 mg to about 250 mg, about 250 mg to about 22500 mg, about 250 mg to about 20000 mg, about 250 mg to about 17500 mg, about 250 mg to about 15000 mg, about 250 mg to about 12500 mg, about 250 mg to about 10000 mg, about 250 mg to about 7500 mg, about 250 mg to about 5000 mg, about 250 mg to about 2500 mg, about 250 mg to about 2250 mg, about 250 mg to about 2000 mg, about 250 mg to about 1750 mg, about 250 mg to about 1500 mg, about 250 mg to about 1250 mg, about 250 mg to about 1000 mg, about 250 mg to about 750 mg, about 250 mg to about 500 mg, about 500 mg to about 22500 mg, about 500 mg to about 20000 mg, about 500 mg to about 17500 mg, about 500 mg to about 15000 mg, about 500 mg to about 12500 mg, about 500 mg to about 10000 mg, about 500 mg to about 7500 mg, about 500 mg to about 5000 mg, about 500 mg to about 2500 mg, about 500 mg to about 2250 mg, about 500 mg to about 2000 mg, about 500 mg to about 1750 mg, about 500 mg to about 1500 mg, about 500 mg to about 1250 mg, about 500 mg to about 1000 mg, about 500 mg to about 750 mg, about 750 mg to about 22500 mg, about 750 mg to about 20000 mg, about 750 mg to about 17500 mg, about 750 mg to about 15000 mg, about 750 mg to about 12500 mg, about 750 mg to about 10000 mg, about 750 mg to about 7500 mg, about 750 mg to about 5000 mg, about 750 mg to about 2500 mg, about 750 mg to about 2250 mg, about 750 mg to about 2000 mg, about 750 mg to about 1750 mg, about 750 mg to about 1500 mg, about 750 mg to about 1250 mg, about 750 mg to about 1000 mg, about 1000 mg to about 22500 mg, about 1000 mg to about 20000 mg, about 1000 mg to about 17500 mg, about 1000 mg to about 15000 mg, about 1000 mg to about 12500 mg, about 1000 mg to about 10000 mg, about 1000 mg to about 7500 mg, about 1000 mg to about 5000 mg, about 1000 mg to about 2500 mg, about 1000 mg to about 2250 mg, about 1000 mg to about 2000 mg, about 1000 mg to about 1750 mg, about 1000 mg to about 1500 mg, about 1000 mg to about 1250 mg, about 1250 mg to about 22500 mg, about 1250 mg to about 20000 mg, about 1250 mg to about 17500 mg, about 1250 mg to about 15000 mg, about 1250 mg to about 12500 mg, about 1250 mg to about 10000 mg, about 1250 mg to about 7500 mg, about 1250 mg to about 5000 mg, about 1250 mg to about 2500 mg, about 1250 mg to about 2250 mg, about 1250 mg to about 2000 mg, about 1250 mg to about 1750 mg, about 1250 mg to about 1500 mg, about 1500 mg to about 22500 mg, about 1500 mg to about 20000 mg, about 1500 mg to about 17500 mg, about 1500 mg to about 15000 mg, about 1500 mg to about 12500 mg, about 1500 mg to about 10000 mg, about 1500 mg to about 7500 mg, about 1500 mg to about 5000 mg, about 1500 mg to about 2500 mg, about 1500 mg to about 2250 mg, about 1500 mg to about 2000 mg, about 1500 mg to about 1750 mg, about 1750 mg to about 22500 mg, about 1750 mg to about 20000 mg, about 1750 mg to about 17500 mg, about 1750 mg to about 15000 mg, about 1750 mg to about 12500 mg, about 1750 mg to about 10000 mg, about 1750 mg to about 7500 mg, about 1750 mg to about 5000 mg, about 1750 mg to about 2500 mg, about 1750 mg to about 2250 mg, about 1750 mg to about 2000 mg, about 2000 mg to about 22500 mg, about 2000 mg to about 20000 mg, about 2000 mg to about 17500 mg, about 2000 mg to about 15000 mg, about 2000 mg to about 12500 mg, about 2000 mg to about 10000 mg, about 2000 mg to about 7500 mg, about 2000 mg to about 5000 mg, about 2000 mg to about 2500 mg, about 2000 mg to about 2250 mg, about 2250 mg to about 22500 mg, about 2250 mg to about 20000 mg, about 2250 mg to about 17500 mg, about 2250 mg to about 15000 mg, about 2250 mg to about 12500 mg, about 2250 mg to about 10000 mg, about 2250 mg to about 7500 mg, about 2250 mg to about 5000 mg, about 2250 mg to about 2500 mg, about 2500 mg to about 22500 mg, about 2500 mg to about 20000 mg, about 2500 mg to about 17500 mg, about 2500 mg to about 15000 mg, about 2500 mg to about 12500 mg, about 2500 mg to about 10000 mg, about 2500 mg to about 7500 mg, about 2500 mg to about 5000 mg, about 5000 mg to about 22500 mg, about 5000 mg to about 20000 mg, about 5000 mg to about 17500 mg, about 5000 mg to about 15000 mg, about 5000 mg to about 12500 mg, about 5000 mg to about 10000 mg, about 5000 mg to about 7500 mg, about 7500 mg to about 22500 mg, about 7500 mg to about 20000 mg, about 7500 mg to about 17500 mg, about 7500 mg to about 15000 mg, about 7500 mg to about 12500 mg, about 7500 mg to about 10000 mg, about 10000 mg to about 22500 mg, about 10000 mg to about 20000 mg, about 10000 mg to about 17500 mg, about 10000 mg to about 15000 mg, about 10000 mg to about 12500 mg, about 12500 mg to about 22500 mg, about 12500 mg to about 20000 mg, about 12500 mg to about 17500 mg, about 12500 mg to about 15000 mg, about 15000 mg to about 22500 mg, about 15000 mg to about 20000 mg, about 15000 mg to about 17500 mg, about 17500 mg to about 22500 mg, about 17500 mg to about 20000 mg, about 20000 mg to about 22500 mg, about 100 mg, about 250 mg, about 500 mg, about 750 mg, about 1000 mg, about 1250 mg, about 1500 mg, about 1750 mg, about 2000 mg, about 2250 mg, about 2500 mg, about 5000 mg, about 7500 mg, about 10000 mg, about 12500 mg, about 15000 mg, about 17500 mg, about 20000 mg, or about 22500 mg.

Certain compositions herein are formulated to facilitate increased energy in a person. In some such cases, the amount of the composition is about 950 mg to about 1350 mg, about 1050 mg to about 1250 mg, or about 1150 mg. An example product is called RH1.

In certain embodiments, RH1 is used to balance cortisol. In certain embodiments, RH1 is used to increase testosterone. In certain embodiments, RH1 is used to support the immune system. In certain embodiments, RH1 is used to stimulate hGH production. In certain embodiments, RH1 is used to increase endurance and performance. In certain embodiments, RH1 is used to increase libido.

Certain compositions herein are formulated to facilitate increased relaxation in a person. In some such cases, the amount of the composition is about 950 mg to about 1350 mg, about 1050 mg to about 1250 mg, or about 1150 mg. An example product is called RH2.

In certain embodiments, RH2 is used to balance cortisol. In certain embodiments, RH2 is used to increase testosterone. In certain embodiments, RH2 is used to support the immune system. In certain embodiments, RH2 is used to stimulate hGH production. In certain embodiments, RH2 is used to increase endurance and performance. In certain embodiments, RH2 is used to increase libido. In certain embodiments, RH1 and RH2 are used in combination to balance cortisol. In certain embodiments, RH1 and RH2 are used in combination to increase testosterone. In certain embodiments, RH1 and RH2 are used in combination to support the immune system. In certain embodiments, RH1 and RH2 are used in combination to stimulate hGH production. In certain embodiments, RH1 and RH2 are used in combination to increase endurance and performance. In certain embodiments, RH1 is used to increase libido.

Certain compositions herein are formulated to improve the sleep health of a person. In some such cases, the amount of the composition is about 225 mg to about 425 mg, about 275 mg to about 375 mg, or about 325 mg. An example product is called RH3.

In certain embodiments, RH3 is used to balance cortisol. In certain embodiments, RH3 is used for restorative sleep. In certain embodiments, RH3 is used to balance hormones. In certain embodiments, RH3 is used to increase testosterone. In certain embodiments, RH3 is used to reduce anxiety. In certain embodiments, RH3 is used to quite a racing mind.

In certain embodiments, the combination of RH1, RH2, and RH3 are used to balance cortisol. In certain embodiments, the combination of RH1, RH2, and RH3 are used to increase energy. In certain embodiments, the combination of RH1, RH2, and RH3 are used to balance testosterone. In certain embodiments, the combination of RH1, RH2, and RH3 are used to support the immune system. In certain embodiments, the combination of RH1, RH2, and RH3 are used to support natural hGH production. In certain embodiments, the combination of RH1, RH2, and RH3 are used to increase endurance and performance. In certain embodiments, the combination of RH1, RH2, and RH3 are used to increase libido. In certain embodiments, the combination of RH1, RH2, and RH3 are used for restorative sleep.

In some embodiments, the duration that a composition is taken may depend upon the person's progress and responsiveness to the composition. For example, in one embodiment, the composition is taken until a person's condition or symptoms reside. In another embodiment, composition is taken until a person's condition or symptoms lessen. In yet another embodiment, the composition is taken for a minimum of two or three times.

Provided below are descriptions of embodiments of regimens for compositions. In one aspect, a composition is taken to improve the health of the person.

In some embodiments, the composition is used by the person in a regimen comprising two or more units, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 units. Frequency of delivery of the units varies in some embodiments; a unit of the composition may be taken twice daily every day, once daily every day, twice daily every other day, once daily every other day, twice daily every two days, once daily every two days, twice daily every three days, once daily every three days, twice daily every four days, once daily every four days, twice daily every five days, once daily every five days, twice daily every six days, once daily every six days, twice daily every seven days, once daily every seven days, twice daily every week, or once daily once a week. Furthermore, in some instances, the regimen may change, e.g., a different regimen of two or more units (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 units) for a number of days may be followed for one or more days where the composition is not taken. In some embodiments, the one or more days is 1, 2, 3, 4, 5, 6, or 7 days. So, as a non-limiting example, a unit may be taken every day for a period of two weeks followed by a period of 7 days wherein the person does not take any units. In some embodiments, a given regimen may be repeated one or more times, e.g., weekly, every two weeks, every three weeks, every four weeks, once a month, twice a month, three times a month, or four times a month.

In some embodiments, a composition herein may be taken in combination with one or more other compositions. For example, a composition associated with an example product RH1 may be taken in combination with a composition associated with an example product RH2 or a composition associated with an example product RH3. The frequency for taking the combination of compositions varies in some embodiments. In some embodiments, the composition associated with the RH1 product may be taken once daily, twice daily, every two days, every three days, every four days, every five days, every 6 days, every week, every two weeks, every 3 weeks, every month, every two months, every 3 months, or every 6 months, while one or more of the composition associated with the RH2 product and the composition associated with the RH3 product may also be taken once daily, twice daily, every two days, every three days, every four days, every five days, every 6 days, every week, every two weeks, every 3 weeks, every month, every two months, every 3 months, or every 6 months. In some embodiments, each composition that is taken in combination may be taken with the same frequency. For example, each of the compositions associated with the RH1, RH2, and RH3 products may be taken once daily. In other embodiments, one or more compositions that are taken in combination may be taken with different frequencies. For example, the composition associated with the RH1 products may be taken once daily, the composition associated with RH2 products may be take twice daily, and the composition associated with RH3 products may be taken three times daily.

In some embodiments, the method comprises the person taking one or more units of a first composition. In some embodiments, each unit comprises about 600 mg to about 1700 mg of the composition, about 800 mg to about 1500 mg, about 1000 mg to about 1300 mg, or about 1150 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg. In some embodiments, each unit comprises about 100 mg to about 400 mg of ashwagandha extract. In some embodiments, each unit comprises about 1 mg to about 500 mg of one or more excipients (e.g., sodium starch glycolate, one or more flavoring agents, one or more sweetening agents, one or more adsorbents, glidants, and/or silicon dioxide, one or more of binders, diluents, and/or cellulose, one or more starch excipients, magnesium stearate, and/or menthol crystals). In some embodiments, each unit comprises about 25 mg to about 75 mg of chelating agent (e.g., EDTA). In some embodiments, each unit comprises about 25 mg to about 75 mg delivery enhancing agent (e.g., AstraGin®). In some embodiments, each unit comprises about 100 mg to about 400 mg of maca. In some embodiments, each unit comprises about 75 mg to about 175 mg Eurycoma longifolia. In some embodiments, each unit comprises about 50 mg to about 175 mg fenugreek seed extract. In some embodiments, each unit comprises about 5 mg to about 25 mg Tribulus terrestris. In some embodiments, each unit comprises about 2.5 mg to about 15 mg cordyceps species. In some embodiments, each unit comprises about 2.5 mg to about 15 mg nitric oxide source. In some embodiments, each unit comprises about 2.5 mg to about 10 mg of one or more vitamins (e.g., vitamin B6). In some embodiments, each unit comprises about 2.5 mg to about 15 mg tea extract (e.g., green tea extract). In some embodiments, each unit comprises about 2.5 mg to about 15 mg amino acid (e.g., D-aspartic acid). In some embodiments, each unit comprises about 2.5 mg to about 15 mg Panax ginseng. In some embodiments, the first composition is RH1. In some embodiments, the person is a male. In some embodiments, one unit of the first composition is taken once a day.

In some embodiments, the method comprises the person using one or more units of a second composition. In some embodiments, each unit comprises about 600 mg to about 1700 mg of the composition, about 800 mg to about 1500 mg, about 1000 mg to about 1300 mg, or about 1150 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg. In some embodiments, each unit comprises about 100 mg to about 400 mg of ashwagandha extract. In some embodiments, each unit comprises about 1 mg to about 450 mg of one or more excipients (e.g., sodium starch glycolate, one or more flavoring agents, one or more sweetening agents, one or more adsorbents, glidants, and/or silicon dioxide, one or more of binders, diluents, and/or cellulose, one or more starch excipients, magnesium stearate, and/or menthol crystals). In some embodiments, each unit comprises about 25 mg to about 75 mg of chelating agent (e.g., EDTA). In some embodiments, each unit comprises about 25 mg to about 75 mg delivery enhancing agent (e.g., AstraGin®). In some embodiments, each unit comprises about 150 mg to about 450 mg of maca. In some embodiments, each unit comprises about 100 mg to about 250 mg fenugreek seed extract. In some embodiments, each unit comprises about 0.5 mg to about 10 mg of one or more vitamins (e.g., vitamin B6). In some embodiments, each unit comprises about 5 mg to about 20 mg cordyceps species. In some embodiments, each unit comprises about 5 mg to about 20 mg tea extract (e.g., green tea extract). In some embodiments, each unit comprises about 5 mg to about 20 mg nitric oxide source. In some embodiments, each unit comprises about 5 mg to about 20 mg bacopa. In some embodiments, each unit comprises about 2.5 mg to about 10 mg amino acid (e.g., D-aspartic acid). In some embodiments, each unit comprises about 2.5 mg to about 10 mg Panax ginseng. In some embodiments, the second composition is RH1. In some embodiments, the person is a female. In some embodiments, one unit of the second composition is taken once a day.

In some embodiments, the method comprises the person using one or more units of a third composition. In some embodiments, each unit comprises about 600 mg to about 1700 mg of the composition, about 800 mg to about 1500 mg, about 1000 mg to about 1300 mg, or about 1150 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg. In some embodiments, each unit comprises about 100 mg to about 400 mg of ashwagandha extract. In some embodiments, each unit comprises about 1 mg to about 500 mg of one or more excipients (e.g., sodium starch glycolate, one or more flavoring agents, one or more sweetening agents, one or more adsorbents, glidants, and/or silicon dioxide, one or more of binders, diluents, and/or cellulose, one or more starch excipients, magnesium stearate, and/or menthol crystals). In some embodiments, each unit comprises about 25 mg to about 75 mg of chelating agent (e.g., EDTA). In some embodiments, each unit comprises about 25 mg to about 75 mg delivery enhancing agent (e.g., AstraGin®). In some embodiments, each unit comprises about 100 mg to about 300 mg of cordyceps species (e.g., cordyceps prime). In some embodiments, each unit comprises about 75 mg to about 200 mg phosphatidylserine. In some embodiments, each unit comprises about 25 mg to about 75 mg supplemental mineral (e.g., magnesium glycinate). In some embodiments, each unit comprises about 25 mg to about 75 mg compound derived from a magnolia tree (e.g., Magnolia officinalis). In some embodiments, each unit comprises about 5 mg to about 25 mg ginger. In some embodiments, each unit comprises about 5 mg to about 25 mg horny goat weed. In some embodiments, each unit comprises about 2.5 mg to about 10 mg sclareolides (e.g., sage leaf powder). In some embodiments, each unit comprises about 2.5 mg to about 10 mg diindolylmethane (DIM). In some embodiments, each unit comprises about 2.5 mg to about 15 mg zinc. In some embodiments, the third composition is RH2. In some embodiments, the person is a male. In some embodiments, one unit of the third composition is taken once a day. In some embodiments, one unit of the first composition is taken once a day, and one unit of the third composition is taken once a day.

In some embodiments, the method comprises the person using one or more units of a fourth composition. In some embodiments, each unit comprises about 600 mg to about 1700 mg of the composition, about 800 mg to about 1500 mg, about 1000 mg to about 1300 mg, or about 1150 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg. In some embodiments, each unit comprises about 100 mg to about 400 mg of ashwagandha extract. In some embodiments, each unit comprises about 1 mg to about 450 mg of one or more excipients (e.g., sodium starch glycolate, one or more flavoring agents, one or more sweetening agents, one or more adsorbents, glidants, and/or silicon dioxide, one or more of binders, diluents, and/or cellulose, one or more starch excipients, magnesium stearate, and/or menthol crystals). In some embodiments, each unit comprises about 25 mg to about 75 mg of chelating agent (e.g., EDTA). In some embodiments, each unit comprises about 25 mg to about 75 mg delivery enhancing agent (e.g., AstraGin®). In some embodiments, each unit comprises about 100 mg to about 300 mg of cordyceps species (e.g., cordyceps prime). In some embodiments, each unit comprises about 75 mg to about 200 mg phosphatidylserine. In some embodiments, each unit comprises about 25 mg to about 75 mg supplemental mineral (e.g., magnesium glycinate). In some embodiments, each unit comprises about 25 mg to about 75 mg compound derived from a magnolia tree (e.g., Magnolia officinalis). In some embodiments, each unit comprises about 5 mg to about 25 mg ginger. In some embodiments, each unit comprises about 5 mg to about 25 mg extraction from aerial parts of an oat plant (e.g., oat straw). In some embodiments, each unit comprises about 2.5 mg to about 10 mg sclareolides (e.g., sage leaf powder). In some embodiments, each unit comprises about 2.5 mg to about 10 mg diindolylmethane (DIM). In some embodiments, each unit comprises about 2.5 mg to about 15 mg zinc. In some embodiments, the fourth composition is RH2. In some embodiments, the person is a female. In some embodiments, one unit of the fourth composition is taken once a day. In some embodiments, one unit of the second composition is taken once a day, and one unit of the fourth composition is taken once a day.

In some embodiments, the method comprises the person using one or more units of a fifth composition. In some embodiments, each unit comprises about 125 mg to about 525 mg of the composition, about 225 mg to about 425 mg, about 275 mg to about 375 mg, or about 325 mg, about 125 mg, about 150 mg, about 175 mg, about 200 mg, about 225 mg, about 250 mg, about 275 mg, about 300 mg, about 350 mg, about 375 mg, about 400 mg, about 425 mg, about 450 mg, about 475 mg, about 500 mg, or about 525 mg. In some embodiments, each unit comprises about 50 mg to about 250 mg of ashwagandha extract. In some embodiments, each unit comprises about 0.1 mg to about 250 mg of one or more excipients (e.g., sodium starch glycolate, one or more flavoring agents, one or more sweetening agents, one or more adsorbents, glidants, and/or silicon dioxide, one or more of binders, diluents, and/or cellulose, one or more starch excipients, magnesium stearate, and/or menthol crystals). In some embodiments, each unit comprises about 10 mg to about 40 mg of chelating agent (e.g., EDTA). In some embodiments, each unit comprises about 10 mg to about 40 mg delivery enhancing agent (e.g., AstraGin®). In some embodiments, each unit comprises about 5 mg to about 25 mg phosphatidylserine. In some embodiments, each unit comprises about 5 mg to about 25 mg compound derived from a magnolia tree (e.g., Magnolia officinalis). In some embodiments, each unit comprises about 2.5 mg to about 20 mg L-theanine. In some embodiments, each unit comprises about 2.5 mg to about 15 mg L-tryptophan. In some embodiments, each unit comprises about 2.5 mg to about 10 mg reishi mushroom. In some embodiments, each unit comprises about 0.5 mg to about 10 mg melatonin. In some embodiments, the fifth composition is RH3. In some embodiments, the person is a female. In some embodiments, one unit of the fifth composition is taken once a day. In some embodiments, one unit of the second composition is taken once a day, one unit of the fourth composition is taken once a day, and one unit of the fifth composition is taken once a day. In some embodiments, the person is a male. In some embodiments, one unit of the fifth composition is taken once a day. In some embodiments, one unit of the first composition is taken once a day, one unit of the third composition is taken once a day, and one unit of the fifth composition is taken once a day.

In some embodiments, the method comprises the person using more than one unit of an ashwagandha extract composition herein. Subsequent units may have the same amount, less than, or greater than the amount of ashwagandha extract as the first unit. A subsequent unit may be taken about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the first unit or another previous unit. In a non-limiting example a person may take a unit daily. In a further nonlimiting example a person may take two units daily. A subsequent unit may be taken about 1 or 2 weeks after the previous unit. The one or more units may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 units. In a non-limiting example, the composition is taken in about 7 daily units. In some embodiments, a unit may be a pill, tablet, lozenge, or the like. In some embodiments, a unit may be more than one pills, tablets, lozenges, or the like, taken together, e.g., a unit comprises taking two pills in the morning, etc.

In some embodiments, the method comprises use of one or more compositions by a person at one or more times in a day. In some cases, one of the one or more times is within about the first hour of the day. In some embodiments, the first hour of the day is the first hour of the day after the person has woken up. In other embodiments, the first hour of the day is a specified hour of the day (e.g., 12:00 AM to 1:00 AM or 6:00 AM to 7:00 AM) that does not vary based on when the person wakes up. Other times of the one or more times are within about the second hour of the day, within about the third hour of the day, within about the fourth hour of the day, within about the fifth hour of the day, within about the sixth hour of the day, within about the seventh hour of the day, within about the eighth hour of the day, within about the ninth hour of the day, within about the tenth hour of the day, within about the eleventh hour of the day, or within about the twelfth hour of the day. In a nonlimiting example embodiment, a unit of the composition may be taken at a time within about the first 12 hours of the day of the person. The composition taken within about the first 12 hours of the day may be the first composition or the second composition described above, and an example product associated with the first composition or the second composition may be called RH1. In some cases, one of the one or more times is within about the thirteenth hour of the day, within about the fourteenth hour of the day, within about the fifteenth hour of the day, within about the sixteenth hour of the day, within about the seventeenth hour of the day, within about the eighteenth hour of the day, within about the nineteenth hour of the day, within about the twentieth hour of the day, within about the twenty-first hour of the day, within about the twenty-second hour of the day, within about the twenty-third hour of the day, or within about the twenty-fourth hour of the day. In a nonlimiting example embodiment, a unit of the composition is taken at a time between the thirteenth hour of the day and the twenty-fourth hour of the day. The composition taken between the thirteenth hour of the day and the twenty-fourth hour of the day may be the third composition or the fourth composition described above, and an example product associated with the third composition or the fourth composition may be called RH2. In some cases, one of the one or more times is within about two hours of the person falling asleep within about one hour and 45 minutes of the person falling asleep, within about one hour and 30 minutes of the person falling asleep, within about one hour and 15 minutes of the person falling asleep, within about one hour of the person falling asleep, within about 45 minutes of the person falling asleep, within about 30 minutes of the person falling asleep, or within about 15 minutes of the person falling asleep. In a nonlimiting example embodiment, the composition is used within about 30 minutes prior to the person falling asleep. The composition taken before the person falling asleep may be the fifth composition described above, and an example product associated with the fifth composition may be called RH3.

In one aspect, the method comprises the receiving information about the person to a system. In some embodiments, the received information may be input by the person to a user interface. In some embodiments, the method includes making a recommendation for a composition based on analysis of the received information of the person. The received information may include data regarding the health, lifestyle, activities, or personal information of the person. The data may be input or provided by the person. In some embodiments, the method includes analyzing the received information and providing a composition recommendation to the person based on the received information and one or more factors determined based on the received information. In some embodiments, recommendations are associated with modulating the cortisol levels of the person. Non-limiting examples of the one or more factors include age of the person, daily sleep of the person, daily exercise of the person, cortisol levels of the person measured in the morning, cortisol levels of the person measured at midday, cortisol levels of the person measured in the evening, cortisol levels of the person measured at night, diet of the person, libido level of the person, mental clarity of the person, fatigue levels of the person, mood of the person, level of energy of the person during exercise, and daily stress levels of the person.

In some embodiments, the system is used one or more times by the person. In some embodiments, the person repeats use of the system about every week, about every two weeks, about every three weeks, about every 4 weeks, about every 5 weeks, about every 6 weeks, about every 7 weeks, about every 8 weeks, about every month, about every two months, about every 3 months, about every 4 months, about every 5 months, about every 6 months, about every year, about every 1.5 years, about every 2 years, about every 2.5 years, or about every 3 years. In a nonlimiting embodiment, the person repeats use of the system about every month, about every 2 months, about every 3 months, or about every 6 months. In some embodiments, the person provides up-to-date data about the person to the system. Non-limiting examples of information received by the system includes the age of the person, sleep data of the person, diet of the person, libido level of the person, mental clarity of the person, fatigue levels of the person, mood of the person, daily exercise of the person, level of energy of the person during exercise, daily stress levels of the person, measured cortisol levels of the person in the morning, measured cortisol levels of the person at midday, measured cortisol levels of the person in the evening, and measured cortisol levels of the person at night. In some embodiments, recommended regimens may change based on new incoming information received by the system.

In some embodiments, the method comprises taking the first unit of the composition at a first time and a second unit of the compound at a second time. In some cases, the second time is about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the first time. In some cases, the second time point is about 1 week after the first time. The second unit may comprise the same amount or a different amount of composition as the first unit. In some cases, the second unit comprises about 350 mg to about 2500 mg of the composition. In some cases, the second unit comprises about 100 mg to about 500 mg of the composition. In example embodiments, the second unit comprises the same unit as the first unit of the composition, taken the day after the first unit.

In some embodiments, the method comprises taking a third unit of the composition at a third time. In some cases, the third time is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the second time point. In some cases, the third time is about 1 or 2 weeks after the second time point. The third unit may comprise the same amount or a different amount of composition as the first unit and/or the second unit. In some cases, the third unit comprises about 350 mg to about 2500 mg of the composition. In some cases, the second unit comprises about 100 mg to about 500 mg of the composition. In example embodiments, the third unit comprises the same unit as the second unit of the composition, taken the day after the second unit.

In some embodiments, the method comprises taking a fourth unit of the composition at a fourth time. In some cases, the fourth time is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the third time. In some cases, the fourth time point is about 1 or 2 weeks after the third time. The fourth unit may comprise the same amount or a different amount of composition as the first unit, the second unit, and/or the third unit. In some cases, the fourth unit comprises about 350 mg to about 2500 mg of the composition. In some cases, the second unit comprises about 100 mg to about 500 mg of the composition. In example embodiments, the fourth unit comprises the same unit as the third unit of the composition, taken the day after the third unit.

In some embodiments, the method comprises taking a fifth unit of the composition at a fifth time. In some cases, the fifth time is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the fourth time. In some cases, the fifth time is about 1 or 2 weeks after the fourth time. The fifth unit may comprise the same amount or a different amount of composition as the first unit, the second unit, the third unit, and/or the fourth unit. In some cases, the fifth unit comprises about 350 mg to about 2500 mg of the composition. In some cases, the second unit comprises about 100 mg to about 500 mg of the composition. In example embodiments, the fifth unit comprises the same unit as the fourth unit of the composition, taken the day after the fourth unit.

In one aspect, the method comprises taking a first unit on a first day, a second unit on a second day, a third unit on a third day, a fourth unit on a fourth day, a fifth unit on a fifth day, a sixth unit on a sixth day, and a seventh unit on a seventh day. In some embodiments this regimen is may be repeated each time period of a number of time periods, e.g., every week, every other week, or every two weeks. For instance, on day 8, the first unit is taken, on day 9 the second unit is taken, and so on.

In one aspect, the method comprises taking a first unit and second unit on a first day, a third unit and fourth unit on a second day, a fifth unit and sixth unit on a third day, a seventh unit and eighth unit on a fourth day, a ninth unit and tenth unit on a fifth day, an eleventh unit and twelfth unit on a sixth day, and a thirteenth unit and fourteenth unit on a seventh day. In some embodiments this regimen may be repeated each time period of a number of time periods, e.g., every week, every other week, or every two weeks. For instance, on an eight day, the first and second unit is taken, on a ninth day the third and fourth unit is taken, and so on.

### Non-Limiting Numbered Embodiments

Embodiment 1. A composition comprising an ashwagandha extract and an excipient.
Embodiment 2. The composition of embodiment 1, wherein the excipient is a penetration enhancer.
Embodiment 3. The composition of embodiment 1 or embodiment 2, wherein the excipient comprises a delivery enhancing agent.
Embodiment 4. The composition of any one of embodiments 1-3, wherein the excipient comprises an extract of Panax notoginseng and an extract of Astragalus membranaceus.
Embodiment 5. The composition of any one of embodiments 1-4, wherein the excipient is present in the composition from about 1% to about 10% (w/w), about 3% to about 8% (w/w/), or from about 5% to about 6% (w/w).
Embodiment 6. The composition of any one of embodiments 1-4, wherein the excipient is present in the composition at about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w).
Embodiment 7. The composition of any one of embodiments 1-4, wherein the excipient is present in the composition from about 1% to about 10% (w/w), about 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 3% to about 10%, about 3% to about 9%, about 3% to about 8%, about 3% to about 7%, about 3% to about 6%, about 3% to about 5%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or from about 5% to about 6% (w/w).
Embodiment 8. The composition of any one of embodiments 1-7, wherein about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 75 mg, about 50 mg to about 70 mg, about 50 mg to about 65 mg, about 50 mg to about 60 mg, or about 50 mg to about 55 mg of the excipient is present in the composition.
Embodiment 9. The composition of any one of embodiments 1-7, wherein about 25 mg, about 30 mg, about 35 mg, about 40 mg, about 45 mg, about 50 mg, about 55 mg, about 60 mg, about 65 mg, about 70 mg, or about 75 mg of the excipient is present in the composition.
Embodiment 10. The composition of any one of embodiments 1-4, wherein the excipient is present in the composition from about 5% to about 15% (w/w), about 7% to about 13% (w/w/), or from about 9% to about 11% (w/w).
Embodiment 11. The composition of any one of embodiments 1-4, wherein the excipient is present in the composition at about 5% (w/w), about 6%, about 7%, about 8%, about 9%, about 10%, about 11%, about 12%, about 13%, about 14%, or about 15% (w/w).
Embodiment 12. The composition of any one of embodiments 1-4, wherein the excipient is present in the composition from about 5% to about 15% (w/w), about 5% to about 14%, about 5% to about 13%, about 5% to about 12%, about 5% to about 11%, about 5% to about 10%, about 6% to about 15%, about 6% to about 14%, about 6% to about 13%, about 6% to about 12%, about 6% to about 11%, about 6% to about 10%, about 7% to about 15%, about 7% to about 14%, about 7% to about 13%, about 7% to about 12%, about 7% to about 11%, about 7% to about 10%, about 8% to about 15%, about 8% to about 14%, about 8% to about 13%, about 8% to about 12%, about 8% to about 11%, about 8% to about 10%, about 9% to about 15%, about 9% to about 14%, about 9% to about 13%, about 9% to about 12%, about 9% to about 11%, or about 9% to about 10% (w/w).
Embodiment 13. The composition of any one of embodiments 1-4 or embodiments 10-12, wherein about 10 mg to about 40 mg, about 10 mg to about 35 mg, about 10 mg to about 30 mg, about 10 mg to about 25 mg, about 10 mg to about 20 mg, about 15 mg to about 40 mg, about 15 mg to about 35 mg, about 15 mg to about 30 mg, about 15 mg to about 25 mg, about 15 mg to about 20 mg, about 20 mg to about 40 mg, about 20 mg to about 35 mg, about 20 mg to about 30 mg, about 20 mg to about 25 mg, about 25 mg to about 40 mg, about 25 mg to about 35 mg, or about 25 mg to about 30 mg of the excipient is present in the composition.
Embodiment 14. The composition of any one of embodiments 1-4 or embodiments 10-12, wherein about 10 mg, about 15 mg, about 20 mg, about 25 mg, about 30 mg, about 35 mg, or about 40 mg of the excipient is present in the composition.
Embodiment 15. The composition of any one of embodiments 1-14, further comprising a chelating agent.
Embodiment 16. A composition comprising an ashwagandha extract and a chelating agent.
Embodiment 17. The composition of embodiment 15 or embodiment 16, wherein the chelating agent enhances the action of antibacterial components and stabilizes the action of antioxidants.
Embodiment 18. The composition of any one of embodiments 15-17, wherein the chelating agent comprises deferoxamine, deferasirox, deferiprone, penicillamine, trientine, dimercaprol, ethylenediamine, porphine, heme, sodium gluconate, tetrasodium glutamate diacetate, phytic acid, ethylenediaminetetraacetic acid (EDTA), or a combination of two or more thereof.
Embodiment 19. The composition of embodiment 18, wherein the chelating agent comprises ethylenediaminetetraacetic acid (EDTA).
Embodiment 20. The composition of any one of embodiments 15-19, wherein the chelating agent is present in the composition from about 1% to about 10% (w/w), about 3% to about 8% (w/w/), or from about 5% to about 6% (w/w).
Embodiment 21. The composition of any one of embodiments 15-19, wherein the chelating agent is present in the composition at about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w).
Embodiment 22. The composition of any one of embodiments 15-19, wherein the chelating agent is present in the composition from about 1% to about 10% (w/w), about 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 3% to about 10%, about 3% to about 9%, about 3% to about 8%, about 3% to about 7%, about 3% to about 6%, about 3% to about 5%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or from about 5% to about 6% (w/w).
Embodiment 23. The composition of any one of embodiments 15-22, wherein about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 75 mg, about 50 mg to about 70 mg, about 50 mg to about 65 mg, about 50 mg to about 60 mg, or about 50 mg to about 55 mg of the chelating agent is present in the composition.
Embodiment 24. The composition of any one of embodiments 15-22, wherein about 25 mg, about 30 mg, about 35 mg, about 40 mg, about 45 mg, about 50 mg, about 55 mg, about 60 mg, about 65 mg, about 70 mg, or about 75 mg of the chelating agent is present in the composition.
Embodiment 25. The composition of any one of embodiments 15-19, wherein the chelating agent is present in the composition from about 1% to about 19% (w/w), about 5% to about 15% (w/w), about 7% to about 13% (w/w/), or from about 9% to about 11% (w/w).
Embodiment 26. The composition of any one of embodiments 15-19, wherein the chelating agent is present in the composition at about 1% (w/w), about 2% (w/w), about 3% (w/w), about 4% (w/w), 5% (w/w), about 6% (w/w), about 7% (w/w), about 8% (w/w), about 9% (w/w), about 10% (w/w), about 11% (w/w), about 12% (w/w), about 13% (w/w), about 14% (w/w), or about 15% (w/w).
Embodiment 27. The composition of any one of embodiments 15-19, wherein the chelating agent is present in the composition from about 5% to about 15% (w/w), about 5% to about 14%, about 5% to about 13%, about 5% to about 12%, about 5% to about 11%, about 5% to about 10%, about 6% to about 15%, about 6% to about 14%, about 6% to about 13%, about 6% to about 12%, about 6% to about 11%, about 6% to about 10%, about 7% to about 15%, about 7% to about 14%, about 7% to about 13%, about 7% to about 12%, about 7% to about 11%, about 7% to about 10%, about 8% to about 15%, about 8% to about 14%, about 8% to about 13%, about 8% to about 12%, about 8% to about 11%, about 8% to about 10%, about 9% to about 15%, about 9% to about 14%, about 9% to about 13%, about 9% to about 12%, about 9% to about 11%, or about 9% to about 10% (w/w).
Embodiment 28. The composition of any one of embodiments 15-19 or embodiments 25-27, wherein about 10 mg to about 50 mg, about 10 mg to about 45 mg, 10 mg to about 40 mg, about 10 mg to about 35 mg, about 10 mg to about 30 mg, about 10 mg to about 25 mg, about 10 mg to about 20 mg, about 15 mg to about 40 mg, about 15 mg to about 35 mg, about 15 mg to about 30 mg, about 15 mg to about 25 mg, about 15 mg to about 20 mg, about 20 mg to about 40 mg, about 20 mg to about 35 mg, about 20 mg to about 30 mg, about 20 mg to about 25 mg, about 25 mg to about 50 mg, about 25 mg to about 45 mg, about 25 mg to about 40 mg, about 25 mg to about 35 mg, or about 25 mg to about 30 mg of the chelating agent is present in the composition.
Embodiment 29. The composition of any one of embodiments 15-19 or embodiments 25-27, wherein about 10 mg, about 15 mg, about 20 mg, about 25 mg, about 30 mg, about 35 mg, or about 40 mg of the chelating agent is present in the composition.
Embodiment 30. The composition of any one of embodiments 1-29, further comprising a delivery enhancing agent.
Embodiment 31. A composition comprising an ashwagandha extract and a delivery enhancing agent.
Embodiment 32. The composition of embodiment 30 or embodiment 31, wherein the delivery enhancing agent improves the transportation and uptake of nutrients.
Embodiment 33. The composition of any one of embodiments 30-32, wherein the delivery enhancing agent comprises AstraGin®.
Embodiment 34. The composition of any one of embodiments 30-33, wherein the delivery enhancing agent is present in the composition from about 1% to about 10% (w/w), about 3% to about 8% (w/w), about 4% to about 7% (w/w), or from about 5% to about 6% (w/w).
Embodiment 35. The composition of any one of embodiments 30-33, wherein the delivery enhancing agent is present in the composition at about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w).
Embodiment 36. The composition of any one of embodiments 30-33, wherein the delivery enhancing agent is present in the composition from about 1% to about 10% (w/w), about 1% to about 9%, about 1% to about 8%, about 1% to about 7%, about 1% to about 6%, about 1% to about 5%, about 2% to about 10%, about 2% to about 9%, about 2% to about 8%, about 2% to about 7%, about 2% to about 6%, about 2% to about 5%, about 3% to about 10%, about 3% to about 9%, about 3% to about 8%, about 3% to about 7%, about 3% to about 6%, about 3% to about 5%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or from about 5% to about 6% (w/w).
Embodiment 37. The composition of any one of embodiments 30-36, wherein about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 75 mg, about 50 mg to about 70 mg, about 50 mg to about 65 mg, about 50 mg to about 60 mg, or about 50 mg to about 55 mg of the delivery enhancing agent is present in the composition.
Embodiment 38. The composition of any one of embodiments 30-36, wherein about 25 mg, about 30 mg, about 35 mg, about 40 mg, about 45 mg, about 50 mg, about 55 mg, about 60 mg, about 65 mg, about 70 mg, or about 75 mg of the delivery enhancing agent is present in the composition.
Embodiment 39. The composition of any one of embodiments 30-33, wherein the delivery enhancing agent is present in the composition from about 1% to about 19% (w/w), about 5% to about 15% (w/w), about 7% to about 13% (w/w), or from about 9% to about 11% (w/w).
Embodiment 40. The composition of any one of embodiments 30-33, wherein the delivery enhancing agent is present in the composition at about 1%, about 2%, about 3%, about 4%, about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), or about 15% (w/w).
Embodiment 41. The composition of any one of embodiments 30-33, wherein the delivery enhancing agent is present in the composition from about 5% to about 15% (w/w), about 5% to about 14%, about 5% to about 13%, about 5% to about 12%, about 5% to about 11%, about 5% to about 10%, about 6% to about 15%, about 6% to about 14%, about 6% to about 13%, about 6% to about 12%, about 6% to about 11%, about 6% to about 10%, about 7% to about 15%, about 7% to about 14%, about 7% to about 13%, about 7% to about 12%, about 7% to about 11%, about 7% to about 10%, about 8% to about 15%, about 8% to about 14%, about 8% to about 13%, about 8% to about 12%, about 8% to about 11%, about 8% to about 10%, about 9% to about 15%, about 9% to about 14%, about 9% to about 13%, about 9% to about 12%, about 9% to about 11%, or about 9% to about 10% (w/w).
Embodiment 42. The composition of any one of embodiments 30-33 or embodiments 39-41, wherein about 10 mg to about 50 mg, about 10 mg to about 45 mg, 10 mg to about 40 mg, about 10 mg to about 35 mg, about 10 mg to about 30 mg, about 10 mg to about 25 mg, about 10 mg to about 20 mg, about 15 mg to about 40 mg, about 15 mg to about 35 mg, about 15 mg to about 30 mg, about 15 mg to about 25 mg, about 15 mg to about 20 mg, about 20 mg to about 40 mg, about 20 mg to about 35 mg, about 20 mg to about 30 mg, about 20 mg to about 25 mg, about 25 mg to about 50 mg, about 25 mg to about 45 mg, about 25 mg to about 40 mg, about 25 mg to about 35 mg, or about 25 mg to about 30 mg of the delivery enhancing agent is present in the composition.
Embodiment 43. The composition of any one of embodiments 30-33 or embodiments 39-41, wherein about 10 mg, about 15 mg, about 20 mg, about 25 mg, about 30 mg, about 35 mg, or about 40 mg of the delivery enhancing agent is present in the composition.
Embodiment 44. The composition of any one of embodiments 1-43, further comprising a cordyceps species.
Embodiment 45. The composition of embodiment 44, wherein the cordyceps species has anti-inflammatory, antioxidative, anticancer, anti-obesity, and antidiabetic effects.
Embodiment 46. The composition of embodiment 44 or embodiment 45, wherein the cordyceps species comprises CordycepsPrime™.
Embodiment 47. The composition of any one of embodiments 44-46, wherein the cordyceps species is present in the composition from about 5% to about 35% (w/w), about 10% to about 30% (w/w), or about 15% to about 25% (w/w).
Embodiment 48. The composition of any one of embodiments 44-46, wherein the cordyceps species is present in the composition at about 5%, about 10%, about 15%, about 20%, about 25%, about 30%, or about 35% (w/w).
Embodiment 49. The composition of any one of embodiments 44-46, wherein the cordyceps species is present in the composition from about 5% to about 35% (w/w), about 5% to about 30%, about 5% to about 25%, about 5% to about 20%, about 5% to about 15%, about 5% to about 10%, about 10% to about 35%, about 10% to about 30%, about 10% to about 25%, about 10% to about 20%, about 10% to about 15%, about 15% to about 35%, about 15% to about 30%, about 15% to about 25%, about 15% to about 20%, about 20% to about 35%, about 20% to about 30%, about 20% to about 25%, about 25% to about 35%, about 25% to about 30%, or from about 30% to about 35% (w/w).
Embodiment 50. The composition of any one of embodiments 44-49, wherein about 100 mg to about 300 mg, about 100 mg to about 250 mg, about 100 mg to about 200 mg, about 100 mg to about 150 mg, about 150 mg to about 300 mg, about 150 mg to about 250 mg, about 150 mg to about 200 mg, about 200 mg to about 300 mg, about 200 mg to about 250 mg, or about 250 mg to about 300 mg of the cordyceps species is present in the composition.
Embodiment 51. The composition of any one of embodiments 44-49, wherein about 100 mg, about 150 mg, about 200 mg, about 250 mg, or about 300 mg of the cordyceps species is present in the composition.
Embodiment 52. The composition of any one of embodiments 1-51, further comprising phosphatidylserine.
Embodiment 53. The composition of embodiment 52, wherein the phosphatidylserine has memory enhancing properties.
Embodiment 54. The composition of embodiment 52 or embodiment 53, wherein the phosphatidylserine is present in the composition from about 5% to about 25% (w/w), about 8% to about 22% (w/w), about 11% to about 19% (w/w), about 13% to about 17% (w/w), or about 15% (w/w).
Embodiment 55. The composition of embodiment 52 or embodiment 53, wherein the phosphatidylserine is present in the composition at about 5% (w/w), about 10% (w/w), about 15% (w/w), about 20% (w/w), or about 25% (w/w).
Embodiment 56. The composition of embodiment 52 or embodiment 53, wherein the phosphatidylserine is present in the composition from about 5% to about 25% (w/w), about 5% (w/w) to about 20% (w/w), about 5% (w/w) to about 15% (w/w), about 5% (w/w) to about 10% (w/w), about 10% (w/w) to about 25% (w/w), about 10% (w/w) to about 20% (w/w), about 10% (w/w) to about 15% (w/w), about 15% (w/w) to about 25% (w/w), about 15% (w/w) to about 20% (w/w), or from about 20% (w/w) to about 25% (w/w).
Embodiment 57. The composition of any one of embodiments 54-56, wherein about 25 mg to about 225 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 225 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 225 mg, about 75 mg to about 200 mg, about 75 mg to about 175 mg, about 75 mg to about 150 mg, about 75 mg to about 125 mg, about 75 mg to about 100 mg, about 100 mg to about 225 mg, about 100 mg to about 200 mg, about 100 mg to about 175 mg, about 100 mg to about 150 mg, about 100 mg to about 125 mg, about 125 mg to about 225 mg, about 125 mg to about 200 mg, about 125 to about 175 mg, about 125 mg to about 150 mg, about 150 mg to about 225 mg, about 150 mg to about 200 mg, about 150 mg to about 175 mg, about 175 mg to about 225 mg, about 175 mg to about 200 mg, or about 200 mg to about 225 mg of phosphatidylserine is present in the composition.
Embodiment 58. The composition of any one of embodiments 54-56, wherein about 25 mg, about 50 mg, about 75 mg, about 100 mg, about 125 mg, about 150 mg, about 175 mg, about 200 mg, or about 225 mg of phosphatidylserine is present in the composition.
Embodiment 59. The composition of any one of embodiments 1-58 further comprising a supplemental mineral.
Embodiment 60. The composition of embodiment 59, wherein the supplemental mineral is essential for maintaining normal organ and cellular function.
Embodiment 61. The composition of embodiment 59 or embodiment 60, wherein the supplemental mineral comprises magnesium, magnesium glycinate, calcium, choline, chromium, cesium, copper, fluoride, iodine, iron, manganese, molybdenum, potassium, selenium, zinc, or a combination of two or more thereof.
Embodiment 62. The composition of embodiment 61, wherein the supplemental mineral comprises magnesium glycinate.
Embodiment 63. The composition of any one of embodiments 59-62, wherein the supplemental mineral is present in the composition from about 0.5% to about 11%, about 2.5% to about 9%, or about 4.5% to about 7% (w/w).
Embodiment 64. The composition of any one of embodiments 59-62, wherein the supplemental mineral is present in the composition at about 0.5% (w/w), about 1.5% (w/w), about 2.5% (w/w), about 3.5% (w/w), about 4.5% (w/w), about 5.5% (w/w), about 6.5% (w/w), about 7.5% (w/w), about 8.5% (w/w), about 9.5% (w/w), or about 10.5% (w/w).
Embodiment 65. The composition of any one of embodiments 59-62, wherein the supplemental mineral is present in the composition from about 0.5% to about 11%, about 0.5% (w/w) to about 10% (w/w), about 0.5% (w/w) to about 9% (w/w), about 0.5% (w/w) to about 8% (w/w), about 0.5% (w/w) to about 7% (w/w), about 0.5% (w/w) to about 6% (w/w), about 0.5% (w/w) to about 5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1.5% (w/w) to about 11% (w/w), about 1.5% (w/w) to about 10% (w/w), about 1.5% (w/w) to about 9% (w/w), about 1.5% (w/w) to about 8% (w/w), about 1.5% (w/w) to about 7% (w/w), about 1.5% (w/w) to about 6% (w/w), about 1.5% (w/w) to about 5% (w/w), about 1.5% (w/w) to about 4% (w/w), about 1.5% (w/w) to about 3% (w/w), about 1.5% (w/w) to about 2% (w/w), about 2.5% (w/w) to about 11% (w/w), about 2.5% (w/w) to about 10% (w/w), about 2.5% (w/w) to about 9% (w/w), about 2.5% (w/w) to about 8% (w/w), about 2.5% (w/w) to about 7% (w/w), about 2.5% (w/w) to about 6% (w/w), about 2.5% (w/w) to about 5% (w/w), about 2.5% (w/w) to about 4% (w/w), about 2.5% (w/w) to about 3% (w/w), about 3.5% (w/w) to about 11% (w/w), about 3.5% (w/w) to about 10% (w/w), about 3.5% (w/w) to about 9% (w/w), about 3.5% (w/w) to about 8% (w/w), about 3.5% (w/w) to about 7% (w/w), about 3.5% (w/w) to about 6% (w/w), about 3.5% (w/w) to about 5% (w/w), about 3.5% (w/w) to about 4% (w/w), about 4.5% (w/w) to about 11% (w/w), about 4.5% (w/w) to about 10% (w/w), about 4.5% (w/w) to about 9% (w/w), about 4.5% (w/w) to about 8% (w/w), about 4.5% (w/w) to about 7% (w/w), about 4.5% (w/w) to about 6% (w/w), about 4.5% (w/w) to about 5% (w/w), about 5.5% (w/w) to about 11% (w/w), about 5.5% (w/w) to about 10% (w/w), about 5.5% (w/w) to about 9% (w/w), about 5.5% (w/w) to about 8% (w/w), about 5.5% (w/w) to about 7% (w/w), about 5.5% (w/w) to about 6% (w/w), about 6.5% (w/w) to about 11% (w/w), about 6.5% (w/w) to about 10% (w/w), about 6.5% (w/w) to about 9% (w/w), about 6.5% (w/w) to about 8% (w/w), about 6.5% (w/w) to about 7% (w/w), about 7.5% (w/w) to about 11% (w/w), about 7.5% (w/w) to about 10% (w/w), about 7.5% (w/w) to about 9% (w/w), about 7.5% (w/w) to about 8% (w/w), about 8.5% (w/w) to about 11% (w/w), about 8.5% (w/w) to about 10% (w/w), about 8.5% (w/w) to about 9% (w/w), about 9.5% (w/w) to about 11% (w/w), about 9.5% (w/w) to about 10% (w/w), or from about 10.5% (w/w) to about 11% (w/w).
Embodiment 66. The composition of any one of embodiments 59-66, wherein about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, 5 mg to about 10 mg, 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 90 mg, about 20 mg to about 80 mg, about 20 mg to about 70 mg, about 20 mg to about 60 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg of the supplemental mineral is present in the composition.
Embodiment 67. The composition of any one of embodiments 59-66, wherein about 5 mg, about 10 mg, about 20 mg, about 30 mg, about 40 mg, about 50 mg, about 60 mg, about 70 mg, about 80 mg, or about 90 mg of the supplemental mineral is present in the composition.
Embodiment 68. The composition of any one of embodiments 1-67 further comprising a compound derived from a magnolia tree.
Embodiment 69. The composition of embodiment 68, wherein the compound derived from the magnolia tree reduces inflammation and stress.
Embodiment 70. The composition of embodiment 68 or embodiment 69, wherein the compound derived from the magnolia tree is Magnolia officinalis.
Embodiment 71. The composition of any one of embodiments 68-70, wherein the compound derived from the magnolia tree is present in the composition from about 0.5% to about 11%, about 2.5% to about 9%, or about 4.5% to about 7% (w/w).
Embodiment 72. The composition of any one of embodiments 68-70, wherein the compound derived from the magnolia tree is present in the composition at about 0.5% (w/w), about 1.5% (w/w), about 2.5% (w/w), about 3.5% (w/w), about 4.5% (w/w), about 5.5% (w/w), about 6.5% (w/w), about 7.5% (w/w), about 8.5% (w/w), about 9.5% (w/w), or about 10.5% (w/w).
Embodiment 73. The composition of any one of embodiments 68-70, wherein the compound derived from the magnolia tree is present in the composition from about 0.5% to about 11%, about 0.5% (w/w) to about 10% (w/w), about 0.5% (w/w) to about 9% (w/w), about 0.5% (w/w) to about 8% (w/w), about 0.5% (w/w) to about 7% (w/w), about 0.5% (w/w) to about 6% (w/w), about 0.5% (w/w) to about 5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1.5% (w/w) to about 11% (w/w), about 1.5% (w/w) to about 10% (w/w), about 1.5% (w/w) to about 9% (w/w), about 1.5% (w/w) to about 8% (w/w), about 1.5% (w/w) to about 7% (w/w), about 1.5% (w/w) to about 6% (w/w), about 1.5% (w/w) to about 5% (w/w), about 1.5% (w/w) to about 4% (w/w), about 1.5% (w/w) to about 3% (w/w), about 1.5% (w/w) to about 2% (w/w), about 2.5% (w/w) to about 11% (w/w), about 2.5% (w/w) to about 10% (w/w), about 2.5% (w/w) to about 9% (w/w), about 2.5% (w/w) to about 8% (w/w), about 2.5% (w/w) to about 7% (w/w), about 2.5% (w/w) to about 6% (w/w), about 2.5% (w/w) to about 5% (w/w), about 2.5% (w/w) to about 4% (w/w), about 2.5% (w/w) to about 3% (w/w), about 3.5% (w/w) to about 11% (w/w), about 3.5% (w/w) to about 10% (w/w), about 3.5% (w/w) to about 9% (w/w), about 3.5% (w/w) to about 8% (w/w), about 3.5% (w/w) to about 7% (w/w), about 3.5% (w/w) to about 6% (w/w), about 3.5% (w/w) to about 5% (w/w), about 3.5% (w/w) to about 4% (w/w), about 4.5% (w/w) to about 11% (w/w), about 4.5% (w/w) to about 10% (w/w), about 4.5% (w/w) to about 9% (w/w), about 4.5% (w/w) to about 8% (w/w), about 4.5% (w/w) to about 7% (w/w), about 4.5% (w/w) to about 6% (w/w), about 4.5% (w/w) to about 5% (w/w), about 5.5% (w/w) to about 11% (w/w), about 5.5% (w/w) to about 10% (w/w), about 5.5% (w/w) to about 9% (w/w), about 5.5% (w/w) to about 8% (w/w), about 5.5% (w/w) to about 7% (w/w), about 5.5% (w/w) to about 6% (w/w), about 6.5% (w/w) to about 11% (w/w), about 6.5% (w/w) to about 10% (w/w), about 6.5% (w/w) to about 9% (w/w), about 6.5% (w/w) to about 8% (w/w), about 6.5% (w/w) to about 7% (w/w), about 7.5% (w/w) to about 11% (w/w), about 7.5% (w/w) to about 10% (w/w), about 7.5% (w/w) to about 9% (w/w), about 7.5% (w/w) to about 8% (w/w), about 8.5% (w/w) to about 11% (w/w), about 8.5% (w/w) to about 10% (w/w), about 8.5% (w/w) to about 9% (w/w), about 9.5% (w/w) to about 11% (w/w), about 9.5% (w/w) to about 10% (w/w), or from about 10.5% (w/w) to about 11% (w/w).
Embodiment 74. The composition of any one of embodiments 68-73, wherein about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 90 mg, about 20 mg to about 80 mg, about 20 mg to about 70 mg, about 20 mg to about 60 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg of the compound derived from the magnolia tree is present in the composition.
Embodiment 75. The composition of any one of embodiments 59-66, wherein about 10 mg, about 20 mg, about 30 mg, about 40 mg, about 50 mg, about 60 mg, about 70 mg, about 80 mg, or about 90 mg of the compound derived from the magnolia tree is present in the composition.
Embodiment 76. The composition of any one of embodiments 1-75, further comprising ginger.
Embodiment 77. The composition of embodiment 76, wherein the ginger has antioxidant, antimicrobial, and anti-neuroinflammation bioactivities.
Embodiment 78. The composition of embodiment 76 or embodiment 77, wherein the ginger is present in the composition from about 0.1% (w/w) to about 2.5% (w/w), about 0.5% (w/w) to about 2.1% (w/w), or from about 0.9% (w/w) to about 1.7% (w/w).
Embodiment 79. The composition of embodiment 76 or embodiment 77, wherein the ginger is present in the composition at about 0.1%, about 0.5%, about 0.9%, about 1.3%, about 1.7%, about 2.1%, or about 2.5% (w/w).
Embodiment 80. The composition of embodiment 76 or embodiment 77, wherein the ginger is present in the composition from about 0.1% to about 2.5%, about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, or from about 2.1% to about 2.5%.
Embodiment 81. The composition of any one of embodiments 76-80, wherein about 1 mg to about 25 mg, about 1 mg to about 21 mg, about 1 mg to about 17 mg, about 1 mg to about 13 mg, about 1 mg to about 9 mg, about 1 mg to about 5 mg, about 5 mg to about 25 mg, about 5 mg to about 21 mg, about 5 mg to about 17 mg, about 5 mg to about 13 mg, about 5 mg to about 9 mg, about 9 mg to about 25 mg, about 9 mg to about 21 mg, about 9 mg to about 17 mg, about 9 mg to about 13 mg, about 13 mg to about 25 mg, about 13 mg to about 21 mg, about 13 mg to about 17 mg, about 17 mg to about 25 mg, or about 21 mg to about 25 mg of the ginger is present in the composition.
Embodiment 82. The composition of any one of embodiments 76-80, wherein about 1 mg, about 5 mg, about 9 mg, about 13 mg, about 17 mg, about 21 mg, or about 25 mg of the ginger is present in the composition.
Embodiment 83. The composition of any one of embodiments 1-82, further comprising horny goat weed.
Embodiment 84. The composition of embodiment 83, wherein the horny goat weed is used to combat diseases of the eye and kidney, impotence, arthritis, asthma, and hypertension.
Embodiment 85. The composition of embodiment 83 or embodiment 84, wherein the horny goat weed is present in the composition from about 0.1% (w/w) to about 2.5% (w/w), about 0.5% (w/w) to about 2.1% (w/w), or from about 0.9% (w/w) to about 1.7% (w/w).
Embodiment 86. The composition of embodiment 83 or embodiment 84, wherein the horny goat weed is present in the composition at about 0.1%, about 0.5%, about 0.9%, about 1.3%, about 1.7%, about 2.1%, or about 2.5% (w/w).
Embodiment 87. The composition of embodiment 83 or embodiment 84, wherein the horny goat weed is present in the composition from about 0.1% to about 2.5%, about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, or from about 2.1% to about 2.5%.
Embodiment 88. The composition of any one of embodiments 83-87, wherein about 1 mg to about 25 mg, about 1 mg to about 21 mg, about 1 mg to about 17 mg, about 1 mg to about 13 mg, about 1 mg to about 9 mg, about 1 mg to about 5 mg, about 5 mg to about 25 mg, about 5 mg to about 21 mg, about 5 mg to about 17 mg, about 5 mg to about 13 mg, about 5 mg to about 9 mg, about 9 mg to about 25 mg, about 9 mg to about 21 mg, about 9 mg to about 17 mg, about 9 mg to about 13 mg, about 13 mg to about 25 mg, about 13 mg to about 21 mg, about 13 mg to about 17 mg, about 17 mg to about 25 mg, or about 21 mg to about 25 mg of the horny goat weed is present in the composition.
Embodiment 89. The composition of any one of embodiments 83-87, wherein about 1 mg, about 5 mg, about 9 mg, about 13 mg, about 17 mg, about 21 mg, or about 25 mg of the horny goat weed is present in the composition.
Embodiment 90. The composition of any one of embodiments 1-89, further comprising sclareolides.
Embodiment 91. The composition of embodiment 90, wherein the sclareolides is used for depression, dementia, diabetes, obesity, lupus, cancer, cardiovascular disease, or a combination of one or more thereof.
Embodiment 92. The composition of embodiment 90 or embodiment 91, wherein the sclareolides is present in the composition from about 0.1% (w/w) to about 1% (w/w), about 0.3% (w/w) to about 0.8% (w/w), or from about 0.4% (w/w) to about 7% (w/w).
Embodiment 93. The composition of embodiment 90 or embodiment 91, wherein the sclareolides is present in the composition at about 0.1%, about 0.2%, about 0.3%, about 0.4%, about 0.5%, about 0.6%, about 0.7%, about 0.8%, about 0.9%, or about 1% (w/w).
Embodiment 94. The composition of embodiment 90 or embodiment 91, wherein the sclareolides is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1% (w/w).
Embodiment 95. The composition of any one of embodiments 90-94, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, about 9.5 mg to about 10 mg of the sclareolides is present in the composition.
Embodiment 96. The composition of any one of embodiments 90-94, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the sclareolides is present in the composition.
Embodiment 97. The composition of any one of embodiments 1-96, further comprising diindolylmethane (DIM).
Embodiment 98. The composition of embodiment 97, where the DIM has natural anti-inflammatory and anticancer effects.
Embodiment 99. The composition of embodiment 97 or embodiment 98, wherein the DIM is present in the composition from about 0.1% (w/w) to about 1% (w/w), about 0.3% (w/w) to about 0.8% (w/w), or from about 0.4% (w/w) to about 0.7% (w/w).
Embodiment 100. The composition of embodiment 97 or embodiment 98, wherein the DIM is present in the composition at about 0.1%, about 0.2%, about 0.3%, about 0.4%, about 0.5%, about 0.6%, about 0.7%, about 0.8%, about 0.9%, or about 1% (w/w).
Embodiment 101. The composition of embodiment 97 or embodiment 98, wherein the DIM is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1% (w/w).
Embodiment 102. The composition of any one of embodiments 97-101, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, about 9.5 mg to about 10 mg of the DIM is present in the composition.
Embodiment 103. The composition of any one of embodiments 97-101, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the DIM is present in the composition.
Embodiment 104. The composition of any one of embodiments 1-103, further comprising zinc.
Embodiment 105. The composition of embodiment 104, wherein the zinc is important for immune system and metabolism function.
Embodiment 106. The composition of embodiment 104 or embodiment 105, wherein the zinc is present in the composition from about 0.1% (w/w) to about 1% (w/w), about 0.3% (w/w) to about 0.8% (w/w), or from about 0.4% (w/w) to about 0.7% (w/w).
Embodiment 107. The composition of embodiment 104 or embodiment 105, wherein the zinc is present in the composition at about 0.1%, about 0.2%, about 0.3%, about 0.4%, about 0.5%, about 0.6%, about 0.7%, about 0.8%, about 0.9%, or about 1% (w/w).
Embodiment 108. The composition of embodiment 104 or embodiment 105, wherein the zinc is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1% (w/w).
Embodiment 109. The composition of any one of embodiments 104-108, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, about 9.5 mg to about 10 mg of the zinc is present in the composition.
Embodiment 110. The composition of any one of embodiments 104-108, wherein about 0.5 mg, about 1.5 mg, about 2.5 mg, about 3.5 mg, about 4.5 mg, about 5.5 mg, about 6.5 mg, about 7.5 mg, about 8.5 mg, about 9.5 mg, about 10.5 mg, or about 11.5 mg of the zinc is present in the composition.
Embodiment 111. The composition of any one of embodiments 1-82 or embodiments 90-110, further comprising an extraction from the aerial parts of oat plants.
Embodiment 112. The composition of embodiment 111, wherein the extraction has properties that improve cognitive function and improve anxiety.
Embodiment 113. The composition of embodiment 111 or embodiment 112, wherein the extraction is oat straw.
Embodiment 114. The composition of any one of embodiments 111-113, wherein the extract is present in the composition from about 0.1% (w/w) to about 2.5% (w/w), about 0.5% (w/w) to about 2.1% (w/w), or from about 0.9% (w/w) to about 1.7% (w/w).
Embodiment 115. The composition of any one of embodiments 111-113, wherein the extraction is present in the composition at about 0.1%, about 0.5%, about 0.9%, about 1.3%, about 1.7%, about 2.1%, or about 2.5% (w/w).
Embodiment 116. The composition of any one of embodiments 111-113, wherein the extraction is present in the composition from about 0.1% to about 2.5%, about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, or from about 2.1% to about 2.5%.
Embodiment 117. The composition of any one of embodiments 111-116, wherein about 1 mg to about 25 mg, about 1 mg to about 21 mg, about 1 mg to about 17 mg, about 1 mg to about 13 mg, about 1 mg to about 9 mg, about 1 mg to about 5 mg, about 5 mg to about 25 mg, about 5 mg to about 21 mg, about 5 mg to about 17 mg, about 5 mg to about 13 mg, about 5 mg to about 9 mg, about 9 mg to about 25 mg, about 9 mg to about 21 mg, about 9 mg to about 17 mg, about 9 mg to about 13 mg, about 13 mg to about 25 mg, about 13 mg to about 21 mg, about 13 mg to about 17 mg, about 17 mg to about 25 mg, or about 21 mg to about 25 mg of the extraction is present in the composition.
Embodiment 118. The composition of any one of embodiments 111-116, wherein about 1 mg, about 5 mg, about 9 mg, about 13 mg, about 17 mg, about 21 mg, or about 25 mg of the extraction is present in the composition.
Embodiment 119. The composition of any one of embodiments 1-46, further comprising maca.
Embodiment 120. The composition of embodiment 119, wherein the maca is used to enhance fertility and improve libido.
Embodiment 121. The composition of embodiment 119 or embodiment 120, wherein the maca is present in the composition from about 15% (w/w) to about 35% (w/w), about 17% (w/w) to about 31% (w/w), or from about 19% (w/w) to about 27% (w/w).
Embodiment 122. The composition of embodiment 119 or embodiment 120, wherein the maca is present in the composition at about 15%, about 20%, about 25%, about 30%, or about 35% (w/w).
Embodiment 123. The composition of embodiment 119 or embodiment 120, wherein the maca is present in the composition from about 15% to about 35%, about 15% to about 30%, about 15% to about 25%, about 15% to about 20%, about 20% to about 35%, about 20% to about 30%, about 20% to about 25%, about 25% to about 35%, about 25% to about 30%, or from about 30% to about 35%.
Embodiment 124. The composition of any one of embodiments 119-123, wherein about 100 mg to about 400 mg, about 100 mg to about 350 mg, about 100 mg to about 300 mg, about 100 mg to about 250 mg, about 150 mg to about 400 mg, about 150 mg to about 350 mg, about 150 mg to about 300 mg, about 150 mg to about 250 mg, about 200 mg to about 400 mg, about 200 mg to about 350 mg, about 200 mg to about 300 mg, about 200 mg to about 250 mg, about 250 mg to about 400 mg, about 250 mg to about 350 mg, about 250 mg to about 300 mg, about 300 mg to about 400 mg, about 300 mg to about 350 mg, or about 350 mg to about 400 mg of the maca is present in the composition.
Embodiment 125. The composition of any one of embodiments 119-123, wherein about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg of maca is present in the composition.
Embodiment 126. The composition of any one of embodiments 1-46 or embodiments 119-125, further comprising Eurycoma longifolia.
Embodiment 127. The composition of embodiment 126, wherein the Eurycoma longifolia is used to improve sexual health in a male.
Embodiment 128. The composition of embodiment 126 or embodiment 127, wherein the Eurycoma longifolia is present in the composition from about 5% to about 25%, about 7% to about 21%, or from about 9% to about 17% (w/w).
Embodiment 129. The composition of embodiment 126 or embodiment 127, wherein the Eurycoma longifolia is present in the composition at about 5%, about 10%, about 15%, about 20%, or about 25%.
Embodiment 130. The composition of embodiment 126 or embodiment 127, wherein the Eurycoma longifolia is present in the composition from about 5% to about 25%, about 5% to about 20%, about 5% to about 15%, about 5% to about 10%, about 10% to about 25%, about 10% to about 20%, about 10% to about 15%, about 15% to about 25%, about 15% to about 20%, or from about 20% to about 25% (w/w).
Embodiment 131. The composition of any one of embodiments 126-130, wherein about 25 mg to about 275 mg, about 25 mg to about 225 mg, about 25 mg to about 175 mg, about 25 mg to about 125 mg, about 75 mg to about 275 mg, about 75 mg to about 225 mg, about 75 mg to about 175 mg, about 75 mg to about 125 mg, about 100 mg to about 275 mg, about 100 mg to about 225 mg, about 100 mg to about 175 mg, about 100 mg to about 125 mg, about 125 mg to about 275 mg, about 125 mg to about 225 mg, about 125 mg to about 175 mg, about 175 mg to about 275 mg, about 175 mg to about 225 mg, or about 225 mg to about 275 mg of the Eurycoma longifolia is present in the composition.
Embodiment 132. The composition of any one of embodiments 126-130, wherein about 25 mg, about 50 mg, about 75 mg, about 100 mg, about 125 mg, about 150 mg, about 175 mg, about 200 mg, about 225 mg, about 250 mg, or about 275 mg of the Eurycoma longifolia is present in the composition.
Embodiment 133. The composition of any one of embodiments 1-46 or embodiments 119-132, further comprising a fenugreek seed extract.
Embodiment 134. The composition of embodiment 133, wherein the fenugreek seed extract is used to improve metabolism and hormonal profile in a person.
Embodiment 135. The composition of embodiment 133 or embodiment 134, wherein the fenugreek seed extract comprises Testosurge®.
Embodiment 136. The composition of any one of embodiments 133-135, wherein the fenugreek seed extract is present in the composition from about 5% to about 25%, about 7% to about 21%, or from about 9% to about 17% (w/w).
Embodiment 137. The composition of any one of embodiments 133-135, wherein the fenugreek seed extract is present in the composition at about 5%, about 10%, about 15%, about 20%, or about 25%.
Embodiment 138. The composition of any one of embodiments 133-135, wherein the fenugreek seed extract is present in the composition from about 5% to about 25%, about 5% to about 20%, about 5% to about 15%, about 5% to about 10%, about 10% to about 25%, about 10% to about 20%, about 10% to about 15%, about 15% to about 25%, about 15% to about 20%, or from about 20% to about 25% (w/w).
Embodiment 139. The composition of any one of embodiments 133-138, wherein about 25 mg to about 275 mg, about 25 mg to about 225 mg, about 25 mg to about 175 mg, about 25 mg to about 125 mg, about 75 mg to about 275 mg, about 75 mg to about 225 mg, about 75 mg to about 175 mg, about 75 mg to about 125 mg, about 100 mg to about 275 mg, about 100 mg to about 225 mg, about 100 mg to about 175 mg, about 100 mg to about 125 mg, about 125 mg to about 275 mg, about 125 mg to about 225 mg, about 125 mg to about 175 mg, about 175 mg to about 275 mg, about 175 mg to about 225 mg, or about 225 mg to about 275 mg of the fenugreek seed extract is present in the composition.
Embodiment 140. The composition of any one of embodiments 133-138, wherein about 25 mg, about 50 mg, about 75 mg, about 100 mg, about 125 mg, about 150 mg, about 175 mg, about 200 mg, about 225 mg, about 250 mg, or about 275 mg of the fenugreek seed extract is present in the composition.
Embodiment 141. The composition of any one of embodiments 1-46 or embodiments 119-140, further comprising Tribulus terrestris.
Embodiment 142. The composition of embodiment 141, wherein the Tribulus terrestris has diuretic, tonic, and aphrodisiac properties.
Embodiment 143. The composition of embodiment 141 or embodiment 142, wherein the Tribulus terrestris is present in the composition from about 0.1% to about 2.5%, about 0.5% to about 2.1%, or from about 0.9% to about 1.7% (w/w).
Embodiment 144. The composition of embodiment 141 or embodiment 142, wherein the Tribulus terrestris is present in the composition at about 0.1%, about 0.5%, about 0.9%, about 1.3%, about 1.7%, about 2.1%, or about 2.5%.
Embodiment 145. The composition of embodiment 141 or embodiment 142, wherein the Tribulus terrestris is present in the composition from about 0.1% to about 2.5% (w/w), about 0.1% to about 2.1%, about 0.1% to about 1.7%, about 0.1% to about 1.3%, about 0.1% to about 0.9%, about 0.1% to about 0.5%, about 0.5% to about 2.5%, about 0.5% to about 2.1%, about 0.5% to about 1.7%, about 0.5% to about 1.3%, about 0.5% to about 0.9%, about 0.9% to about 2.5%, about 0.9% to about 2.1%, about 0.9% to about 1.7%, about 0.9% to about 1.3%, about 1.3% to about 2.5%, about 1.3% to about 2.1%, about 1.3% to about 1.7%, about 1.7% to about 2.5%, about 1.7% to about 2.1%, or from about 2.1% to about 2.5% (w/w).
Embodiment 146. The composition of any one of embodiments 141-145, wherein about 1 mg to about 25 mg, about 1 mg to about 21 mg, about 1 mg to about 17 mg, about 1 mg to about 13 mg, about 1 mg to about 9 mg, about 1 mg to about 5 mg, about 5 mg to about 25 mg, about 5 mg to about 21 mg, about 5 mg to about 17 mg, about 5 mg to about 13 mg, about 5 mg to about 9 mg, about 9 mg to about 25 mg, about 9 mg to about 21 mg, about 9 mg to about 17 mg, about 9 mg to about 13 mg, about 13 mg to about 25 mg, about 13 mg to about 21 mg, about 13 mg to about 17 mg, about 17 mg to about 25 mg, about 17 mg to about 21 mg, about 21 mg to about 25 mg of the Tribulus terrestris is present in the composition.
Embodiment 147. The composition of any one of embodiments 141-145, wherein about 1 mg, about 5 mg, about 9 mg, about 13 mg, about 17 mg, about 21 mg, or about 25 mg of the Tribulus terrestris is present in the composition.
Embodiment 148. The composition of any one of embodiments 1-46 or embodiments 119-147, wherein the cordyceps species is present in the composition from about 0.1% to about 1.5%, about 0.3% to about 1.1%, or from about 0.5% to about 0.7% (w/w).
Embodiment 149. The composition of embodiment 148, wherein the cordyceps species is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5%.
Embodiment 150. The composition of embodiment 148, wherein the cordyceps species is present in the composition from about 0.1% to about 1.5%, about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1.5%, about 0.2% to about 1.4%, about 0.2% to about 1.3%, about 0.2% to about 1.2%, about 0.2% to about 1.1%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1.5%, about 0.3% to about 1.4%, about 0.3% to about 1.3%, about 0.3% to about 1.2%, about 0.3% to about 1.1%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1.5%, about 0.4% to about 1.4%, about 0.4% to about 1.3%, about 0.4% to about 1.2%, about 0.4% to about 1.1%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1.5%, about 0.5% to about 1.4%, about 0.5% to about 1.3%, about 0.5% to about 1.2%, about 0.5% to about 1.1%, about 0.5% to about 1⁰%, about 0.5% to about 0.9%, about 0.5% to about 0.8%, about 0.5% to about 0.7%, about 0.5% to about 0.6%, about 0.6% to about 1.5%, about 0.6% to about 1.4%, about 0.6% to about 1.3%, about 0.6% to about 1.2%, about 0.6% to about 1.1%, about 0.6% to about 1%, about 0.6% to about 0.9%, about 0.6% to about 0.8%, about 0.6% to about 0.7%, about 0.7% to about 1.5%, about 0.7% to about 1.4%, about 0.7% to about 1.3%, about 0.7% to about 1.2%, about 0.7% to about 1.1%, about 0.7% to about 1%, about 0.7% to about 0.9%, about 0.7% to about 0.8%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.5%, about 1% to about 1.4%, about 1% to about 1.3%, about 1% to about 1.2%, about 1% to about 1.1%, about 1.1% to about 1.5%, about 1.1% to about 1.4%, about 1.1% to about 1.3%, about 1.1% to about 1.2%, about 1.2% to about 1.5%, about 1.2% to about 1.4%, about 1.2% to about 1.3%, about 1.3% to about 1.5%, about 1.3% to about 1.4%, or from about 1.4% to about 1.5% (w/w).
Embodiment 151. The composition of any one of embodiments 148-150, wherein about 0.5 mg to about 15 mg, about 0.5 mg to about 14 mg, about 0.5 mg to about 13 mg, about 0.5 mg to about 12 mg, about 0.5 mg to about 11 mg, about 0.5 mg to about 11 mg, about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 15 mg, about 1.5 mg to about 14 mg, about 1.5 mg to about 13 mg, about 1.5 mg to about 12 mg, about 1.5 mg to about 11 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 15 mg, about 2.5 mg to about 14 mg, about 2.5 mg to about 13 mg, about 2.5 mg to about 12 mg, about 2.5 mg to about 11 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 15 mg, about 3.5 mg to about 14 mg, about 3.5 mg to about 13 mg, about 3.5 mg to about 12 mg, about 3.5 mg to about 11 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, about 14.5 mg to about 15 mg of the cordyceps species is present in the composition.
Embodiment 152. The composition of any one of embodiments 148-150, wherein about 0.5 mg, about 1.5 mg, about 2.5 mg, about 3.5 mg, about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg of the cordyceps species is present in the composition.
Embodiment 153. The composition of any one of embodiments 1-46 or embodiments 119-152, further comprising a tea extract.
Embodiment 154. The composition of embodiment 153, wherein the tea extract has antiviral, antioxidant, and potential chemopreventive properties.
Embodiment 155. The composition of embodiment 153 or embodiment 154, wherein the tea extract comprises a green tea extract, a black tea extract, an oolong tea extract, or a combination of two or more thereof.
Embodiment 156. The composition of embodiment 155, wherein the tea extract comprises green tea extract.
Embodiment 157. The composition of any one of embodiments 153-156, wherein the tea extract is present in the composition from about 0.1% to about 1%, about 0.2% to about 0.9%, or from about 0.4% to about 0.7% (w/w).
Embodiment 158. The composition of any one of embodiments 153-156, wherein the tea extract is present in the composition at about 0.1%, about 0.2%, about 0.3%, about 0.4%, about 0.5%, about 0.6%, about 0.7%, about 0.8%, about 0.9%, or about 1% (w/w).
Embodiment 159. The composition of any one of embodiments 153-156, wherein the tea extract is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1%.
Embodiment 160. The composition of any one of embodiments 153-159, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the tea extract is present in the composition.
Embodiment 161. The composition of any one of embodiments 153-159, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the tea extract is present in the composition.
Embodiment 162. The composition of any one of embodiments 1-46 or 119-161, further comprising a vitamin.
Embodiment 163. The composition of embodiment 162, wherein the vitamin is an antioxidant and is important for general cellular metabolism.
Embodiment 164. The composition of embodiment 162 or embodiment 163, wherein the vitamin comprises vitamin A, vitamin C, vitamin D, vitamin E, vitamin K, vitamin B1, vitamin B2, vitamin B3, vitamin B5, vitamin B6, vitamin B7, vitamin B9, vitamin B12, pyridoxine, cyanocobalamin, ascorbic acid, biotin niacin, folate, pantothenic acid, riboflavin, thiamine, choline, carnitine, folic acid, or a combination of two or more thereof.
Embodiment 165. The composition of embodiment 164, wherein the vitamin comprises vitamin B6.
Embodiment 166. The composition of any one of embodiments 162-165, wherein the vitamin is present in the composition from about 0.1% to about 1%, about 0.2% to about 0.9%, or from about 0.4% to about 0.7% (w/w).
Embodiment 167. The composition of any one of embodiments 162-165, wherein the vitamin is present in the composition at about 0.1%, about 0.2%, about 0.3%, about 0.4%, about 0.5%, about 0.6%, about 0.7%, about 0.8%, about 0.9%, or about 1% (w/w).
Embodiment 168. The composition of any one of embodiments 162-165, wherein the vitamin is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1%.
Embodiment 169. The composition of any one of embodiments 162-168, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the vitamin is present in the composition.
Embodiment 170. The composition of any one of embodiments 162-168, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the vitamin is present in the composition.
Embodiment 171. The composition of any one of embodiments 1-46 or 119-170, further comprising a nitric oxide source.
Embodiment 172. The composition of embodiment 171, wherein the nitric oxide source is used against pathologies such as hypertension and endothelial dysfunction.
Embodiment 173. The composition of embodiment 171 or embodiment 172, wherein the nitric oxide source comprises beet root powder.
Embodiment 174. The composition of any one of embodiments 171-173, wherein the nitric oxide source is present in the composition from about 0.1% to about 1%, about 0.2% to about 0.9%, or from about 0.4% to about 0.7% (w/w).
Embodiment 175. The composition of any one of embodiments 171-173, wherein the nitric oxide source is present in the composition at about 0.1%, about 0.2%, about 0.3%, about 0.4%, about 0.5%, about 0.6%, about 0.7%, about 0.8%, about 0.9%, or about 1% (w/w).
Embodiment 176. The composition of any one of embodiments 171-173, wherein the nitric oxide source is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1%.
Embodiment 177. The composition of any one of embodiments 171-176, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the nitric oxide source is present in the composition.
Embodiment 178. The composition of any one of embodiments 171-176, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the nitric oxide source is present in the composition.
Embodiment 179. The composition of any one of embodiments 1-46 or embodiments 119-178, further comprising an amino acid.
Embodiment 180. The composition of embodiment 179, wherein the amino acid is used to supplement the production of testosterone and sexual hormones.
Embodiment 181. The composition of embodiment 179 or embodiment 180, wherein the amino acid comprises D-aspartic acid.
Embodiment 182. The composition of any one of embodiments 179-181, wherein the amino acid is present in the composition from about 0.1% to about 1%, about 0.2% to about 0.9%, or from about 0.4% to about 0.7% (w/w).
Embodiment 183. The composition of any one of embodiments 179-181, wherein the amino acid is present in the composition at about 0.1%, about 0.2%, about 0.3%, about 0.4%, about 0.5%, about 0.6%, about 0.7%, about 0.8%, about 0.9%, or about 1% (w/w).
Embodiment 184. The composition of any one of embodiments 179-181, wherein the amino acid is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1%.
Embodiment 185. The composition of any one of embodiments 179-184, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the amino acid is present in the composition.
Embodiment 186. The composition of any one of embodiments 179-184, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the amino acid is present in the composition.
Embodiment 187. The composition of any one of embodiments 1-46 or embodiments 119-186, further comprising Panax ginseng.
Embodiment 188. The composition of embodiment 187, wherein the Panax ginseng is used to improve psychologic function, exercise performance, and immune function.
Embodiment 189. The composition of embodiment 187 or embodiment 188, wherein the Panax ginseng is present in the composition from about 0.1% to about 1%, about 0.2% to about 0.9%, or from about 0.4% to about 0.7% (w/w).
Embodiment 190. The composition of embodiment 187 or embodiment 188, wherein the Panax ginseng is present in the composition at about 0.1%, about 0.2%, about 0.3%, about 0.4%, about 0.5%, about 0.6%, about 0.7%, about 0.8%, about 0.9%, or about 1% (w/w).
Embodiment 191. The composition of embodiment 187 or embodiment 188, wherein the Panax ginseng is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1%.
Embodiment 192. The composition of any one of embodiments 187-191, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the Panax ginseng is present in the composition.
Embodiment 193. The composition of any one of embodiments 187-191, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the Panax ginseng is present in the composition.
Embodiment 194. The composition of any one of embodiments 1-46, further comprising maca.
Embodiment 195. The composition of embodiment 194, wherein the maca is used to enhance fertility and improve libido.
Embodiment 196. The composition of embodiment 194 or embodiment 195, wherein the maca is present in the composition from about 15% (w/w) to about 35% (w/w), about 17% (w/w) to about 31% (w/w), or from about 19% (w/w) to about 27% (w/w).
Embodiment 197. The composition of embodiment 119 or embodiment 120, wherein the maca is present in the composition at about 15%, about 20%, about 25%, about 30%, or about 35% (w/w).
Embodiment 198. The composition of embodiment 194 or embodiment 195, wherein the maca is present in the composition from about 15% to about 35%, about 15% to about 30%, about 15% to about 25%, about 15% to about 20%, about 20% to about 35%, about 20% to about 30%, about 20% to about 25%, about 25% to about 35%, about 25% to about 30%, or from about 30% to about 35%.
Embodiment 199. The composition of any one of embodiments 194-198, wherein about 100 mg to about 400 mg, about 100 mg to about 350 mg, about 100 mg to about 300 mg, about 100 mg to about 250 mg, about 150 mg to about 400 mg, about 150 mg to about 350 mg, about 150 mg to about 300 mg, about 150 mg to about 250 mg, about 200 mg to about 400 mg, about 200 mg to about 350 mg, about 200 mg to about 300 mg, about 200 mg to about 250 mg, about 250 mg to about 400 mg, about 250 mg to about 350 mg, about 250 mg to about 300 mg, about 300 mg to about 400 mg, about 300 mg to about 350 mg, or about 350 mg to about 400 mg of the maca is present in the composition.
Embodiment 200. The composition of any one of embodiments 194-198, wherein about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg of maca is present in the composition.
Embodiment 201. The composition of embodiments 1-45 or embodiments 194-200, further comprising a fenugreek seed extract.
Embodiment 202. The composition of embodiment 201, wherein the fenugreek seed extract is used to improve metabolism and hormonal profile in a person.
Embodiment 203. The composition of embodiment 200 or embodiment 201, wherein the fenugreek seed extract comprises Testosurge®.
Embodiment 204. The composition of any one of embodiments 201-203, wherein the fenugreek seed extract is present in the composition from about 5% to about 25%, about 7% to about 21%, or from about 9% to about 17% (w/w).
Embodiment 205. The composition of any one of embodiments 201-203, wherein the fenugreek seed extract is present in the composition at about 5%, about 10%, about 15%, about 20%, or about 25%.
Embodiment 206. The composition of any one of embodiments 201-203, wherein the fenugreek seed extract is present in the composition from about 5% to about 25%, about 5% to about 20%, about 5% to about 15%, about 5% to about 10%, about 10% to about 25%, about 10% to about 20%, about 10% to about 15%, about 15% to about 25%, about 15% to about 20%, or from about 20% to about 25% (w/w).
Embodiment 207. The composition of any one of embodiments 201-206, wherein about 25 mg to about 275 mg, about 25 mg to about 225 mg, about 25 mg to about 175 mg, about 25 mg to about 125 mg, about 75 mg to about 275 mg, about 75 mg to about 225 mg, about 75 mg to about 175 mg, about 75 mg to about 125 mg, about 100 mg to about 275 mg, about 100 mg to about 225 mg, about 100 mg to about 175 mg, about 100 mg to about 125 mg, about 125 mg to about 275 mg, about 125 mg to about 225 mg, about 125 mg to about 175 mg, about 175 mg to about 275 mg, about 175 mg to about 225 mg, or about 225 mg to about 275 mg of the fenugreek seed extract is present in the composition.
Embodiment 208. The composition of any one of embodiments 201-206, wherein about 25 mg, about 50 mg, about 75 mg, about 100 mg, about 125 mg, about 150 mg, about 175 mg, about 200 mg, about 225 mg, about 250 mg, or about 275 mg of the fenugreek seed extract is present in the composition.
Embodiment 209. The composition of embodiments 1-46 or embodiments 194-208, further comprising a vitamin.
Embodiment 210. The composition of embodiment 209, wherein the vitamin is an antioxidant and is important for general cellular metabolism.
Embodiment 211. The composition of embodiment 209 or embodiment 210, wherein the vitamin comprises vitamin A, vitamin C, vitamin D, vitamin E, vitamin K, vitamin B1, vitamin B2, vitamin B3, vitamin B5, vitamin B6, vitamin B7, vitamin B9, vitamin B12, pyridoxine, cyanocobalamin, ascorbic acid, biotin niacin, folate, pantothenic acid, riboflavin, thiamine, choline, carnitine, folic acid, or a combination of two or more thereof.
Embodiment 212. The composition of embodiment 211, wherein the vitamin comprises vitamin B6.
Embodiment 213. The composition of any one of embodiments 209-212, wherein the vitamin is present in the composition from about 0.8% to about 1.6%, about 1% to about 1.4%, or from about 1.1% to about 1.3% (w/w).
Embodiment 214. The composition of any one of embodiments 209-212, wherein the vitamin is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, or about 1.6% (w/w).
Embodiment 215. The composition of any one of embodiments 209-212, wherein the vitamin is present in the composition from about 0.8% to about 1.6%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.6%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.6%, about 1% to about 1.5%, about 1% to about 1.4%, about 1% to about 1.3%, about 1% to about 1.2%, about 1% to about 1.1%, about 1.1% to about 1.6%, about 1.1% to about 1.5%, about 1.1% to about 1.4%, about 1.1% to about 1.3%, about 1.1% to about 1.2%, about 1.2% to about 1.6%, about 1.2% to about 1.5%, about 1.2% to about 1.4%, about 1.2% to about 1.3%, about 1.3% to about 1.6%, about 1.3% to about 1.5%, about 1.3% to about 1.4%, about 1.4% to about 1.6%, about 1.4% to about 1.5%, or from about 1.5% to about 1.6% (w/w).
Embodiment 216. The composition of any one of embodiments 209-215, wherein about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, or about 14.5 mg to about 15 mg of the vitamin is present in the composition.
Embodiment 217. The composition of any one of embodiments 209-215, wherein about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg of the vitamin is present in the composition.
Embodiment 218. The composition of any one of embodiments 1-46 or embodiments 194-217, wherein the cordyceps species is present in the composition from about 0.8% to about 1.6%, about 1% to about 1.4%, or from about 1.1% to about 1.3% (w/w).
Embodiment 219. The composition of embodiment 218, wherein the cordyceps species is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, or about 1.6% (w/w).
Embodiment 220. The composition of embodiment 218, wherein the cordyceps species is present in the composition from about 0.8% to about 1.6%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.6%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.6%, about 1% to about 1.5%, about 1% to about 1.4%, about 1% to about 1.3%, about 1% to about 1.2%, about 1% to about 1.1%, about 1.1% to about 1.6%, about 1.1% to about 1.5%, about 1.1% to about 1.4%, about 1.1% to about 1.3%, about 1.1% to about 1.2%, about 1.2% to about 1.6%, about 1.2% to about 1.5%, about 1.2% to about 1.4%, about 1.2% to about 1.3%, about 1.3% to about 1.6%, about 1.3% to about 1.5%, about 1.3% to about 1.4%, about 1.4% to about 1.6%, about 1.4% to about 1.5%, or from about 1.5% to about 1.6% (w/w).
Embodiment 221. The composition of any one of embodiments 218-220, wherein about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, or about 14.5 mg to about 15 mg of the cordyceps species is present in the composition.
Embodiment 222. The composition of any one of embodiments 218-220, wherein about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg of the cordyceps species is present in the composition.
Embodiment 223. The composition of any one of embodiments 1-46 or embodiments 194-222, further comprising a tea extract.
Embodiment 224. The composition of embodiment 223, wherein the tea extract has antiviral, antioxidant, and potential chemopreventive properties.
Embodiment 225. The composition of embodiment 223 or embodiment 224, wherein the tea extract comprises a green tea extract, a black tea extract, an oolong tea extract, or a combination of two or more thereof.
Embodiment 226. The composition of embodiment 225, wherein the tea extract comprises the green tea extract.
Embodiment 227. The composition of any one of embodiments 223-226, wherein the tea extract is present in the composition from about 0.8% to about 1.6%, about 1% to about 1.4%, or from about 1.1% to about 1.3% (w/w).
Embodiment 228. The composition of any one of embodiments 223-226, wherein the tea extract is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, or about 1.6% (w/w).
Embodiment 229. The composition of any one of embodiments 223-226, wherein the tea extract is present in the composition from about 0.8% to about 1.6%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.6%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.6%, about 1% to about 1.5%, about 1% to about 1.4%, about 1% to about 1.3%, about 1% to about 1.2%, about 1% to about 1.1%, about 1.1% to about 1.6%, about 1.1% to about 1.5%, about 1.1% to about 1.4%, about 1.1% to about 1.3%, about 1.1% to about 1.2%, about 1.2% to about 1.6%, about 1.2/to about 1.5%, about 1.2% to about 1.4%, about 1.2% to about 1.3%, about 1.3% to about 1.6%, about 1.3% to about 1.5%, about 1.3% to about 1.4%, about 1.4% to about 1.6%, about 1.4% to about 1.5%, or from about 1.5% to about 1.6% (w/w).
Embodiment 230. The composition of any one of embodiments 223-229, wherein about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, or about 14.5 mg to about 15 mg of the tea extract is present in the composition.
Embodiment 231. The composition of any one of embodiments 223-229, wherein about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg of the tea extract is present in the composition.
Embodiment 232. The composition of any one of embodiments 1-46 or embodiments 194-231, further comprising a nitric oxide source.
Embodiment 233. The composition of embodiment 232, wherein the nitric oxide source is used for pathologies such as hypertension and endothelial dysfunction.
Embodiment 234. The composition of embodiment 232 or embodiment 233, wherein the nitric oxide source comprises beet root powder.
Embodiment 235. The composition of any one of embodiments 232-234, wherein the nitric oxide source is present in the composition from about 0.8% to about 1.6%, about 1% to about 1.4%, or from about 1.1% to about 1.3% (w/w).
Embodiment 236. The composition of any one of embodiments 232-234, wherein the nitric oxide source is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, or about 1.6% (w/w).
Embodiment 237. The composition of any one of embodiments 232-234, wherein the nitric oxide source is present in the composition from about 0.8% to about 1.6%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.6%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.6%, about 1% to about 1.5%, about 1% to about 1.4%, about 1% to about 1.3%, about 1% to about 1.2%, about 1% to about 1.1%, about 1.1% to about 1.6%, about 1.1% to about 1.5%, about 1.1% to about 1.4%, about 1.1% to about 1.3%, about 1.1% to about 1.2%, about 1.2% to about 1.6%, about 1.2% to about 1.5%, about 1.2% to about 1.4%, about 1.2% to about 1.3%, about 1.3% to about 1.6%, about 1.3% to about 1.5%, about 1.3% to about 1.4%, about 1.4% to about 1.6%, about 1.4% to about 1.5%, or from about 1.5% to about 1.6% (w/w).
Embodiment 238. The composition of any one of embodiments 232-237, wherein about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, or about 14.5 mg to about 15 mg of the nitric oxide source is present in the composition.
Embodiment 239. The composition of any one of embodiments 232-237, wherein about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg of the nitric oxide source is present in the composition.
Embodiment 240. The composition of any one of embodiments 1-46 or embodiments 194-239, further comprising Bacopa monieri.
Embodiment 241. The composition of embodiment 240, where the Bacopa monieri has memory enhancing properties.
Embodiment 242. The composition of embodiment 240 or embodiment 241, wherein the Bacopa monieri is present in the composition from about 0.8% to about 1.6%, about 1% to about 1.4%, or from about 1.1% to about 1.3% (w/w).
Embodiment 243. The composition of embodiment 240 or embodiment 241, wherein the Bacopa monieri is present in the composition at about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, or about 1.6% (w/w).
Embodiment 244. The composition of embodiment 240 or embodiment 241, wherein the Bacopa monieri is present in the composition from about 0.8% to about 1.6%, about 0.8% to about 1.5%, about 0.8% to about 1.4%, about 0.8% to about 1.3%, about 0.8% to about 1.2%, about 0.8% to about 1.1%, about 0.8% to about 1%, about 0.8% to about 0.9%, about 0.9% to about 1.6%, about 0.9% to about 1.5%, about 0.9% to about 1.4%, about 0.9% to about 1.3%, about 0.9% to about 1.2%, about 0.9% to about 1.1%, about 0.9% to about 1%, about 1% to about 1.6%, about 1% to about 1.5%, about 1% to about 1.4%, about 1% to about 1.3%, about 1% to about 1.2%, about 1% to about 1.1%, about 1.1% to about 1.6%, about 1.1% to about 1.5%, about 1.1% to about 1.4%, about 1.1% to about 1.3%, about 1.1% to about 1.2%, about 1.2% to about 1.6%, about 1.2% to about 1.5%, about 1.2% to about 1.4%, about 1.2% to about 1.3%, about 1.3% to about 1.6%, about 1.3% to about 1.5%, about 1.3% to about 1.4%, about 1.4% to about 1.6%, about 1.4% to about 1.5%, or from about 1.5% to about 1.6% (w/w).
Embodiment 245. The composition of any one of embodiments 240-244, wherein about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, or about 14.5 mg to about 15 mg of the Bacopa monieri is present in the composition.
Embodiment 246. The composition of any one of embodiments 240-244, wherein about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg of the Bacopa monieri is present in the composition.
Embodiment 247. The composition of any one of embodiments 1-46 or embodiments 194-247, further comprising an amino acid.
Embodiment 248. The composition of embodiment 247, wherein the amino acid is used to supplement the production of testosterone and sexual hormones.
Embodiment 249. The composition of embodiment 247 or embodiment 248, wherein the amino acid comprises D-aspartic acid.
Embodiment 250. The composition of any one of embodiments 247-249, wherein the amino acid is present in the composition from about 0.1% to about 1%, about 0.2% to about 0.9%, or from about 0.4% to about 0.7% (w/w).
Embodiment 251. The composition of any one of embodiments 247-249, wherein the amino acid is present in the composition at about 0.1%, about 0.2%, about 0.3%, about 0.4%, about 0.5%, about 0.6%, about 0.7%, about 0.8%, about 0.9%, or about 1% (w/w).
Embodiment 252. The composition of any one of embodiments 247-249, wherein the amino acid is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1%.
Embodiment 253. The composition of any one of embodiments 247-252, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the amino acid is present in the composition.
Embodiment 254. The composition of any one of embodiments 247-252, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the amino acid is present in the composition.
Embodiment 255. The composition of any one of embodiments 1-46 or embodiments 194-254, further comprising Panax ginseng.
Embodiment 256. The composition of embodiment 255, wherein the Panax ginseng is used to improve psychologic function, exercise performance, and immune function.
Embodiment 257. The composition of embodiment 255 or embodiment 256, wherein the Panax ginseng is present in the composition from about 0.1% to about 1%, about 0.2% to about 0.9%, or from about 0.4% to about 0.7% (w/w).
Embodiment 258. The composition of embodiment 255 or embodiment 256, wherein the Panax ginseng is present in the composition at about 0.1%, about 0.2%, about 0.3%, about 0.4%, about 0.5%, about 0.6%, about 0.7%, about 0.8%, about 0.9%, or about 1% (w/w).
Embodiment 259. The composition of embodiment 255 or embodiment 256, wherein the Panax ginseng is present in the composition from about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, about 0.1% to about 0.2%, about 0.2% to about 1%, about 0.2% to about 0.9%, about 0.2% to about 0.8%, about 0.2% to about 0.7%, about 0.2% to about 0.6%, about 0.2% to about 0.5%, about 0.2% to about 0.4%, about 0.2% to about 0.3%, about 0.3% to about 1%, about 0.3% to about 0.9%, about 0.3% to about 0.8%, about 0.3% to about 0.7%, about 0.3% to about 0.6%, about 0.3% to about 0.5%, about 0.3% to about 0.4%, about 0.4% to about 1%, about 0.4% to about 0.9%, about 0.4% to about 0.8%, about 0.4% to about 0.7%, about 0.4% to about 0.6%, about 0.4% to about 0.5%, about 0.5% to about 1%, about 0.5% to about 0.9%, about 0.5% to about 0.8%, about 0.5% to about 0.7%, about 0.5% to about 0.6%, about 0.6% to about 1%, about 0.6% to about 0.9%, about 0.6% to about 0.8%, about 0.6% to about 0.7%, about 0.7% to about 1%, about 0.7% to about 0.9%, about 0.7% to about 0.8%, about 0.8% to about 1%, about 0.8% to about 0.9%, or from about 0.9% to about 1%.
Embodiment 260. The composition of any one of embodiments 255-259, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the Panax ginseng is present in the composition.
Embodiment 261. The composition of any one of embodiments 255-259, wherein about 0.5 mg, about 1.5 mg, about 2.5 mg, about 3.5 mg, about 4.5 mg, about 5.5 mg, about 6.5 mg, about 7.5 mg, about 8.5 mg, or about 9.5 mg of the Panax ginseng is present in the composition.
Embodiment 262. The composition of any one of embodiments 1-43, further comprising phosphatidylserine.
Embodiment 263. The composition of embodiment 262, wherein the phosphatidylserine has memory enhancing properties.
Embodiment 264. The composition of embodiment 262 or embodiment 263, wherein the phosphatidylserine is present in the composition from about 0.5% to about 10%, about 2% to about 8%, or from about 4% to about 6% (w/w).
Embodiment 265. The composition of embodiment 262 or embodiment 263, wherein the phosphatidylserine is present in the composition at about 0.5% (w/w), about 1.5% (w/w), about 2.5% (w/w), about 3.5% (w/w), about 4.5% (w/w), about 5.5% (w/w), about 6.5% (w/w), about 7.5% (w/w), about 8.5% (w/w), or about 9.5% (w/w).
Embodiment 266. The composition of embodiment 262 or embodiment 263, wherein the phosphatidylserine is present in the composition from about 0.5% to about 10%, about 0.5% (w/w) to about 9% (w/w), about 0.5% (w/w) to about 8% (w/w), about 0.5% (w/w) to about 7% (w/w), about 0.5% (w/w) to about 6% (w/w), about 0.5% (w/w) to about 5% (w/w), about 0.5% (w/w) to about 4% (w/w), about 0.5% (w/w) to about 3% (w/w), about 0.5% (w/w) to about 2% (w/w), about 0.5% (w/w) to about 1% (w/w), about 1.5% (w/w) to about 10% (w/w), about 1.5% (w/w) to about 9% (w/w), about 1.5% (w/w) to about 8% (w/w), about 1.5% (w/w) to about 7% (w/w), about 1.5% (w/w) to about 6% (w/w), about 1.5% (w/w) to about 5% (w/w), about 1.5% (w/w) to about 4% (w/w), about 1.5% (w/w) to about 3% (w/w), about 1.5% (w/w) to about 2% (w/w), about 2.5% (w/w) to about 10% (w/w), about 2.5% (w/w) to about 9% (w/w), about 2.5% (w/w) to about 8% (w/w), about 2.5% (w/w) to about 7% (w/w), about 2.5% (w/w) to about 6% (w/w), about 2.5% (w/w) to about 5% (w/w), about 2.5% (w/w) to about 4% (w/w), about 2.5% (w/w) to about 3% (w/w), about 3.5% (w/w) to about 10% (w/w), about 3.5% (w/w) to about 9% (w/w), about 3.5% (w/w) to about 8% (w/w), about 3.5% (w/w) to about 7% (w/w), about 3.5% (w/w) to about 6% (w/w), about 3.5% (w/w) to about 5% (w/w), about 3.5% (w/w) to about 4% (w/w), about 4.5% (w/w) to about 10% (w/w), about 4.5% (w/w) to about 9% (w/w), about 4.5% (w/w) to about 8% (w/w), about 4.5% (w/w) to about 7% (w/w), about 4.5% (w/w) to about 6% (w/w), about 4.5% (w/w) to about 5% (w/w), about 5.5% (w/w) to about 10% (w/w), about 5.5% (w/w) to about 9% (w/w), about 5.5% (w/w) to about 8% (w/w), about 5.5% (w/w) to about 7% (w/w), about 5.5% (w/w) to about 6% (w/w), about 6.5% (w/w) to about 10% (w/w), about 6.5% (w/w) to about 9% (w/w), about 6.5% (w/w) to about 8% (w/w), about 6.5% (w/w) to about 7% (w/w), about 7.5% (w/w) to about 10% (w/w), about 7.5% (w/w) to about 9% (w/w), about 7.5% (w/w) to about 8% (w/w), about 8.5% (w/w) to about 10% (w/w), about 8.5% (w/w) to about 9% (w/w), or from about 9.5% (w/w) to about 10% (w/w).
Embodiment 267. The composition of any one of embodiments 262-266, wherein about 1 mg to about 25 mg, about 1 mg to about 21 mg, about 1 mg to about 17 mg, about 1 mg to about 13 mg, about 1 mg to about 9 mg, about 1 mg to about 5 mg, about 5 mg to about 25 mg, about 5 mg to about 21 mg, about 5 mg to about 17 mg, about 5 mg to about 13 mg, about 5 mg to about 9 mg, about 9 mg to about 25 mg, about 9 mg to about 21 mg, about 9 mg to about 17 mg, about 9 mg to about 13 mg, about 13 mg to about 25 mg, about 13 mg to about 21 mg, about 13 mg to about 17 mg, about 17 mg to about 25 mg, of about 21 mg to about 25 mg of the phosphatidylserine is present in the composition.
Embodiment 268. The composition of any one of embodiments 262-266, wherein about 1 mg, about 5 mg, about 9 mg, about 13 mg, about 17 mg, about 21 mg, or about 25 mg of the phosphatidylserine is present in the composition.
Embodiment 269. The composition of any one of embodiments 1-43 or embodiments 262-268, further comprising a flavoring agent.
Embodiment 270. The composition of embodiment 269, wherein the flavoring agent comprises peppermint flavor, methanol crystals, or a combination thereof.
Embodiment 271. The composition of embodiment 269 or embodiment 270, wherein the flavoring agent is present in the composition from about 1% to about 15% (w/w).
Embodiment 272. The composition of embodiment 269 or embodiment 270, wherein the flavoring agent is present in the composition at about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w).
Embodiment 273. The composition of embodiment 269 or embodiment 270, wherein the flavoring agent is present in the composition from about 1% to about 2% (w/w), from about 2% to about 3% (w/w), from about 3% to about 4% (w/w), from about 4% to about 5% (w/w), from about 5% to about 6% (w/w), from about 6% to about 7% (w/w), from about 7% to about 8% (w/w), from about 8% to about 9% (w/w), or from about 9% to about 10% (w/w).
Embodiment 274. The composition of any one of embodiments 269-273, wherein about 4 mg to about 800 mg, about 4 mg to about 700 mg, about 4 mg to about 600 mg, about 4 mg to about 500 mg, about 4 mg to about 400 mg, about 4 mg to about 300 mg, about 10 mg to about 800 mg, about 10 mg to about 700 mg, about 10 mg to about 600 mg, about 10 mg to about 500 mg, about 10 mg to about 400 mg, about 10 mg to about 300 mg, about 50 mg to about 800 mg, about 50 mg to about 700 mg, about 50 mg to about 600 mg, about 50 mg to about 500 mg, about 50 mg to about 400 mg, about 50 mg to about 300 mg, about 100 mg to about 800 mg, about 100 mg to about 700 mg, about 100 mg to about 600 mg, about 100 mg to about 500 mg, about 100 mg to about 400 mg, or about 100 mg to about 300 mg of the flavoring agent is present in the composition.
Embodiment 275. The composition of any one of embodiments 269-273, wherein about 4 mg, about 5 mg, about 10 mg, about 20 mg, about 25 mg, about 30 mg, or about 40 mg of the flavoring agent is present in the composition.
Embodiment 276. The composition of any one of embodiments 1-43 or embodiments 262-275, further comprising L-theanine.
Embodiment 277. The composition of embodiment 276, wherein the L-theanine has relaxant and antioxidant properties.
Embodiment 278. The composition of embodiment 276 or embodiment 277, wherein the L-theanine is present in the composition from about 0.1% to about 8% (w/w), about 2.1% to about 6%, or from about 3.1% to about 5% (w/w).
Embodiment 279. The composition of embodiment 276 or embodiment 277, wherein the L-theanine is present in the composition at about 0.1% (w/w), about 1.1% (w/w), about 2.1% (w/w), about 3.1% (w/w), about 4.1% (w/w), about 5.1% (w/w), about 6.1% (w/w), or about 7.1% (w/w).
Embodiment 280. The composition of embodiment 276 or embodiment 277, wherein the L-theanine is present in the composition from about 0.1% to about 8%, about 0.1% (w/w) to about 7% (w/w), about 0.1% (w/w) to about 6% (w/w), about 0.1% (w/w) to about 5% (w/w), about 0.1% (w/w) to about 4% (w/w), about 0.1% (w/w) to about 3% (w/w), about 0.1% (w/w) to about 2% (w/w), about 0.1% (w/w) to about 1% (w/w), about 1.1% (w/w) to about 8% (w/w), about 1.1% (w/w) to about 7% (w/w), about 1.1% (w/w) to about 6% (w/w), about 1.1% (w/w) to about 5% (w/w), about 1.1% (w/w) to about 4% (w/w), about 1.1% (w/w) to about 3% (w/w), about 1.1% (w/w) to about 2% (w/w), about 2.1% (w/w) to about 8% (w/w), about 2.1% (w/w) to about 7% (w/w), about 2.1% (w/w) to about 6% (w/w), about 2.1% (w/w) to about 5% (w/w), about 2.1% (w/w) to about 4% (w/w), about 2.1% (w/w) to about 3% (w/w), about 3.1% (w/w) to about 8% (w/w), about 3.1% (w/w) to about 7% (w/w), about 3.1% (w/w) to about 6% (w/w), about 3.1% (w/w) to about 5% (w/w), about 3.1% (w/w) to about 4% (w/w), about 4.1% (w/w) to about 8% (w/w), about 4.1% (w/w) to about 7% (w/w), about 4.1% (w/w) to about 6% (w/w), about 4.1% (w/w) to about 5% (w/w), about 5.1% (w/w) to about 8% (w/w), about 5.1% (w/w) to about 7% (w/w), about 5.1% (w/w) to about 6% (w/w), about 6.1% (w/w) to about 8% (w/w), about 6.1% (w/w) to about 7% (w/w), or from about 7.1% (w/w) to about 8% (w/w).
Embodiment 281. The composition of any one of embodiments 276-280, wherein about 4.5 mg to about 15 mg, about 4.5 mg, to about 14 mg, about 4.5 mg to about 13 mg, about 4.5 mg to about 12 mg, about 4.5 mg to about 11 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 15 mg, about 5.5 mg to about 14 mg, about 5.5 mg to about 13 mg, about 5.5 mg to about 12 mg, about 5.5 mg to about 11 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 15 mg, about 6.5 mg to about 14 mg, about 6.5 mg to about 13 mg, about 6.5 mg to about 12 mg, about 6.5 mg to about 11 mg, about 6.5 mg to about 10 mg, about 6.5 mg to about 9 mg, about 6.5 mg to about 8 mg, about 6.5 mg to about 7 mg, about 7.5 mg to about 15 mg, about 7.5 mg to about 14 mg, about 7.5 mg to about 13 mg, about 7.5 mg to about 12 mg, about 7.5 mg to about 12 mg, about 7.5 mg to about 11 mg, about 7.5 mg to about 10 mg, about 7.5 mg to about 9 mg, about 7.5 mg to about 8 mg, about 8.5 mg to about 15 mg, about 8.5 mg to about 14 mg, about 8.5 mg to about 13 mg, about 8.5 mg to about 12 mg, about 8.5 mg to about 11 mg, about 8.5 mg to about 10 mg, about 8.5 mg to about 9 mg, about 9.5 mg to about 15 mg, about 9.5 mg to about 14 mg, about 9.5 mg to about 13 mg, about 9.5 mg to about 12 mg, about 9.5 mg to about 11 mg, about 9.5 mg to about 10 mg, about 10.5 mg to about 15 mg, about 10.5 mg to about 14 mg, about 10.5 mg to about 13 mg, about 10.5 mg to about 12 mg, about 10.5 mg to about 11 mg, about 11.5 mg to about 15 mg, about 11.5 mg to about 14 mg, about 11.5 mg to about 13 mg, about 11.5 mg to about 12 mg, about 12.5 mg to about 15 mg, about 12.5 mg to about 14 mg, about 12.5 mg to about 13 mg, about 13.5 mg to about 15 mg, about 13.5 mg to about 14 mg, or about 14.5 mg to about 15 mg of the L-theanine is present in the composition.
Embodiment 282. The composition of any one of embodiments 276-280, wherein about 4.5 mg, about 5.5 mg, about 6.5 mg, about 7.5 mg, about 8.5 mg, about 9.5 mg. about 10.5 mg, about 11.5 mg, about 12.5 mg, about 13.5 mg, or about 14.5 mg of the L-theanine is present in the composition.
Embodiment 283. The composition of any one of embodiments 1-43 or embodiments 262-282, further comprising L-tryptophan.
Embodiment 284. The composition of embodiment 283, wherein the L-tryptophan has anti-anxiety and anti-depression properties.
Embodiment 285. The composition of embodiment 283 or embodiment 284, wherein the L-tryptophan is present in the composition from about 0.1% to about 7% (w/w), about 1.1% to about 6%, or from about 2.1% to about 5% (w/w).
Embodiment 286. The composition of embodiment 283 or embodiment 284, wherein the L-tryptophan is present in the composition at about 0.1% (w/w), about 1.1% (w/w), about 2.1% (w/w), about 3.1% (w/w), about 4.1% (w/w), or about 5.1% (w/w), or about 6.1% (w/w).
Embodiment 287. The composition of embodiment 283 or embodiment 284, wherein the L-tryptophan is present in the composition from about 0.1% to about 7% (w/w), about 0.1% (w/w) to about 6% (w/w), about 0.1% (w/w) to about 5% (w/w), about 0.1% (w/w) to about 4% (w/w), about 0.1% (w/w) to about 3% (w/w), about 0.1% (w/w) to about 2% (w/w), about 0.1% (w/w) to about 1% (w/w), about 1.1% (w/w) to about 7% (w/w), about 1.1% (w/w) to about 6% (w/w), about 1.1% (w/w) to about 5% (w/w), about 1.1% (w/w) to about 4% (w/w), about 1.1% (w/w) to about 3% (w/w), about 1.1% (w/w) to about 2% (w/w), about 2.1% (w/w) to about 7% (w/w), about 2.1% (w/w) to about 6% (w/w), about 2.1% (w/w) to about 5% (w/w), about 2.1% (w/w) to about 4% (w/w), about 2.1% (w/w) to about 3% (w/w), about 3.1% (w/w) to about 7% (w/w), about 3.1% (w/w) to about 6% (w/w), about 3.1% (w/w) to about 5% (w/w), about 3.1% (w/w) to about 4% (w/w), about 4.1% (w/w) to about 7% (w/w), about 4.1% (w/w) to about 6% (w/w), about 4.1% (w/w) to about 5% (w/w), about 5.1% (w/w) to about 7% (w/w), about 5.1% (w/w) to about 6% (w/w), or from about 6.1% (w/w) to about 7% (w/w).
Embodiment 288. The composition of any one of embodiments 283-287, wherein about 2.5 mg to about 12.5 mg, about 2.5 mg to about 11.5 mg, about 2.5 mg to about 10.5 mg, about 2.5 mg to about 9.5 mg, about 2.5 mg to about 8.5 mg, about 2.5 mg to about 7.5 mg, about 2.5 mg to about 6.5 mg, about 2.5 mg to about 5.5 mg, about 2.5 mg to about 4.5 mg, about 2.5 mg to about 3.5 mg, about 3.5 mg to about 12.5 mg, about 3.5 mg to about 11.5 mg, about 3.5 mg to about 10.5 mg, about 3.5 mg to about 9.5 mg, about 3.5 mg to about 8.5 mg, about 3.5 mg to about 7.5 mg, about 3.5 mg to about 6.5 mg, about 3.5 mg to about 5.5 mg, about 3.5 mg to about 4.5 mg, about 4.5 mg to about 12.5 mg, about 4.5 mg to about 11.5 mg, about 4.5 mg top about 10.5 mg, about 4.5 mg to 9.5 mg, about 4.5 mg to about 8.5 mg, about 4.5 mg to about 7.5 mg, about 4.5 mg to about 6.5 mg, about 4.5 mg to about 5.5 mg, about 5.5 mg to about 12.5 mg, about 5.5 mg to about 11.5 mg, about 5.5 mg to about 10.5 mg, about 5.5 mg to about 9.5 mg, about 5.5 mg to about 8.5 mg, about 5.5 mg to about 7.5 mg, about 5.5 mg to about 6.5 mg, about 6.5 mg to about 12.5 mg, about 6.5 mg to about 11.5 mg, about 6.5 mg to about 10.5 mg, about 6.5 mg to about 9.5 mg, about 6.5 mg to about 8.5 mg, about 6.5 mg to about 7.5 mg, about 7.5 mg to about 12.5 mg, about 7.5 mg to about 11.5 mg, about 7.5 mg to about 10.5 mg, about 7.5 mg to about 9.5 mg, about 7.5 mg to about 8.5 mg, about 8.5 mg to about 12.5 mg, about 8.5 mg to about 11.5 mg, about 8.5 mg to about 10.5 mg, about 8.5 mg to about 9.5 mg, about 9.5 mg to about 12.5 mg, about 9.5 mg to about 11.5 mg, about 9.5 mg to about 10.5 mg, about 10.5 mg to about 12.5 mg, about 10.5 mg to about 11.5 mg, or about 11.5 mg to about 12.5 mg of the L-tryptophan is present in the composition.
Embodiment 289. The composition of any one of embodiments 283-287, wherein about 2.5 mg, about 3.5 mg, about 4.5 mg, about 5.5 mg, about 6.5 mg, about 7.5 mg, about 8.5 mg, about 9.5 mg. about 10.5 mg, about 11.5 mg, or about 12.5 mg of the L-tryptophan is present in the composition.
Embodiment 290. The composition of any one of embodiments 1-43 or embodiments 262-289, further comprising reishi mushroom.
Embodiment 291. The composition of embodiment 290, wherein the reishi mushroom has anticancer properties.
Embodiment 292. The composition of embodiment 290 or embodiment 291, wherein the reishi mushroom is present in the composition from about 1% to about 3%, about 1.25% to about 2.75%, or from about 1.5% to about 2.5% (w/w).
Embodiment 293. The composition of embodiment 290 or embodiment 291, wherein the reishi mushroom is present in the composition at about 1% (w/w), about 1.25% (w/w), about 1.5% (w/w), about 1.75% (w/w), about 2% (w/w), about 2.25% (w/w), about 2.5% (w/w), about 2.75% (w/w), or about 3% (w/w).
Embodiment 294. The composition of embodiment 290 or embodiment 291, wherein the reishi mushroom is present in the composition from about 1% to about 3%, about 1% (w/w) to about 2.75% (w/w), about 1% (w/w) to about 2.5% (w/w), about 1% (w/w) to about 2.25% (w/w), about 1% (w/w) to about 2% (w/w), about 1% (w/w) to about 1.75% (w/w), about 1% (w/w) to about 1.5% (w/w), about 1% (w/w) to about 1.25% (w/w), about 1.25% (w/w) to about 3% (w/w), about 1.25% (w/w) to about 2.75% (w/w), about 1.25% (w/w) to about 2.5% (w/w), about 1.25% (w/w) to about 2.25% (w/w), about 1% (w/w) to about 2% (w/w), about 1.25% (w/w) to about 1.75% (w/w), about 1.25% (w/w) to about 1.5% (w/w), about 1.5% (w/w) to about 3% (w/w), about 1.5% (w/w) to about 2.75% (w/w), about 1.5% (w/w) to about 2.5% (w/w), about 1.5% (w/w) to about 2.25% (w/w), about 1.5% (w/w) to about 2% (w/w), about 1.5% (w/w) to about 1.75% (w/w), about 1.75% (w/w) to about 3% (w/w), about 1.75% (w/w) to about 2.75% (w/w), 1.75% (w/w) to about 2.5% (w/w), about 1.75% (w/w) to about 2.25% (w/w), about 1.75% (w/w) to about 2% (w/w), about 2% (w/w) to about 3% (w/w), about 2% (w/w) to about 2.75% (w/w), about 2% (w/w) to about 2.5% (w/w), about 2% (w/w) to about 2.25% (w/w), about 2.25% (w/w) to about 3% (w/w), about 2.25% (w/w) to about 2.75% (w/w), about 2.25% (w/w) to about 2.5% (w/w), about 2.5% (w/w) to about 3% (w/w), about 2.5% (w/w) to about 2.75% (w/w), or from about 2.75% (w/w) to about 3% (w/w).
Embodiment 295. The composition of any one of embodiments 290-294, wherein about 0.5 mg to about 10 mg, about 0.5 mg to about 9 mg, about 0.5 mg to about 8 mg, about 0.5 mg to about 7 mg, about 0.5 mg to about 6 mg, about 0.5 mg to about 5 mg, about 0.5 mg to about 4 mg, about 0.5 mg to about 3 mg, about 0.5 mg to about 2 mg, about 1.5 mg to about 10 mg, about 1.5 mg to about 9 mg, about 1.5 mg to about 8 mg, about 1.5 mg to about 7 mg, about 1.5 mg to about 6 mg, about 1.5 mg to about 5 mg, about 1.5 mg to about 4 mg, about 1.5 mg to about 3 mg, about 1.5 mg to about 2 mg, about 2.5 mg to about 10 mg, about 2.5 mg to about 9 mg, about 2.5 mg to about 8 mg, about 2.5 mg to about 7 mg, about 2.5 mg to about 6 mg, about 2.5 mg to about 5 mg, about 2.5 mg to about 4 mg, about 2.5 mg to about 3 mg, about 3.5 mg to about 10 mg, about 3.5 mg to about 9 mg, about 3.5 mg to about 8 mg, about 3.5 mg to about 7 mg, about 3.5 mg to about 6 mg, about 3.5 mg to about 5 mg, about 3.5 mg to about 5 mg, about 3.5 mg to about 4 mg, about 4.5 mg top about 10 mg, about 4.5 mg to 9 mg, about 4.5 mg to about 8 mg, about 4.5 mg to about 7 mg, about 4.5 mg to about 6 mg, about 4.5 mg to about 5 mg, about 5.5 mg to about 10 mg, about 5.5 mg to about 9 mg, about 5.5 mg to about 8 mg, about 5.5 mg to about 7 mg, about 5.5 mg to about 6 mg, about 6.5 mg to about 10 mg, about 6.5 mg to about 9 mg, about 6.5 mg to about 8 mg, about 6.5 mg to about 7 mg, about 7.5 mg to about 10 mg, about 7.5 mg to about 9 mg, about 7.5 mg to about 8 mg, about 8.5 mg to about 10 mg, about 8.5 mg to about 9 mg, or about 9.5 mg to about 10 mg of the reishi mushroom is present in the composition.
Embodiment 296. The composition of any one of embodiments 290-294, wherein about 5 mg, about 1.5 mg, about 2.5 mg, about 3.5 mg, about 4.5 mg, about 5.5 mg, about 6.5 mg, about 7.5 mg, about 8.5 mg, or about 9.5 mg of the reishi mushroom is present in the composition.
Embodiment 297. The composition of any one of embodiments 1-43 or embodiments 262-296, further comprising melatonin.
Embodiment 298. The composition of embodiment 297, wherein the melatonin induces fatigue.
Embodiment 299. The composition of embodiment 297, wherein the melatonin reduces the time needed for a person taking the composition to fall asleep.
Embodiment 300. The composition of embodiment 297, wherein the melatonin reduces insomnia.
Embodiment 301. The composition of embodiment 297, wherein the melatonin is bioavailable for up to 240 minutes.
Embodiment 302. The composition of embodiment 297, wherein the melatonin has components used for regulation of circadian rhythms.
Embodiment 303. The composition of embodiment 302, wherein a component used for regulation of circadian rhythms is neuroendocrine rhythms.
Embodiment 304. The composition of embodiment 302, wherein a component used for regulation of circadian rhythms is neuroendocrine rhythms is sleep-wake rhythms.
Embodiment 305. The composition of embodiment 302, wherein a component used for regulation of circadian rhythms is body temperature cycles.
Embodiment 306. The composition of any one of embodiments 297-305, wherein the melatonin is present in the composition from about 0.01% to about 2%, about 0.26% to about 1.75%, or from about 0.51% to about 1.5% (w/w).
Embodiment 307. The composition of any one of embodiments 297-305, wherein the melatonin is present in the composition at about 0.01% (w/w), about 0.26% (w/w), about 0.51% (w/w), about 0.76% (w/w), about 1.01% (w/w), about 1.26% (w/w), about 1.51% (w/w), or about 1.76% (w/w).
Embodiment 308. The composition of any one of embodiments 297-305, wherein the melatonin is present in the composition from about 0.01% to about 2%, about 0.01% (w/w) to about 1.75% (w/w), about 0.01% (w/w) to about 1.5%, about 0.01% to about 1.25%, about 0.01% to about 1%, about 0.01% to about 0.75%, about 0.01% to about 0.5%, about 0.01% to about 0.25%, about 0.26% (w/w) to about 2% (w/w), about 0.26% (w/w) to about 1.75% (w/w), about 0.26% (w/w) to about 1.5% (w/w), about 0.26% (w/w) to about 1.25% (w/w), about 0.26% (w/w) to about 1% (w/w), about 0.26% (w/w) to about 0.75% (w/w), about 0.26% (w/w) to about 0.5% (w/w), about 0.51% (w/w) to about 2% (w/w), about 0.51% (w/w) to about 1.75% (w/w), about 0.51% (w/w) to about 1.5% (w/w), about 0.51% (w/w) to about 1.25% (w/w), about 0.51% (w/w) to about 1% (w/w), about 0.51% (w/w) to about 0.75% (w/w), about 0.76% (w/w) to about 2% (w/w), about 0.76% (w/w) to about 1.75% (w/w), about 0.76% (w/w) to about 1.5% (w/w), about 0.76% (w/w) to about 1.25% (w/w), about 0.76% (w/w) to about 1% (w/w), about 1.01% (w/w) to about 2% (w/w), about 1.01% (w/w) to about 1.75% (w/w), about 1.01% (w/w) to about 1.5% (w/w), about 1.01% (w/w) to about 1.25% (w/w), about 1.26% (w/w) to about 2% (w/w), about 1.26% (w/w) to about 1.75% (w/w), about 1.26% (w/w) to about 1.5% (w/w), about 1.51% (w/w) to about 2% (w/w), about 1.51% (w/w) to about 1.75% (w/w), or from about 1.76% (w/w) to about 2% (w/w).
Embodiment 309. The composition of any one of embodiments 297-308, wherein about 0.01 mg to about 5 mg, about 0.01 mg to about 4.5 mg, about 0.01 mg to about 4 mg, about 0.01 mg to about 3.5 mg, about 0.01 mg to about 3 mg, about 0.01 mg to about 2.5 mg, about 0.01 mg to about 2 mg, about 0.01 mg to about 1.5 mg, about 0.01 mg to about 1 mg, about 0.01 mg to about 0.5 mg, about 0.51 mg to about 5 mg, about 0.51 mg to about 4.5 mg, about 0.51 mg to about 4 mg, about 0.51 mg to about 3.5 mg, about 0.51 mg to about 3 mg, about 0.51 mg to about 2.5 mg, about 0.51 mg to about 2 mg, about 0.51 mg to about 1.5 mg, about 0.51 mg to about 1 mg, about 1.01 mg to about 5 mg, about 1.01 mg to about 4.5 mg, about 1.01 mg to about 4 mg, about 1.01 mg to about 3.5 mg, about 1.01 mg to about 3 mg, about 1.01 mg to about 2.5 mg, about 1.01 mg to about 2 mg, about 1.01 mg to about 1.5 mg, about 1.51 mg to about 5 mg, about 1.51 mg to about 4.5 mg, about 1.51 mg to about 4 mg, about 1.51 mg to about 3.5 mg, about 1.51 mg to about 3 mg, about 1.51 mg to about 2.5 mg, about 1.51 mg to about 2 mg, about 2.01 mg to about 5 mg, about 2.01 mg to about 4.5 mg, about 2.01 mg to about 4 mg, about 2.01 mg to about 3.5 mg, about 2.01 mg to about 3 mg, about 2.01 mg to about 2.5 mg, about 2.51 mg to about 5 mg, about 2.51 mg to about 4.5 mg, about 2.51 mg to about 4 mg, about 2.51 mg to about 3.5 mg, about 2.51 mg to about 3 mg, about 3.01 mg to about 5 mg, about 3.01 mg to about 4.5 mg, about 3.01 mg to about 4 mg, about 3.01 mg to about 3.5 mg, about 3.51 mg to about 5 mg, about 3.51 mg to about 4.5 mg, about 3.51 mg to about 4 mg, about 4.01 mg to about 5 mg, about 4.01 mg to about 4.5 mg, or about 4.51 mg to about 5 mg of the melatonin is present in the composition.
Embodiment 310. The composition of any one of embodiments 297-308, wherein about 0.01 mg, about 0.51 mg, about 1.01 mg, about 1.51 mg, about 2.01 mg, about 2.51 mg, about 3.01 mg, about 3.51 mg, about 4.01 mg, or about 4.51 mg of the melatonin is present in the composition.
Embodiment 311. The composition of any one of embodiments 1-310, wherein the ashwagandha extract comprises KSM-66® D extract.
Embodiment 312. The composition of embodiment 311, wherein the KSM-66® D extract comprises: alkaloids, steroidal lactones, saponins, or anti-stress agents, or any combination of two or more thereof.
Embodiment 313. The composition of embodiment 311 or embodiment 312, wherein the ashwagandha extract is present in the composition from about 20% to about 40% (w/w), about 25% to about 35% (w/w), or from about 27.5% to about 32.5% (w/w).
Embodiment 314. The composition of embodiment 311 or embodiment 312, wherein the ashwagandha extract is present in the composition at about 20%, about 25%, about 30%, about 35%, or about 40% (w/w).
Embodiment 315. The composition of embodiment 311 or embodiment 312, wherein the ashwagandha extract is present in the composition from about 20% to about 40% (w/w), about 20% to about 35%, about 20% to about 30%, about 20% to about 25%, about 25% to about 40%, about 25% to about 35%, about 25% to about 30%, about 30% to about 40%, about 30% to about 35%, or from about 35% to about 40% (w/w).
Embodiment 316. The composition of any one of embodiments 311-315, wherein about 100 mg to about 400 mg, about 100 mg to about 350 mg, about 100 mg to about 300 mg, about 100 mg to about 250 mg, about 150 mg to about 400 mg, about 150 mg to about 350 mg, about 150 mg to about 300 mg, about 150 mg to about 250 mg, about 200 mg to about 400 mg, about 200 mg to about 350 mg, about 200 mg to about 300 mg, about 200 mg to about 250 mg, about 250 mg to about 400 mg, about 250 mg to about 350 mg, about 250 mg to about 300 mg, about 300 mg to about 400 mg, about 300 mg to about 350 mg, about 350 mg to about 400 mg of the ashwagandha extract is present in the composition.
Embodiment 317. The composition of any one of embodiments 311-315, wherein about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg of the ashwagandha extract is present in the composition.
Embodiment 318. The composition of any one of embodiments 1-312, wherein the ashwagandha extract is present in the composition from about 40% to about 60% (w/w), about 45% to about 55% (w/w), or from about 47.5% to about 52.5% (w/w).
Embodiment 319. The composition of any one of embodiments 1-312, wherein the ashwagandha extract is present in the composition at about 40%, about 45%, about 50%, about 55%, or about 60% (w/w).
Embodiment 320. The composition of any one of embodiments 1-312, wherein the ashwagandha extract is present in the composition from about 40% to about 60% (w/w), about 40% to about 55%, about 40% to about 50%, about 40% to about 45%, about 45% to about 60%, about 45% to about 55%, about 45% to about 50%, about 50% to about 60%, about 50% to about 55%, or from about 55% to about 60%.
Embodiment 321. The composition of any one of embodiments 318-320, wherein about 50 mg to about 250 mg, about 50 mg to about 200 mg, about 50 mg to about 150 mg, about 50 mg to about 125 mg, about 100 mg to about 250 mg, about 100 mg to about 200 mg, about 100 mg to about 150 mg, about 100 mg to about 125 mg, about 125 mg to about 250 mg, about 125 mg to about 200 mg, about 125 mg to about 150 mg, about 125 mg to about 175 mg, about 125 mg to about 150 mg, about 150 mg to about 250 mg, about 150 mg to about 200 mg, or about 200 mg to about 250 mg of the ashwagandha extract is present in the composition.
Embodiment 322. The composition of any one of embodiments 318-320, wherein about 50 mg, about 75 mg, about 100 mg, about 125 mg, about 150 mg, about 175 mg, about 200 mg, about 225 mg, or about 250 mg of the ashwagandha extract is present in the composition.
Embodiment 323. A composition comprising an ashwagandha extract and a cordyceps species.
Embodiment 324. A composition comprising an ashwagandha extract and phosphatidylserine.
Embodiment 325. A composition comprising an ashwagandha extract and a supplemental mineral.
Embodiment 326. A composition comprising an ashwagandha extract and a compound derived from a magnolia tree.
Embodiment 327. A composition comprising an ashwagandha extract and ginger.
Embodiment 328. A composition comprising an ashwagandha extract and horny goat weed.
Embodiment 329. A composition comprising an ashwagandha extract and sclareolides.
Embodiment 330. A composition comprising an ashwagandha extract and DIM.
Embodiment 331. A composition comprising an ashwagandha extract and zinc.
Embodiment 332. A composition comprising an ashwagandha extract and an extraction from the aerial parts of oat plants.
Embodiment 333. A composition comprising an ashwagandha extract and maca.
Embodiment 334. A composition comprising an ashwagandha extract and Eurycoma longifolia.
Embodiment 335. A composition comprising an ashwagandha extract and fenugreek seed extract.
Embodiment 336. A composition comprising an ashwagandha extract and Tribulus terrestris.
Embodiment 337. A composition comprising an ashwagandha extract and a tea extract.
Embodiment 338. A composition comprising an ashwagandha extract and a vitamin.
Embodiment 339. A composition comprising an ashwagandha extract and a nitric oxide source.
Embodiment 340. A composition comprising an ashwagandha extract and an amino acid.
Embodiment 341. A composition comprising an ashwagandha extract and Panax ginseng.
Embodiment 342. A composition comprising an ashwagandha extract and maca.
Embodiment 343. A composition comprising an ashwagandha extract and Bacopa monieri.
Embodiment 344. A composition comprising an ashwagandha extract and a dihydrohonokiol-B source.
Embodiment 345. A composition comprising an ashwagandha extract and L-theanine.
Embodiment 346. A composition comprising an ashwagandha extract and L-tryptophan.
Embodiment 347. A composition comprising an ashwagandha extract and reishi mushroom.
Embodiment 348. A composition comprising an ashwagandha extract and white willow bark.
Embodiment 349. A composition comprising an ashwagandha extract and melatonin.
Embodiment 350. A composition formulated to modulate cortisol levels in a person comprising an ashwagandha extract.
Embodiment 351. The composition of any one of embodiments 1-349, formulated in a tablet.
Embodiment 352. The composition of embodiment 350, formulated in an oral dissolving tablet.
Embodiment 353. The composition of any one of embodiments 1-351, wherein the composition disintegrates in water in less than about 10 minutes, 9 minutes, 8 minutes, 7 minutes, 6 minutes, 5 minutes, 4 minutes, 3 minutes, 2 minutes, or 1 minute.
Embodiment 354. The composition of embodiment 1-351, wherein the composition disintegrates in water in 10 minutes or 5 minutes.
Embodiment 355. The composition of any one of embodiments 1-353, wherein the composition is prepared in a process comprising lyophilization.
Embodiment 356. The composition of any one of embodiments 1-354, wherein the composition is prepared in a unit having about 350 mg to about 1350 mg weight of an ingredient.
Embodiment 357. The composition of any one of embodiments 1-354, wherein the composition is prepared in a unit having about 50 mg to about 450 mg weight of an ingredient.
Embodiment 358. The composition of embodiment 355, wherein the ingredient comprises the ashwagandha extract, and one or more of the following: excipient, chelating agent, delivery enhancing agent, cordyceps species, phosphatidylserine, supplemental mineral, compound derived from the magnolia tree, ginger, horny goat weed, sclareolides, DIM, or zinc.
Embodiment 359. The composition of embodiment 355, wherein the ingredient comprises the ashwagandha extract, and one or more of the following: EDTA, AstraGin®, CordycepsPrime™, phosphatidylserine, magnesium glycinate, Magnolia officinalis, ginger, horny goat weed, sclareolides, DIM, or zinc.
Embodiment 360. The composition of embodiment 355, wherein the ingredient comprises the ashwagandha extract, and one or more of the following: excipient, chelating agent, delivery enhancing agent, cordyceps species, phosphatidylserine, supplemental mineral, compound derived from the magnolia tree, ginger, extraction from aerial parts of oat plants, sclareolides, DIM, or zinc.
Embodiment 361. The composition of embodiment 355, wherein the ingredient comprises the ashwagandha extract, and one or more of the following: EDTA, AstraGin®, CordycepsPrime™, phosphatidylserine, magnesium glycinate, Magnolia officinalis, ginger, oat straw, sclareolides, DIM, or zinc.
Embodiment 362. The composition of embodiment 355, wherein the ingredient comprises the ashwagandha extract, and one or more of the following: excipient, chelating agent, delivery enhancing agent, maca, Eurycoma longifolia, fenugreek seed extract, Tribulus terrestris, cordyceps species, tea extract, vitamin, nitric oxide source, amino acid, or Panax ginseng.
Embodiment 363. The composition of embodiment 355, wherein the ingredient comprises the ashwagandha extract, and one or more of the following: EDTA, AstraGin®, maca, Eurycoma longifolia, Testosurge®, Tribulus terrestris, CordycepsPrime™, green tea extract, vitamin B6, beet root powder, D-aspartic acid, or Panax ginseng.
Embodiment 364. The composition of embodiment 355, wherein the ingredient comprises the ashwagandha extract, and one or more of the following: excipient, chelating agent, delivery enhancing agent, maca, fenugreek seed extract, vitamin, cordyceps species, tea extract, nitric oxide source, Bacopa monieri, amino acid, or Panax ginseng.
Embodiment 365. The composition of embodiment 355, wherein the ingredient comprises the ashwagandha extract, and one or more of the following: EDTA, AstraGin®, maca, Testosurge®, vitamin B6, CordycepsPrime™, green tea extract, beet root powder, bacopa, D-aspartic acid, or Panax ginseng.
Embodiment 366. The composition of embodiment 356, wherein the ingredient comprises the ashwagandha extract, and one or more of the following: excipient, chelating agent, delivery enhancing agent, phosphatidylserine, L-theanine, L-tryptophan, reishi mushroom, or melatonin.
Embodiment 367. The composition of embodiment 356, wherein the ingredient comprises the ashwagandha extract, and one or more of the following: EDTA, AstraGin®, phosphatidylserine, L-theanine, L-tryptophan, reishi mushroom, or melatonin.
Embodiment 368. The composition of embodiment 355 or any one of embodiments 357-364, wherein the unit has about 550 mg, 650 mg, 750 mg, 850 mg, 950 mg, 1050 mg, or about 1150 mg of ingredient.
Embodiment 369. The composition of embodiment 356 or embodiment 365 or embodiment 366, wherein the unit has about 150 mg, 175 mg, 200 mg, 225 mg, 250 mg, 275 mg, 300 mg, 325 mg, or about 350 mg of ingredient.
Embodiment 370. The composition of any one of embodiments 1-368, wherein the composition is formulated as a tablet.
Embodiment 371. A method comprising taking a composition of any one of embodiments 1-370, wherein data from the person taking the composition has been evaluated.
Embodiment 372. The method of embodiment 370, wherein the data comprises measurable data.
Embodiment 373. The method of embodiment 370 or embodiment 371, wherein the measurable data comprises one or more of the following: age, daily sleep (e.g., 5 hours each night), hours spent exercising daily, cortisol levels of the person measured when the person wakes up in the morning, cortisol levels of the person measured at midday, cortisol levels of the person measured in the evening, and cortisol levels of the person measured at night.
Embodiment 374. The method of any one of embodiments 370-372, wherein the data comprises qualitative data.
Embodiment 375. The method of any one of embodiments 370-373, wherein the qualitative data comprises one or more of the following: daily diet, libido, mood, energy during daily exercise, and stress levels of the person.
Embodiment 376. The method of any one of embodiments 370-374, wherein determining comprises inputting the data into a computer program.
Embodiment 377. A method of improving the health of a person, the method comprising taking a combination of one or more compositions.
Embodiment 378. The method of embodiment 376, wherein the one or more compositions comprise the composition of any one of embodiments 1-369.
Embodiment 379. The method of embodiment 376 or embodiment 377, wherein the one or more compositions is 1, 2, or 3 compositions.
Embodiment 380. The method of any one of embodiments 376-378, wherein the one or more compositions comprises a composition comprising RH1.
Embodiment 381. The method of embodiment 379, wherein the composition comprising RH1 is taken in the morning.
Embodiment 382. The method of any one of embodiments 376-380, wherein the one or more compositions comprises a composition comprising RH2.
Embodiment 383. The method of embodiment 381, wherein the composition comprising RH2 is taken in the evening.
Embodiment 384. The method of any one embodiments 376-382, wherein the one or more compositions comprises a composition comprising RH3.
Embodiment 385. The method of embodiment 384, wherein the composition comprising RH3 is taken before the person goes to sleep.
Embodiment 386. A method of improving the health of a person, the method comprising taking a composition of any one of embodiments 1-371.
Embodiment 387. The method of embodiment 372, wherein the delivery is oral.
Embodiment 388. The method of any one of embodiments 372-374, wherein the composition increases energy, as compared to the person prior to taking the composition.
Embodiment 389. The method of any one of embodiments 372-374, wherein the composition facilitates relaxation, as compared to the person prior to taking the composition.
Embodiment 390. The method of any one of embodiments 372-374, wherein the composition improves sleep, as compared to the person prior to taking the composition.
Embodiment 391. The method of any one of embodiments 372-376, wherein the composition is taken a regimen comprises two or more units.
Embodiment 392. The method of embodiment 377, wherein the two or more units is 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 units.
Embodiment 393. The method of embodiment 377 or embodiment 378, wherein each unit is taken daily or twice daily.
Embodiment 394. The method of any one of embodiments 377-379, wherein each unit is taken every other day, every two days, every three days, every four days, every five days, every six days, every seven days, once a week, or once every two weeks.
Embodiment 395. The method of any one of embodiments 377-380, wherein the regimen comprises taking the composition once daily for a week.
Embodiment 396. The method of any one of embodiments 377-380, wherein the regimen comprises taking the composition once daily for a month.
Embodiment 397. The method of any one of embodiments 377-382, wherein the regimen is repeated about or at least about 2, 3, 4, 5, 6, 7, 8, 9, or 10 times.
Embodiment 398. A composition comprising:
   an organic plant extract;
   a delivery enhancing agent; and
   a chelating agent.
Embodiment 399. The composition of embodiment 398, further comprising at least one of one or more excipients.
Embodiment 400. The composition of embodiment 399, wherein the one or more excipients comprise:
   a sodium starch glycolate source;
   an adsorbent;
   a glidant;
   a silicon dioxide source;
   a cellulose source;
   a binder;
   a diluent;
   a sweetening agent;
   a flavoring agent;
   a starch excipient; or
   magnesium stearate; or a combination of two or more thereof.
Embodiment 401. The composition of embodiment 398 or embodiment 399, further comprising at least one of the following:
   Maca;
   Eurycoma longifolia;
   a fenugreek extract source;
   a Tribulus terrestris source;
   a cordyceps mushroom source;
   at least one vitamin;
   a nitric oxide source;
   a tea extract source;
   an amino acid; or
   Panax ginseng.
Embodiment 402. The composition of embodiment 398 or 399, further comprising at least one of the following:
   Maca;
   a fenugreek extract source;
   a cordyceps mushroom source;
   at least one vitamin;
   a nitric oxide source;
   a bacopa source;
   a tea extract source;
   an amino acid; or
   Panax ginseng.
Embodiment 403. The composition of embodiment 398 or 399 or 401, further comprising at least one of the following:
   a cordyceps species;
   phosphatidylserine;
   magnesium glycinate;
   Magnolia officinalis;
   ginger;
   horny goat weed;
   sclareolides;
   DIM; or
   Zinc.
Embodiment 404. The composition of embodiment 398 or 399 or 401, further comprising at least one of the following:
   a cordyceps species;
   phosphatidylserine;
   magnesium glycinate;
   Magnolia officinalis;
   ginger;
   an oat straw source;
   sclareolides;
   DIM; or
   Zinc.
Embodiment 405. The composition of embodiment 398 or 399, further comprising at least one of the follow:
   phosphatidylserine;
   Magnolia officinalis;
   L-theanine;
   L-tryptophan;
   reishi mushroom; or
   melatonin.
Embodiment 406. The composition of any one of embodiments 398-405, wherein the organic plan extract comprises ashwagandha.
Embodiment 407. The composition of any one of embodiments 398-406, wherein the delivery enhancing agent comprises an extract of Panax notoginseng or an extract of Astragalus membranaceus.
Embodiment 408. The composition of any one of embodiments 398-407, wherein the chelating agent comprises EDTA.
Embodiment 409. The composition of any one of embodiments 398-408, wherein the organic plant extract is present in the composition from about 20% to about 40% (w/w).
Embodiment 410. The composition of any one of embodiments 398-409, wherein about 100 mg to about 400 mg of the organic plant extract is present in the composition;
Embodiment 411. The composition of any one of embodiments 398-410, wherein the delivery enhancing agent is present in the composition from about 1% to about 10% (w/w).
Embodiment 412. The composition of any one of embodiments 398-411, wherein about 25 mg to about 75 mg of the delivery enhancing agent is present in the composition.
Embodiment 413. The composition of any one of embodiments 398-412, wherein the chelating agent is present in the composition from about 1% to about 10% (w/w).
Embodiment 414. The composition of any one of embodiments 398-413, wherein about 25 mg to about 75 mg of the chelating agent is present in the composition.
Embodiment 415. The composition of any one of embodiments 398-414, wherein the composition further comprises one or more of cordyceps species, phosphatidylserine, supplemental mineral, compound derived from the magnolia tree, ginger, horny goat weed, sclareolides, DIM, zinc, extraction from aerial parts of oat plants, Eurycoma longifolia, fenugreek seed extract, Tribulus terrestris, tea extract, vitamin, nitric oxide source, amino acid, Panax ginseng, maca, Bacopa monieri, dihydrohonokiol-B, L-theanine, L-tryptophan, reishi mushroom, white willow bark, and melatonin.

### Examples

The following examples are illustrative of the embodiments described herein and are not to be interpreted as limiting the scope of this disclosure. To the extent that specific materials are mentioned, it is merely for purposes of illustration and is not intended to be limiting. One skilled in the art may develop equivalent means or reactants without the exercise of inventive capacity and without departing from the scope of this disclosure.

### Example 1: Example Composition

In one embodiment an ashwagandha extract is formulated in a composition for oral delivery. Ingredients of the composition are as shown in Table 1 below. There is an ashwagandha extract component of KSM-66®. KSM-66® D comprises 250 mg of the 1150 mg tablet composition, or 21.74%. Various excipient components are present in the composition. A first excipient is sodium starch glycolate comprising 55 mg of the 1150 mg tablet composition, or 4.78%. A second excipient is colloidal silicon dioxide comprising about 3 mg of the 1150 mg tablet composition, or about 0.26%. The third excipient is microcrystalline cellulose comprising about 132 mg of the 1150 mg tablet composition, or about 11.51%. The fourth excipient is sucralose comprising 22 mg of the 1150 composition, or about 1.91%. The fifth excipient is peppermint flavor spray dried P-183 comprising about 40 mg of the 1150 mg tablet composition, or about 3.51%. The sixth excipient is natural peppermint flavor comprising about 40 mg of the 1150 mg tablet composition, or about 3.51%. The seventh excipient is menthol crystals comprising about 4.5 mg of the 1150 mg tablet composition, or about 0.40%. The eighth excipient is starch excipient comprising 50 mg of the 1150 mg tablet composition, or about 4.35%. The ninth excipient is magnesium stearate comprising 6 mg of the 1150 mg tablet composition, or 0.52%. The chelating agent component of the composition is EDTA. EDTA comprises 50 mg of the 1150 mg tablet composition, or 4.35%. The delivery enhancing agent component of the composition is AstraGin®. AstraGin® comprises 50 mg of the 1150 mg tablet composition, or 4.35%. The composition comprises a maca component. Maca comprises 187.5 mg of the 1150 mg tablet composition, or 16.31%. The composition comprises a Eurycoma longifolia component. Eurycoma longifolia comprises 112.5 mg of the 1150 mg tablet composition, or 9.78%. The composition comprises a fenugreek seed extract component. Fenugreek seed extract comprises 107.5 mg of the 1150 mg tablet composition, or 9.35%. The composition comprises a tribulus terrestirs component. Tribulus terrestris comprises about 12.5 mg of the 1150 mg tablet composition, or 1.09%. A cordyceps species component of the composition comprises cordyceps prime. Cordyceps prime comprises 5 mg of the 1150 mg tablet composition, or 0.43%. The nitric oxide source component of the composition comprises beet root powder. Beet root powder comprises 5 mg of the 1150 mg tablet composition, or 0.43%. The vitamin component of the composition comprises vitamin B6. Vitamin B6 comprises 1.3 mg of the 1150 mg tablet composition, or 0.11%. A tea extract component of the composition comprises green tea extract. Green tea extract comprises 5 mg of the 1150 mg tablet composition, or 0.43%. An amino acid component of the composition comprises D-aspartic acid. D-aspartic acid comprises 5 mg of the 1150 mg tablet composition, or 0.43%. The composition comprises a Panax ginseng component. Panax ginseng comprises 5 mg of the 1150 mg tablet composition, or 0.43%. In certain examples, this composition is formulated for a male.

### Example 2: Example Composition

In one embodiment an ashwagandha extract is formulated in a composition for oral delivery. Ingredients of the composition are as shown in Table 2 below. There is an ashwagandha extract component of KSM-66®. KSM-66® D comprises 250 mg of the 1150 mg tablet composition, or 21.74%. Various excipient components are present in the composition. A first excipient is sodium starch glycolate comprising 55 mg of the 1150 mg tablet composition, or 4.78%. A second excipient is colloidal silicon dioxide comprising about 3 mg of the 1150 mg tablet composition, or about 0.26%. The third excipient is microcrystalline cellulose comprising about 135 mg of the 1150 mg tablet composition, or about 11.77%. The fourth excipient is sucralose comprising about 24 mg of the 1150 composition, or about 2.10%. The fifth excipient is peppermint flavor spray dried P-183 comprising about 40 mg of the 1150 mg tablet composition, or about 3.50%. The sixth excipient is natural peppermint flavor comprising about 40 mg of the 1150 mg tablet composition, or about 3.50%. The seventh excipient is menthol crystals comprising about 4.6 mg of the 1150 mg tablet composition, or about 0.40%. The eighth excipient is starch excipient comprising 50 mg of the 1150 mg tablet composition, or about 4.35%. The ninth excipient is magnesium stearate comprising 6 mg of the 1150 mg tablet composition, or 0.52%. The chelating agent component of the composition is EDTA. EDTA comprises 50 mg of the 1150 mg tablet composition, or 4.35%. The delivery enhancing agent component of the composition is AstraGin®. AstraGin® comprises 50 mg of the 1150 mg tablet composition, or 4.35%. The composition comprises a maca component. Maca comprises 220 mg of the 1150 mg tablet composition, or 19.13%. The composition comprises a fenugreek seed extract component. The fenugreek seed extract comprises 170 mg of the 1150 mg tablet composition, or about 14.78%. The vitamin component of the composition comprises vitamin B6. Vitamin B6 comprises 1.3 mg of the 1150 mg tablet composition, or 0.11%. A cordyceps species component of the composition comprises cordyceps prime. Cordyceps prime comprises 10 mg of the 1150 mg tablet composition, or 0.87%%. A tea extract component of the composition comprises green tea extract. Green tea extract comprises 10 mg of the 1150 mg tablet composition, or 0.87%. The nitric oxide source component of the composition comprises beet root powder. Beet root powder comprises 10 mg of the 1150 mg tablet composition, or 0.87%. The composition comprises a bacopa component. Bacopa comprises 10 mg of the 1150 mg tablet composition, or 0.87%. An amino acid component of the composition comprises D-aspartic acid. D-aspartic acid comprises 5 mg of the 1150 mg tablet composition, or 0.43%. The composition comprises a Panax ginseng component. Panax ginseng comprises 5 mg of the 1150 mg tablet composition, or 0.43%. In certain examples, this composition is formulated for a female.

### Example 3: Example Composition

In one embodiment an ashwagandha extract is formulated in a composition for oral delivery. Ingredients of the composition are as shown in Table 3 below. There is an ashwagandha extract component of KSM-66®. KSM-66® D comprises 250 mg of the 1150 mg tablet composition, or 21.74%. Various excipient components are present in the composition. A first excipient is sodium starch glycolate comprising 55 mg of the 1150 mg tablet composition, or 4.78%. A second excipient is colloidal silicon dioxide comprising about 3 mg of the 1150 mg tablet composition, or about 0.26%. The third excipient is microcrystalline cellulose comprising about 122 mg of the 1150 mg tablet composition, or about 10.60%. The fourth excipient is sucralose comprising 23 mg of the 2250 composition, or about 2.00%. The fifth excipient is peppermint flavor spray dried P-183 comprising about 40 mg of the 1150 mg tablet composition, or about 3.50%. The sixth excipient is natural peppermint flavor comprising about 40 mg of the 1150 mg tablet composition, or about 3.50%. The seventh excipient is menthol crystals comprising about 4.6 mg of the 1150 mg tablet composition, or about 0.40%. The eighth excipient is starch excipient comprising 50 mg of the 1150 mg tablet composition, or about 4.35%. The ninth excipient is magnesium stearate comprising 6 mg of the 1150 mg tablet composition, or 0.52%. The chelating agent component of the composition is EDTA. EDTA comprises 50 mg of the 1150 mg tablet composition, or 4.35%. The delivery enhancing agent component of the composition is AstraGin®. AstraGin® comprises 50 mg of the 1150 mg tablet composition, or 4.35%. A cordyceps species component of the composition comprises cordyceps prime. CordycepsPrime comprises 185 mg of the 1150 mg tablet composition, or 16.09%. The composition comprises a phosphatidylserine component. Phosphatidylserine comprises 125 mg of the 1150 mg tablet composition, or about 10.87%. A supplemental mineral component of the composition comprises magnesium glycinate. Magnesium glycinate comprises 50 mg of the 1150 mg tablet composition, or 4.35%. A compound derived from a magnolia tree component of the composition comprises Magnolia officinalis. Magnolia officinalis comprises 50 mg of the 1150 mg tablet composition, or 4.35%. The composition comprises a ginger component. Ginger comprises 12.5 mg of the 1150 mg tablet composition, or 1.09%. The composition comprises a horny goat weed component. Horny goat weed comprises 12.5 mg of the 1150 mg tablet composition, or 1.09%. The composition comprises a sclareolides component. Sclareolides comprises 5 mg of the 1150 mg tablet composition, or 0.43%. The composition comprises a DIM component. DIM comprises 5 mg of the 1150 mg tablet composition, or 0.43%. The composition comprises a zinc component. Zinc comprises 11 mg of the 1150 mg tablet composition, or 0.96%. In certain examples, this composition is formulated for a male.

### Example 4: Example Composition

In one embodiment an ashwagandha extract is formulated in a composition for oral delivery. Ingredients of the composition are as shown in Table 4 below. There is an ashwagandha extract component of KSM-66®. KSM-66® D comprises 250 mg of the 1150 mg tablet composition, or 21.74%. Various excipient components are present in the composition. A first excipient is sodium starch glycolate comprising 55 mg of the 1150 mg tablet composition, or 4.78%. A second excipient is colloidal silicon dioxide comprising about 3 mg of the 1150 mg tablet composition, or about 0.26%. The third excipient is microcrystalline cellulose comprising about 125 mg of the 1150 mg tablet composition, or about 10.91%. The fourth excipient is sucralose comprising about 22 mg of the 1150 composition, or about 1.91%. The fifth excipient is peppermint flavor spray dried P-183 comprising about 40 mg of the 1150 mg tablet composition, or about 3.52%. The sixth excipient is natural peppermint flavor comprising about 40 mg of the 1150 mg tablet composition, or about 3.52%. The seventh excipient is menthol crystals comprising about 4.6 mg of the 1150 mg tablet composition, or about 0.40%. The eighth excipient is starch excipient comprising 50 mg of the 1150 mg tablet composition, or about 4.35%. The ninth excipient is magnesium stearate comprising 6 mg of the 1150 mg tablet composition, or 0.52%. The chelating agent component of the composition is EDTA. EDTA comprises 50 mg of the 1150 mg tablet composition, or 5.88%. The delivery enhancing agent component of the composition is AstraGin®. AstraGin® comprises 50 mg of the 1150 mg tablet composition, or 4.35%. A cordyceps species component of the composition comprises cordyceps prime. Cordyceps prime comprises 185 mg of the 1150 mg tablet composition, or 16.09%. The composition comprises a phosphatidylserine component. Phosphatidylserine comprises 125 mg of the 1150 mg tablet composition, or 10.87%. A supplemental mineral component of the composition comprises magnesium glycinate. Magnesium glycinate comprises 50 mg of the 1150 mg tablet composition, or 4.35%. The compound derived from the magnolia tree component comprises magnolia officinalis. Magnolia officinalis comprises 50 mg of the 1150 mg tablet composition, or 4.35%. The composition comprises a ginger component. Ginger comprises 12.5 mg of the 1150 mg tablet composition, or 1.09%. The extraction from aerial parts of oat plants component of the composition comprises oat straw. Oat straw comprises 12.5 mg of the 1150 mg tablet composition, or 1.09%. The composition comprises a sclareolides component. Sclareolides comprises 5 mg of the 1150 mg tablet composition, or 0.43%. The composition comprises a DIM component. DIM comprises 5 mg of the 1150 mg tablet composition, or 0.43%. The composition comprises a zinc component. Zinc comprises 8 mg of the 1150 mg tablet composition, or 0.70%. In certain examples, this composition is formulated for a female.

### Example 5: Example Composition

In one embodiment an ashwagandha extract is formulated in a composition for oral delivery. Ingredients of the composition are as shown in Table 5 below. There is an ashwagandha extract component of KSM-66®. KSM-66® D comprises 125 mg of the 325 mg tablet composition, or 38.46%. Various excipient components are present in the composition. A first excipient is sodium starch glycolate comprising about 16 mg of the 325 mg tablet composition, or 5.00%. A second excipient is colloidal silicon dioxide comprising about 1 mg of the 325 mg tablet composition, or about 0.30%. The third excipient is microcrystalline cellulose comprising about 40 mg of the 325 mg tablet composition, or about 12.22%. The fourth excipient is sucralose comprising about 7 mg of the 325 composition, or about 2.15%. The fifth excipient is peppermint flavor spray dried P-183 comprising about 11.5 mg of the 325 mg tablet composition, or about 3.50%. The sixth excipient is natural peppermint flavor comprising about 11.5 mg of the 325 mg tablet composition, or about 3.50%. The seventh excipient is menthol crystals comprising about 1.3 mg of the 325 mg tablet composition, or about 0.40%. The eighth excipient is starch excipient comprising 10.5 mg of the 325 mg tablet composition, or about 3.23%. The ninth excipient is magnesium stearate comprising 1.5 mg of the 325 mg tablet composition, or 0.46%. The chelating agent component of the composition is EDTA. EDTA comprises 25 mg of the 325 mg tablet composition, or 7.69%. The delivery enhancing agent component of the composition is AstraGin®. AstraGin® comprises 25 mg of the 325 mg tablet composition, or 7.69%. The composition comprises a phosphatidylserine component. Phosphatidylserine comprises 12.5 mg of the 325 mg tablet composition, or 3.85%. A compound derived from the magnolia tree component of the composition comprises Magnolia officinalis. Magnolia officinalis comprises 12.5 mg of the 325 mg tablet composition, or 3.85%. The composition comprises a L-theanine component. L-theanine comprises 10 mg of the 325 mg tablet composition, or 3.08%. The composition comprises a L-tryptophan component. L-tryptophan comprises 7.5 mg of the 325 mg tablet composition, or 2.31%. The composition comprises a reishi mushroom component. Reishi mushroom comprises 5 mg of the 325 mg tablet composition, or 1.54%. The composition comprises a melatonin component. Melatonin comprises 2.5 mg of the 325 mg tablet composition, 0.77%.

TABLE 1a
Example Composition RH1

| | RH1a | RH1b | RH1c | RH1d | RH1e | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 250,000 | 200,000 | 400,000 | 300,000 | 150,000 | |
| EDTA | 50,000 | 40,000 | 45,000 | 35,000 | 30,000 | |
| AstraGin ® | 50,000 | 40,000 | 45,000 | 35,000 | 30,000 | |
| Maca | 187,500 | 170,000 | 180,000 | 200,000 | 150,000 | |
| Eurycoma Longifolia | 112,500 | 100,000 | 125,000 | 137,500 | 87,500 | |
| Fenugreek (Testosurge ®) | 107,500 | 97,500 | 117,500 | 132,500 | 82,500 | |
| Tribulus Terrestris | 12,540 | 10,000 | 15,000 | 17,500 | 20,000 | |
| Cordyceps Mushroom | 5,000 | 6,000 | 4,000 | 10,000 | 12,000 | |
| (CordycepsPrime ™) | | | | | | |
| Vitamin B6 | 1,300 | 1,500 | 1,100 | 2,500 | 3,500 | |
| Nitric Oxide (Beet Root | 5,000 | 6,000 | 4,000 | 10,000 | 12,000 | |
| Powder) | | | | | | |
| Green Tea Extract | 5,000 | 6,000 | 4,000 | 10,000 | 12,000 | |
| D-Aspartic Acid | 5,000 | 6,000 | 4,000 | 10,000 | 12,000 | |
| Panax Ginseng | 5,000 | 6,000 | 4,000 | 10,000 | 12,000 | |
| Sodium Starch Glycolate | 55,000 | 60,000 | 45,000 | 38,000 | 110,000 | |
| Colloidal Silicon Dioxide | 2,970 | 4,000 | 2,000 | 2,500 | 3,500 | |
| Microcrystalline Cellulose | 132,300 | 145,000 | 120,000 | 125,000 | 140,000 | |
| Sucralose | 22,000 | 24,000 | 20,000 | 21,000 | 25,000 | |
| Peppermint Flavor Spray | 40,300 | 42,000 | 37,000 | 38,000 | 45,000 | |
| Dried P-183 | | | | | | |
| Natural Peppermint Flavor | 40,300 | 42,000 | 37,000 | 38,000 | 45,000 | |
| Menthol Crystals | 4,550 | 5,000 | 4,000 | 4,250 | 4,750 | |
| Starch Excipient | 50,000 | 40,000 | 45,000 | 35,000 | 30,000 | |
| Magnesium Stearate | 6,000 | 7,000 | 5,000 | 4,000 | 3,500 | |
| | 1,150,000 | 1,058,000 | 1,263,600 | 1,215,750 | 1,020,250 | mcg |
| | 1150.00 | 1058.00 | 1,263.60 | 1,215.750 | 1,020.250 | mg |
| | 1.15 | 1.058 | 1.264 | 1.216 | 1.020 | g |

TABLE 1b
Example Composition RH1

| | RH1f | RH1g | RH1h | RH1i | RH1j | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 350,000 | 200,000 | 175,000 | 325,000 | 225,000 | |
| EDTA | 60,000 | 70,000 | 75,000 | 25,000 | 37,500 | |
| AstraGin ® | 60,000 | 70,000 | 75,000 | 25,000 | 37,500 | |
| Maca | 135,000 | 220,000 | 250,000 | 90,000 | 100,000 | |
| Eurycoma Longifolia | 90,000 | 150,000 | 180,000 | 65,000 | 70,000 | |
| Fenugreek (Testosurge ®) | 100,000 | 150,000 | 160,000 | 60,000 | 75,000 | |
| Tribulus Terrestris | 11,000 | 14,000 | 18,000 | 6,000 | 5,000 | |
| Cordyceps Mushroom | 4,500 | 6,500 | 8,000 | 2,500 | 2,000 | |
| (CordycepsPrime ™) | | | | | | |
| Vitamin B6 | 1,000 | 1,750 | 2,300 | 650 | 500 | |
| Nitric Oxide (Beet Root | 4,500 | 6,500 | 8,000 | 2,500 | 2,000 | |
| Powder) | | | | | | |
| Green Tea Extract | 4,500 | 6,500 | 8,000 | 2,500 | 2,000 | |
| D-Aspartic Acid | 4,500 | 6,500 | 8,000 | 2,500 | 2,000 | |
| Panax Ginseng | 4,500 | 6,500 | 8,000 | 2,500 | 2,000 | |
| Sodium Starch Glycolate | 50,000 | 70,000 | 85,000 | 30,000 | 24,000 | |
| Colloidal Silicon Dioxide | 2,750 | 5,000 | 5,500 | 2,200 | 1,800 | |
| Microcrystalline Cellulose | 130,000 | 160,000 | 165,000 | 80,000 | 60,000 | |
| Sucralose | 22,000 | 27,500 | 28,000 | 14,000 | 12,000 | |
| Peppermint Flavor Spray | 40,000 | 50,000 | 55,000 | 25,000 | 22,000 | |
| Dried P-183 | | | | | | |
| Natural Peppermint Flavor | 40,000 | 50,000 | 55,000 | 25,000 | 22,000 | |
| Menthol Crystals | 4,500 | 5,000 | 5,500 | 2,000 | 1,800 | |
| Starch Excipient | 60,000 | 70,000 | 75,000 | 25,000 | 37,500 | |
| Magnesium Stearate | 7,000 | 8,000 | 8,500 | 3,000 | 4,500 | |
| | 1,185,750 | 1,353,750 | 1,457,800 | 815,350 | 746,100 | mcg |
| | 1,185.750 | 1,353.750 | 1,457.800 | 815.350 | 746.100 | mg |
| | 1.186 | 1.354 | 1.458 | 0.815 | 0.746 | g |

TABLE 2a
Example Composition RH1

| | RH1k | RH1l | RH1m | RH1n | RH1o | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 250,000 | 200,000 | 400,000 | 300,000 | 500,000 | |
| EDTA | 50,000 | 40,000 | 45,000 | 35,000 | 100,000 | |
| AstraGin ® | 50,000 | 40,000 | 45,000 | 35,000 | 100,000 | |
| MACA | 220,000 | 240,000 | 200,000 | 260,000 | 440,000 | |
| Fenugreek (Testosurge ®) | 170,000 | 170,000 | 180,000 | 200,000 | 340,000 | |
| Vitamin B6 | 1,300 | 1,500 | 1,100 | 2,500 | 3,500 | |
| Cordyceps Mushroom | 10,000 | 11,000 | 9,000 | 15,000 | 22,000 | |
| (CordycepsPrime ™) | | | | | | |
| Green Tea Extract | 10,000 | 11,000 | 9,000 | 15,000 | 22,000 | |
| Nitric Oxide (Beet Root | 10,000 | 11,000 | 9,000 | 15,000 | 22,000 | |
| Powder) | | | | | | |
| Bacopa | 10,000 | 11,000 | 9,000 | 15,000 | 22,000 | |
| D-Aspartic Acid | 5,000 | 6,000 | 4,000 | 10,000 | 12,000 | |
| Panax Ginseng | 5,000 | 6,000 | 4,000 | 10,000 | 12,000 | |
| Sodium Starch Glycolate | 55,000 | 60,000 | 45,000 | 38,000 | 110,000 | |
| Colloidal Silicon Dioxide | 2,970 | 4,000 | 2,000 | 2,500 | 6,000 | |
| Microcrystalline Cellulose | 135,330 | 145,000 | 200,000 | 160,000 | 280,000 | |
| Sucralose | 24,200 | 25,000 | 45,000 | 23,000 | 50,000 | |
| Peppermint Flavor | 40,300 | 42,000 | 37,000 | 38,000 | 80,000 | |
| Spray Dried P-183 | | | | | | |
| Natural Peppermint Flavor | 40,300 | 42,000 | 37,000 | 38,000 | 80,000 | |
| Menthol Crystals | 4,600 | 5,000 | 4,000 | 4,500 | 12,000 | |
| Starch Excipient | 50,000 | 40,000 | 45,000 | 60,000 | 100,000 | |
| Magnesium Stearate | 6,000 | 7,000 | 5,000 | 4,000 | 3,500 | |
| | 1,150,000 | 1,117,500 | 1,335,100 | 1,280,500 | 2,317,000 | mcg |
| | 1,150.00 | 1,117.500 | 1,335.100 | 1,280.500 | 2,317.000 | mg |
| | 1.15 | 1.118 | 1.335 | 1.281 | 2.317 | g |

TABLE 2b
Example Composition RH1

| | RH1p | RH1q | RH1r | RH1s | RH1t | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 350,000 | 100,000 | 175,000 | 325,000 | 225,000 | |
| EDTA | 60,000 | 70,000 | 75,000 | 25,000 | 37,500 | |
| AstraGin ® | 60,000 | 70,000 | 75,000 | 25,000 | 37,500 | |
| MACA | 280,000 | 100,000 | 150,000 | 260,000 | 200,000 | |
| Fenugreek (Testosurge ®) | 135,000 | 100,000 | 250,000 | 90,000 | 100,000 | |
| Vitamin B6 | 1,000 | 1,750 | 2,300 | 650 | 500 | |
| Cordyceps Mushroom | 10,000 | 12,000 | 15,000 | 5,000 | 6,000 | |
| (CordycepsPrime ™) | | | | | | |
| Green Tea Extract | 10,000 | 12,000 | 15,000 | 5,000 | 6,000 | |
| Nitric Oxide (Beet Root | 10,000 | 12,000 | 15,000 | 5,000 | 6,000 | |
| Powder) | | | | | | |
| Bacopa | 10,000 | 12,000 | 15,000 | 5,000 | 6,000 | |
| D-Aspartic Acid | 4,500 | 6,500 | 8,000 | 2,500 | 2,000 | |
| Panax Ginseng | 4,500 | 6,500 | 8,000 | 2,500 | 2,000 | |
| Sodium Starch Glycolate | 50,000 | 70,000 | 85,000 | 30,000 | 24,000 | |
| Colloidal Silicon Dioxide | 2,750 | 5,000 | 5,500 | 2,200 | 1,800 | |
| Microcrystalline Cellulose | 190,000 | 160,000 | 165,000 | 180,000 | 120,000 | |
| Sucralose | 30,000 | 30,000 | 31,000 | 30,000 | 20,000 | |
| Peppermint Flavor | 40,000 | 50,000 | 55,000 | 25,000 | 22,000 | |
| Spray Dried P-183 | | | | | | |
| Natural Peppermint Flavor | 40,000 | 50,000 | 55,000 | 25,000 | 22,000 | |
| Menthol Crystals | 5,500 | 7,000 | 7,750 | 3,500 | 3,000 | |
| Starch Excipient | 60,000 | 70,000 | 75,000 | 25,000 | 37,500 | |
| Magnesium Stearate | 7,000 | 8,000 | 8,500 | 3,000 | 4,500 | |
| | 1,360,250 | 952,750 | 1,291,050 | 1,074,350 | 883,300 | mcg |
| | 1,360.25- | 952.750 | 1,291.050 | 1,074.350 | 883.300 | mg |
| | 1.360 | 0.953 | 1.291 | 1.074 | 0.883 | g |

TABLE 2c
Example Composition RH1

| | RH1u | RH1v | RH1w | |
|---|---|---|---|---|
| | By Weight | By Weight | By Weight | |
| Ingredients | (mcg) | (mcg) | (mcg) | |
| Ashwagandha (KSM-66 ®) | 321,300 | 200,000 | 250,000 | |
| EDTA | 64,260 | 30,000 | 20,000 | |
| AstraGin ® | 64,260 | 75,000 | 75,000 | |
| MACA | 240,975 | 100,000 | 100,000 | |
| Eurycoma Longifolia | 141,750 | 250,000 | 200,000 | |
| Fenugreek (Testosurge ®) | 135,450 | 100,000 | 150,000 | |
| Tribulus Terrestris | 15,800 | 10,000 | 20,000 | |
| Cordyceps Mushroom | 6,300 | 10,000 | 5,000 | |
| (CordycepsPrime ™) | | | | |
| Vitamin B6 | 1,972 | 5,000 | 1,500 | |
| Beet Root | 6,300 | 12,000 | 10,000 | |
| Green Tea Extract | 6,300 | 12,000 | 10,000 | |
| D-Aspartic Acid | 6,300 | 12,000 | 10,000 | |
| Panax Ginseng | 6,300 | 12,000 | 10,000 | |
| Sodium Starch Glycolate | 6,300 | 12,000 | 10,000 | |
| (Explotab) | | | | |
| Anhydrous Silicon Dioxide | 90,000 | 40,000 | 120,000 | |
| FUJI FMELT TYPE C | 250,000 | 500,000 | 100,000 | |
| MONK FRUIT MV50 | 48,000 | 25,000 | 75,000 | |
| STEVIA | 25,200 | 10,000 | 35,000 | |
| Peppermint Oil/Spearmint | 108,290 | 150,000 | 125,000 | |
| Oil Blend | | | | |
| Masking Agent 1 | 2,050 | 1,000 | 1,000 | |
| Masking Agent 2 | 1,030 | 2,000 | 2,000 | |
| Starcap 1500 | 63,000 | 85,000 | 50,000 | |
| Magnesium Stearate | 7,600 | 5,000 | 10,000 | |
| | 1,618,737 | 1,658,000 | 1,389,500 | mcg |
| | 1,619 | 1,658 | 1,390 | mg |
| | 1.62 | 1.66 | 1.39 | g |

TABLE 3a
Example Composition RH2

| | RH2a | RH2b | RH2c | RH2d | RH2e | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 250,000 | 200,000 | 400,000 | 300,000 | 500,000 | |
| EDTA | 50,000 | 40,000 | 45,000 | 35,000 | 100,000 | |
| AstraGin ® | 50,000 | 40,000 | 45,000 | 35,000 | 100,000 | |
| Cordyceps Mushroom | 185,000 | 170,000 | 180,000 | 200,000 | 320,000 | |
| (CordycepsPrime ™) | | | | | | |
| Phosphatidylserine | 125,000 | 130,000 | 120,000 | 150,000 | 240,000 | |
| (Sunflower Powder) | | | | | | |
| Magnesium Glycinate | 50,000 | 40,000 | 45,000 | 35,000 | 100,000 | |
| Magnolia Officinalis | 50,000 | 40,000 | 45,000 | 35,000 | 100,000 | |
| Ginger | 12,500 | 13,000 | 12,000 | 15,000 | 25,000 | |
| Horny Goat Weed | 12,500 | 13,000 | 12,000 | 15,000 | 25,000 | |
| Sclareolides (Sage | 5,000 | 6,000 | 4,000 | 10,000 | 12,000 | |
| Leaf Powder) | | | | | | |
| DIM | 5,000 | 6,000 | 4,000 | 10,000 | 12,000 | |
| Zinc | 11,000 | 12,000 | 20,000 | 22,000 | 27,000 | |
| Sodium Starch Glycolate | 55,000 | 60,000 | 45,000 | 38,000 | 110,000 | |
| Colloidal Silicon Dioxide | 2,970 | 4,000 | 2,000 | 2,500 | 3,500 | |
| Microcrystalline Cellulose | 1,21,930 | 145,000 | 120,000 | 125,000 | 140,000 | |
| Sucralose | 23,000 | 25,000 | 22,500 | 23,000 | 26,000 | |
| Peppermint Flavor | 40,250 | 42,000 | 37,000 | 38,000 | 45,000 | |
| Spray Dried P-183 | | | | | | |
| Natural Peppermint Flavor | 40,250 | 42,000 | 37,000 | 38,000 | 45,000 | |
| Menthol Crystals | 4,600 | 5,000 | 4,000 | 4,500 | 6,000 | |
| Starch Excipient | 50,000 | 40,000 | 45,000 | 35,000 | 30,000 | |
| Magnesium Stearate | 6,000 | 7,000 | 5,000 | 4,000 | 3,500 | |
| | 1,150,000 | 1,080,000 | 1,249,500 | 1,170,000 | 1,970,000 | mcg |
| | 1,150.00 | 1,080.000 | 1,249.500 | 1,170.000 | 1,970.000 | mg |
| | 1.15 | 1.080 | 1.250 | 1.170 | 1.970 | g |

TABLE 3b
Example Composition RH2

| | RH2f | RH2g | RH2h | RH2i | RH2j | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 350,000 | 250,000 | 175,000 | 325,000 | 225,000 | |
| EDTA | 60,000 | 70,000 | 75,000 | 25,000 | 37,500 | |
| AstraGin ® | 60,000 | 70,000 | 75,000 | 25,000 | 37,500 | |
| Cordyceps Mushroom | 135,000 | 220,000 | 250,000 | 90,000 | 100,000 | |
| (CordycepsPrime ™) | | | | | | |
| Phosphatidylserine | 80,000 | 160,000 | 190,000 | 60,000 | 65,000 | |
| (Sunflower Powder) | | | | | | |
| Magnesium Glycinate | 60,000 | 70,000 | 75,000 | 25,000 | 37,500 | |
| Magnolia Officinalis | 60,000 | 70,000 | 75,000 | 25,000 | 37,500 | |
| Ginger | 8,000 | 16,000 | 19,000 | 6,000 | 6,500 | |
| Horny Goat Weed | 8,000 | 16,000 | 19,000 | 6,000 | 6,500 | |
| Sclareolides (Sage | 4,500 | 6,500 | 8,000 | 2,500 | 2,000 | |
| Leaf Powder) | | | | | | |
| DIM | 4,500 | 6,500 | 8,000 | 2,500 | 2,000 | |
| Zinc | 17,000 | 13,000 | 16,000 | 15,000 | 7,000 | |
| Sodium Starch Glycolate | 50,000 | 70,000 | 85,000 | 30,000 | 24,000 | |
| Colloidal Silicon Dioxide | 2,750 | 5,000 | 5,500 | 2,200 | 1,800 | |
| Microcrystalline Cellulose | 130,000 | 160,000 | 165,000 | 80,000 | 60,000 | |
| Sucralose | 21,000 | 30,000 | 31,000 | 15,000 | 12,000 | |
| Peppermint Flavor | 40,000 | 50,000 | 55,000 | 25,000 | 22,000 | |
| Spray Dried P-183 | | | | | | |
| Natural Peppermint Flavor | 40,000 | 50,000 | 55,000 | 25,000 | 22,000 | |
| Menthol Crystals | 5,500 | 7,000 | 7,750 | 3,500 | 3,000 | |
| Starch Excipient | 60,000 | 70,000 | 75,000 | 25,000 | 37,500 | |
| Magnesium Stearate | 7,000 | 8,000 | 8,500 | 3,000 | 4,500 | |
| | 1,203,250 | 1,418,000 | 1,472,750 | 815,700 | 750,800 | mcg |
| | 1,203.250 | 1,418.000 | 1,472.750 | 815.700 | 750.800 | mg |
| | 1.203 | 1.418 | 1.473 | 0.816 | 0.751 | g |

TABLE 3c
Example Composition RH2

| | RH2k | RH2l | RH2m | |
|---|---|---|---|---|
| | By Weight | By Weight | By Weight | |
| Ingredients | (mcg) | (mcg) | (mcg) | |
| Ashwagandha (KSM-66 ®) | 250,000 | 200,000 | 400,000 | |
| EDTA | 50,000 | 60,000 | 75,000 | |
| AstraGin ® | 50,000 | 60,000 | 55,000 | |
| Cordyceps Mushroom | 185,000 | 190,000 | 175,000 | |
| (CordycepsPrime ™) | | | | |
| Phosphatidylserine | 125,000 | 150,000 | 145,000 | |
| (Sunflower Powder) | | | | |
| Magnesium Glycinate | 50,000 | 60,000 | 70,000 | |
| Magnolia Officinalis | 50,000 | 70,000 | 80,000 | |
| Ginger | 12,500 | 15,000 | 20,000 | |
| Horny Goat Weed | 12,500 | 15,000 | 20,000 | |
| Sclareolides (Sage Leaf | 5,000 | 15,000 | 4,000 | |
| Powder) | | | | |
| DIM | 5,000 | 7,000 | 3,000 | |
| Zinc | 11,000 | 5,000 | 20,000 | |
| Sodium Starch Glycolate | 55,000 | 60,000 | 45,000 | |
| Monk Fruit MV50 | 60,000 | 90,000 | 25,000 | |
| FUJI FMELT TYPE C | 500,000 | 250,000 | — | |
| Silica | 48,000 | — | — | |
| PPO, SPO, Menthol | 68,325 | 250,000 | 10,000 | |
| Microcrystalline Cellulose | — | — | — | |
| Sucralose | — | — | — | |
| Peppermint Flavor Spray | — | — | — | |
| Dried P-183 | | | | |
| Masking Agent | 3,050 | 5,000 | 1,000 | |
| Starcap 1500 | 50,000 | 40,000 | 45,000 | |
| Magnesium Stearate | 8,000 | 7,000 | 5,000 | |
| | 1,598,375 | 1,549,000 | 1,198,000 | mcg |
| | 1,598.00 | 1,549.00 | 1,198.00 | mg |
| | 1.60 | 1.55 | 1.20 | g |

TABLE 4a
Example Composition RH2

| | RH2n | RH2o | RH2p | RH2q | RH2r | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 250,000 | 200,000 | 400,000 | 300,000 | 500,000 | |
| EDTA | 50,000 | 40,000 | 45,000 | 35,000 | 30,000 | |
| AstraGin ® | 50,000 | 40,000 | 45,000 | 35,000 | 30,000 | |
| Cordyceps Mushroom | 185,000 | 170,000 | 180,000 | 200,000 | 150,000 | |
| (CordycepsPrime ™) | | | | | | |
| Phosphatidylserine | 125,000 | 130,000 | 120,000 | 150,000 | 120,000 | |
| (Sunflower Powder) | | | | | | |
| Magnesium Glycinate | 50,000 | 40,000 | 45,000 | 35,000 | 30,000 | |
| Magnolia Officinalis | 50,000 | 40,000 | 45,000 | 35,000 | 30,000 | |
| Ginger | 12,500 | 13,000 | 12,000 | 15,000 | 12,000 | |
| Oat Straw | 12,500 | 13,000 | 12,000 | 15,000 | 12,000 | |
| Sclareolides (Sage Leaf | 5,000 | 6,000 | 4,000 | 10,000 | 12,000 | |
| Powder) | | | | | | |
| DIM | 5,000 | 6,000 | 4,000 | 10,000 | 12,000 | |
| Zinc | 8,000 | 9,000 | 7,000 | 16,000 | 18,000 | |
| Sodium Starch Glycolate | 55,000 | 60,000 | 45,000 | 38,000 | 110,000 | |
| Colloidal Silicon Dioxide | 2,970 | 4,000 | 2,000 | 2,500 | 3,500 | |
| Microcrystalline Cellulose | 125,430 | 145,000 | 120,000 | 125,000 | 140,000 | |
| Sucralose | 22,000 | 25,000 | 22,500 | 23,000 | 26,000 | |
| Peppermint Flavor | 40,500 | 42,000 | 37,000 | 38,000 | 45,000 | |
| Spray Dried P-183 | | | | | | |
| Natural Peppermint Flavor | 40,500 | 42,000 | 37,000 | 38,000 | 45,000 | |
| Menthol Crystals | 4,600 | 5,000 | 4,000 | 4,500 | 6,000 | |
| Starch Excipient | 50,000 | 40,000 | 45,000 | 35,000 | 30,000 | |
| Magnesium Stearate | 6,000 | 7,000 | 5,000 | 4,000 | 3,500 | |
| | 1,150,000 | 1,077,000 | 1,236,500 | 1,164,000 | 1,365,000 | mcg |
| | 1,150.00 | 1,077.000 | 1,236.500 | 1,164.000 | 1,365.000 | mg |
| | 1.15 | 1.077 | 1.237 | 1.164 | 1.365 | g |

TABLE 4b
Example Composition RH2

| | RH2s | RH2t | RH2u | RH2v | RH2w | |
|---|---|---|---|---|---|---|
| | By | By | By | By | By | |
| | Weight | Weight | Weight | Weight | Weight | |
| Ingredients | (mcg) | (mcg) | (mcg) | (mcg) | (mcg) | |
| Ashwagandha (KSM-66 ®) | 350,000 | 100,000 | 175,000 | 325,000 | 225,000 | |
| EDTA | 60,000 | 70,000 | 75,000 | 25,000 | 37,500 | |
| AstraGin ® | 60,000 | 70,000 | 75,000 | 25,000 | 37,500 | |
| Cordyceps Mushroom | 135,000 | 220,000 | 250,000 | 90,000 | 100,000 | |
| (CordycepsPrime ™) | | | | | | |
| Phosphatidylserine | 80,000 | 160,000 | 190,000 | 60,000 | 65,000 | |
| (Sunflower Powder) | | | | | | |
| Magnesium Glycinate | 60,000 | 70,000 | 75,000 | 25,000 | 37,500 | |
| Magnolia Officinalis | 60,000 | 70,000 | 75,000 | 25,000 | 37,500 | |
| Ginger | 8,000 | 16,000 | 19,000 | 6,000 | 6,500 | |
| Oat Straw | 8,000 | 16,000 | 19,000 | 6,000 | 6,500 | |
| Sclareolides (Sage Leaf | 4,500 | 6,500 | 8,000 | 2,500 | 2,000 | |
| Powder) | | | | | | |
| DIM | 4,500 | 6,500 | 8,000 | 2,500 | 2,000 | |
| Zinc | 7,000 | 10,000 | 14,000 | 4,000 | 3,500 | |
| Sodium Starch Glycolate | 50,000 | 70,000 | 85,000 | 30,000 | 24,000 | |
| Colloidal Silicon Dioxide | 2,750 | 5,000 | 5,500 | 2,200 | 1,800 | |
| Microcrystalline Cellulose | 130,000 | 160,000 | 165,000 | 80,000 | 60,000 | |
| Sucralose | 21,000 | 30,000 | 31,000 | 15,000 | 12,000 | |
| Peppermint Flavor | 40,000 | 50,000 | 55,000 | 25,000 | 22,000 | |
| Spray Dried P-183 | | | | | | |
| Natural Peppermint Flavor | 40,000 | 50,000 | 55,000 | 25,000 | 22,000 | |
| Menthol Crystals | 5,500 | 7,000 | 7,750 | 3,500 | 3,000 | |
| Starch Excipient | 60,000 | 70,000 | 75,000 | 25,000 | 37,500 | |
| Magnesium Stearate | 7,000 | 8,000 | 8,500 | 3,000 | 4,500 | |
| | 1,193,250 | 1,265,000 | 1,470,750 | 804,700 | 747,300 | mcg |
| | 1,193.250 | 1,265.000 | 1,470.750 | 804.700 | 747.300 | mg |
| | 1.193 | 1.265 | 1.471 | 0.805 | 0.747 | g |

TABLE 4c
Example Composition RH2

| | RH2x | RH2y | RH2z | |
|---|---|---|---|---|
| | By Weight | By Weight | By Weight | |
| Ingredients | (mcg) | (mcg) | (mcg) | |
| Ashwagandha (KSM-66 ®) | 250,000 | 200,000 | 400,000 | |
| EDTA | 50,000 | 60,000 | 75,000 | |
| AstraGin ® | 50,000 | 60,000 | 55,000 | |
| Cordyceps Mushroom | 185,000 | 190,000 | 175,000 | |
| (CordycepsPrime ™) | | | | |
| Phosphatidylserine | 125,000 | 150,000 | 145,000 | |
| (Sunflower Powder) | | | | |
| Magnesium Glycinate | 50,000 | 60,000 | 70,000 | |
| Magnolia Officinalis | 50,000 | 70,000 | 80,000 | |
| Ginger | 12,500 | 15,000 | 20,000 | |
| Oat straw | 12,500 | 15,000 | 20,000 | |
| Sclareolides (Sage Leaf | 5,000 | 15,000 | 4,000 | |
| Powder) | | | | |
| DIM | 5,000 | 7,000 | 3,000 | |
| Zinc | 8,000 | 5,000 | 20,000 | |
| Sodium Starch Glycolate | 55,000 | 60,000 | 45,000 | |
| Monk Fruit MV50 | 60,000 | 90,000 | 25,000 | |
| FUJI FMELT TYPE C | 500,000 | 250,000 | — | |
| Silica | 48,000 | — | — | |
| PPO, SPO, Menthol | 68,325 | 250,000 | 10,000 | |
| Microcrystalline Cellulose | — | — | — | |
| Sucralose | — | — | — | |
| Peppermint Flavor | — | — | — | |
| Spray Dried P-183 | | | | |
| Masking Agent | 3,050 | 5,000 | 1,000 | |
| Starcap 1500 | 50,000 | 40,000 | 45,000 | |
| Magnesium Stearate | 8,000 | 7,000 | 5,000 | |
| | 1,595,375 | 1,549,000 | 1,198,000 | mcg |
| | 1,595.00 | 1,549.00 | 1,198.00 | mg |
| | 1.60 | 1.55 | 1.20 | g |

TABLE 5a
Example Composition RH3

| | RH3a | RH3b | RH3c | RH3d | RH3e |
|---|---|---|---|---|---|
| | By | By | By | By | By |
| | Weight | Weight | Weight | Weight | Weight |
| Ingredients | (mcg) | (mcg) | (mcg) | (mcg) | (mcg) |
| Ashwagandha (KSM-66 ®) | 125,000 | 100,000 | 150,000 | 200,000 | 175,000 |
| EDTA | 25,000 | 27,000 | 23,000 | 40,000 | 37,500 |
| AstraGin ® | 25,000 | 27,000 | 23,000 | 40,000 | 37,500 |
| Phosphatidylserine | 12,500 | 13,000 | 12,000 | 15,000 | 12,000 |
| (Sunflower Powder) | | | | | |
| Magnolia Officinalis | 12,500 | 13,000 | 12,000 | 15,000 | 12,000 |
| L-Theanine | 10,000 | 11,000 | 9,000 | 15,000 | 20,000 |
| L-Tryptophan | 7,500 | 8,000 | 6,000 | 12,000 | 15,000 |
| Reishi Mushroom | 5,000 | 6,000 | 4,000 | 10,000 | 12,000 |
| Melatonin | 2,500 | 3,000 | 2,000 | 5,000 | 6,000 |
| Sodium Starch Glycolate | 16,250.0 | 17,000 | 15,500 | 33,000 | 35,000 |
| Colloidal Silicon Dioxide | 975.0 | 1,500 | 750 | 2,000 | 2,250 |
| Microcrystalline Cellulose | 39,715.0 | 44,000 | 36,000 | 80,000 | 90,000 |
| Sucralose | 7,000.0 | 8,000 | 6,000 | 14,000 | 16,000 |
| Peppermint Flavor | 11,380.0 | 13,000 | 10,000 | 25,000 | 27,000 |
| Spray Dried P-183 | | | | | |
| Natural Peppermint Flavor | 11,380.0 | 13,000 | 10,000 | 25,000 | 27,000 |
| Menthol Crystals | 1,300.0 | 1,500 | 1,100 | 2,500 | 3,500 |
| Starch Excipient | 10,500.0 | 12,000 | 9,500 | 22,000 | 24,000 |
| Magnesium Stearate | 1,500.0 | 1,700 | 1,300 | 3,400 | 4,000 |
| mcg | 325,000 | 319,700 | 331,150 | 558,900 | 555,750 |
| mg | 325.00 | 319.700 | 331.150 | 558.900 | 555.750 |
| g | 0.325 | 0.320 | 0.331 | 0.559 | 0.556 |

TABLE 5b
Example Composition RH3

| | RH3f | RH3g | RH3h | RH3i | RH3j |
|---|---|---|---|---|---|
| | By | By | By | By | By |
| | Weight | Weight | Weight | Weight | Weight |
| Ingredients | (mcg) | (mcg) | (mcg) | (mcg) | (mcg) |
| Ashwagandha (KSM-66 ®) | 125,000 | 100,000 | 75,000 | 140,000 | 110,000 |
| EDTA | 25,000 | 20,000 | 15,000 | 30,000 | 24,000 |
| AstraGin ® | 25,000 | 20,000 | 15,000 | 30,000 | 24,000 |
| Phosphatidylserine | 8,000 | 16,000 | 19,000 | 6,000 | 6,500 |
| (Sunflower Powder) | | | | | |
| Magnolia Officinalis | 8,000 | 16,000 | 19,000 | 6,000 | 6,500 |
| L-Theanine | 10,000 | 12,000 | 15,000 | 5,000 | 6,000 |
| L-Tryptophan | 7,500 | 9,000 | 13,000 | 4,000 | 4,500 |
| Reishi Mushroom | 4,500 | 6,500 | 8,000 | 2,500 | 2,000 |
| Melatonin | 2,000 | 3,500 | 4,000 | 1,500 | 1,000 |
| Sodium Starch Glycolate | 14,000 | 20,000 | 23,000 | 10,000 | 8,000 |
| Colloidal Silicon Dioxide | 750 | 1,250 | 1,500 | 600 | 500 |
| Microcrystalline Cellulose | 38,000 | 42,000 | 44,000 | 20,000 | 18,000 |
| Sucralose | 6,500 | 7,500 | 8,000 | 4,000 | 3,500 |
| Peppermint Flavor | 11,000 | 12,000 | 13,000 | 6,000 | 6,000 |
| Spray Dried P-183 | | | | | |
| Natural Peppermint Flavor | 11,000 | 12,000 | 13,000 | 6,000 | 6,000 |
| Menthol Crystals | 1,000 | 1,750 | 2,300 | 650 | 500 |
| Starch Excipient | 10,000 | 16,000 | 20,000 | 5,000 | 4,750 |
| Magnesium Stearate | 1,500 | 2,250 | 2,500 | 500 | 450 |
| mcg | 308,750 | 317,750 | 310,300 | 277,750 | 232,200 |
| mg | 308.750 | 317.750 | 310.300 | 277.750 | 232.200 |
| g | 0.309 | 0.318 | 0.310 | 0.278 | 0.232 |

### Example 6: Manufacture of a Ashwagandha Extract Formulation

The ashwagandha formulations of Examples 1-5 are manufactured into tablets.

### Example 7: Method of Use

An example method of modulating cortisol levels in a person begins when a person provides information regarding the person's health, lifestyle, activities, or personal information of the person (e.g., information regarding date of birth, age, daily sleep, daily exercise, diet, libido, mental clarity, fatigue level, mood, energy level, and/or daily stress level) to a user interface associated with a processing device. The processing device receives the information provided by the user and analyzes the received information in order to determine one or more factors based on the received information. Based on the one or more factors, the system recommends taking one, two, or all three compositions: RH1, RH2, and RH3, which in one example, are the compositions of Tables 1-5. The system further provides a recommendation indicating a regimen where the person takes the RH1 unit at a first time within twelve hours of waking up, that the person takes the RH2 tablet within thirteen to twenty-four hours of waking up, and that the person takes the RH3 tablet up to two hours before falling asleep. The system further provides a recommendation indicating that the person should continue the regiment daily.

### Example 8: Method of Use

The person of Example 7 follows the regimen indicated by the system of Example 7 daily for one month. Afterward, the person inputs new data into the system of Example 7 indicating current information regarding the person and is provided a recommendation associated with at least one of the RH1, RH2, and/or RH3 products, and/or a recommendation on a lifestyle change.

### Example 9: Method of Use

The person repeats the method of Example 7 daily for one year.

### Example 10: Comparison of Melatonin Availability with a Composition Herein Vs. Market Leader Products

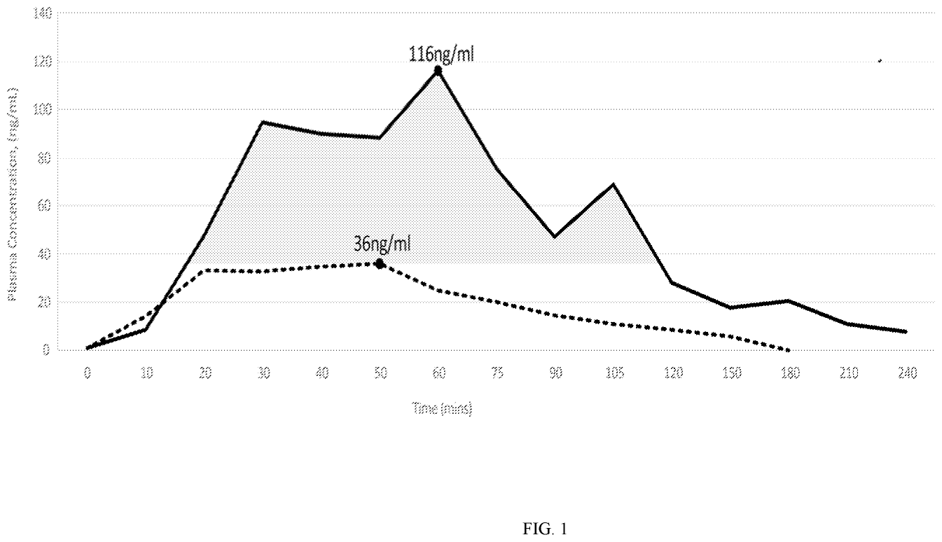

FIG. 1 depicts an example dataset of concentration of melatonin over time in the blood of a person in minutes after the person took a composition manufactured by a market leader (second formula) having 5 mg of melatonin on a first day and took a first formula comprising 5 mg of melatonin on a second day, where the top data points (solid line) are associated with melatonin levels after the person took the first formula and the bottom data points (dotted line) are associated with melatonin levels after the person took the market leader composition. In this example, the first formula comprises 5 mg of melatonin, KSM-66®, AstraGin®, and EDTA. As shown in FIG. 1, the plasma concentration of melatonin in the person's body was recorded at various points once each composition was taken as well as after the composition was taken (e.g., at the time of taking the formula, 10 minutes after taking the formula, 20 minutes after taking the formula, 30 minutes after taking the formula, 40 minutes after taking the formula, 50 minutes after taking the formula, 60 minutes after taking the formula, 75 minutes after taking the formula, 90 minutes after taking the formula, 105 minutes after taking the formula, 120 minutes after taking the formula, 150 minutes after taking the formula, 180 minutes after taking the formula, 210 minutes after taking the formula, and 240 minutes after taking the formula). The concentration levels were recorded by drawing the person's blood and measuring the concentration of melatonin in the blood.

As shown in this example, the person had a rise in concentration in the body after taking the respective compositions at the starting point (e.g., at t=0) on each day. However, the first formula allows for more bioavailability of melatonin, as shown in this example, because not only was a higher concentration of melatonin reached, but further, the concentration of melatonin in the person taking the first formula remained higher for a studied duration of time (e.g., 240 minutes after taking the first formula) than the concentration of melatonin in the person taking the second formula during the studied duration of time (e.g., 240 minutes after taking the market leader composition). For example, in the initial concentration spike after t=0 after the person took the second formula, the market leader composition, the person's concentration level reached a maximum melatonin concentration of about 36 ng/mL. Meanwhile, when the person took the first formula, the person's concentration level reached a maximum melatonin concentration of about 116 ng/mL. Additionally, after the initial spike, more melatonin remained in the person's body with the first formula than the second formula.

Thus, the first formula increased the bioavailability of melatonin in the person's system per unit of melatonin taken when compared to the bioavailability of melatonin in the person's system when the market leader composition is taken. Further, the first formula not only increased bioavailability overall, but increased the amount of time in which melatonin was bioavailable in the person's system when the first formula was taken as compared to when the market leader composition. Therefore, since ingestion and bioavailability of melatonin allow humans to fall asleep faster, stay asleep longer, and achieve a better quality of sleep, intake of the first formula may result in an increased ability to fall asleep quickly, increased ability stay asleep for longer periods of time, and increased ability to achieve a better quality of sleep. As shown, a dotted line marks about 36 ng/mL, and the shaded portion of the graph covers an area of the graph that is both above the dotted line and below the line of the person's concentration level on the second day. As such, the shaded area highlights the extra bioavailability of melatonin for the person that was a result of taking the first formula along with the ability to keep a higher concentration of melatonin in the blood over time.

### Example 11: Comparison of Fenugreek Availability with a Composition Herein Vs. Market Leader Product

A person takes two compositions, a first composition comprising 150 mg of Fenugreek, KSM-66®, AstraGin®, and EDTA, on a first day and a second, market leader composition comprising 150 mg of Fenugreek on a second day. The levels of Fenugreek are measured in the person after taking both compositions to determine a higher bioavailability of Fenugreek in the first formula.

The foregoing description of various embodiments known to the applicant at this time of filing the application has been presented and is intended for the purposes of illustration and description. The present description is not intended to be exhaustive nor limited to the precise form disclosed and many modifications and variations are possible in the light of the above teachings. The embodiments described serve to explain principles and practical applications, and to enable others skilled in the art to utilize the various embodiments, optionally with various modifications, as are suited to the particular use contemplated. Therefore, it is intended that the disclosure not be limited to the particular embodiments disclosed.

### Prophetic Example 1: Method of Use

A perimenopausal or menopausal woman takes one or more of the RH1, RH2, and RH3 compositions, which in one example, are the compositions of Tables 1-5, to modulate hormone levels, hot flashes, mood changes, and/or difficulty sleeping.

What is claimed is:

1. A composition comprising an ashwagandha extract, a first excipient comprising an extract of Astragalus membranaceus and an extract of Panax notoginseng, and a second excipient comprising a chelating agent.

2. The composition of claim 1, wherein the ashwagandha extract is present in the composition at about 20% to about 40% (w/w) or about 40% to about 60% (w/w); and/or wherein about 100 mg to about 400 mg or about 50 mg to about 250 mg of the ashwagandha extract is present in the composition.

3. The composition of claim 1 or claim 2, wherein the first excipient is present in the composition at about 1% to about 19% (w/w), about 5% to about 15% (w/w), or about 1% to about 10% (w/w); and/or wherein the composition comprises about 25 mg to about 75 mg, about 10 mg to about 50 mg, or about 10 mg to about 40 mg of the first excipient.

4. The composition of claim 1 or claim 2, wherein the second excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient.

5. The composition of claim 3, wherein the second excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient.

6. The composition of claim 1 or claim 2, wherein the chelating agent comprises ethylenediaminetetraacetic acid (EDTA), deferoxamine, deferasirox, deferiprone, penicillamine, trientine, dimercaprol, ethylenediamine, porphine, heme, sodium gluconate, tetrasodium glutamate diacetate, phytic acid, or a combination of two or more thereof.

7. The composition of any one of claims 3-5, wherein the chelating agent comprises ethylenediaminetetraacetic acid (EDTA), deferoxamine, deferasirox, deferiprone, penicillamine, trientine, dimercaprol, ethylenediamine, porphine, heme, sodium gluconate, tetrasodium glutamate diacetate, phytic acid, or a combination of two or more thereof.

8. The composition of claim 1 or claim 2, comprising (a) a melatonin, optionally present in the composition at about 0.01% (w/w) to about 2% (w/w), and optionally about 0.01 mg to about 5 mg of the melatonin is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; or (y) a combination of two or more selected from (a)-(x).

9. The composition of any one of claims 3-7, comprising: (a) a melatonin, optionally present in the composition at about 0.01% (w/w) to about 2% (w/w), and optionally about 0.01 mg to about 5 mg of the melatonin is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; or (y) a combination of two or more selected from (a)-(x).

10. The composition of any one of claims 1-9, comprising a flavoring agent, optionally present in the composition at about 1% to about 15% (w/w), and optionally about 4 mg to about 800 mg of the flavoring agent is present in the composition.

11. A composition comprising an ashwagandha extract and an excipient comprising an extract of Astragalus membranaceus and an extract of Panax notoginseng.

12. The composition of claim 11, wherein the ashwagandha extract is present in the composition at about 20% to about 40% (w/w) or about 40% to about 60% (w/w); and/or wherein about 100 mg to about 400 mg or about 50 mg to about 250 mg of the ashwagandha extract is present in the composition.

13. The composition of claim 11 or claim 12, wherein the excipient is present in the composition at about 1% to about 19% (w/w), about 5% to about 15% (w/w), or about 1% to about 10% (w/w); and/or wherein the composition comprises about 25 mg to about 75 mg, about 10 mg to about 50 mg, or about 10 mg to about 40 mg of the first excipient.

14. The composition of any one of claims 11-13, comprising (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x).

15. The composition of any one of claims 11-14, comprising a flavoring agent, optionally present in the composition at about 1% to about 15% (w/w), and optionally about 4 mg to about 800 mg of the flavoring agent is present in the composition.

16. A composition comprising an ashwagandha extract and an excipient comprising a chelating agent.

17. The composition of claim 16, wherein the ashwagandha extract is present in the composition at about 20% to about 40% (w/w) or about 40% to about 60% (w/w); and/or wherein about 100 mg to about 400 mg or about 50 mg to about 250 mg of the ashwagandha extract is present in the composition.

18. The composition of claim 16 or claim 17, wherein the excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient.

19. The composition of any one of claims 16-18, wherein the chelating agent comprises ethylenediaminetetraacetic acid (EDTA), deferoxamine, deferasirox, deferiprone, penicillamine, trientine, dimercaprol, ethylenediamine, porphine, heme, sodium gluconate, tetrasodium glutamate diacetate, phytic acid, or a combination of two or more thereof.

20. The composition of any one of claims 16-19, comprising (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x).

21. The composition of any one of claims 16-20, comprising a flavoring agent, optionally present in the composition at about 1% to about 15% (w/w), and optionally about 4 mg to about 800 mg of the flavoring agent is present in the composition.

22. A composition comprising a first excipient comprising an extract of Astragalus membranaceus and an extract of Panax notoginseng, and a second excipient comprising a chelating agent.

23. The composition of claim 22, wherein the first excipient is present in the composition at about 1% to about 19% (w/w), about 5% to about 15% (w/w), or about 1% to about 10% (w/w); and/or wherein the composition comprises about 25 mg to about 75 mg, about 10 mg to about 50 mg, or about 10 mg to about 40 mg of the first excipient.

24. The composition of claim 22 or claim 23, wherein the second excipient is present in the composition at about 1% to about 19% (w/w), about 5% to about 15% (w/w), or about 1% to about 10% (w/w); and/or wherein the composition comprises about 25 mg to about 75 mg or about 10 mg to about 50 mg of the second excipient.

25. The composition of any one of claims 22-24, wherein the chelating agent comprises ethylenediaminetetraacetic acid (EDTA), deferoxamine, deferasirox, deferiprone, penicillamine, trientine, dimercaprol, ethylenediamine, porphine, heme, sodium gluconate, tetrasodium glutamate diacetate, phytic acid, or a combination of two or more thereof.

26. The composition of any one of claims 22-25, comprising (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x).

27. The composition of any one of claims 22-26, comprising a flavoring agent, optionally present in the composition at about 1% to about 15% (w/w), and optionally about 4 mg to about 800 mg of the flavoring agent is present in the composition.

28. The composition of any one of claims 1-27, (a) formulated in a tablet; (b) formulated in an oral dissolving tablet, optionally wherein the composition disintegrates in water in less than about 10 minutes, 9 minutes, 8 minutes, 7 minutes, 6 minutes, 5 minutes, 4 minutes, 3 minutes, 2 minutes, or 1 minute; (c) wherein the composition is prepared in a process comprising lyophilization; (d) wherein the composition is prepared in a unit having a weight of about 350 mg to about 1350 mg; or (e) a combination of two or more of (a) to (d).

29. A method of modulating cortisol in a person, the method comprising taking a composition of any one of claims 1-28; optionally (a) wherein the delivery is oral; (b) wherein the composition increases energy, as compared to the person prior to taking the composition; (c) wherein the composition facilitates relaxation, as compared to the person prior to taking the composition; (d) wherein the composition improves sleep, as compared to the person prior to taking the composition; (e) wherein the composition is taken a regimen comprises two or more units, further optionally wherein each unit is taken daily or twice daily; further optionally wherein the regimen comprises taking the composition once daily for a week; (f) wherein the person is a female and the composition reduces a symptom of perimenopause or menopause in the female, as compared to the female prior to taking the composition; or (g) a combination of two or more of (a) to (f).

30. A method of improving the health of a person, the method comprising taking a composition of any one of claims 1-28; optionally (a) wherein the delivery is oral; (b) wherein the composition increases energy, as compared to the person prior to taking the composition; (c) wherein the composition facilitates relaxation, as compared to the person prior to taking the composition; (d) wherein the composition improves sleep, as compared to the person prior to taking the composition; (e) wherein the composition is taken a regimen comprises two or more units, further optionally wherein each unit is taken daily or twice daily; further optionally wherein the regimen comprises taking the composition once daily for a week; (f) wherein the person is a female and the composition reduces a symptom of perimenopause or menopause in the female, as compared to the female prior to taking the composition; or (g) a combination of two or more of (a) to (f).

31. A method of enhancing oral delivery of a composition comprising an ashwagandha extract, the method comprising combining with the ashwagandha extract (a) a first excipient comprising an extract of Astragalus membranaceus and an extract of Panax notoginseng, and/or (b) a second excipient comprising a chelating agent.

32. A method of enhancing oral delivery of an agent, the method comprising combining with the agent (a) a first excipient comprising an extract of Astragalus membranaceus and an extract of Panax notoginseng, and/or (b) a second excipient comprising a chelating agent; wherein the agent comprises: (a) a cordyceps species, optionally present in the composition at about 5% to about 35% (w/w) or about 0.1% to about 1.5% (w/w) or about 0.8% to about 1.6% (w/w), and optionally about 100 mg to about 300 mg or about 0.5 mg to about 15 mg or about 4.5 mg to about 15 mg of the cordyceps species is present in the composition; (b) a phosphatidylserine, optionally present in the composition at about 5% to about 25% (w/w) or about 0.5% to about 10% (w/w), and optionally about 25 mg to about 225 mg or about 1 mg to about 25 mg of the phosphatidylserine is present in the composition; (c) a supplemental mineral, optionally present in the composition at about 0.5% to about 11%, and optionally about 5 mg to about 90 mg is present in the composition; (d) a compound derived from a magnolia tree, optionally present in the composition at about 0.5% to about 11% (w/w), and optionally about 10 mg to about 90 mg of the compound derived from a magnolia tree is present in the composition; (e) a ginger, optionally present in the composition at about 0.1% (w/w) to about 2.5% (w/w), and optionally about 1 mg to about 25 mg of the ginger is present in the composition; (f) a horny goat weed, optionally present in the composition at about 0.1% (w/w) to about 2.5% (w/w), and optionally about 1 mg to about 25 mg of the horny goat weed is present in the composition; (g) a sclareolides, optionally present in the composition at about 0.1% (w/w) to about 1% (w/w), and optionally about 0.5 mg to about 10 mg of the sclareolides is present in the composition; (h) a diindolylmethane (DIM), optionally present in the composition at about 0.1% (w/w) to about 1% (w/w), and optionally about 0.5 mg to about 10 mg of the diindolylmethane (DIM) is present in the composition; (i) a zinc, optionally present in the composition at about 0.1% (w/w) to about 1% (w/w), and optionally about 0.5 mg to about 20 mg of the zinc is present in the composition; (j) a an extraction from the aerial parts of oat plants, optionally present in the composition at about 0.1% (w/w) to about 2.5% (w/w), and optionally about 1 mg to about 25 mg of the an extraction from the aerial parts of oat plants is present in the composition; (k) a maca, optionally present in the composition at about 5% (w/w) to about 35% (w/w), and optionally about 100 mg to about 400 mg of the maca is present in the composition; (l) a Eurycoma longifolia, optionally present in the composition at about 5% (w/w) to about 25% (w/w), and optionally about 25 mg to about 275 mg of the Eurycoma longifolia is present in the composition; (m) a fenugreek seed extract, optionally present in the composition at about 5% (w/w) to about 25% (w/w), and optionally about 25 mg to about 275 mg of the fenugreek seed extract is present in the composition; (n) a Tribulus terrestris, optionally present in the composition at about 0.1% (w/w) to about 2.5% (w/w), and optionally about 1 mg to about 25 mg of the Tribulus terrestris is present in the composition; (o) a tea extract, optionally present in the composition at about 0.1% (w/w) to about 1% (w/w) or about 0.8% to about 1.6%, and optionally about 0.5 mg to about 10 mg or about 4.5 mg to about 15 mg of the tea extract is present in the composition; (p) a vitamin, optionally present in the composition at about 0.1% (w/w) to about 1% (w/w) or about 0.8% (w/w) to about 1.6% (w/w), and optionally about 0.5 mg to about 10 mg or about 4.5 mg to about 15 mg of the vitamin is present in the composition; (q) a nitric oxide source, optionally present in the composition at about 0.1% (w/w) to about 1% (w/w) or about 0.8% to about 1.6% (w/w), and optionally about 0.5 mg to about 10 mg or about 4.5 mg to about 15 mg of the nitric oxide source is present in the composition; (r) an amino acid, optionally present in the composition at about 0.1% (w/w) to about 1% (w/w), and optionally about 0.5 mg to about 10 mg of the amino acid is present in the composition; (s) a Panax ginseng, optionally present in the composition at about 0.1% (w/w) to about 1% (w/w), and optionally about 0.5 mg to about 10 mg of the Panax ginseng is present in the composition; (t) a Bacopa monieri, optionally present in the composition at about 0.5% (w/w) to about 1.6% (w/w), and optionally about 4.5 mg to about 15 mg of the Bacopa monieri is present in the composition; (u) a L-theanine, optionally present in the composition at about 0.1% (w/w) to about 8% (w/w), and optionally about 4.5 mg to about 15 mg of the L-theanine is present in the composition; (v) a L-tryptophan, optionally present in the composition at about 0.1% (w/w) to about 7% (w/w), and optionally about 2.5 mg to about 12.5 mg of the L-tryptophan is present in the composition; (w) a reishi mushroom, optionally present in the composition at about 1% (w/w) to about 3% (w/w), and optionally about 0.5 mg to about 10 mg of the reishi mushroom is present in the composition; (x) a melatonin, optionally present in the composition at about 0.01% (w/w) to about 2% (w/w), and optionally about 0.01 mg to about 5 mg of the melatonin is present in the composition; or (y) a combination of two or more selected from (a)-(x).

33. The method of claim 30 or 31, wherein the composition is selected from the composition of any one of claims 1-28.
